(19) United States
(12) Patent Application Publication
(10) Pub. No.: US 2003/0153521 A1
(43) Pub. Date: Aug. 14, 2003

(54) Nucleic acid treatment of diseases or conditions related to levels of Ras

(76) Inventor: James McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/238,700

(22) Filed: Sep. 10, 2002

Related U.S. Application Data
(60) Provisional application No. 60/318,471, filed on Sep. 10, 2001.

(30) Foreign Application Priority Data
May 29, 2002 (WO) PCT/US02/16840

(51) Int. Cl. A61K 48/00; C12Q 1/68; C12N 5/08; A61K 9/127; C12N 15/88

(52) U.S. Cl. 514/44; 435/6; 435/455; 435/366; 435/458; 424/450

(57) ABSTRACT

The present invention relates to nucleic acid molecules, including enzymatic nucleic acid molecules, such as DNAzymes (e.g. DNA enzymes, catalytic DNA), that modulate the expression of Ras genes such as K-Ras, H-Ras, and/or N-Ras.

This patent application claims priority from U.S.S. No. 60/318,471, filed Sep. 10, 2001, entitled ‘Enzymatic Nucleic Acid Treatment of Diseases or Conditions Related to Levels of Ras,” and this application also claims priority to PCT application PCT/US02/16840 filed May 29, 2002. Each of these applications is hereby incorporated by reference herein in its entirety including the drawings and tables.

### FIELD OF THE INVENTION

The present invention relates to novel nucleic acid compounds and methods for the treatment or diagnosis of diseases or conditions related to Ras expression, such as K-Ras, H-Ras, and/or N-Ras expression.

### BACKGROUND OF THE INVENTION

Transformation is a cumulative process whereby normal control of cell growth and differentiation is interrupted, usually through the accumulation of mutations affecting the expression of genes that regulate cell growth and differentiation.

The platelet derived growth factor (PDGF) system has served as a prototype for identification of substrates of the receptor tyrosine kinases. Certain enzymes become activated by the PDGF receptor kinase, including phospholipase C and phosphatidylinositol 3′ kinase, Ras guanosine triphosphate (GTPase) activating protein (GAP) and src-like tyrosine kinases. GAP regulates the function of the Ras protein by stimulating the GTPase activity of the 21 kD Ras protein. Barbacid, 56 Ann. Rev. Biochem. 779, 1987. Microinjection of oncogenically activated Ras into NIH 3T3 cells has been shown to induce DNA synthesis. Mutations that cause oncogenic activation of Ras lead to accumulation of Ras bound to GTP, the active form of the molecule. These mutations block the ability of GAP to convert Ras to the inactive form. Mutations that impair the interactions of Ras with GAP also block the biological function of Ras.

While a number of Ras alleles exist (N-Ras, K-Ras, H-Ras) which have been implicated in carcinogenesis, the type most often associated with colon and pancreatic carcinomas is K-Ras. Nucleic acid molecules which are targeted to certain regions of the K-Ras allelic mRNAs may also prove inhibitory to the function of the other allelic mRNAs of the N-Ras and H-Ras genes.

Scanlon, International PCT Publication Nos. WO 91/18625, WO 91/18624, and WO 91/18913 describes a ribozyme effective to cleave oncogene RNA from the H-Ras gene. This ribozyme is said to inhibit H-ras expression in response to exogenous stimuli. Reddy WO92/00080 describes the use of ribozymes as therapeutic agents for leukemias, such as chronic myelogenous leukemia (CML) by targeting specific portions of the BCR-ABL gene transcript.

Thompson et al., International PCT publication No. WO 99/54459, describe nucleic acid molecules that modulate gene expression, including Ras gene expression.

Zhang et al., 2000, Gene Ther., 7, 2041; Takunaga et al., 2000, Br. J. Cancer., 83, 833; Zhang et al., 2000, Mol. Biotechnol., 15, 39; Irie et al., 2000, Mol. Urol. 4, 61; Kijima and Scanlon, 2000, Mol. Biotechnol., 14, 59; Funato et al., 2000, Cancer Gene Ther., 7, 495; Tsuchida et al., 2000, Cancer Gene Ther., 7, 373; Zhang et al., 2000, Methods Mol. Med., 35, 261; Irie et al., 1999, Antisense Nucleic Acid Drug Dev., 9, 341; Giannini et al., 1999, Nucleic Acids Res., 27, 2737; Fang et al., 1999, J. Med. Coll. PLA, 14, 25; Tong et al., 1998, Methods Mol. Med., 11, 209; Ohkawa and Kashani-Sabet, 1998, Methods Mol. Med., 11, 153; Scherr et al., 1999, Gene Ther., 6, 152; Tsuchida et al., 1998, Biochem. Biophys. Res. Commun., 252, 368; Scherr et al., 1998, Gene Ther., 5, 1227; Uhlmann et al., European Patent Application EP 808898; Scherr et al., 1997, J. Biol. Chem., 272, 14304; Chang et al., 1997, J. Cancer Res. Clin. Oncol., 123, 91; Ohta et al., 1996, Nucleic Acids Res., 24, 938; Ohta et al., 1994, Ann. N.Y. Acad. Sci., 716, 242; and Funato et al., 1994, Biochem. Pharmacol., 48, 1471 all describe specific ribozymes targeting certain K-Ras, H-Ras, or N-Ras RNA sequences.

Todd, International PCT Publication Nos. WO 01/49877, WO 99/50452, and WO 99/45146 describes specific DNAzymes targeting K-Ras for diagnostic applications.

### SUMMARY OF THE INVENTION

The present invention features nucleic acid molecules, including, for example, antisense oligonucleotides, siRNA, aptamers, decoys, and enzymatic nucleic acid molecules such as DNAzyme enzymatic nucleic acid molecules, which modulate expression of sequences encoding Ras oncogenes, such as K-Ras, H-Ras, and N-Ras. In one embodiment, the invention features an enzymatic nucleic acid molecule comprising a sequence of SEQ ID NOs: 1322-2642 or 3650-4655.

In another embodiment, the invention features an enzymatic nucleic acid molecule comprising at least one binding arm having a sequence complementary to a sequence of SEQ ID NOs: 1-1321 or 2643-3649.

In another aspect of the invention, the enzymatic nucleic acid of the invention is adapted to treat cancer.

In one embodiment, the enzymatic nucleic acid molecule of the invention has an endonuclease activity to cleave RNA having a K-Ras sequence.

In another embodiment, the enzymatic nucleic acid molecule of the invention has an endonuclease activity to cleave RNA having an H-Ras sequence. In another embodiment, the enzymatic nucleic acid molecule of the invention has an endonuclease activity to cleave RNA having an N-Ras sequence.

In one embodiment, the siRNA molecule of the invention has RNA interference activity to K-Ras expression.

In another embodiment, the siRNA molecule of the invention has RNA interference activity to H-Ras expression.

In another embodiment, the siRNA molecule of the invention has RNA interference activity to N-Ras expression.

In one embodiment, a siRNA molecule of the invention comprises a double stranded RNA wherein one strand of the RNA is complementary to the RNA of K-Ras, H-Ras, and/or N-Ras gene. In another embodiment, a siRNA molecule of the invention comprises a double stranded RNA wherein one strand of the RNA comprises a portion of a sequence of RNA of K-Ras, H-Ras, and/or N-Ras gene sequence. In yet another embodiment, a siRNA molecule of the invention comprises a double stranded RNA wherein both strands of RNA are connected by a non-nucleotide linker. Alternately, a siRNA molecule of the invention comprises a double stranded RNA wherein both strands of RNA are connected by a nucleotide linker, such as a loop or stem loop structure.

In one embodiment, a single strand component of a siRNA molecule of the invention is from about 14 to about 50 nucleotides in length. In another embodiment, a single strand component of a siRNA molecule of the invention is about 14, 15, 16, 17, 18, 19, 20, 21, 22, 23, 24, 25, 26, 27, or 28 nucleotides in length. In yet another embodiment, a single strand component of a siRNA molecule of the invention is about 23 nucleotides in length. In one embodiment, a siRNA molecule of the invention is from about 28 to about 56 nucleotides in length. In another embodiment, a siRNA molecule of the invention is about 40, 41, 42, 43, 44, 45, 46, 47, 48, 49, 50, 51, or 52 nucleotides in length. In yet another embodiment, a siRNA molecule of the invention is about 46 nucleotides in length.

In one embodiment, the invention features a siRNA nucleic acid molecule comprising a sequence complementary to a sequence of SEQ ID NOs: 1-1321 or 2643-3649. In another embodiment, the invention features a siRNA nucleic acid molecule having antisense region complementary to a sequence of SEQ ID NOs: 1-1321 or 2643-3649 and a sense region complementary to the antisense region.

In one embodiment, the DNAzyme molecule of the invention is in a “10-23” configuration (see for example Santoro et al., 1997, PNAS, 94, 4262 and Joyce et al., U.S. Pat. No. 5,807,718). In another embodiment, the DNAzyme comprises a sequence complementary to a sequence of SEQ ID NOs: 1-1321 or 2643-3649. In yet another embodiment, the DNAzyme comprises a sequence of SEQ ID NOs: 1322-2642 or 3650-4655.

In another embodiment, the nucleic acid molecule of the invention comprises between 12 and 100 bases complementary to a RNA having a K-Ras sequence. In yet another embodiment, the nucleic acid comprises between 14 and 24 bases complementary to a RNA having a K-Ras sequence.

In another embodiment, the nucleic acid molecule of the invention comprises between 12 and 100 bases complementary to a RNA having an H-Ras sequence. In yet another embodiment, the nucleic acid molecule of the invention comprises between 14 and 24 bases complementary to an RNA having an H-Ras sequence.

In another embodiment, the nucleic acid molecule of the invention comprises between 12 and 100 bases complementary to an RNA having an N-Ras sequence. In yet another embodiment, the nucleic acid molecule of the invention comprises between 14 and 24 bases complementary to an RNA having an N-Ras sequence.

In yet another embodiment, the nucleic acid molecule of the invention is chemically synthesized. The nucleic acid molecule can comprise at least one 2′-sugar modification, at least one nucleic acid base modification, and/or at least one phosphate backbone modification.

In one embodiment, the invention features a mammalian cell including the nucleic acid molecule of the invention. In another embodiment, the mammalian cell of the invention is a human cell.

In another embodiment, the invention features a method of modulating K-Ras activity in a cell, comprising contacting the cell with the nucleic acid molecule of the invention, under conditions suitable for the modulation of K-Ras activity.

In another embodiment, the invention features a method of modulating H-Ras activity in a cell, comprising contacting the cell with the nucleic acid molecule of the invention, under conditions suitable for the modulation of H-Ras activity.

In another embodiment, the invention features a method of modulating N-Ras activity in a cell, comprising contacting the cell with the nucleic acid molecule of the invention, under conditions suitable for the modulation of N-Ras activity.

In another embodiment, the invention features a method of treatment of a subject having a condition associated with the level of K-Ras, comprising contacting cells of the subject with the nucleic acid molecule of the invention, under conditions suitable for the treatment.

In another embodiment, the invention features a method of treatment of a subject having a condition associated with the level of H-Ras, comprising contacting cells of the subject with the nucleic acid molecule of the invention, under conditions suitable for the treatment.

In another embodiment, the invention features a method of treatment of a subject having a condition associated with the level of N-Ras, comprising contacting cells of the subject with the nucleic acid molecule of the invention, under conditions suitable for the treatment.

In one embodiment, a method of treatment of the invention further comprises the use of one or more drug therapies under conditions suitable for the treatment.

In another embodiment, the invention features a method of cleaving RNA having a K-Ras sequence comprising contacting the K-Ras RNA with the enzymatic nucleic acid molecule of the invention under conditions suitable for the cleavage, for example, where the cleavage is carried out in the presence of a divalent cation, such as Mg²⁺.

In another embodiment, the invention features a method of cleaving RNA having an H-Ras sequence comprising contacting the H-Ras RNA with the enzymatic nucleic acid molecule of the invention under conditions suitable for the cleavage, for example, where the cleavage is carried out in the presence of a divalent cation, such as Mg²⁺.

In another embodiment, the invention features a method of cleaving RNA having an N-Ras sequence comprising contacting the N-Ras RNA with the enzymatic nucleic acid molecule of the invention under conditions suitable for the cleavage, for example, where the cleavage is carried out in the presence of a divalent cation, such as Mg²⁺.

In one embodiment, the nucleic acid molecule of the invention comprises a cap structure, for example, a 3′,3′-linked or 5′,5′-linked deoxyabasic ribose derivative, wherein the cap structure is at the 5′-end, or 3′-end, or both the 5′-end and the 3′-end of the nucleic acid molecule.

In another embodiment, the invention features an expression vector comprising a nucleic acid sequence encoding at least one nucleic acid molecule of the invention in a manner that allows expression of the nucleic acid molecule. For example, the invention features an expression vector comprising a nucleic acid encoding a DNAzyme in a manner that allows expression of the DNAzyme.

In yet another embodiment, the invention features a mammalian cell, for example a human cell, including an expression vector of the invention.

In another embodiment, the expression vector of the invention further comprises a sequence for an nucleic acid molecule complementary to an RNA having K-Ras sequence.

In another embodiment, the expression vector of the invention further comprises a sequence for an nucleic acid molecule complementary to an RNA having H-Ras sequence.

In another embodiment, the expression vector of the invention further comprises a sequence for an nucleic acid molecule complementary to an RNA having N-Ras sequence.

In one embodiment, an expression vector of the invention comprises a nucleic acid sequence encoding two or more nucleic acid molecules of the invention, which can be the same or different. In another embodiment, an expression vector of the invention further comprises a sequence encoding an antisense nucleic acid molecule complementary to an RNA having a K-Ras, H-Ras or N-Ras sequence.

In another embodiment, the invention features a method for treating cancer, for example colorectal cancer, bladder cancer, lung cancer, pancreatic cancer, breast cancer, or prostate cancer, comprising administering to a patient a nucleic acid molecule of the invention under conditions suitable for the treatment. A method of treatment of cancer of the invention can further comprise administering to a patient one or more other therapies, for example, monoclonal antibody therapy, such as Herceptin (trastuzumab); chemotherapy, such as paclitaxel (Taxol), docetaxel, cisplatin, methotrexate, cyclophosphamide, doxorubin, fluorouracil carboplatin, Leucovorin, Irinotecan (CAMPTOSAR® or CPT-11 or Camptothecin-11 or Campto), Carboplatin, edatrexate, gemcitabine, or vinorelbine; radiation therapy, or analgesic therapy and/or any combination thereof.

In another embodiment, the invention features a pharmaceutical composition comprising a nucleic acid molecule of the invention in a pharmaceutically acceptable carrier.

In one embodiment, the invention features a method of administering to a cell, for example a mammalian cell or human cell, the nucleic acid molecule of the invention comprising contacting the cell with the nucleic acid molecule under conditions suitable for administration. The method of administration can be in the presence of a delivery reagent, for example a lipid, cationic lipid, phospholipid, or liposome.

### DETAILED DESCRIPTION OF THE INVENTION

First the drawings will be described briefly.

### Drawings

FIG. 1 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig et al., International PCT Publication No. WO 98/58058 and U.S. patent application Ser. No. 08/878,640); G-Cleaver, represents G-cleaver ribozyme motif (Kore et al., 1998, Nucleic Acids Research 26, 4116-4120, Eckstein et al., U.S. Pat. No. 6,127,173). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

FIG. 2 shows an example of an Amberzyme enzymatic nucleic acid molecule motif that is chemically stabilized (see, for example, Beigelman et al., International PCT publication No.

WO 99/55857 and U.S. patent application Ser. No. 09/476,387).

FIG. 3 shows an example of an Zinzyme A enzymatic nucleic acid molecule motif that is chemically stabilized (see for example Beigelman et al., Beigelman et al., International PCT publication No. WO 99/55857 and U.S. patent application Ser. No. 09/918,728).

FIG. 4 shows an example of a DNAzyme motif (e.g., “10-23”) described by Santoro et al., 1997, PNAS, 94, 4262 and Joyce et al., U.S. Pat. No. 5,807,718.

FIG. 5 shows non-limiting examples of different siRNA constructs of the invention. The examples shown (constructs 1, 2, and 3) have 19 representative base pairs, however, different embodiments of the invention include any number of base pairs described herein. Bracketed regions represent nucleotide overhangs, for example comprising between about 1, 2, 3, or 4 nucleotides in length, preferably about 2 nucleotides. Constructs 1 and 2 can be used independently for RNAi activity. Construct 2 can comprise a polynucleotide or non-nucleotide linker, which can optionally be designed as a biodegradable linker. In one embodiment, the loop structure shown in construct 2 can comprise a biodegradable linker that results in the formation of construct 1 in vivo and/or in vitro. In another example, construct 3 can be used to generate construct 2 under the same principle wherein a linker is used to generate the active siRNA construct 2 in vivo and/or in vitro, which can optionally utilize another biodegradable linker to generate the active siRNA construct 1 in vivo and/or in vitro. As such, the stability and/or activity of the siRNA constructs can be modulated based on the design of the siRNA construct for use in vivo or in vitro and/or in vitro.

The invention features novel nucleic acid molecules, including antisense oligonucleotides, siRNA, and enzymatic nucleic acid molecules, and methods to modulate gene expression, for example, genes encoding K-Ras, H-Ras and/or N-Ras. In particular, the instant invention features nucleic-acid based molecules and methods to down-regulate the expression of K-Ras, H-Ras and/or N-Ras gene sequences.

The invention features one or more nucleic acid-based molecules and methods that independently or in combination modulate the expression of a gene or genes encoding Ras proteins. In particular embodiments, the invention features nucleic acid-based molecules and methods that modulate the expression of K-Ras gene, for example, Genbank Accession No. NM_{—}004985; H-Ras gene, for example, Genbank Accession No. NM_{—}005343; and/or N-Ras gene, for example, Genbank Accession No. NM_{—}002524.

The description below of the various aspects and embodiments is provided with reference to exemplary K-Ras, H-Ras, and N-Ras genes, referred to hereinafter collectively as Ras. However, the various aspects and embodiments are directed to equivalent sequences and also to other genes which encode K-Ras, H-Ras and/or N-Ras proteins and similar proteins to K-Ras, H-Ras and/or N-Ras. For example, the invention relates to genes with homology to genes that encode K-Ras, H-Ras and/or N-Ras and genes that encode proteins with similar function to K-Ras, H-Ras, and N-Ras proteins. Those additional genes can be analyzed for target sites using the methods described herein. Thus, the modulation and the effects of such modulation of the other genes can be determined as described herein.

In one embodiment, the invention features the use of an enzymatic nucleic acid molecule, including those in the hammerhead, NCH, G-cleaver, amberzyme, zinzyme and/or DNAzyme motif, to modulate the expression of a Ras gene or inhibit Ras activity. In one embodiment, the invention features the use of these enzymatic nucleic acid molecules to down-regulate the expression of a Ras gene or inhibit Ras activity. In another embodiment, the invention features the use of an antisense oligonucleotide molecule to modulate, for example, down-regulate, the expression of a Ras gene or inhibit Ras activity.

By “modulate” is meant that the expression of the gene, or level of RNAs or equivalent RNAs encoding one or more protein subunits or components, or activity of one or more proteins is up-regulated or down-regulated, such that the expression, level, or activity is greater than or less than that observed in the absence of the nucleic acid molecules of the invention.

By “inhibit” or “down-regulate” it is meant that the expression of the gene, or level of RNAs or equivalent RNAs encoding one or more protein subunits or components, or activity of one or more protein subunits or components, such as Ras protein or proteins, is reduced below that observed in the absence of the nucleic acid molecules of the invention. In one embodiment, inhibition or down-regulation with the enzymatic nucleic acid molecule preferably is below that level observed in the presence of an enzymatically inactive or attenuated enzymatic nucleic acid molecule that is able to bind to the same site on the target RNA, but is unable to cleave that RNA. In another embodiment, inhibition or down-regulation with an antisense oligonucleotide is preferably below that level observed in the presence of, for example, an oligonucleotide with scrambled sequence or with mismatches. In another embodiment, inhibition or down-regulation of Ras with the nucleic acid molecule of the instant invention is greater in the presence of the nucleic acid molecule than in its absence.

By “up-regulate” is meant that the expression of the gene, or level of RNAs or equivalent RNAs encoding one or more protein subunits or components, or activity of one or more protein subunits or components, such as Ras protein or proteins, is greater than that observed in the absence of the nucleic acid molecules of the invention. For example, the expression of a gene, such as Ras gene, can be increased in order to treat, prevent, ameliorate, or modulate a pathological condition caused or exacerbated by an absence or low level of gene expression.

By “enzymatic nucleic acid molecule” as used herein, is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term DNAzyme-based enzymatic nucleic acid is used interchangeably with phrases such as catalytic DNA, aptazyme or aptamer-binding DNAzyme, regulatable DNAzyme, catalytic oligonucleotides, nucleozyme, DNAzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving and/or ligation activity to the molecule.

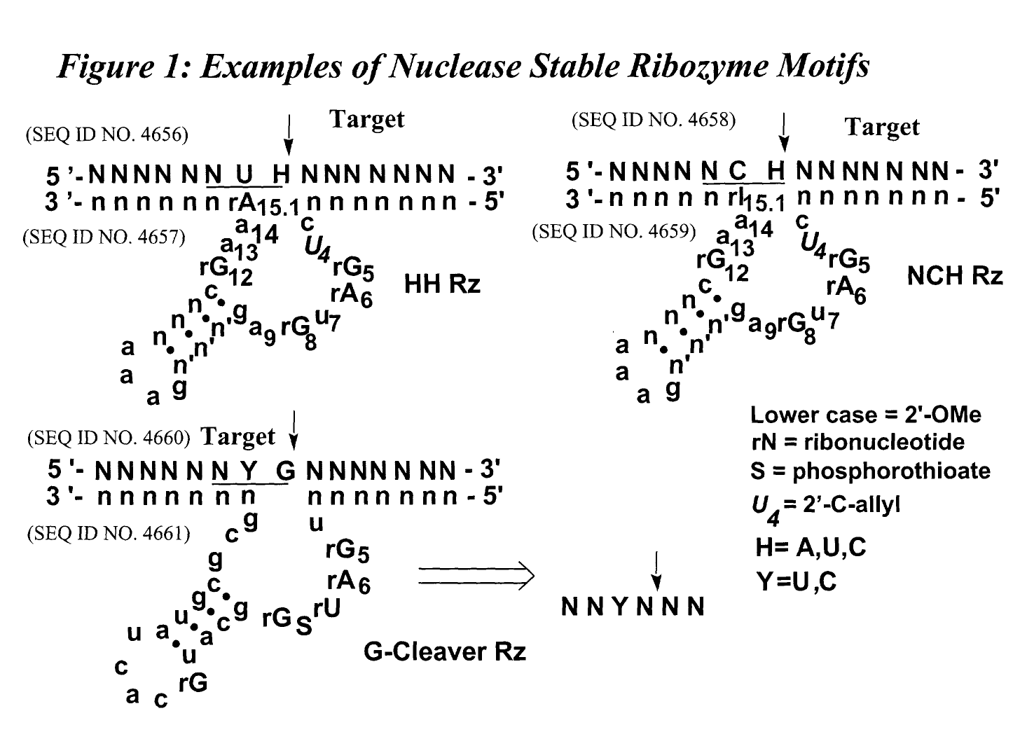

By “nucleic acid molecule” as used herein is meant a molecule having nucleotides. The nucleic acid can be single, double, or multiple stranded and can comprise modified or unmodified nucleotides or non-nucleotides or various mixtures and combinations thereof By “enzymatic portion” or “catalytic domain” is meant that portion/region of the enzymatic nucleic acid molecule essential for cleavage of a nucleic acid substrate (for example see FIGS. 1-4).

By “substrate binding arm” or “substrate binding domain” is meant that portion/region of a enzymatic nucleic acid which is able to interact, for example via complementarity (i.e., able to base-pair with), with a portion of its substrate. Such complementarity can be 100%, but less than 100% complementarity is also encompassed within the scope of the invention. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-3. That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and can be of varying lengths. The length of the binding arm(s) can be greater than or equal to four nucleotides and of sufficient length to stably interact with the target RNA; including a length of 12-100 nucleotides; and further including a length of 14-24 nucleotides long (see for example Werner and Uhlenbeck, supra; Hamman et al., supra; Hampel et al., EP0360257; Berzal-Herranz et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” or “NCH” motif or configuration is meant, an enzymatic nucleic acid molecule comprising a motif as is generally described as NCH Rz in FIG. 1 and in Ludwig et al., International PCT Publication No. WO 98/58058 and U.S. patent application Ser. No. 08/878,640. Inozymes possess endonuclease activity to cleave nucleic acid substrates having a cleavage triplet NCH/, where N is a nucleotide, C is cytidine and H is adenosine, uridine or cytidine, and “/” represents the cleavage site. H is used interchangeably with X. Inozymes can also possess endonuclease activity to cleave nucleic acid substrates having a cleavage triplet NCN/, where N is a nucleotide, C is cytidine, and “/” represents the cleavage site. “I” in FIG. 1 represents an Inosine nucleotide, preferably a ribo-Inosine or xylo-Inosine nucleoside.

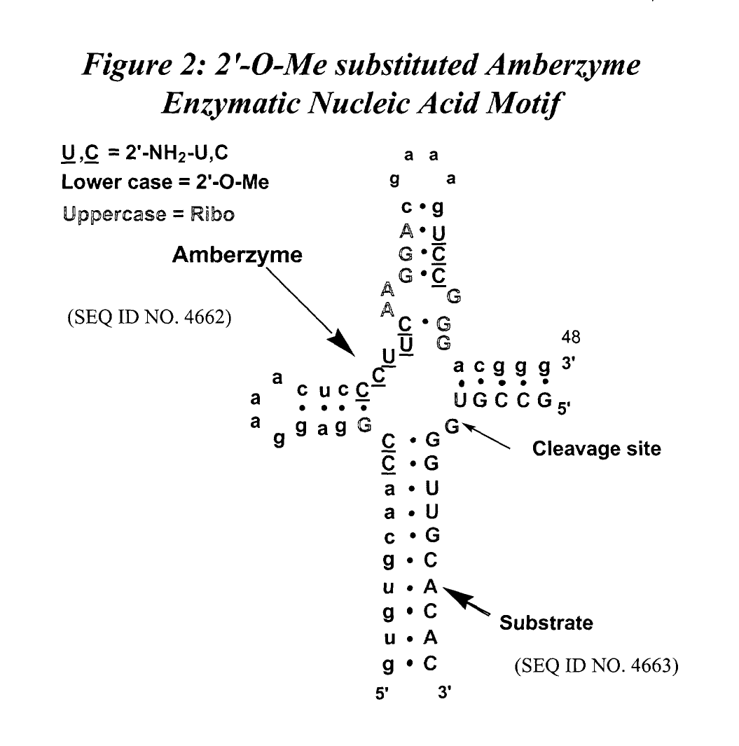

By “G-cleaver” motif or configuration is meant, an enzymatic nucleic acid molecule comprising a motif as is generally described as G-cleaver Rz in FIG. 1 and in Eckstein et al., U.S. Pat. No. 6,127,173. G-cleavers possess endonuclease activity to cleave nucleic acid substrates having a cleavage triplet NYN/, where N is a nucleotide, Y is uridine or cytidine and “/” represents the cleavage site. G-cleavers can be chemically modified as is generally shown in FIG. 1. By “amberzyme” motif or configuration is meant, an enzymatic nucleic acid molecule comprising a motif as is generally described in FIG. 2 and in Beigelman et al., International PCT publication No. WO 99/55857 and U.S. patent application Ser. No. 09/476,387. Amberzymes possess endonuclease activity to cleave nucleic acid substrates having a cleavage triplet NG/N, where N is a nucleotide, G is guanosine, and “/” represents the cleavage site. Amberzymes can be chemically modified to increase nuclease stability through substitutions as are generally shown in FIG. 2. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

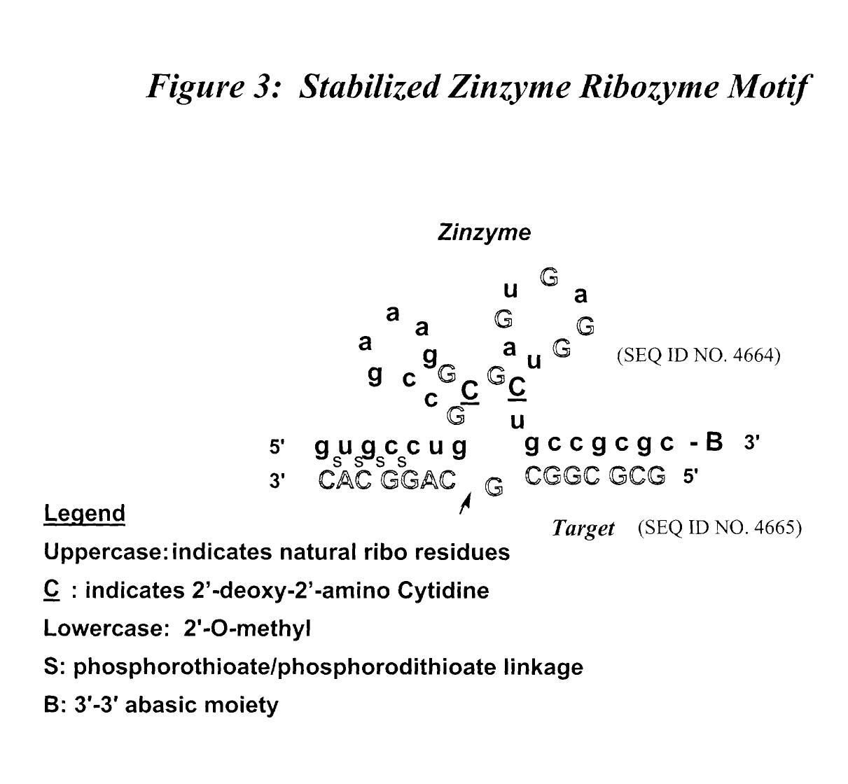

By “zinzyme” motif or configuration is meant, an enzymatic nucleic acid molecule comprising a motif as is generally described in FIG. 3 and in Beigelman et al., International PCT publication No. WO 99/55857 and U.S. patent application Ser. No. 09/918,728. Zinzymes possess endonuclease activity to cleave nucleic acid substrates having a cleavage triplet including but not limited to YG/Y, where Y is uridine or cytidine, and G is guanosine and “/” represents the cleavage site. Zinzymes can be chemically modified to increase nuclease stability through substitutions as are generally shown in FIG. 3, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

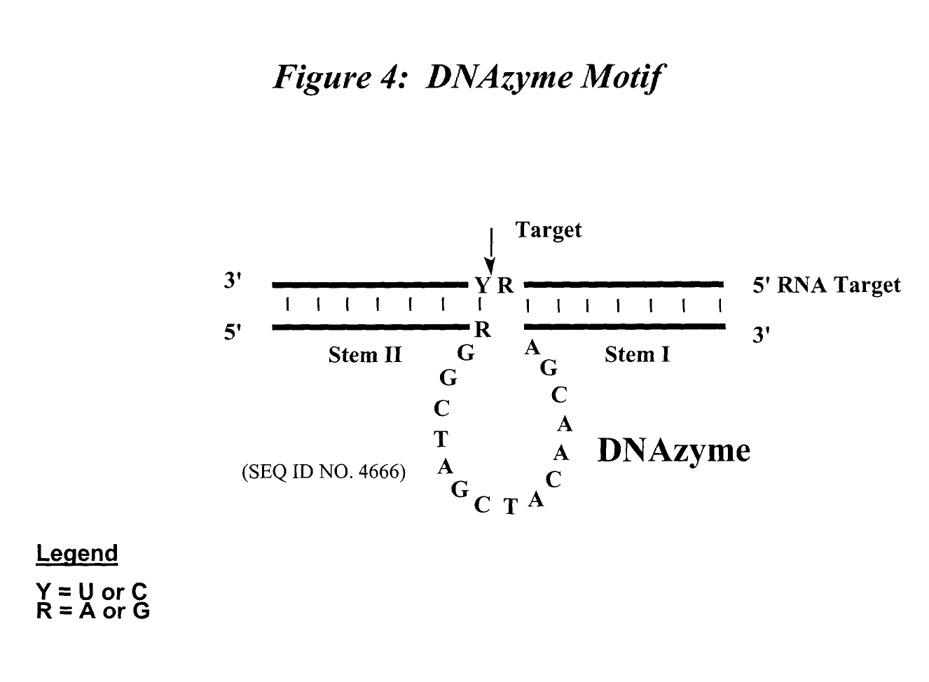

By ‘DNAzyme’ is meant, an enzymatic nucleic acid molecule that does not require the presence of a 2′-OH group within its own nucleic acid sequence for activity. In particular embodiments the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 4 and is generally reviewed in Usman et al., US patent No., 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection see Santoro et al., supra and as generally described in Joyce et al., U.S. Pat. No. 5,807,718. Additional DNAzyme motifs can be selected for using techniques similar to those described in these references, and hence, are within the scope of the present invention. DNAzymes of the invention can comprise nucleotides modified at the nucleic acid base, sugar, or phosphate backbone. Non-limiting examples of sugar modifications that can be used in DNAzymes of the invention include 2′-O-alkyl modifications such as 2′-O-methyl or 2′-O-allyl, 2′-C-alkyl modifications such as 2′-C-allyl, 2′-deoxy-2′-amino, 2′-halo modifications such as 2′-fluoro, 2′-chloro, or 2′-bromo, isomeric modifications such as arabinofuranose or xylofuranose based nucleic acids, and other sugar modifications such as 4′-thio or 4′-carbocyclic nucleic acids. Non-limiting examples of nucleic acid based modifications that can be used in DNAzymes of the invention include modified purine heterocycles, G-clamp heterocycles, and various modified pyrimidine cycles. Non-limiting examples of backbone modifications that can be used in DNAzymes of the invention include phosphorothioate, phosphorodithioate, phosphoramidate, and methylphosphonate internucleotide linkages. DNAzymes of the invention can comprise naturally occurring nucleic acids, chimeras of chemically modified and naturally occurring nucleic acids, or completely modified nucleic acids.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. In certain embodiments, the binding arms are not so long as to prevent useful turnover of the nucleic acid molecule.

By “stably interact” is meant interaction of oligonucleotides with target nucleic acid molecules (e.g., by forming hydrogen bonds with complementary nucleotides in the target under physiological conditions) that is sufficient to the intended purpose (e.g., cleavage of target RNA by an enzyme).

By “equivalent” RNA to Ras is meant to include those naturally occurring RNA molecules having homology (partial or complete) to Ras proteins or encoding for proteins with similar function as Ras proteins in various organisms, including humans, rodents, primates, rabbits, pigs, protozoans, fungi, plants, and other microorganisms and parasites. The equivalent RNA sequence can also include, in addition to the coding region, regions such as a 5′-untranslated region, a 3′-untranslated region, introns, a intron-exon junction and the like.

By “homology” is meant the nucleotide sequence of two or more nucleic acid molecules is partially or completely identical.

By “component” of Ras is meant a peptide or protein subunit expressed from a Ras gene.

By “gene” it is meant a nucleic acid that encodes an RNA, for example, nucleic acid sequences including but not limited to structural genes encoding a polypeptide.

“Complementarity” refers to the ability of a nucleic acid to form hydrogen bond or bonds with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2“-OH” is meant a nucleotide with a hydroxyl group at the 2′ position of a β-D-ribo-furanose moiety.

By “decoy “is meant a nucleic acid molecule, for example RNA or DNA, or aptamer that is designed to preferentially bind to a predetermined ligand. Such binding can result in the inhibition or activation of a target molecule. A decoy or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy can be designed to bind to Ras and block the binding of Ras or a decoy can be designed to bind to Ras and prevent interaction with the Ras protein.

By “aptamer” or “nucleic acid aptamer” as used herein is meant a nucleic acid molecule that binds specifically to a target molecule wherein the nucleic acid molecule has sequence that is distinct from sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand binding domain of a protein, thereby preventing interaction of the naturally occurring ligand with the protein. Similarly, the nucleic acid molecules of the instant invention can bind to RAS-encoded RNA or proteins receptors to block activity of the activity of target protein or nucleic acid. This is a non-limiting example and those in the art will recognize that other embodiments can be readily generated using techniques generally known in the art, see for example Gold et al., U.S. Pat. Nos. 5,475,096 and 5,270,163; Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

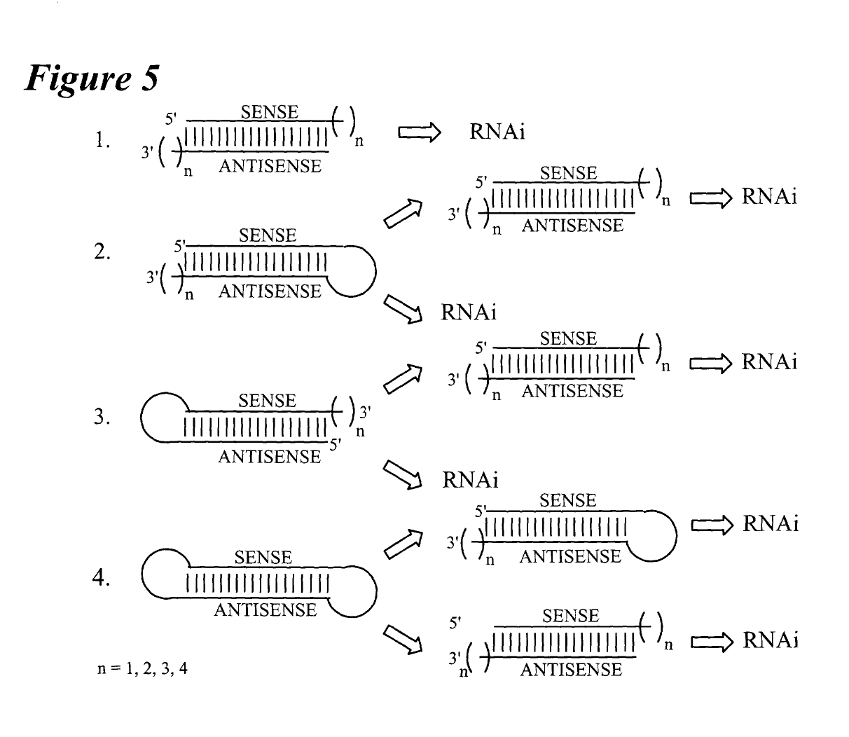

The term “short interfering RNA” or “siRNA”, or “short interfering nucleic acid molecule” or “short interfering nucleic acid”, or “siNA” or “short interfering oligonucleotide molecule” or “chemically modified short interfering nucleic acid molecule” as used herein refers to any nucleic acid molecule capable of mediating RNA interference “RNAi” or gene silencing; see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. Non limiting examples of siRNA molecules of the invention are shown in FIG. 5. For example the siRNA can be a double stranded polynucleotide molecule comprising self complementary sense and antisense regions, wherein the antisense region comprises complementarity to a target nucleic acid molecule. The siRNA can be a single stranded hairpin polynucleotide having self complementary sense and antisense regions, wherein the antisense region comprises complementarity to a target nucleic acid molecule. The siRNA can be a circular single stranded polynucleotide having two or more loop structures and a stem comprising self complementary sense and antisense regions, wherein the antisense region comprises complementarity to a target nucleic acid molecule, and wherein the circular polynucleotide can be processed either in vivo or in vitro to generate an active siRNA capable of mediating RNAi. As used herein, siRNA molecules need not be limited to those molecules containing only RNA, but further encompasses chemically modified nucleotides and non-nucleotides. In certain embodiments, the short interfering nucleic acid molecules of the invention lack 2′-hydroxy (2′-OH) containing nucleotides. In certain embodiments, the invention features short interfering nucleic acids that do not require the presence of nucleotides having a 2′-hydroxy group for mediating RNAi and as such, short interfering nucleic acid molecules of the invention optionally do not contain any ribonucleotides (e.g., nucleotides having a 2′-OH group). The modified short interfering nucleic acid molecules of the invention can also be referred to as short interfering modified oligonucleotides “siMON”. As used herein, the term siRNA is meant to be equivalent to other terms used to describe nucleic acid molecules that are capable of mediating sequence specific RNAi, for example double stranded RNA (dsRNA), micro-RNA, short hairpin RNA (shRNA), short interfering oligonucleotide, short interfering nucleic acid (siNA), short interfering modified oligonucleotide, chemically modified siRNA, post transcriptional gene silencing RNA (ptgsRNA), and others. In addition, as used herein, the term RNAi is meant to be equivalent to other terms used to describe sequence specific RNA interference, such as post transciptional gene silencing.

In general, enzymatic nucleic acids act by first binding to a target RNA. Such binding occurs through the target binding portion of a enzymatic nucleic acid that is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single enzymatic nucleic acid molecule is able to cleave many molecules of target RNA. In addition, the enzymatic nucleic acid molecule is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of an enzymatic nucleic acid molecule.

Nucleic acid molecules that modulate expression of Ras-specific RNAs represent a therapeutic approach to treat cancer, including, but not limited to, colorectal cancer, bladder cancer, lung cancer, pancreatic cancer, breast cancer, or prostate cancer and any other cancer, disease or condition that responds to the modulation of Ras expression.

In one embodiment of the inventions described herein, an enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, US. Patent No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as Aptazyme (Breaker et al., WO 98/43993), Amberzyme (Class I motif; FIG. 2; Beigelman et al., U.S. Ser. No. 09/301,511) or Zinzyme (FIG. 3) (Beigelman et al., U.S. Ser. No. 09/301,511), all included by reference herein including drawings, can also be used in the present invention. These specific motifs or configurations are not limiting in the invention and those skilled in the art will recognize that all that is important in an enzymatic nucleic acid molecule of this invention is that it has a specific substrate binding site that is complementary to one or more target gene RNA regions, and that it have nucleotide sequences within or surrounding a substrate binding site that impart an RNA cleaving activity to the molecule (Cech et al., U.S. Pat. No. 4,987,071).

In one embodiment of the present invention, a nucleic acid molecule of the instant invention can be between about 10 and 100 nucleotides in length. Exemplary enzymatic nucleic acid molecules of the invention are shown in Tables II and III. For example, enzymatic nucleic acid molecules of the invention are between about 15 and 50 nucleotides in length, including a length between about 25 and 40 nucleotides in length, e.g., 34, 36, or 38 nucleotides in length (for example see Jarvis et al., 1996, J. Biol. Chem., 271, 29107-29112). Exemplary DNAzymes of the invention are between about 15 and 40 nucleotides in length, including a length between about 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are between about 15 and 75 nucleotides in length, including a lengthbetween about 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are between about 10 and 40 nucleotides in length, including a lengthbetween about 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for a nucleic acid molecule to be of length and conformation sufficient and suitable for the nucleic acid molecule to interact with its target and/or catalyze a reaction contemplated herein. The length of nucleic acid molecules of the instant invention are not limiting within the general limits stated.

In certain embodiments, a nucleic acid molecule that modulates, for example down-regulates, Ras expression and/or activity, comprises between 12 and 100 bases complementary to a RNA molecule of Ras. In other embodiments, a nucleic acid molecule that modulates Ras expression comprises between 14 and 24 bases complementary to a RNA molecule of Ras.

The invention provides a method for producing a class of nucleic acid-based gene modulating agents that exhibit a high degree of specificity for RNA of a desired target. For example, an enzymatic nucleic acid molecule can be targeted to a highly conserved sequence region of target RNAs encoding Ras (and specifically a Ras gene) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., enzymatic nucleic acid molecules, siRNA, antisense, and/or DNAzymes) can be expressed from DNA and/or RNA vectors that are delivered to specific cells.

As used in herein “cell” is used in its usual biological sense, and does not refer to an entire multicellular organism. A cell can, for example, be in vitro, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

By “Ras proteins” is meant, a peptide or protein comprising Ras tyrosine kinase-type cell surface receptor or a peptide or protein encoded by a Ras gene, such as K-Ras, H-Ras, or N-Ras.

By “highly conserved sequence region” is meant, a nucleotide sequence of one or more regions in a target gene that does not vary significantly from one generation to the other or from one biological system to the other.

Nucleic acid-based modulators, including inhibitors, of Ras expression are useful for the prevention and/or treatment of cancer, including but not limited to breast cancer and ovarian cancer and any other disease or condition that respond to the modulation of Ras expression.

By “related” is meant that the reduction of RAS, HIV, or HER2 expression (specifically RAS, HIV, or HER2 genes respectively) RNA levels and thus reduction in the level of the respective protein relieves, to some extent, the symptoms of the disease or condition.

The nucleic acid-based molecules of the invention can be added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers. In certain embodiments, the enzymatic nucleic acid molecules comprise sequences that are complementary to the substrate sequences in Tables II and III. Examples of such enzymatic nucleic acid molecules also are shown in Tables II and III. Examples of such enzymatic nucleic acid molecules consist essentially of sequences defined in these tables.

In another embodiment, the invention features siRNA, antisense nucleic acid molecules and 2-5A chimera comprising sequences complementary to the substrate sequences shown in Tables II and III. Such nucleic acid molecules can comprise sequences as shown for the binding arms of the enzymatic nucleic acid molecules in Tables II and III. Similarly, triplex molecules can be targeted to corresponding DNA target regions; such molecules can comprise the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to a substrate such that the substrate molecule forms a loop, and/or an antisense molecule can bind such that the antisense molecule forms a loop. Thus, the antisense molecule can be complementary to two or more non-contiguous substrate sequences. In addition, two or more non-contiguous sequence portions of an antisense molecule can be complementary to a target sequence.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present that do not interfere with such cleavage. Thus, a core region of an enzymatic nucleic acid molecule can, for example, include one or more loop, stem-loop structure, or linker that does not prevent enzymatic activity. Thus, various regions in the sequences in Tables II and III can be such a loop, stem-loop, nucleotide linker, and/or non-nucleotide linker and can be represented generally as sequence “X”. The nucleic acid molecules of the instant invention, such as Hammerhead, Inozyme, G-cleaver, amberzyme, zinzyme, DNAzyme, antisense, 2-5A antisense, triplex forming nucleic acid, and decoy nucleic acids, can contain other sequences or non-nucleotide linkers that do not interfere with the function of the nucleic acid molecule.

Sequence X can be a linker of ≧2 nucleotides in length, preferably 3, 4, 5, 6, 7, 8, 9, 10, 15, 20, 26, 30, where the nucleotides can be internally base-paired to form a stem of ≧2 base pairs. Alternatively or in addition, sequence X can be a non-nucleotide linker. In yet another embodiment, the nucleotide linker X can be a nucleic acid aptamer, such as an ATP aptamer, Ras Rev aptamer (RRE), Ras Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, a non-nucleotide linker X is as defined herein. Non-nucleotides as can include abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No.

WO 95/11910 and Ferentz and Verdine, J. Am. Chem. Soc. 1991, 113:4000, all hereby incorporated by reference herein. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in certain embodiments, the invention features an enzymatic nucleic acid molecule having one or more non-nucleotide moieties, and having enzymatic activity to cleave an RNA or DNA molecule.

In another aspect of the invention, enzymatic nucleic acid molecules, siRNA molecules, or antisense molecules that interact with target RNA molecules and modulate Ras (and specifically a Ras gene) activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, adeno-associated virus, retrovirus, adenovirus, or alphavirus as well as others known in the art.Recombinant vectors capable of expressing enzymatic nucleic acid molecules or antisense can be delivered as described below, and persist in target cells. Alternatively, viral vectors can be used that provide for transient expression of enzymatic nucleic acid molecules or antisense. Such vectors can be repeatedly administered as necessary. Once expressed, the enzymatic nucleic acid molecules or antisense bind to target RNA and modulate its function or expression. Delivery of enzymatic nucleic acid molecule or antisense expressing vectors can be systemic, such as by intravenous or intramuscular administration, by administration to target cells ex-planted from the patient followed by reintroduction into the patient, or by any other means that allows for introduction into a desired target cell. Antisense DNA and DNAzymes can be expressed via the use of a single stranded DNA intracellular expression vector.

By “vectors” is meant any nucleic acid- and/or viral-based technique used to deliver a desired nucleic acid.

By “patient” is meant an organism that is a donor or recipient of explanted cells or the cells of the organism. “Patient” also refers to an organism to which the nucleic acid molecules of the invention can be administered. A patient can be a mammal or mammalian cells. A patient can be a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme, for example, with a nucleic acid molecule comprising chemical modifications. In some cases, the activity or stability of the nucleic acid molecule can be decreased (i.e., less than ten-fold), but the overall activity of the nucleic acid molecule is enhanced, in vivo.

Nucleic acid molecules of the instant invention, individually, or in combination or in conjunction with other drugs, can be used to treat diseases or conditions discussed above. For example, to treat a disease or condition associated with the levels of Ras, a patient can be treated, or other appropriate cells can be treated, as is evident to those skilled in the art, individually or in combination with one or more drugs under conditions suitable for the treatment.

In a further embodiment, the described molecules, such as antisense, siRNA molecules, or enzymatic nucleic acid molecules, can be used in combination with other known treatments to treat conditions or diseases discussed above. For example, the described molecules can be used in combination with one or more known therapeutic agents to treat cancer, for example colorectal cancer, bladder cancer, lung cancer, pancreatic cancer, breast cancer, or prostate cancer, and any other disease or condition that respond to the modulation of Ras expression.

In another embodiment, the invention features nucleic acid-based inhibitors (e.g., enzymatic nucleic acid molecules, (including DNAzymes), siRNA molecules, and methods for their use to down regulate or inhibit the expression of genes (e.g., Ras) capable of progression and/or maintenance of cancer and/or other disease states that respond to the modulation of Ras expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”.

Other features and advantages of the invention will be apparent from the following description of the preferred embodiments thereof, and from the claims.

Mechanism of Action of Nucleic Acid Molecules of the Invention as is Know in the Art

Antisense: Antisense molecules can be modified or unmodified RNA, DNA, or mixed polymer oligonucleotides and primarily function by specifically binding to matching sequences resulting in inhibition of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). Backbone modified DNA chemistry which have been thus far been shown to act as substrates for RNase H are phosphorothioates, phosphorodithioates, and borontrifluoridates. In addition, 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. S No. 60/101,174, filed on Sep. 21, 1998). All of these references are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

RNA interference: RNA interference refers to the process of sequence specific post-transcriptional gene silencing in animals mediated by short interfering RNAs (siRNA) (Fire et al., 1998, Nature, 391, 806). The corresponding process in plants is commonly referred to as post-transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response through a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence homologous to the siRNA. Cleavage of the target RNA takes place in the middle of the region complementary to the guide sequence of the siRNA duplex (Elbashir et al., 2001, Genes Dev., 15, 188).

Short interfering RNA mediated RNAi has been studied in a variety of systems. Fire et al., 1998, Nature, 391, 806, were the first to observe RNAi in C. Elegans. Wianny and Goetz, 1999, Nature Cell Biol., 2, 70, describes RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309), however siRNA molecules lacking a 5′-phosphate are active when introduced exogenously, suggesting that 5′-phosphorylation of siRNA constructs may occur in vivo.

Enzymatic Nucleic Acid: Several varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention can modulate, e.g., down-regulate, Ras protein expression and can be used to treat disease or diagnose disease associated with the levels of Ras. Enzymatic nucleic acid sequences targeting Ras RNA and sequences that can be targeted with nucleic acid molecules of the invention to down-regulate Ras expression are shown in Tables II and III.

The enzymatic nature of an enzymatic nucleic acid molecule allows the concentration of enzymatic nucleic acid molecule necessary to affect a therapeutic treatment to be lower than a nucleic acid molecule lacking enzymatic activity. This reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a enzymatic nucleic acid molecule.

Nucleic acid molecules having an endonuclease enzymatic activity are able to repeatedly cleave other separate RNA molecules in a nucleotide base sequence-specific manner. With proper design and construction, such enzymatic nucleic acid molecules can be targeted to virtually any RNA transcript, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al, 1999, Chemistry and Biology, 6, 237-250).

Enzymatic nucleic acid molecules of the invention that are allosterically regulated (“allozymes”) can be used to modulate, including down-regulate, Ras expression. These allosteric enzymatic nucleic acids or allozymes (see for example George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, for example, mutant Ras protein, wild-type Ras protein, mutant Ras RNA, wild-type Ras RNA, other proteins and/or RNAs involved in Ras activity, compounds, metals, polymers, molecules and/or drugs that are targeted to Ras expressing cells etc., which, in turn, modulate the activity of the enzymatic nucleic acid molecule. In response to interaction with a predetermined signaling agent, the activity of the allosteric enzymatic nucleic acid molecule is activated or inhibited such that the expression of a particular target is selectively regulated, including down-regulated. The target can comprise wild-type Ras, mutant Ras, a component of Ras, and/or a predetermined cellular component that modulates Ras activity. For example, allosteric enzymatic nucleic acid molecules that are activated by interaction with a RNA encoding Ras protein can be used as therapeutic agents in vivo. The presence of RNA encoding the Ras protein activates the allosteric enzymatic nucleic acid molecule that subsequently cleaves the RNA encoding Ras protein, resulting in the inhibition of Ras protein expression. In this manner, cells that express the the Ras protein are selectively targeted.

In another non-limiting example, an allozyme can be activated by a Ras protein, peptide, or mutant polypeptide that causes the allozyme to inhibit the expression of Ras gene, by, for example, cleaving RNA encoded by Ras gene. In this non-limiting example, the allozyme acts as a decoy to inhibit the function of Ras and also inhibit the expression of Ras once activated by the Ras protein.

Target Sites

Targets for useful enzymatic nucleic acid molecules and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific non-limiting examples of such methods. Enzymatic nucleic acid molecules to such targets are designed as described in the above applications and synthesized to be tested in vitro and in vivo, as also described. The sequences of human K-Ras and H-Ras RNAs were screened for optimal enzymatic nucleic acid target sites using a computer-folding algorithm. Nucleic acid molecule binding/cleavage sites were identified. These sites are shown in Tables II and III (all sequences are 5′ to 3′ in the tables). The nucleotide base position is noted in the Tables as that site to be cleaved by the designated type of enzymatic nucleic acid molecule. Human sequences can be screened and enzymatic nucleic acid molecule and/or antisense thereafter designed, as discussed in Stinchcomb et al., WO 95/23225. In addition, mouse targeted nucleic acid molecules can be used to test efficacy of action of the enzymatic nucleic acid molecule, siRNA, and/or antisense prior to testing in humans.

In addition, enzymatic nucleic acid, siRNA, and antisense nucleic acid molecule binding/cleavage sites are identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions, such as between, for example, the binding arms and the catalytic core of an enzymatic nucleic acid, are eliminated from consideration. Varying binding arm lengths can be chosen to optimize activity.

Antisense, hammerhead, DNAzyme, NCH, amberzyme, zinzyme or G-Cleaver enzymatic nucleic acid molecule, siRNA, and antisense nucleic acid binding/cleavage sites are identified and are designed to anneal to various sites in the RNA target. The enzymatic nucleic acid binding arms or siRNA and antisense nucleic acid sequences are complementary to the target site sequences described above. The nucleic acid molecules can be chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic acid Molecules

Synthesis of nucleic acids greater than 100 nucleotides in length can be difficult using automated methods, and the therapeutic cost of such molecules can be prohibitive. In this invention, small nucleic acid motifs (“small” refers to nucleic acid motifs less than about 100 nucleotides in length, less than about 80 nucleotides in length, and also including less than about 50 nucleotides in length; e.g., DNAzymes) are used for exogenous delivery. The simple structure of these molecules increases the ability of the nucleic acid to invade targeted regions of RNA structure. Exemplary molecules of the instant invention are chemically synthesized as described herein, and others can similarly be synthesized.

Oligonucleotides (e.g., DNAzymes) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table I outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methylimidazole in THF (ABI) and 10% acetic anhydride/10% 2,6-lutidine in THF (ABI); and oxidation solution is 16.9 mM 12, 49 mM pyridine, 9% water in THF (PERSEPTIVET™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNAzymes is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the oligoribonucleotide, are dried to a white powder.

The method of synthesis used for RNA and chemically modified RNA or DNA, including certain enzymatic nucleic acid molecules and siRNA molecules, follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table I outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methylimidazole in THF (ABI) and 10% acetic anhydride/10% 2,6-lutidine in THF (ABI); oxidation solution is 16.9 mM 12, 49 mM pyridine, 9% water in THF (PERSEPTIVETM). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min.

After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA-3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA-3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive DNAzymes or binding attenuated control (BAC) oligonucleotides can be synthesized by substituting one or more nucleotides in the DNAzyme to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Enzymatic nucleic acid molecules are purified by gel electrophoresis using known methods or are purified by high pressure liquid chromatography (HPLC; See Wincott et al., Supra, the totality of which is hereby incorporated herein by reference) and are re-suspended in water.

The sequences of the nucleic acid molecules, including enzymatic nucleic acid molecules and antisense, that are chemically synthesized, are shown in Tables II and III. These sequences are representative only of many more such sequences where the enzymatic portion of the enzymatic nucleic acid molecule (all but the binding arms) is modified to affect activity. For example, the enzymatic nucleic acid sequences listed in Tables II and III can be formed of deoxyribonucleotides or other nucleotides or non-nucleotides. Such enzymatic nucleic acid molecules with enzymatic activity are equivalent to the enzymatic nucleic acid molecules described specifically in the Tables.

Optimizing Activity of the Nucleic acid Molecule of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al, supra, all of which are hereby incorporated by reference in their entirety). All of the above references describe various chemical modifications that can be made to the base, phosphate and/or sugar moieties of the nucleic acid molecules described herein. Modifications which enhance their efficacy in cells, and removal of bases from nucleic acid molecules to shorten oligonucleotide synthesis times and reduce chemical requirements are desired.

There are several examples of sugar, base and phosphate modifications that can be introduced into nucleic acid molecules with significant enhancement in their nuclease stability and efficacy. For example, oligonucleotides can be modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules are also known to increase efficacy (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. S No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). The publications describe general methods and strategies to determine the location of incorporation of sugar, base and/or phosphate modifications and the like into enzymatic nucleic acid molecules without inhibiting catalysis. Similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, excessive modifications can cause some toxicity. Therefore, when designing nucleic acid molecules, the amount of these internucleotide linkages should be minimized. The reduction in the concentration of these linkages can lower toxicity, resulting in increased efficacy and higher specificity of the therapeutic nucleic acid molecules.

Nucleic acid molecules having chemical modifications that maintain or enhance activity are provided. Such nucleic acid molecules are also generally more resistant to nucleases than unmodified nucleic acid molecules. Thus, the in vitro and/or in vivo activity should not be significantly lowered. Therapeutic nucleic acid molecules delivered exogenously are optimally stable within cells until translation of the target RNA has been inhibited long enough to reduce the levels of the undesirable protein. This period of time varies between hours to days, depending upon the disease state. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein)) have expanded the ability to modify nucleic acid molecules by introducing nucleotide modifications to enhance their nuclease stability as described above.

In one embodiment, nucleic acid molecules of the invention include one or more G-clamp nucleotides. A G-clamp nucleotide is a modified cytosine analog wherein modifications result in the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substation within an oligonucleotide can result in substantially enhanced helical thermal stability and mismatch discrimination when hybridized to complementary oligonucleotides. The inclusion of such nucleotides in nucleic acid molecules of the invention can enable both enhanced affinity and specificity to nucleic acid targets.

In another embodiment, the invention features conjugates and/or complexes of nucleic acid molecules targeting Ras genes such as K-Ras, H-Ras, and/or N-Ras. Compositions and conjugates are used to facilitate delivery of molecules into a biological system, such as cells. The conjugates provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel agents for the delivery of molecules, including but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example, enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys and analogs thereof. Biologically active molecules of the invention also include molecules capable of modulating the pharmacokinetics and/or pharmacodynamics of other biologically active molecules, for example lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus containing group and saturated or unsaturated alkyl group, optionally substituted with OH, COOH, oxo, amine, or substituted or unsubstituted aryl groups.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

In the case that down-regulation of the target is desired, therapeutic nucleic acid molecules (e.g., DNAzymes) delivered exogenously are optimally stable within cells until translation of the target RNA has been inhibited long enough to reduce the levels of the targeted protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and others known in the art have expanded the ability to modify nucleic acid molecules by introducing nucleotide modifications to enhance their nuclease stability as described above.

In another embodiment, nucleic acid catalysts having chemical modifications that maintain or enhance enzymatic activity are provided. Such nucleic acids are also generally more resistant to nucleases than unmodified nucleic acid. Thus, the in vitro and/or in vivo the activity of the nucleic acid should not be significantly lowered. As exemplified herein, such enzymatic nucleic acids are useful for in vitro and/or in vivo techniques even if activity over all is reduced 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al, International PCT publication No. WO 97/26270, incorporated by reference herein).

In another embodiment, the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate;

1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

The term “alkyl” as used herein refers to a saturated aliphatic hydrocarbon, including straight-chain, branched-chain “isoalkyl”, and cyclic alkyl groups. The term “alkyl” also comprises alkoxy, alkyl-thio, alkyl-thio-alkyl, alkoxyalkyl, alkylamino, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Preferably, the alkyl group has 1 to 12 carbons. More preferably it is a lower alkyl of from about 1 to 7 carbons, more preferably about 1 to 4 carbons. The alkyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkenyl groups containing at least one carbon-carbon double bond, including straight-chain, branched-chain, and cyclic groups. Preferably, the alkenyl group has about 2 to 12 carbons. More preferably it is a lower alkenyl of from about 2 to 7 carbons, more preferably about 2 to 4 carbons. The alkenyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups.

The term “alkyl” also includes alkynyl groups containing at least one carbon-carbon triple bond, including straight-chain, branched-chain, and cyclic groups. Preferably, the alkynyl group has about 2 to 12 carbons. More preferably it is a lower alkynyl of from about 2 to 7 carbons, more preferably about 2 to 4 carbons. The alkynyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Alkyl groups or moieties of the invention can also include aryl, alkylaryl, carbocyclic aryl, heterocyclic aryl, amide and ester groups. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from about 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

The term “alkoxyalkyl” as used herein refers to an alkyl-O-alkyl ether, for example, methoxyethyl or ethoxymethyl.

The term “alkyl-thio-alkyl” as used herein refers to an alkyl-S-alkyl thioether, for example, methylthiomethyl or methylthioethyl.

The term “amino” as used herein refers to a nitrogen containing group as is known in the art derived from ammonia by the replacement of one or more hydrogen radicals by organic radicals. For example, the terms “aminoacyl” and “aminoalkyl” refer to specific N-substituted organic radicals with acyl and alkyl substituent groups respectively.

The term “amination” as used herein refers to a process in which an amino group or substituted amine is introduced into an organic molecule.

The term “exocyclic amine protecting moiety” as used herein refers to a nucleobase amino protecting group compatible with oligonucleotide synthesis, for example, an acyl or amide group.

The term “alkenyl” as used herein refers to a straight or branched hydrocarbon of a designed number of carbon atoms containing at least one carbon-carbon double bond. Examples of “alkenyl” include vinyl, allyl, and 2-methyl-3-heptene.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkenyl” as used herein refers to a C3-C8 cyclic hydrocarbon containing at least one carbon-carbon double bond. Examples of cycloalkenyl include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “cycloalkyl” as used herein refers to a C3-C8 cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C3-C7 cycloalkyl group attached to the parent molecular moiety through an alkyl group, as defined above. Examples of cycloalkylalkyl groups include cyclopropylmethyl and cyclopentylethyl.

The terms “halogen” or “halo” as used herein refers to indicate fluorine, chlorine, bromine, and iodine.

The term “heterocycloalkyl,” as used herein refers to a non-aromatic ring system containing at least one heteroatom selected from nitrogen, oxygen, and sulfir. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl. The term “C1-C6 hydrocarbyl” as used herein refers to straight, branched, or cyclic alkyl groups having 1-6 carbon atoms, optionally containing one or more carbon-carbon double or triple bonds. Examples of hydrocarbyl groups include, for example, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, vinyl, 2-pentene, cyclopropylmethyl, cyclopropyl, cyclohexylmethyl, cyclohexyl and propargyl. When reference is made herein to C1-C6 hydrocarbyl containing one or two double or triple bonds it is understood that at least two carbons are present in the alkyl for one double or triple bond, and at least four carbons for two double or triple bonds.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein. There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine,

5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O— NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., DNAzyme) structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of patients with nucleic acid molecules can also include combinations of different types of nucleic acid molecules. Therapies can be devised which include a mixture of enzymatic nucleic acid molecules (including different enzymatic nucleic acid molecule motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995, which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. Neuro Virol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819, all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means described herein and known in the art, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for exaple the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes, which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example, sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example, ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example, sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

In another aspect of the invention, nucleic acid molecules of the present invention are expressed from transcription units (see for example Couture et al., 1996, TIG., 12, 510, Skillern et al., International PCT Publication No. WO 00/22113, Conrad, International PCT Publication No. WO 00/22114, and Conrad, U.S. Pat. No. 6,054,299) inserted into DNA or RNA vectors. The recombinant vectors can be DNA plasmids or viral vectors. Enzymatic nucleic acid expressing viral vectors can be constructed based on, but not limited to, adeno-associated virus, retrovirus, adenovirus, or alphavirus.The recombinant vectors capable of expressing the nucleic acid molecules can be delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intra-muscular administration, by administration to target cells ex-planted from the patient followed by reintroduction into the patient, or by any other means that would allow for introduction into the desired target cell (for a review see Couture et al., 1996, TIG., 12, 510).

One aspect of the invention features an expression vector comprising a nucleic acid sequence encoding at least one of the nucleic acid molecules of the instant invention. The nucleic acid sequence encoding the nucleic acid molecule of the instant invention is operably linked in a manner that allows expression of that nucleic acid molecule.

Another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner that allows expression and/or delivery of said nucleic acid molecule.

In another embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of DNAzyme molecules and binding/cleavage sites within Ras RNA.

### Example 1

### Identification of Potential Target Sites in Human Ras RNA

The sequence of human Ras genes are screened for accessible sites using a computer-folding algorithm. Regions of the RNA that do not form secondary folding structures and contained potential enzymatic nucleic acid molecule and/or antisense binding/cleavage sites are identified. The sequences of K-Ras and H-Ras binding/cleavage sites are shown in Tables II and III.

### Example 2

### Selection of Enzymatic Nucleic Acid Cleavage Sites in Human Ras RNA

Enzymatic nucleic acid molecule target sites are chosen by analyzing sequences of Human K-Ras and H-Ras (for example, Genbank accession Nos: NM_{—}004985 and NM_{—}005343 respectively) and prioritizing the sites on the basis of folding. Enzymatic nucleic acid molecules are designed that can bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Enzymatic Nucleic Acid Molecules for Efficient Cleavage and/or Blocking of Ras RNA

DNAzyme molecules are designed to anneal to various sites in the RNA message. The binding arms of the DNAzyme molecules are complementary to the target site sequences described above. The DNAzymes were chemically synthesized. The method of synthesis used followed the procedure for nucleic acid synthesis as described herein and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%. The sequences of the chemically synthesized DNAzyme molecules used in this study are shown below in Tables II and III.

### Example 4

### DNAzyme Cleavage of Ras RNA Target in vitro

DNAzymes targeted to the human K-Ras and H-Ras RNA are designed and synthesized as described above. These enzymatic nucleic acid molecules can be tested for cleavage activity in vitro, for example, using the following procedure. The target sequences and the nucleotide location within the K-Ras and H-Ras RNA are given in Tables II and III respectively.

Cleavage Reactions:

DNAzymes and substrates were synthesized in 96-well format using 0.2 μmol scale. Substrates were 5′-³²P labeled and gel purified using 7.5% polyacrylamide gels, and eluting into water. Assays were done by combining trace substrate with 500 nM DNAzyme or greater, and initiated by adding final concentrations of 40 mM Mg⁺², and 50 mM Tris-Ci pH 8.0. For each DNAzyme/substrate combination a control reaction was done to ensure cleavage was not the result of non-specific substrate degradation. A single three hour time point was taken and run on a 15% polyacrylamide gel to asses cleavage activity. Gels were dried and scanned using a Molecular Dynamics Phosphorimager and quantified using Molecular Dynamics ImageQuant software. Percent cleaved was determined by dividing values for cleaved substrate bands by full-length (uncleaved) values plus cleaved values and multiplying by 100 (% cleaved=[C/(U+C)]* 100).

### Example 4

### DNAzyme Cleavage of Ras RNA Target in vivo

Cell Culture

Wickstrom, 2001, Mol. Biotechnol., 18, 35-35, describes a cell culture system in which antisense oligonucleotides targeting H-Ras were studied in transformed mouse cells that form solid tumors. Treatment of cells with antisense targeting H-Ras resulted in the sequence specific and dose dependent inhibition of H-Ras expression. In this study, it was determined that antisense targeting the first intron region of H-Ras were more effective than antisense targeting the initiation codon region.

Kita et al., 1999, Int. J. Cancer, 80, 553-558, describes the growth inhibition of human pancreatic cancer cell lines by antisense oligonucleotides specific to mutated K-Ras genes. Antisense oligonucleotides were transfected to the transformed cells using liposomes. Cellular proliferation, K-Ras mRNA expression, and K-Ras protein synthesis were all evaluated as endpoints. Sato et al., 2000, Cancer Lett., 155, 153-161, describes another human pancreatic cancer cell line, HOR-P1, that is characterized by high angiogenic activity and metastatic potential. Genetic and molecular analysis of this cell line revealed both increased telomerase activity and a mutation in the K-Ras oncogene.

A variety of endpoints have been used in cell culture models to look at Ras-mediated effects after treatment with anti-Ras agents. Phenotypic endpoints include inhibition of cell proliferation, RNA expression, and reduction of Ras protein expression. Because Ras oncogene mutations are directly associated with increased proliferation of cetain tumor cells, a proliferation endpoint for cell culture assays are preferably be used as the primary screen. There are several methods by which this endpoint can be measured. Following treatment of cells with DNAzymes, cells are allowed to grow (typically 5 days) after which either the cell viability, the incorporation of [³H] thymidine into cellular DNA and/or the cell density can be measured. The assay of cell density can be done in a 96-well format using commercially available fluorescent nucleic acid stains (such as Syto® 13 or CyQuant®). As a secondary, confirmatory endpoint a DNAzyme-mediated decrease in the level of Ras protein expression can be evaluated using a Ras-specific ELISA.

Animal Models

Evaluating the efficacy of anti-Ras agents in animal models is an important prerequisite to human clinical trials. As in cell culture models, the most Ras sensitive mouse tumor xenografts are those derived from cancer cells that express mutant Ras proteins. Nude mice bearing H-Ras transformed bladder cancer cell xenografts were sensitive to an anti-Ras antisense nucleic acid, resulting in an 80% inhibition of tumor growth after a 31 day treatment period (Wickstrom, 2001, Mol. Biotechnol., 18, 35-35). Zhang et al., 2000, Gene Ther., 7, 2041, describes an anti-K-Ras ribozyme adenoviral vector (KRbz-ADV) targeting a K-Ras mutant (K-Ras codon 12 GGT to GTT; H441 and H1725 cells respectively). Non-small cell lung cancer cells (NSCLC H441 and H1725 cells) that express the mutant K-Ras protein were used in nude mouse xenografts compared to NSCLC H1650 cells that lack the relevant mutation. Pre-treatment with KRbz-ADV completely abrogated engraftment of both H441 and H1725 cells and compared to 100% engraftment and tumor growth in animals that received untreated tumor cells or a control vector. The above studies provide proof that inhibition of Ras expression by anti-Ras agents causes inhibition of tumor growth in animals. Anti-Ras DNAzymes chosen from in vitro assays can be further tested in similar mouse xenograft models. Active DNAzymes can be subsequently tested in combination with standard chemotherapies.

Indications

Particular degenerative and disease states that are associated with Ras expression modulation include but are not limited to cancer, for example lung cancer, colorectal cancer, bladder cancer, pancreatic cancer, breast cancer, prostate cancer and/or any other diseases or conditions that are related to or will respond to the levels of Ras in a cell or tissue, alone or in combination with other therapies.

The present body of knowledge in Ras research indicates the need for methods to assay Ras activity and for compounds that can regulate Ras expression for research, diagnostic, and therapeutic use.

The use of monoclonal antibodies, chemotherapy, radiation therapy, and analgesics, are all non-limiting examples of methods that can be combined with or used in conjunction with the nucleic acid molecules (e.g. DNAzymes) of the instant invention. Common chemotherapies that can be combined with nucleic acid molecules of the instant invention include various combinations of cytotoxic drugs to kill cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the nucleic acid molecules of the instant invention (e.g. DNAzyme molecules) are hence within the scope of the instant invention.

Diagnostic Uses

The nucleic acid molecules of this invention (e.g., enzymatic nucleic acid molecules) can be used as diagnostic tools to examine genetic drift and mutations within diseased cells or to detect the presence of Ras RNA in a cell. The close relationship between enzymatic nucleic acid molecule activity and the structure of the target RNA allows the detection of mutations in any region of the molecule that alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are known in the art, and include detection of the presence of mRNAs associated with Ras-related condition. Such RNA is detected by determining the presence of a cleavage product after treatment with an enzymatic nucleic acid molecule using standard methodology.

In a specific example, enzymatic nucleic acid molecules that cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., Ras) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is described, for example, in George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

Additional Uses

Potential uses of sequence-specific enzymatic nucleic acid molecules of the instant invention can have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to modulate gene expression of target genes in bacterial, microbial, fungal, viral, and eukaryotic systems including plant or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims. The invention illustratively described herein suitably can be practiced in the absence of any element or elements, limitation or limitations which is not specifically disclosed herein. Thus, for example, in each instance herein any of the terms “comprising”, “consisting essentially of” and “consisting of” can be replaced with either of the other two terms. The terms and expressions that have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed can be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the claims that follow.

TABLE I
A. 2.5 μmol Synthesis Cycle ABI 394 Instrument

| Reagent | Equivalents | Amount | | Wait Time* DNA | | Wait Time* 2′-O-methyl | | Wait Time* RNA | |
|---|---|---|---|---|---|---|---|---|---|
| Phosphoramidites | 6.5 | 163 | μL | 45 | sec | 2.5 | min | 7.5 | min |
| S-Ethyl Tetrazole | 23.6 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| Reagent | Equivalents | Amount | | Wait Time* DNA | | Wait Time* 2′-O-methyl | | Wait Time* RNA | |
|---|---|---|---|---|---|---|---|---|---|
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | Equivalents:DNA/ | Amount: DNA/2′-O- | | Wait Time* | | Wait Time* | | Wait Time* | |
|---|---|---|---|---|---|---|---|---|---|
| Reagent | 2′-O-methyl/Ribo | methyl/Ribo | | DNA | | 2′-O-methyl | | Ribo | |
| Phosphoramidites | 22/33/66 | 40/60/120 | μL | 60 | sec | 180 | sec | 360 | sec |
| S-Ethyl Tetrazole | 70/105/210 | 40/60/120 | μL | 60 | sec | 180 | min | 360 | sec |
| Acetic Anhydride | 265/265/265 | 50/50/50 | μL | 10 | sec | 10 | sec | 10 | sec |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

*Wait time does not include contact time during delivery.

TABLE II

Human K-Ras DNAzyme and Substrate Sequence

| | | | | |
|---|---|---|---|---|
| | | Seq | | Seq |
| Pos | Substrate | ID | DNAzyme | ID |

| | | | | |
|---|---|---|---|---|
| 10 | CCUAGGCG G CGGCCGCG | 1 | CGCGGCCG GGCTAGCTACAACGA CGCCTAGG | 1322 |
| 13 | AGGCGGCG G CCGCGGCG | 2 | CGCCGCGG GGCTAGCTACAACGA CGCCGCCT | 1323 |
| 16 | CGGCGGCC G CGGCGGCG | 3 | CGCCGCCG GGCTAGCTACAACGA GGCCGCCG | 1324 |
| 19 | CGGCCGCG G CGGCGGAG | 4 | CTCCGCCG GGCTAGCTACAACGA CGCGGCCG | 1325 |
| 22 | CCGCGGCG G CGGACGCA | 5 | TGCCTCCG GGCTAGCTACAACGA CGCCGCGG | 1326 |
| 28 | CGGCGGCA G CAGCAGCG | 6 | CGCTGCTG GGCTAGCTACAACGA CTCCGCGG | 1327 |
| 31 | CGGAGGCA G CACCGGCG | 7 | CGCCGCTG GGCTAGCTACAACGA TGCCTCCG | 1328 |
| 34 | AGGCAGCA G CGGCGGCG | 8 | CGCCGCCG GGCTAGCTACAACGA TGCTGCCT | 1329 |
| 37 | CAGCAGCG G CGGCGGCA | 9 | TGCCGCCG GGCTAGCTACAACGA CGCTGCTG | 1330 |
| 40 | CAGCGGCC G CGGCAGUG | 10 | CACTGCCG GGCTAGCTACAACGA CGCCGCTG | 1331 |
| 43 | CGGCGGCG G CAGUGGCG | 11 | CGCCACTG GGCTAGCTACAACGA CGCCGCCG | 1332 |
| 46 | CGGCGGCA G UGGCGGCG | 12 | CGCCGCCA GGCTAGCTACAACGA TGCCGCCG | 1333 |
| 49 | CGGCAGUG G CGGCCGCG | 13 | CGCCGCCG GGCTAGCTACAACGA CACTGCCG | 1334 |
| 52 | CAGUGCCG G CGGCCAAG | 14 | CTTCGCCG GGCTAGCTACAACGA CGCCACTG | 1335 |
| 55 | UGGCGGCG G CGAAGGUG | 15 | CACCTTCG GGCTAGCTACAACGA CGCCGCCA | 1336 |
| 61 | CGGCGAAG G UGGCGGCG | 16 | CGCCGCCA GGCTAGCTACAACGA CTTCGCCG | 1337 |
| 64 | CGAAGGUG G CGGCGGCU | 17 | AGCCGCCG GGCTAGCTACAACGA CACCTTCG | 1338 |
| 67 | AGGUGGCG G CGGCUGGC | 18 | CCGAGCCG GGCTAGCTACAACGA CGCCACCT | 1339 |
| 70 | UGGCGGCG G CUCGGCCA | 19 | TGGCCGAG GGCTAGCTACAACGA CGCCGCCA | 1340 |
| 75 | GCGGCUCG G CCAGUACU | 20 | AGTACTGG GGCTAGCTACAACGA CGAGCCGC | 1341 |
| 79 | CUCGCCCA G UACUCCCG | 21 | CGGGAGTA GGCTAGCTACAACGA TGGCCGAG | 1342 |
| 81 | CGGCCAGU A CUCCCGGC | 22 | GCCGGGAG GGCTAGCTACAACGA ACTGGCCG | 1343 |
| 88 | UACUCCCG G CCCCCGCC | 23 | GGCGGGGG GGCTAGCTACAACGA CCCGACTA | 1344 |
| 94 | CGGCCCCC G CCAUUUCG | 24 | CGAAATGG GGCTAGCTACAACGA GGGGCCCG | 1345 |
| 97 | CCCCCGCC A UUUCGCAC | 25 | GTCCGAAA GGCTAGCTACAACGA GGCGGGGG | 1346 |
| 104 | CAUUUCGG A CUGGGAGC | 26 | GCTCCCAG GGCTAGCTACAACGA CCGAAATG | 1347 |
| 111 | GACUGGGA G CGAGCGCG | 27 | CGCGCTCG GGCTAGCTACAACGA TCCCAGTC | 1348 |
| 115 | GGGAGCGA G CGCGGCGC | 28 | GCGCCGCG GGCTAGCTACAACGA TCGCTCCC | 1349 |
| 117 | GAGCGAGC G CGGCCCAG | 29 | CTGCGCCG GGCTAGCTACAACGA GCTCGCTC | 1350 |
| 120 | CGAGCGCG G CGCAGGCA | 30 | TGCCTGCG GGCTAGCTACAACGA CGCGCTCG | 1351 |
| 122 | AGCGCGGC G CAGGCACU | 31 | AGTCCCTG GGCTAGCTACAACGA GCCGCGCT | 1352 |
| 126 | CGGCGCAG G CACUGAAG | 32 | CTTCAGTG GGCTAGCTACAACGA CTGCGCCG | 1353 |
| 128 | GCGCAGGC A CUGAAGGC | 33 | GCCTTCAG GGCTAGCTACAACGA GCCTGCGC | 1354 |
| 135 | CACUGAAG G CGGCGGCG | 34 | CGCCGCCG GGCTAGCTACAACGA CTTCAGTG | 1355 |
| 138 | UGAAGGCG G CGGCGGGG | 35 | CCCCGCCG GGCTAGCTACAACGA CGCCTTCA | 1356 |
| 141 | AGGCGGCG G CGGGGCCA | 36 | TGGCCCCG GGCTAGCTACAACGA CGCCGCCT | 1357 |
| 146 | GCGGCGGG G CCAGAGGC | 37 | GCCTCTGG GGCTAGCTACAACGA CCCGCCGC | 1358 |
| 153 | GGCCAGAG G CUCAGCGG | 38 | CCCCTCAC CCCTAGCTACAACGA CTCTGGCC | 1359 |
| 158 | GAGGCUCA G CGGCUCCC | 39 | GGGAGCCG GGCTAGCTACAACGA TGAGCCTC | 1360 |
| 161 | GCUCAGCG G CUCCCAGG | 40 | CCTGGGAG GGCTAGCTACAACGA CGCTGAGC | 1361 |
| 169 | GCUCCCAG G UGCGGGAG | 41 | CTCCCGCA GGCTAGCTACAACGA CTGGGAGC | 1362 |
| 171 | UCCCAGGU G CGGGAGAG | 42 | CTCTCCCG GGCTAGCTACAACGA ACCTGGGA | 1363 |
| 182 | GGAGAGAG G CCUGCUGA | 43 | TCAGCAGG GGCTAGCTACAACGA CTCTCTCC | 1364 |
| 186 | AGAGGCCU G CUGAAAAU | 44 | ATTTTCAG GGCTAGCTACAACGA AGGCCTCT | 1365 |
| 193 | UGCUGAAA A UGACUGAA | 45 | TTCAGTCA GGCTAGCTACAACGA TTTCAGCA | 1366 |
| 196 | UGAAAAUG A CUCAAUAU | 46 | ATATTCAG GGCTAGCTACAACGA CATTTTCA | 1367 |
| 201 | AUGACUGA A UAUAAACU | 47 | AGTTTATA GGCTAGCTACAACGA TCAGTCAT | 1368 |
| 203 | GACUGAAU A UAAACUUG | 48 | CAAGTTTA GGCTAGCTACAACGA ATTCAGTC | 1369 |
| 207 | GAAUAUAA A CUUGUGGU | 49 | ACCACAAG GGCTAGCTACAACGA TTATATTC | 1370 |
| 211 | AUAAACUU G UGGUAGUU | 50 | AACTACCA GGCTAGCTACAACGA AAGTTTAT | 1371 |
| 214 | AACUUGUG G UAGUUGGA | 51 | TCCAACTA GGCTAGCTACAACGA CACAAGTT | 1372 |
| 217 | UUGUCGUA G UUGGAGCU | 52 | AGCTCCAA GGCTAGCTACAACGA TACCACAA | 1373 |
| 223 | UAGUUGGA G CUUGUGGC | 53 | GCCACAAG GGCTAGCTACAACGA TCCAACTA | 1374 |
| 227 | UGGAGCUU G UGGCGUAG | 54 | CTACGCCA GGCTAGCTACAACGA AAGCTCCA | 1375 |
| 230 | AGCUUGUG G CGUAGGCA | 55 | TGCCTACG GGCTAGCTACAACGA CACAAGCT | 1376 |
| 232 | CUUGUCGC G UAGGCAAG | 56 | CTTGCCTA GGCTAGCTACAACGA GCCACAAG | 1377 |
| 236 | UGGCCUAG G CAAGAGUG | 57 | CACTCTTG GGCTAGCTACAACGA CTACGCCA | 1378 |
| 242 | AGGCAACA G UGCCUUGA | 58 | TCAAGGCA GGCTAGCTACAACGA TCTTGCCT | 1379 |
| 244 | GCAAGAGU G CCUUGACG | 59 | CGTCAAGG GGCTAGCTACAACGA ACTCTTGC | 1380 |
| 250 | GUGCCUUG A CGAUACAG | 60 | CTCTATCG GGCTAGCTACAACGA CAAGGCAC | 1381 |
| 253 | CCUUGACG A UACAGCUA | 61 | TAGCTCTA GGCTAGCTACAACGA CGTCAAGG | 1382 |
| 255 | UUGACCAU A CAGCUAAU | 62 | ATTAGCTG GGCTAGCTACAACGA ATCGTCAA | 1383 |
| 258 | ACGAUACA G CUAAUUCA | 63 | TGAATTAG GGCTAGCTACAACGA TGTATCGT | 1384 |
| 262 | UACAGCUA A UUCAGAAU | 64 | ATTCTGAA GGCTAGCTACAACGA TAGCTGTA | 1385 |
| 269 | AAUUCAGA A UCAUUUUC | 65 | CAAAATGA GGCTAGCTACAACGA TCTGAATT | 1386 |
| 272 | UCAGAAUC A UUUUGUGG | 66 | CCACAAAA GGCTAGCTACAACGA GATTCTGA | 1387 |
| 277 | AUCAUUUU G UGGACGAA | 67 | TTCGTCCA GGCTAGCTACAACGA AAAATGAT | 1388 |
| 281 | UUUUGUGG A CGAAUAUG | 68 | CATATTCG GGCTAGCTACAACGA CCACAAAA | 1389 |
| 285 | GUGCACGA A UAUGAUCC | 69 | GGATCATA GGCTAGCTACAACGA TCGTCCAC | 1390 |
| 287 | GGACGAAU A UGAUCCAA | 70 | TTGGATCA GGCTAGCTACAACGA ATTCGTCC | 1391 |
| 290 | CGAAUAUG A UCCAACAA | 71 | TTGTTGCA GGCTAGCTACAACGA CATATTCG | 1392 |
| 295 | AUGAUCCA A CAAUAGAG | 72 | CTCTATTG GGCTAGCTACAACGA TGGATCAT | 1393 |
| 298 | AUCCAACA A UAGAGGAU | 73 | ATCCTCTA GGCTAGCTACAACGA TGTTGGAT | 1394 |
| 305 | AAUAGAGG A UUCCUACA | 74 | TGTACGAA GGCTAGCTACAACGA CCTCTATT | 1395 |
| 311 | GGAUUCCU A CAGCAAGC | 75 | GCTTCCTG GGCTAGCTACAACGA AGGAATCC | 1396 |
| 318 | UACAGGAA G CAAGUACU | 76 | ACTACTTG GGCTAGCTACAACGA TTCCTGTA | 1397 |
| 322 | GGAAGCAA G UAGUAAUU | 77 | AATTACTA GGCTAGCTACAACGA TTGCTTGC | 1398 |
| 325 | AGCAAGUA G UAAUUGAU | 78 | ATCAATTA GGCTAGCTACAACGA TACTTGCT | 1399 |
| 328 | AAGUAGUA A UUGAUGGA | 79 | TCCATCAA GGCTAGCTACAACGA TACTACTT | 1400 |
| 332 | AGUAAUUG A UGGAGAAA | 80 | TTTCTCCA GGCTAGCTACAACGA CAATTACT | 1401 |
| 340 | AUGGAGAA A CCUGUCUC | 81 | GAGACAGG GGCTAGCTACAACGA TTCTCCAT | 1402 |
| 344 | AGAAACCU G UCUCUUGG | 82 | CGAAGAGA GGCTAGCTACAACGA AGGTTTCT | 1403 |
| 353 | UCUCUUGG A UAUUCUCG | 83 | CGAGAATA GGCTAGCTACAACGA CCAAGAGA | 1404 |
| 355 | UCUUGGAU A UUCUCGAC | 84 | GTCGAGAA GGCTAGCTACAACGA ATCCAAGA | 1405 |
| 362 | UAUUCUCG A CACAGCAG | 85 | CTGCTGTG GGCTAGCTACAACGA CGAGAATA | 1406 |
| 364 | UUCUCGAC A CAGCAGGU | 86 | ACCTGCTG GGCTAGCTACAACGA GTCGAGAA | 1407 |
| 367 | UCGACACA G CAGGUCAA | 87 | TTGACCTG GGCTAGCTACAACGA TGTGTCGA | 1408 |
| 371 | CACAGCAG G UCAAGAGG | 88 | CCTCTTGA GGCTAGCTACAACGA CTGCTGTG | 1409 |
| 381 | CAAGAGGA G UACAGUGC | 89 | GCACTCTA GGCTAGCTACAACGA TCCTCTTG | 1410 |
| 383 | AGAGGAGU A CAGUGCAA | 90 | TTGCACTG GGCTAGCTACAACGA ACTCCTCT | 1411 |
| 386 | GGAGUACA G UGCAAUGA | 91 | TCATTGCA GGCTAGCTACAACGA TGTACTCC | 1412 |
| 388 | AGUACAGU G CAAUGAGG | 92 | CCTCATTG GGCTAGCTACAACGA ACTGTACT | 1413 |
| 391 | ACAGUGCA A UGAGGGAC | 93 | GTCCCTCA GGCTAGCTACAACGA TGCACTGT | 1414 |
| 398 | AAUGAGGG A CCACUACA | 94 | TGTACTGG GGCTAGCTACAACGA CCCTCATT | 1415 |
| 402 | AGGGACCA G UACAUGAG | 95 | CTCATGTA GGCTAGCTACAACGA TGGTCCCT | 1416 |
| 404 | GGACCAGU A CAUGAGGA | 96 | TCCTCATG GGCTAGCTACAACGA ACTGGTCC | 1417 |
| 406 | ACCAGUAC A UCAGGACU | 97 | AGTCCTCA GGCTAGCTACAACGA GTACTGGT | 1418 |
| 412 | ACAUGAGG A CUGGGGAG | 98 | CTCCCCAG GGCTAGCTACAACGA CCTCATGT | 1419 |
| 422 | UGGGGAGG G CUUUCUUU | 99 | AAAGAAAG GGCTAGCTACAACGA CCTCCCCA | 1420 |
| 431 | CUUUCUUU G UGUAUUUG | 100 | CAAATACA GGCTAGCTACAACGA AAAGAAAG | 1421 |
| 433 | UUCUUUGU G UAUUUGCC | 101 | GGCAAATA GGCTAGCTACAACGA ACAAAGAA | 1422 |
| 435 | CUUUGUGU A UUUGCCAU | 102 | ATGGCAAA GGCTAGCTACAACGA ACACAAAG | 1423 |
| 439 | GUGUAUUU G CCAUAAAU | 103 | ATTTATGG GGCTAGCTACAACGA AAATACAC | 1424 |
| 442 | UAUUUGCC A UAAAUAAU | 104 | ATTATTTA GGCTAGCTACAACGA GGCAAATA | 1425 |
| 446 | UGCCAUAA A UAAUACUA | 105 | TAGTATTA GGCTAGCTACAACGA TTATGGCA | 1426 |
| 449 | CAUAAAUA A UACUAAAU | 106 | ATTTAGTA GGCTAGCTACAACGA TATTTATG | 1427 |
| 451 | UAAAUAAU A CUAAAUCA | 107 | TGATTTAG GGCTAGCTACAACGA ATTATTTA | 1428 |
| 456 | AAUACUAA A UCAUUUGA | 108 | TCAAATGA GGCTAGCTACAACGA TTAGTATT | 1429 |
| 459 | ACUAAAUC A UUUGAAGA | 109 | TCTTCAAA GGCTAGCTACAACGA GATTTAGT | 1430 |
| 467 | AUUUGAAG A UAUUCACC | 110 | GGTGAATA GGCTAGCTACAACGA CTTCAAAT | 1431 |
| 469 | UUGAAGAU A UUCACCAU | 111 | ATGGTGAA GGCTAGCTACAACGA ATCTTCAA | 1432 |
| 473 | AGAUAUUC A CCAUUAUA | 112 | TATAATGG GGCTAGCTACAACGA GAATATCT | 1433 |
| 476 | UAUUCACC A UUAUAGAG | 113 | CTCTATAA GGCTAGCTACAACGA GGTGAATA | 1434 |
| 479 | UCACCAUU A UAGAGAAC | 114 | GTTCTCTA GGCTAGCTACAACGA AATGGTGA | 1435 |
| 486 | UAUAGAGA A CAAAUUAA | 115 | TTAATTTG GGCTAGCTACAACGA TCTCTATA | 1436 |
| 490 | GAGAACAA A UUAAAAGA | 116 | TCTTTTAA GGCTAGCTACAACGA TTGTTCTC | 1437 |
| 499 | UUAAAAGA G UUAAGGAC | 117 | GTCCTTAA GGCTAGCTACAACGA TCTTTTAA | 1438 |
| 506 | AGUUAAGG A CUCUGAAG | 118 | CTTCAGAG GGCTAGCTACAACGA CCTTAACT | 1439 |
| 515 | CUCUGAAG A UGUACCUA | 119 | TAGGTACA GGCTAGCTACAACGA CTTCAGAG | 1440 |
| 517 | CUGAAGAU G UACCUAUG | 120 | CATAGGTA GGCTAGCTACAACGA ATCTTCAG | 1441 |
| 519 | GAAGAUGU A CCUAUGGU | 121 | ACCATAGG GGCTAGCTACAACGA ACATCTTC | 1442 |
| 523 | AUGUACCU A UGGUCCUA | 122 | TAGGACCA GGCTAGCTACAACGA AGGTACAT | 1443 |
| 526 | UACCUAUG G UCCUAGUA | 123 | TACTAGGA GGCTAGCTACAACGA CATAGGTA | 1444 |
| 532 | UGGUCCUA G UAGGAAAU | 124 | ATTTCCTA GGCTAGCTACAACGA TAGGACCA | 1445 |
| 539 | AGUAGGAA A UAAAUGUG | 125 | CACATTTA GGCTAGCTACAACGA TTCCTACT | 1446 |
| 543 | GGAAAUAA A UGUGAUUU | 126 | AAATCACA GGCTAGCTACAACGA TTATTTCC | 1447 |
| 545 | AAAUAAAU G UGAUUUGC | 127 | GCAAATCA GGCTAGCTACAACGA ATTTATTT | 1448 |
| 548 | UAAAUGUG A UUUGCCUU | 128 | AAGGCAAA GGCTAGCTACAACGA CACATTTA | 1449 |
| 552 | UGUGAUUU G CCUUCUAG | 129 | CTAGAAGG GGCTAGCTACAACGA AAATCACA | 1450 |
| 562 | CUUCUAGA A CAGUAGAC | 130 | GTCTACTG GGCTAGCTACAACGA TCTAGAAG | 1451 |
| 565 | CUACAACA G UAGACACA | 131 | TGTGTCTA GGCTAGCTACAACGA TGTTCTAG | 1452 |
| 569 | AACACUAG A CACAAAAC | 132 | GTTTTGTG GGCTAGCTACAACGA CTACTGTT | 1453 |
| 571 | CAGUAGAC A CAAAACAG | 133 | CTGTTTTG GGCTAGCTACAACGA GTCTACTG | 1454 |
| 576 | GACACAAA A CAGGCUCA | 134 | TGAGCCTG GGCTAGCTACAACGA TTTGTGTC | 1455 |
| 580 | CAAAACAG G CUCAGGAC | 135 | GTCCTGAG GGCTAGCTACAACGA CTGTTTTG | 1456 |
| 587 | GGCUCAGG A CUUAGCAA | 136 | TTGCTAAG GGCTAGCTACAACGA CCTGAGCC | 1457 |
| 592 | AGGACUUA G CAAGAAGU | 137 | ACTTCTTG GGCTAGCTACAACGA TAAGTCCT | 1458 |
| 599 | AGCAAGAA G UUAUGGAA | 138 | TTCCATAA GGCTAGCTACAACGA TTCTTGCT | 1459 |
| 602 | AAGAAGUU A UGGAAUUC | 139 | GAATTCCA GGCTAGCTACAACGA AACTTCTT | 1460 |
| 607 | GUUAUGGA A UUCCUUUU | 140 | AAAAGGAA GGCTAGCTACAACGA TCCATAAC | 1461 |
| 616 | UUCCUUUU A UUCAAACA | 141 | TGTTTCAA GGCTAGCTACAACGA AAAAGGAA | 1462 |
| 622 | UUAUUGAA A CAUCAGCA | 142 | TGCTGATG GGCTAGCTACAACGA TTCAATAA | 1463 |
| 624 | AUUGAAAC A UCAGCAAA | 143 | TTTGCTGA GGCTAGCTACAACGA GTTTCAAT | 1464 |
| 628 | AAACAUCA G CAAAGACA | 144 | TCTCTTTG GGCTAGCTACAACGA TGATGTTT | 1465 |
| 634 | CAGCAAAG A CAAGACAG | 145 | CTGTCTTG GGCTAGCTACAACGA CTTTGCTG | 1466 |
| 639 | AAGACAAG A CAGGGUGU | 146 | ACACCCTG GGCTAGCTACAACGA CTTGTCTT | 1467 |
| 644 | AAGACAGG G UGUUGAUG | 147 | CATCAACA GGCTAGCTACAACGA CCTGTCTT | 1468 |
| 646 | GACAGGGU G UUGAUGAU | 148 | ATCATCAA GGCTAGCTACAACGA ACCCTGTC | 1469 |
| 650 | GGGUGUUG A UGAUGCCU | 149 | AGGCATCA GGCTAGCTACAACGA CAACACCC | 1470 |
| 653 | UGUUGAUG A UGCCUUCU | 150 | AGAAGGCA GGCTAGCTACAACGA CATCAACA | 1471 |
| 655 | UUGAUGAU G CCUUCUAU | 151 | ATAGAAGG GGCTAGCTACAACGA ATCATCAA | 1472 |
| 662 | UGCCUUCU A UACAUUAG | 152 | CTAATGTA GGCTAGCTACAACGA AGAAGGCA | 1473 |
| 664 | CCUUCUAU A CAUUAGUU | 153 | AACTAATG GGCTAGCTACAACGA ATAGAAGG | 1474 |
| 666 | UUCUAUAC A UUAGUUCG | 154 | CGAACTAA GGCTAGCTACAACGA GTATAGAA | 1475 |
| 670 | AUACAUUA G UUCGAGAA | 155 | TTCTCGAA GGCTAGCTACAACGA TAATGTAT | 1476 |
| 679 | UUCGAGAA A UUCGAAAA | 156 | TTTTCGAA GGCTAGCTACAACGA TTCTCGAA | 1477 |
| 687 | AUUCGAAA A CAUAAAGA | 157 | TCTTTATG GGCTAGCTACAACGA TTTCGAAT | 1478 |
| 689 | UCGAAAAC A UAAAGAAA | 158 | TTTCTTTA GGCTAGCTACAACGA GTTTTCGA | 1479 |
| 700 | AAGAAAAG A UGAGCAAA | 159 | TTTGCTCA GGCTAGCTACAACGA CTTTTCTT | 1480 |
| 704 | AAAGAUGA G CAAAGAUG | 160 | CATCTTTG GGCTAGCTACAACGA TCATCTTT | 1481 |
| 710 | GAGCAAAG A UGGUAAAA | 161 | TTTTACCA GGCTAGCTACAACGA CTTTGCTC | 1482 |
| 713 | CAAAGAUG G UAAAAAGA | 162 | TCTTTTTA GGCTAGCTACAACGA CATCTTTG | 1483 |
| 732 | AAAAAGAA G UCAAAGAC | 163 | GTCTTTGA GGCTAGCTACAACGA TTCTTTTT | 1484 |
| 739 | AGUCAAAG A CAAAGUGU | 164 | ACACTTTG GGCTAGCTACAACGA CTTTGACT | 1485 |
| 744 | AAGACAAA G UGUGUAAU | 165 | ATTACACA GGCTAGCTACAACGA TTTCTCTT | 1486 |
| 746 | GACAAACU G UGUAAUUA | 166 | TAATTACA GGCTAGCTACAACGA ACTTTGTC | 1487 |
| 748 | CAAAGUGU G UAAUUAUG | 167 | CATAATTA GGCTAGCTACAACGA ACACTTTG | 1488 |
| 751 | AGUGUGUA A UUAUGUAA | 168 | TTACATAA GGCTAGCTACAACGA TACACACT | 1489 |
| 754 | GUGUAAUU A UGUAAAUA | 169 | TATTTACA GGCTAGCTACAACGA AATTACAC | 1490 |
| 756 | GUAAUUAU G UAAAUACA | 170 | TGTATTTA GGCTAGCTACAACGA ATAATTAC | 1491 |
| 760 | UUAUGUAA A UACAAUUU | 171 | AAATTGTA GGCTAGCTACAACGA TTACATAA | 1492 |
| 762 | AUGUAAAU A CAAUUUGU | 172 | ACAAATTG GGCTAGCTACAACGA ATTTACAT | 1493 |
| 765 | UAAAUACA A UUUGUACU | 173 | AGTACAAA GGCTAGCTACAACGA TGTATTTA | 1494 |
| 769 | UACAAUUU G UACUUUUU | 174 | AAAAAGTA GGCTAGCTACAACGA AAATTGTA | 1495 |
| 771 | CAAUUUCU A CUUUUUUC | 175 | GAAAAAAG GGCTAGCTACAACGA ACAAATTG | 1496 |
| 785 | UUCUUAAG G CAUACUAG | 176 | CTAGTATG GGCTAGCTACAACGA CTTAAGAA | 1497 |
| 787 | CUUAAGCC A UACUAGUA | 177 | TACTAGTA GGCTAGCTACAACGA GCCTTAAG | 1498 |
| 789 | UAAGGCAU A CUAGUACA | 178 | TGTACTAG GGCTAGCTACAACGA ATGCCTTA | 1499 |
| 793 | GCAUACUA G UACAAGUG | 179 | CACTTCTA GGCTAGCTACAACGA TAGTATGC | 1500 |
| 795 | AUACUAGU A CAAGUGGU | 180 | ACCACTTG GGCTAGCTACAACGA ACTACTAT | 1501 |
| 799 | UAGUACAA G UGGUAAUU | 181 | AATTACCA GGCTAGCTACAACGA TTGTACTA | 1502 |
| 802 | UACAAGUG G UAAUUUUU | 182 | AAAAATTA GGCTAGCTACAACGA CACTTGTA | 1503 |
| 805 | AAGUGCUA A UUUUUGUA | 183 | TACAAAAA GGCTAGCTACAACGA TACCACTT | 1504 |
| 811 | UAAUUUUU G UACAUUAC | 184 | GTAATGTA GGCTAGCTACAACGA AAAAATTA | 1505 |
| 813 | AUUUUUGU A CAUUACAC | 185 | GTGTAATG GGCTAGCTACAACGA ACAAAAAT | 1506 |
| 815 | UUUUGUAC A UUACACUA | 186 | TAGTGTAA GGCTAGCTACAACGA GTACAAAA | 1507 |
| 818 | UGUACAUU A CACUAAAU | 187 | ATTTAGTG GGCTAGCTACAACGA AATGTACA | 1508 |
| 820 | UACAUUAC A CUAAAUUA | 188 | TAATTTAG GGCTAGCTACAACGA GTAATGTA | 1509 |
| 825 | UACACUAA A UUAUUAGC | 189 | GCTAATAA GGCTAGCTACAACGA TTAGTGTA | 1510 |
| 828 | ACUAAAUU A UUAGCAUU | 190 | AATGCTAA GGCTAGCTACAACGA AATTTAGT | 1511 |
| 832 | AAUUAUUA G CAUUUGUU | 191 | AACAAATG GGCTAGCTACAACGA TAATAATT | 1512 |
| 834 | UUAUUAGC A UUUGUUUU | 192 | AAAACAAA GGCTAGCTACAACGA GCTAATAA | 1513 |
| 838 | UAGCAUUU G UUUUAGCA | 193 | TGCTAAAA GGCTAGCTACAACGA AAATGCTA | 1514 |
| 844 | UUGUUUUA G CAUUACCU | 194 | AGGTAATG GGCTAGCTACAACGA TAAAACAA | 1515 |
| 846 | GUUUUAGC A UUACCUAA | 195 | TTAGGTAA GGCTAGCTACAACGA GCTAAAAC | 1516 |
| 849 | UUACCAUU A CCUAAUUU | 196 | AAATTAGG GGCTAGCTACAACGA AATGCTAA | 1517 |
| 854 | AUUACCUA A UUUUUUUC | 197 | GAAAAAAA GGCTAGCTACAACGA TAGGTAAT | 1518 |
| 865 | UUUUUCCU G CUCCAUGC | 198 | GCATGGAG GGCTAGCTACAACGA AGGAAAAA | 1519 |
| 870 | CCUGCUCC A UGCAGACU | 199 | AGTCTGCA GGCTAGCTACAACGA GGAGCAGG | 1520 |
| 872 | UGCUCCAU G CAGACUGU | 200 | ACAGTCTG GGCTAGCTACAACGA ATGGAGCA | 1521 |
| 876 | CCAUGCAG A CUGUUAGC | 201 | GCTAACAG GGCTAGCTACAACGA CTGCATGG | 1522 |
| 879 | UGCAGACU G UUAGCUUU | 202 | AAAGCTAA GGCTAGCTACAACGA AGTCTGCA | 1523 |
| 883 | GACUGUUA G CUUUUACC | 203 | GGTAAAAG GGCTAGCTACAACGA TAACAGTC | 1524 |
| 889 | UAGCUUUU A CCUUAAAU | 204 | ATTTAAGG GGCTAGCTACAACGA AAAAGCTA | 1525 |
| 896 | UACCUUAA A UGCUUAUU | 205 | AATAAGCA GGCTAGCTACAACGA TTAAGGTA | 1526 |
| 898 | CCUUAAAU G CUUAUUUU | 206 | AAAATAAG GGCTAGCTACAACGA ATTTAAGG | 1527 |
| 902 | AAAUGCUU A UUUUAAAA | 207 | TTTTAAAA GGCTAGCTACAACGA AAGCATTT | 1528 |
| 910 | AUUUUAAA A UGACAGUG | 208 | CACTGTCA GGCTAGCTACAACGA TTTAAAAT | 1529 |
| 913 | UUAAAAUG A CAGUGGAA | 209 | TTCCACTG GGCTAGCTACAACGA CATTTTAA | 1530 |
| 916 | AAAUGACA G UGGAAGUU | 210 | AACTTGGA GGCTAGCTACAACGA TGTCATTT | 1531 |
| 922 | CAGUGGAA G UUUUUUUU | 211 | AAAAAAAA GGCTAGCTACAACGA TTCCACTG | 1532 |
| 939 | UCCUCGAA G UGCCAGUA | 212 | TACTGGCA GGCTAGCTACAACGA TTCGAGGA | 1533 |
| 941 | CUCGAAGU G CCAGUAUU | 213 | AATACTGG GGCTAGCTACAACGA ACTTCGAG | 1534 |
| 945 | AAGUGCCA G UAUUCCCA | 214 | TGGCAATA GGCTAGCTACAACGA TGGCACTT | 1535 |
| 947 | GUGCCAGU A UUCCCAGA | 215 | TCTGGGAA GGCTAGCTACAACGA ACTGGCAC | 1536 |
| 956 | UUCCCAGA G UUUUGGUU | 216 | AACCAAAA GGCTAGCTACAACGA TCTGGGAA | 1537 |
| 962 | GAGUUUUG G UUUUUGAA | 217 | TTCAAAAA GGCTAGCTACAACGA CAAAACTC | 1538 |
| 970 | GUUUUUGA A CUAGCAAU | 218 | ATTGCTAG GGCTAGCTACAACGA TCAAAAAC | 1539 |
| 974 | UUGAACUA G CAAUGCCU | 219 | AGCCATTG GGCTAGCTACAACGA TAGTTCAA | 1540 |
| 977 | AACUAGCA A UGCCUGUG | 220 | CACAGGCA GGCTAGCTACAACGA TGCTAGTT | 1541 |
| 979 | CUAGCAAU G CCUGUGAA | 221 | TTCACAGG GGCTAGCTACAACGA ATTGCTAG | 1542 |
| 983 | CAAUGCCU G UGAAAAAG | 222 | CTTTTTCA GGCTAGCTACAACGA AGGCATTG | 1543 |
| 994 | AAAAAGAA A CUGAAUAC | 223 | GTATTCAG GGCTAGCTACAACGA TTCTTTTT | 1544 |
| 999 | GAAACUGA A UACCUAAG | 224 | CTTAGGTA GGCTAGCTACAACGA TCAGTTTC | 1545 |
| 1001 | AACUGAAU A CCUAAGAU | 225 | ATCTTAGG GGCTACCTACAACGA ATTCAGTT | 1546 |
| 1008 | UACCUAAG A UUUCUGUC | 226 | GACAGAAA GGCTAGCTACAACGA CTTAGGTA | 1547 |
| 1014 | AGAUUUCU G UCUUGGGG | 227 | CCCCAAGA GGCTAGCTACAACGA AGAAATCT | 1548 |
| 1022 | GUCUUGGG G UUUUUGGU | 228 | ACCAAAAA GGCTAGCTACAACGA CCCAAGAC | 1549 |
| 1029 | GGUUUUUG G UGCAUGCA | 229 | TGCATGCA GGCTAGCTACAACGA CAAAAACC | 1550 |
| 1031 | UUUUUGGU G CAUGCAGU | 230 | ACTGCATG GGCTAGCTACAACGA ACCAAAAA | 1551 |
| 1033 | UUUGGUGC A UGCAGUUG | 231 | CAACTGCA GGCTAGCTACAACGA GCACCAAA | 1552 |
| 1035 | UGGUGCAU G CAGUUGAU | 232 | ATCAACTG GGCTAGCTACAACGA ATGCACCA | 1553 |
| 1038 | UGCAUGCA G UUGAUUAC | 233 | GTAATCAA GGCTAGCTACAACGA TGCATGCA | 1554 |
| 1042 | UGCAGUUG A UUACUUCU | 234 | AGAACTAA GGCTAGCTACAACGA CAACTGCA | 1555 |
| 1045 | AGUUGAUU A CUUCUUAU | 235 | ATAAGAAG GGCTAGCTACAACGA AATCAACT | 1556 |
| 1052 | UACUUCUU A UUUUUCUU | 236 | AAGAAAAA GGCTAGCTACAACGA AAGAAGTA | 1557 |
| 1061 | UUUUUCUU A CCAACUCU | 237 | ACACTTGG GGCTAGCTACAACGA AAGAAAAA | 1558 |
| 1066 | CUUACCAA G UGUCAAUG | 238 | CATTCACA GGCTAGCTACAACGA TTGGTAAG | 1559 |
| 1068 | UACCAAGU G UGAAUGUU | 239 | AACATTCA GGCTAGCTACAACGA ACTTGGTA | 1560 |
| 1072 | AAGUGUGA A UGUUGGUG | 240 | CACCAACA GGCTAGCTACAACGA TCACACTT | 1561 |
| 1074 | GUGUGAAU G UUGGUGUG | 241 | CACACCAA GGCTAGCTACAACGA ATTCACAC | 1562 |
| 1078 | GAAUGUUG G UGUGAAAC | 242 | CTTTCACA GGCTAGCTACAACGA CAACATTC | 1563 |
| 1080 | AUGUUGGU G UGAAACAA | 243 | TTGTTTCA GGCTAGCTACAACGA ACCAACAT | 1564 |
| 1085 | GGUGUGAA A CAAAUUAA | 244 | TTAATTTG GGCTAGCTACAACGA TTCACACC | 1565 |
| 1089 | UGAAACAA A UUAAUCAA | 245 | TTCATTAA GGCTAGCTACAACGA TTGTTTCA | 1566 |
| 1093 | ACAAAUUA A UGAAGCUU | 246 | AAGCTTCA GGCTAGCTACAACGA TAATTTGT | 1567 |
| 1098 | UUAAUGAA G CUUUUGAA | 247 | TTCAAAAG GGCTAGCTACAACGA TTCATTAA | 1568 |
| 1106 | GCUUUUCA A UCAUCCCU | 248 | AGGGATGA GGCTAGCTACAACGA TCAAAAGC | 1569 |
| 1109 | UUUGAAUC A UCCCUAUU | 249 | AATAGGGA GGCTAGCTACAACGA GATTCAAA | 1570 |
| 1115 | UCAUCCCU A UUCUGUCU | 250 | ACACAGAA GGCTAGCTACAACGA AGGGATGA | 1571 |
| 1120 | CCUAUUCU G UGUUUUAU | 251 | ATAAAACA GGCTAGCTACAACGA AGAATAGG | 1572 |
| 1122 | UAUUCUGU G UUUUAUCU | 252 | AGATAAAA GGCTAGCTACAACGA ACAGAATA | 1573 |
| 1127 | UGUGUUUU A UCUAGUCA | 253 | TGACTAGA GGCTAGCTACAACGA AAAACACA | 1574 |
| 1132 | UUUAUCUA G UCACAUAA | 254 | TTATGTGA GGCTAGCTACAACGA TAGATAAA | 1575 |
| 1135 | AUCUAGUC A CAUAAAUG | 255 | CATTTATG GGCTAGCTACAACGA GACTAGAT | 1576 |
| 1137 | CUAGUCAC A UAAAUGGA | 256 | TCCATTTA GGCTAGCTACAACGA GTGACTAG | 1577 |
| 1141 | UCACAUAA A UGGAUUAA | 257 | TTAATCCA GGCTAGCTACAACGA TTATGTGA | 1578 |
| 1145 | AUAAAUGG A UUAAUUAC | 258 | GTAATTAA GGCTAGCTACAACGA CCATTTAT | 1579 |
| 1149 | AUGGAUUA A UUACUAAU | 259 | ATTAGTAA GGCTAGCTACAACGA TAATCCAT | 1580 |
| 1152 | GAUUAAUU A CUAAUUUC | 260 | GAAATTAG GGCTAGCTACAACGA AATTAATC | 1581 |
| 1156 | AAUUACUA A UUUCAGUU | 261 | AACTGAAA GGCTAGCTACAACGA TAGTAATT | 1582 |
| 1162 | UAAUUUCA G UUGAGACC | 262 | GGTCTCAA GGCTAGCTACAACGA TGAAATTA | 1583 |
| 1168 | CAGUUGAG A CCUUCUAA | 263 | TTAGAAGG GGCTAGCTACAACGA CTCAACTG | 1584 |
| 1176 | ACCUUCUA A UUGGUUUU | 264 | AAAACCAA GGCTAGCTACAACGA TAGAAGGT | 1585 |
| 1180 | UCUAAUUC G UUUUUACU | 265 | AGTAAAAA GGCTAGCTACAACGA CAATTAGA | 1586 |
| 1186 | UGGUUUUU A CUGAAACA | 266 | TGTTTCAG GGCTAGCTACAACGA AAAAACCA | 1587 |
| 1192 | UUACUGAA A CAUUGAGG | 267 | CCTCAATG GGCTAGCTACAACGA TTCAGTAA | 1588 |
| 1194 | ACUGAAAC A UUGAGGGA | 268 | TCCCTCAA GGCTAGCTACAACGA GTTTCAGT | 1589 |
| 1202 | AUUGAGGG A CACAAAUU | 269 | AATTTGTG GGCTAGCTACAACGA CCCTCAAT | 1590 |
| 1204 | UGAGGGAC A CAAAUUUA | 270 | TAAATTTG GGCTAGCTACAACGA GTCCCTCA | 1591 |
| 1208 | GGACACAA A UUUAUGGG | 271 | CCCATAAA GGCTAGCTACAACGA TTGTGTCC | 1592 |
| 1212 | ACAAAUUU A UGGGCUUC | 272 | GAAGCCCA GGCTAGCTACAACGA AAATTTGT | 1593 |
| 1216 | AUUUAUGG G CUUCCUGA | 273 | TCAGGAAG GGCTAGCTACAACGA CCATAAAT | 1594 |
| 1224 | GCUUCCUG A UGAUGAUU | 274 | AATCATCA GGCTAGCTACAACGA CAGGAAGC | 1595 |
| 1227 | UCCUCAUG A UGAUUCUU | 275 | AAGAATCA GGCTAGCTACAACGA CATCAGGA | 1596 |
| 1230 | UGAUGAUG A UUCUUCUA | 276 | TAGAAGAA GGCTAGCTACAACGA CATCATCA | 1597 |
| 1240 | UCUUCUAG G CAUCAUGU | 277 | ACATGATG GGCTAGCTACAACGA CTAGAAGA | 1598 |
| 1242 | UUCUAGCC A UCAUGUCC | 278 | GGACATGA GGCTAGCTACAACGA GCCTAGAA | 1599 |
| 1245 | UAGGCAUC A UGUCCUAU | 279 | ATAGGACA GGCTAGCTACAACGA GATGCCTA | 1600 |
| 1247 | GGCAUCAU G UCCUAUAG | 280 | CTATAGGA GGCTAGCTACAACGA ATGATGCC | 1601 |
| 1252 | CAUGUCCU A UACUUUGU | 281 | ACAAACTA GGCTAGCTACAACGA AGGACATG | 1602 |
| 1255 | GUCCUAUA G UUUGUCAU | 282 | ATGACAAA GGCTAGCTACAACGA TATAGGAC | 1603 |
| 1259 | UAUAGUUU G UCAUCCCU | 283 | AGGGATCA GGCTAGCTACAACGA AAACTATA | 1604 |
| 1262 | AGUUUGUC A UCCCUGAU | 284 | ATCAGGGA GGCTAGCTACAACGA GACAAACT | 1605 |
| 1269 | CAUCCCUG A UGAAUGUA | 285 | TACATTCA GGCTAGCTACAACGA CAGGGATG | 1606 |
| 1273 | CCUGAUGA A UGUAAAGU | 286 | ACTTTACA GGCTAGCTACAACGA TCATCAGG | 1607 |
| 1275 | UGAUGAAU G UAAAGUUA | 287 | TAACTTTA GGCTAGCTACAACGA ATTCATCA | 1608 |
| 1280 | AAUGUAAA G UUACACUG | 288 | CAGTGTAA GGCTAGCTACAACGA TTTACATT | 1609 |
| 1283 | GUAAAGUU A CACUGUUC | 289 | GAACAGTG GGCTAGCTACAACGA AACTTTAC | 1610 |
| 1285 | AAAGUUAC A CUGUUCAC | 290 | GTGAACAG GGCTAGCTACAACGA GTAACTTT | 1611 |
| 1288 | GUUACACU G UUCACAAA | 291 | TTTGTGAA GGCTAGCTACAACGA AGTGTAAC | 1612 |
| 1292 | CACUGUUC A CAAAGGUU | 292 | AACCTTTG GGCTAGCTACAACGA GAACAGTG | 1613 |
| 1298 | UCACAAAG G UUUUGUGU | 293 | AGACAAAA GGCTAGCTACAACGA CTTTGTGA | 1614 |
| 1303 | AAGGUUUU G UCUCCUUU | 294 | AAAGGAGA GGCTAGCTACAACGA AAAACCTT | 1615 |
| 1314 | UCCUUUCC A CUGCUAUU | 295 | AATAGCAG GGCTAGCTACAACGA GGAAAGGA | 1616 |
| 1317 | UUUCCACU G CUAUUAGU | 296 | ACTAATAG GGCTAGCTACAACGA AGTGGAAA | 1617 |
| 1320 | CCACUGCU A UUAGUCAU | 297 | ATGACTAA GGCTAGCTACAACGA AGCAGTGG | 1618 |
| 1324 | UGCUAUUA G UCAUGGUC | 298 | GACCATGA GGCTAGCTACAACGA TAATAGCA | 1619 |
| 1327 | UAUUAGUC A UGGUCACU | 299 | AGTGACCA GGCTAGCTACAACGA GACTAATA | 1620 |
| 1330 | UAGUCAUG G UCACUCUC | 300 | GAGAGTGA GGCTAGCTACAACGA CATGACTA | 1621 |
| 1333 | UCAUGGUC A CUCUCCCC | 301 | GGGGAGAG GGCTAGCTACAACGA GACCATGA | 1622 |
| 1345 | UCCCCAAA A UAUUAUAU | 302 | ATATAATA GGCTAGCTACAACGA TTTGGGGA | 1623 |
| 1347 | CCCAAAAU A UUAUAUUU | 303 | AAATATAA GGCTAGCTACAACGA ATTTTGGG | 1624 |
| 1350 | AAAAUAUU A UAUUUUUU | 304 | AAAAAATA GGCTAGCTACAACGA AATATTTT | 1625 |
| 1352 | AAUAUUAU A UUUUUUUU | 305 | AGAAAAAA GGCTAGCTACAACGA ATAATATT | 1626 |
| 1361 | UUUUUUCU A UAAAAAGA | 306 | TCTTTTTA GGCTAGCTACAACGA AGAAAAAA | 1627 |
| 1375 | AGAAAAAA A UGGAAAAA | 307 | TTTTTCCA GGCTAGCTACAACGA TTTTTTCT | 1628 |
| 1385 | GGAAAAAA A UUACAAGG | 308 | CCTTGTAA GGCTAGCTACAACGA TTTTTTCC | 1629 |
| 1388 | AAAAAAUU A CAAGGCAA | 309 | TTGCCTTG GGCTAGCTACAACGA AATTTTTT | 1630 |
| 1393 | AUUACAAG G CAAUGGAA | 310 | TTCCATTG GGCTAGCTACAACGA CTTGTAAT | 1631 |
| 1396 | ACAAGGCA A UGGAAACU | 311 | AGTTTCCA GGCTAGCTACAACGA TGCCTTGT | 1632 |
| 1402 | CAAUGGAA A CUAUUAUA | 312 | TATAATAG GGCTAGCTACAACGA TTCCATTG | 1633 |
| 1405 | UGGAAACU A UUAUAAGG | 313 | CCTTATAA GGCTAGCTACAACGA AGTTTCCA | 1634 |
| 1408 | AAACUAUU A UAAGGCCA | 314 | TGGCCTTA GGCTAGCTACAACGA AATAGTTT | 1635 |
| 1413 | AUUAUAAG G CCAUUUCC | 315 | GGAAATGG GGCTAGCTACAACGA CTTATAAT | 1636 |
| 1416 | AUAAGGCC A UUUCCUUU | 316 | AAAGGAAA GGCTAGCTACAACGA GGCCTTAT | 1637 |
| 1427 | UCCUUUUC A CAUUAGAU | 317 | ATCTAATG GGCTAGCTACAACGA GAAAAGGA | 1638 |
| 1429 | CUUUUCAC A UUAGAUAA | 318 | TTATCTAA GGCTAGCTACAACGA GTGAAAAG | 1639 |
| 1434 | CACAUUAG A UAAAUUAC | 319 | GTAATTTA GGCTAGCTACAACGA CTAATGTG | 1640 |
| 1438 | UUAGAUAA A UUACUAUA | 320 | TATAGTAA GGCTAGCTACAACGA TTATCTAA | 1641 |
| 1441 | GAUAAAUU A CUAUAAAG | 321 | CTTTATAG GGCTAGCTACAACGA AATTTATC | 1642 |
| 1444 | AAAUUACU A UAAAGACU | 322 | AGTCTTTA GGCTAGCTACAACGA AGTAATTT | 1643 |
| 1450 | CUAUAAAG A CUCCUAAU | 323 | ATTAGGAG GGCTAGCTACAACGA CTTTATAG | 1644 |
| 1457 | GACUCCUA A UAGCUUUU | 324 | AAAAGCTA GGCTAGCTACAACGA TAGGAGTC | 1645 |
| 1460 | UCCUAAUA G CUUUUUCC | 325 | GGAAAAAG GGCTAGCTACAACGA TATTAGGA | 1646 |
| 1470 | UUUUUCCU G UUAAGGCA | 326 | TGCCTTAA GGCTAGCTACAACGA AGGAAAAA | 1647 |
| 1476 | CUGUUAAG G CAGACCCA | 327 | TGGGTCTG GGCTAGCTACAACGA CTTAACAG | 1648 |
| 1480 | UAAGGCAG A CCCAGUAU | 328 | ATACTGGG GGCTAGCTACAACGA CTGCCTTA | 1649 |
| 1485 | CAGACCCA G UAUGAAUG | 329 | CATTCATA GGCTAGCTACAACGA TGGGTCTG | 1650 |
| 1487 | GACCCAGU A UGAAUGGG | 330 | CCCATTCA GGCTAGCTACAACGA ACTGGGTC | 1651 |
| 1491 | CAGUAUGA A UGGGAUUA | 331 | TAATCCCA GGCTAGCTACAACGA TCATACTG | 1652 |
| 1496 | UGAAUGGG A UUAUUAUA | 332 | TATAATAA GGCTAGCTACAACGA CCCATTCA | 1653 |
| 1499 | AUGGGAUU A UUAUAGCA | 333 | TGCTATAA GGCTAGCTACAACGA AATCCCAT | 1654 |
| 1502 | GGAUUAUU A UAGCAACC | 334 | GGTTGCTA GGCTAGCTACAACGA AATAATCC | 1655 |
| 1505 | UUAUUAUA G CAACCAUU | 335 | AATGGTTG GGCTAGCTACAACGA TATAATAA | 1656 |
| 1508 | UUAUAGCA A CCAUUUUG | 336 | CAAAATGG GGCTAGCTACAACGA TGCTATAA | 1657 |
| 1511 | UAGCAACC A UUUUGGGG | 337 | CCCCAAAA GGCTAGCTACAACGA GGTTGCTA | 1658 |
| 1519 | AUUUUGGG G CUAUAUUU | 338 | AAATATAG GGCTAGCTACAACGA CCCAAAAT | 1659 |
| 1522 | UUGGGGCU A UAUUUACA | 339 | TGTAAATA GGCTAGCTACAACGA ACCCCCAA | 1660 |
| 1524 | GGGGCUAU A UUUACAUG | 340 | CATGTAAA GGCTAGCTACAACGA ATAGCCCC | 1661 |
| 1528 | CUAUAUUU A CAUGCUAC | 341 | GTACCATG GGCTAGCTACAACGA AAATATAG | 1662 |
| 1530 | AUAUUUAC A UGCUACUA | 342 | TAGTAGCA GGCTAGCTACAACGA GTAAATAT | 1663 |
| 1532 | AUUUACAU G CUACUAAA | 343 | TTTAGTAG GGCTAGCTACAACGA ATGTAAAT | 1664 |
| 1535 | UACAUGCU A CUAAAUUU | 344 | AAATTTAG GGCTAGCTACAACGA AGCATGTA | 1665 |
| 1540 | GCUACUAA A UUUUUAUA | 345 | TATAAAAA GGCTAGCTACAACGA TTAGTAGC | 1666 |
| 1546 | AAAUUUUU A UAAUAAUU | 346 | AATTATTA GGCTAGCTACAACGA AAAAATTT | 1667 |
| 1549 | UUUUUAUA A UAAUUGAA | 347 | TTCAATTA GGCTAGCTACAACGA TATAAAAA | 1668 |
| 1552 | UUAUAAUA A UUCAAAAG | 348 | CTTTTCAA GGCTAGCTACAACGA TATTATAA | 1669 |
| 1561 | UUGAAAAG A UUUUAACA | 349 | TGTTAAAA GGCTAGCTACAACGA CTTTTCAA | 1670 |
| 1567 | AGAUUUUA A CAAGUAUA | 350 | TATACTTG GGCTAGCTACAACGA TAAAATCT | 1671 |
| 1571 | UUUAACAA G UAUAAAAA | 351 | TTTTTATA GGCTAGCTACAACGA TTGTTAAA | 1672 |
| 1573 | UAACAAGU A UAAAAAAA | 352 | TTTTTTTA GGCTAGCTACAACGA ACTTGTTA | 1673 |
| 1581 | AUAAAAAA A UUCUCAUA | 353 | TATGAGAA GGCTAGCTACAACGA TTTTTTAT | 1674 |
| 1587 | AAAUUCUC A UAGGAAUU | 354 | AATTCCTA GGCTAGCTACAACGA GAGAATTT | 1675 |
| 1593 | UCAUAGGA A UUAAAUGU | 355 | ACATTTAA GGCTAGCTACAACGA TCCTATGA | 1676 |
| 1598 | GGAAUUAA A UGUAGUCU | 356 | AGACTACA GGCTAGCTACAACGA TTAATTCC | 1677 |
| 1600 | AAUUAAAU G UAGUCUCC | 357 | GGAGACTA GGCTAGCTACAACGA ATTTAATT | 1678 |
| 1603 | UAAAUGUA G UCUCCCUC | 358 | CAGGGAGA GGCTAGCTACAACGA TACATTTA | 1679 |
| 1611 | GUCUCCCU G UGUCAGAC | 359 | GTCTGACA GGCTAGCTACAACGA AGGGAGAC | 1680 |
| 1613 | CUCCCUGU G UCAGACUG | 360 | CAGTCTGA GGCTAGCTACAACGA ACAGGGAG | 1681 |
| 1618 | UGUGUCAG A CUGCUCUU | 361 | AAGAGCAG GGCTAGCTACAACGA CTGACACA | 1682 |
| 1621 | GUCAGACU G CUCUUUCA | 362 | TGAAAGAG GGCTAGCTACAACGA AGTCTGAC | 1683 |
| 1629 | GCUCUUUC A UAGUAUAA | 363 | TTATACTA GGCTAGCTACAACGA GAAAGAGC | 1684 |
| 1632 | CUUUCAUA G UAUAACUU | 364 | AAGTTATA GGCTAGCTACAACGA TATGAAAG | 1685 |
| 1634 | UUCAUAGU A UAACUUUA | 365 | TAAAGTTA GGCTAGCTACAACGA ACTATGAA | 1686 |
| 1637 | AUAGUAUA A CUUUAAAU | 366 | ATTTAAAG GGCTAGCTACAACGA TATACTAT | 1687 |
| 1644 | AACUUUAA A UCUUUUCU | 367 | AGAAAAGA GGCTAGCTACAACGA TTAAAGTT | 1688 |
| 1656 | UUUCUUCA A CUUGAGUC | 368 | GACTCAAG GGCTAGCTACAACGA TGAAGAAA | 1689 |
| 1662 | CAACUUGA G UCUUUGAA | 369 | TTCAAAGA GGCTAGCTACAACGA TCAAGTTG | 1690 |
| 1672 | CUUUGAAG A UAGUUUUA | 370 | TAAAACTA GGCTAGCTACAACGA CTTCAAAG | 1691 |
| 1675 | UGAAGAUA G UUUUAAUU | 371 | AATTAAAA GGCTAGCTACAACGA TATCTTCA | 1692 |
| 1681 | UAGUUUUA A UUCUGCUU | 372 | AAGCAGAA GGCTAGCTACAACGA TAAAACTA | 1693 |
| 1686 | UUAAUUCU G CUUGUCAC | 373 | GTCACAAG GGCTAGCTACAACGA AGAATTAA | 1694 |
| 1690 | UUCUGCUU G UGACAUUA | 374 | TAATGTCA GGCTAGCTACAACGA AAGCAGAA | 1695 |
| 1693 | UGCUUGUG A CAUUAAAA | 375 | TTTTAATG GGCTAGCTACAACGA CACAAGCA | 1696 |
| 1695 | CUUGUGAC A UUAAAAGA | 376 | TCTTTTAA GGCTAGCTACAACGA GTCACAAG | 1697 |
| 1703 | AUUAAAAG A UUAUUUGG | 377 | CCAAATAA GGCTAGCTACAACGA CTTTTAAT | 1698 |
| 1706 | AAAAGAUU A UUUGGGCC | 378 | GGCCCAAA GGCTAGCTACAACGA AATCTTTT | 1699 |
| 1712 | UUAUUUGG G CCAGUUAU | 379 | ATAACTGG GGCTAGCTACAACGA CCAAATAA | 1700 |
| 1716 | UUGGGCCA G UUAUAGCU | 380 | AGCTATAA GGCTAGCTACAACGA TGGCCCAA | 1701 |
| 1719 | GGCCAGUU A UAGCUUAU | 381 | ATAAGCTA GGCTAGCTACAACGA AACTGGCC | 1702 |
| 1722 | CAGUUAUA G CUUAUUAG | 382 | CTAATAAG GGCTAGCTACAACGA TATAACTG | 1703 |
| 1726 | UAUAGCUU A UUAGGUGU | 383 | ACACCTAA GGCTAGCTACAACGA AAGCTATA | 1704 |
| 1731 | CUUAUUAG G UGUUGAAG | 384 | CTTCAACA GGCTAGCTACAACGA CTAATAAG | 1705 |
| 1733 | UAUUAGGU G UUGAAGAG | 385 | CTCTTCAA GGCTAGCTACAACGA ACCTAATA | 1706 |
| 1742 | UUGAAGAG A CCAAGGUU | 386 | AACCTTGG GGCTAGCTACAACGA CTCTTCAA | 1707 |
| 1748 | AGACCAAG G UUGCAAGC | 387 | GCTTGCAA GGCTAGCTACAACGA CTTGGTCT | 1708 |
| 1751 | CCAAGGUU G CAAGCCAG | 388 | CTCGCTTG GGCTAGCTACAACGA AACCTTGG | 1709 |
| 1755 | GGUUGCAA G CCAGGCCC | 389 | GGCCCTCG GGCTAGCTACAACGA TTCCAACC | 1710 |
| 1760 | CAACCCAG G CCCUGUGU | 390 | ACACACCG GGCTAGCTACAACGA CTGCCTTG | 1711 |
| 1765 | CAGGCCCU G UGUGAACC | 391 | CGTTCACA GGCTAGCTACAACGA AGGGCCTG | 1712 |
| 1767 | GGCCCUGU G UGAACCUU | 392 | AAGGTTCA GGCTAGCTACAACGA ACAGGGCC | 1713 |
| 1771 | CUGUGUGA A CCUUGAGC | 393 | GCTCAAGG GGCTAGCTACAACGA TCACACAG | 1714 |
| 1778 | AACCUUGA G CUUUCAUA | 394 | TATGAAAG GGCTAGCTACAACGA TCAAGGTT | 1715 |
| 1784 | GAGCUUUC A UAGAGAGU | 395 | ACTCTCTA GGCTAGCTACAACGA GAAAGCTC | 1716 |
| 1791 | CAUAGAGA G UUUCACAG | 396 | CTGTGAAA GGCTAGCTACAACGA TCTCTATG | 1717 |
| 1796 | AGAGUUUC A CAGCAUGG | 397 | CCATGCTC GGCTAGCTACAACGA GAAACTCT | 1718 |
| 1799 | GUUUCACA G CAUGGACU | 398 | AGTCCATC GGCTAGCTACAACGA TGTGAAAC | 1719 |
| 1801 | UUCACAGC A UGGACUGU | 399 | ACAGTCCA GGCTAGCTACAACGA GCTGTGAA | 1720 |
| 1805 | CAGCAUGG A CUGUGUGC | 400 | GCACACAG GGCTAGCTACAACGA CCATGCTC | 1721 |
| 1808 | CAUGGACU G UGUGCCCC | 401 | GGGGCACA GGCTAGCTACAACGA AGTCCATG | 1722 |
| 1810 | UGGACUCU G UGCCCCAC | 402 | GTGGGGCA GGCTAGCTACAACGA ACAGTCCA | 1723 |
| 1812 | GACUGUGU G CCCCACGG | 403 | CCGTGGGG GGCTAGCTACAACGA ACACAGTC | 1724 |
| 1817 | UGUGCCCC A CGGUCAUC | 404 | GATGACCG GGCTAGCTACAACGA GGGGCACA | 1725 |
| 1820 | GCCCCACG G UCAUCCGA | 405 | TCGGATGA GGCTAGCTACAACGA CGTGGGGC | 1726 |
| 1823 | CCACGGUC A UCCGAGUG | 406 | CACTCGGA GGCTAGCTACAACGA GACCGTGG | 1727 |
| 1829 | UCAUCCGA G UGGUUGUA | 407 | TACAACCA GGCTAGCTACAACGA TCGGATGA | 1728 |
| 1832 | UCCGAGUG G UUGUACGA | 408 | TCGTACAA GGCTAGCTACAACGA CACTCGGA | 1729 |
| 1835 | GAGUGGUU G UACGAUGC | 409 | GCATCGTA GGCTAGCTACAACGA AACCACTC | 1730 |
| 1837 | GUGGUUGU A CGAUGCAU | 410 | ATGCATCG GGCTAGCTACAACGA ACAACCAC | 1731 |
| 1840 | GUUGUACG A UGCAUUGG | 411 | CCAATGCA GGCTAGCTACAACGA CGTACAAC | 1732 |
| 1842 | UGUACGAU G CAUUGCUU | 412 | AACCAATG GGCTAGCTACAACGA ATCGTACA | 1733 |
| 1844 | UACGAUGC A UUGGUUAG | 413 | CTAACCAA GGCTAGCTACAACGA GCATCGTA | 1734 |
| 1848 | AUGCAUUG G UUAGUCAA | 414 | TTGACTAA GGCTAGCTACAACGA CAATGCAT | 1735 |
| 1852 | AUUGGUUA G UCAAAAAU | 415 | ATTTTTGA GGCTAGCTACAACGA TAACCAAT | 1736 |
| 1859 | AGUCAAAA A UGGGGAGG | 416 | CCTCCCCA GGCTAGCTACAACGA TTTTGACT | 1737 |
| 1869 | GGGGAGGG A CUAGGGCA | 417 | TGCCCTAG GGCTAGCTACAACGA CCCTCCCC | 1738 |
| 1875 | GGACUAGG G CAGUUUGG | 418 | CCAAACTC GGCTAGCTACAACGA CCTACTCC | 1739 |
| 1878 | CUAGGGCA G UUUGGAUA | 419 | TATCCAAA GGCTAGCTACAACGA TGCCCTAC | 1740 |
| 1884 | CAGUUUGG A UAGCUCAA | 420 | TTGACCTA GGCTAGCTACAACGA CCAAACTG | 1741 |
| 1887 | UUUGGAUA G CUCAACAA | 421 | TTGTTGAG GGCTAGCTACAACGA TATCCAAA | 1742 |
| 1892 | AUAGCUCA A CAAGAUAC | 422 | GTATCTTG GGCTAGCTACAACGA TGAGCTAT | 1743 |
| 1897 | UCAACAAG A UACAAUCU | 423 | AGATTGTA GGCTAGCTACAACGA CTTGTTGA | 1744 |
| 1899 | AACAAGAU A CAAUCUCA | 424 | TGAGATTC GGCTAGCTACAACGA ATCTTGTT | 1745 |
| 1902 | AAGAUACA A UCUCACUC | 425 | GAGTGAGA GGCTAGCTACAACGA TGTATCTT | 1746 |
| 1907 | ACAAUCUC A CUCUGUGG | 426 | CCACAGAG GGCTAGCTACAACGA GAGATTGT | 1747 |
| 1912 | CUCACUCU G UGGUGGUC | 427 | GACCACCA GGCTAGCTACAACGA AGAGTGAG | 1748 |
| 1915 | ACUCUGUG G UGGUCCUG | 428 | CAGGACCA GGCTAGCTACAACGA CACAGAGT | 1749 |
| 1918 | CUGUGGUG G UCCUGCUG | 429 | CAGCAGGA GGCTAGCTACAACGA CACCACAG | 1750 |
| 1923 | GUGGUCCU G CUGACAAA | 430 | TTTGTCAG GGCTAGCTACAACGA AGGACCAC | 1751 |
| 1927 | UCCUGCUG A CAAAUCAA | 431 | TTGATTTG GGCTAGCTACAACGA CAGCAGGA | 1752 |
| 1931 | GCUGACAA A UCAAGAGC | 432 | GCTCTTGA GGCTAGCTACAACGA TTGTCAGC | 1753 |
| 1938 | AAUCAAGA G CAUUGCUU | 433 | AAGCAATG GGCTAGCTACAACGA TCTTGATT | 1754 |
| 1940 | UCAAGAGC A UUGCUUUU | 434 | AAAAGCAA GGCTAGCTACAACGA GCTCTTGA | 1755 |
| 1943 | AGAGCAUU G CUUUUGUU | 435 | AACAAAAG GGCTAGCTACAACGA AATGCTCT | 1756 |
| 1949 | UUCCUUUU G UUUCUUAA | 436 | TTAAGAAA GGCTAGCTACAACGA AAAAGCAA | 1757 |
| 1962 | UUAAGAAA A CAAACUCU | 437 | AGAGTTTG GGCTAGCTACAACGA TTTCTTAA | 1758 |
| 1966 | GAAAACAA A CUCUUUUU | 438 | AAAAAGAG GGCTAGCTACAACGA TTGTTTTC | 1759 |
| 1980 | UUUUAAAA A UUACUUUU | 439 | AAAAGTAA GGCTAGCTACAACGA TTTTAAAA | 1760 |
| 1983 | UAAAAAUU A CUUUUAAA | 440 | TTTAAAAG GGCTAGCTACAACGA AATTTTTA | 1761 |
| 1991 | ACUUUUAA A UAUUAACU | 441 | AGTTAATA GGCTAGCTACAACGA TTAAAAGT | 1762 |
| 1993 | UUUUAAAU A UUAACUCA | 442 | TCAGTTAA GGCTAGCTACAACGA ATTTAAAA | 1763 |
| 1997 | AAAUAUUA A CUCAAAAG | 443 | CTTTTGAG GGCTAGCTACAACGA TAATATTT | 1764 |
| 2005 | ACUCAAAA G UUGAGAUU | 444 | AATCTCAA GGCTAGCTACAACGA TTTTGAGT | 1765 |
| 2011 | AAGUUCAG A UUUUCCCG | 445 | CCCCAAAA GGCTAGCTACAACGA CTCAACTT | 1766 |
| 2019 | AUUUUGGG G UGGUGGUG | 446 | CACCACCA GGCTAGCTACAACGA CCCAAAAT | 1767 |
| 2022 | UUGGGGUG G UGGUGUGC | 447 | GCACACCA GGCTAGCTACAACGA CACCCCAA | 1768 |
| 2025 | GGGUGGUG G UGUGCCAA | 448 | TTGGCACA GGCTAGCTACAACGA CACCACCC | 1769 |
| 2027 | GUGGUGCU G UGCCAAGA | 449 | TCTTGGCA GGCTAGCTACAACGA ACCACCAC | 1770 |
| 2029 | GGUGGUGU G CCAAGACA | 450 | TGTCTTCG GGCTAGCTACAACGA ACACCACC | 1771 |
| 2035 | GUGCCAAG A CAUUAAUU | 451 | AATTAATC GGCTAGCTACAACGA CTTGGCAC | 1772 |
| 2037 | GCCAAGAC A UUAAUUUU | 452 | AAAATTAA GGCTAGCTACAACGA GTCTTGGC | 1773 |
| 2041 | AGACAUUA A UUUUUUUU | 453 | AAAAAAAA GGCTAGCTACAACGA TAATGTCT | 1774 |
| 2054 | UUUUUUAA A CAAUGAAG | 454 | CTTCATTG GGCTAGCTACAACGA TTAAAAAA | 1775 |
| 2057 | UUUAAACA A UGAAGUGA | 455 | TCACTTCA GGCTAGCTACAACGA TGTTTAAA | 1776 |
| 2062 | ACAAUGAA G UGAAAAAG | 456 | CTTTTTCA GGCTAGCTACAACGA TTCATTGT | 1777 |
| 2070 | GUGAAAAA G UUUUACAA | 457 | TTGTAAAA GGCTAGCTACAACGA TTTTTCAC | 1778 |
| 2075 | AAAGUUUU A CAAUCUCU | 458 | AGAGATTG GGCTAGCTACAACGA AAAACTTT | 1779 |
| 2078 | GUUUUACA A UCUCUAGG | 459 | CCTAGAGA GGCTAGCTACAACGA TGTAAAAC | 1780 |
| 2086 | AUCUCUAG G UUUGGCUA | 460 | TAGCCAAA GGCTAGCTACAACGA CTAGAGAT | 1781 |
| 2091 | UAGGUUUG G CUAGUUCU | 461 | AGAACTAG GGCTAGCTACAACGA CAAACCTA | 1782 |
| 2095 | UUUGGCUA G UUCUCUUA | 462 | TAAGAGAA GGCTAGCTACAACGA TAGCCAAA | 1783 |
| 2104 | UUCUCUUA A CACUGGUU | 463 | AACCAGTG GGCTAGCTACAACGA TAAGAGAA | 1784 |
| 2106 | CUCUUAAC A CUGGUUAA | 464 | TTAACCAG GGCTAGCTACAACGA GTTAAGAG | 1785 |
| 2110 | UAACACUG G UUAAAUUA | 465 | TAATTTAA GGCTAGCTACAACGA CAGTGTTA | 1786 |
| 2115 | CUGGUUAA A UUAACAUU | 466 | AATGTTAA GGCTAGCTACAACGA TTAACCAG | 1787 |
| 2119 | UUAAAUUA A CAUUGCAU | 467 | ATGCAATG GGCTAGCTACAACGA TAATTTAA | 1788 |
| 2121 | AAAUUAAC A UUGCAUAA | 468 | TTATGCAA GGCTAGCTACAACGA GTTAATTT | 1789 |
| 2124 | UUAACAUU G CAUAAACA | 469 | TGTTTATG GGCTAGCTACAACGA AATGTTAA | 1790 |
| 2126 | AACAUUGC A UAAACACU | 470 | AGTGTTTA GGCTAGCTACAACGA GCAATGTT | 1791 |
| 2130 | UUGCAUAA A CACUUUUC | 471 | GAAAAGTG GGCTAGCTACAACGA TTATGCAA | 1792 |
| 2132 | GCAUAAAC A CUUUUCAA | 472 | TTCAAAAG GGCTAGCTACAACGA GTTTATGC | 1793 |
| 2141 | CUUUUCAA G UCUGAUCC | 473 | GGATCAGA GGCTAGCTACAACGA TTGAAAAG | 1794 |
| 2146 | CAAGUCUG A UCCAUAUU | 474 | AATATGGA GGCTAGCTACAACGA CAGACTTG | 1795 |
| 2150 | UCUGAUCC A UAUUUAAU | 475 | ATTAAATA GGCTAGCTACAACGA GGATCAGA | 1796 |
| 2152 | UGAUCCAU A UUUAAUAA | 476 | TTATTAAA GGCTAGCTACAACGA ATGGATCA | 1797 |
| 2157 | CAUAUUUA A UAAUGCUU | 477 | AACCATTA GGCTAGCTACAACGA TAAATATG | 1798 |
| 2160 | AUUUAAUA A UGCUUUAA | 478 | TTAAAGCA GGCTAGCTACAACGA TATTAAAT | 1799 |
| 2162 | UUAAUAAU G CUUUAAAA | 479 | TTTTAAAG GGCTAGCTACAACGA ATTATTAA | 1800 |
| 2170 | GCUUUAAA A UAAAAAUA | 480 | TATTTTTA GGCTAGCTACAACGA TTTAAAGC | 1801 |
| 2176 | AAAUAAAA A UAAAAACA | 481 | TGTTTTTA GGCTAGCTACAACGA TTTTATTT | 1802 |
| 2182 | AAAUAAAA A CAAUCCUU | 482 | AAGGATTG GGCTAGCTACAACGA TTTTATTT | 1803 |
| 2185 | UAAAAACA A UCCUUUUG | 483 | CAAAAGGA GGCTAGCTACAACGA TGTTTTTA | 1804 |
| 2194 | UCCUUUUC A UAAAUUUA | 484 | TAAATTTA GGCTAGCTACAACGA CAAAAGGA | 1805 |
| 2198 | UUUGAUAA A UUUAAAAU | 485 | ATTTTAAA GGCTAGCTACAACGA TTATCAAA | 1806 |
| 2205 | AAUUUAAA A UGUUACUU | 486 | AAGTAACA GGCTAGCTACAACGA TTTAAATT | 1807 |
| 2207 | UUUAAAAU G UUACUUAU | 487 | ATAAGTAA GGCTAGCTACAACGA ATTTTAAA | 1808 |
| 2210 | AAAAUGUU A CUUAUUUU | 488 | AAAATAAC GGCTAGCTACAACGA AACATTTT | 1809 |
| 2214 | UGUUACUU A UUUUAAAA | 489 | TTTTAAAA GGCTAGCTACAACGA AAGTAACA | 1810 |
| 2222 | AUUUUAAA A UAAAUGAA | 490 | TTCATTTA GGCTAGCTACAACGA TTTAAAAT | 1811 |
| 2226 | UAAAAUAA A UGAAGUGA | 491 | TCACTTCA GGCTAGCTACAACGA TTATTTTA | 1812 |
| 2231 | UAAAUGAA G UGAGAUGG | 492 | CCATCTCA GGCTAGCTACAACGA TTCATTTA | 1813 |
| 2236 | GAAGUGAG A UGGCAUGG | 493 | CCATGCCA GGCTAGCTACAACGA CTCACTTC | 1814 |
| 2239 | GUGAGAUG G CAUCCUGA | 494 | TCACCATG GGCTAGCTACAACGA CATCTCAC | 1815 |
| 2241 | GAGAUGGC A UGGUGAGG | 495 | CCTCACCA GGCTAGCTACAACGA GCCATCTC | 1816 |
| 2244 | AUGGCAUG G UGAGGUGA | 496 | TCACCTCA GGCTAGCTACAACGA CATGCCAT | 1817 |
| 2249 | AUGGUGAG G UGAAAGUA | 497 | TACTTTCA GGCTAGCTACAACGA CTCACCAT | 1818 |
| 2255 | AGGUGAAA G UAUCACUG | 498 | CAGTGATA GGCTAGCTACAACGA TTTCACCT | 1819 |
| 2257 | GUCAAAGU A UCACUGGA | 499 | TCCAGTGA GGCTAGCTACAACGA ACTTTCAC | 1820 |
| 2260 | AAAGUAUC A CUGGACUA | 500 | TAGTCCAG GGCTAGCTACAACGA GATACTTT | 1821 |
| 2265 | AUCACUGG A CUAGGUUG | 501 | CAACCTAC GGCTAGCTACAACGA CCACTGAT | 1822 |
| 2270 | UGGACUAG G UUGUUGGU | 502 | ACCAACAA GGCTAGCTACAACGA CTAGTCCA | 1823 |
| 2273 | ACUAGGUU G UUGGUGAC | 503 | GTCACCAA GGCTAGCTACAACGA AACCTAGT | 1824 |
| 2277 | GGUUGUUG G UGACUUAG | 504 | CTAAGTCA GGCTAGCTACAACGA CAACAACC | 1825 |
| 2280 | UGUUGGUG A CUUAGGUU | 505 | AACCTAAG GGCTAGCTACAACGA CACCAACA | 1826 |
| 2286 | UGACUUAG G UUCUAGAU | 506 | ATCTAGAA GGCTAGCTACAACGA CTAAGTCA | 1827 |
| 2293 | GGUUCUAG A UACGUCUC | 507 | GACACCTA GGCTAGCTACAACGA CTAGAACC | 1828 |
| 2297 | CUACAUAG G UCUCUUUU | 508 | AAAACACA GGCTAGCTACAACGA CTATCTAG | 1829 |
| 2299 | AGAUAGCU G UCUUUUAG | 509 | CTAAAAGA GGCTAGCTACAACGA ACCTATCT | 1830 |
| 2309 | CUUUUAGC A CUCUGAUU | 510 | AATCAGAG GGCTAGCTACAACGA CCTAAAAG | 1831 |
| 2315 | GGACUCUG A UUUUGAGG | 511 | CCTCAAAA GGCTAGCTACAACGA CAGAGTCC | 1832 |
| 2324 | UUUUGAGG A CAUCACUU | 512 | AAGTGATG GGCTAGCTACAACGA CCTCAAAA | 1833 |
| 2326 | UGGAGGAC A UCACUUAC | 513 | GTAAGTGA GGCTAGCTACAACGA GTCCTCAA | 1834 |
| 2329 | AGGACAUC A CUUACUAU | 514 | ATAGTAAG GGCTAGCTACAACCA GATGTCCT | 1835 |
| 2333 | CAUCACUU A CUAUCCAU | 515 | ATGGATAG GGCTAGCTACAACGA AAGTGATG | 1836 |
| 2336 | CACUUACU A UCCAUUUC | 516 | GAAATGGA GGCTAGCTACAACGA AGTAAGTG | 1837 |
| 2340 | UACUAUCC A UUUCUUCA | 517 | TGAAGAAA GGCTAGCTACAACGA GGATAGTA | 1838 |
| 2348 | AUUUCUUC A UGUUAAAA | 518 | TTTTAACA GGCTAGCTACAACGA GAAGAAAT | 1839 |
| 2350 | UUCUUCAU G UUAAAAGA | 519 | TCTTTTAA GGCTAGCTACAACGA ATGAAGAA | 1840 |
| 2360 | UAAAAGAA G UCAUCUCA | 520 | TGAGATGA GGCTAGCTACAACGA TTCTTTTA | 1841 |
| 2363 | AAGAAGUC A UCUCAAAC | 521 | GTTTGAGA GGCTAGCTACAACGA GACTTCTT | 1842 |
| 2370 | CAUCUCAA A CUCUUAGU | 522 | ACTAAGAC GGCTAGCTACAACGA TTGAGATG | 1843 |
| 2377 | AACUCUUA G UUUUUUUU | 523 | AAAAAAAA GGCTAGCTACAACGA TAAGAGTT | 1844 |
| 2390 | UUUUUUUU A CACUAUGU | 524 | ACATACTG GGCTAGCTACAACGA AAAAAAAA | 1845 |
| 2392 | UUUUUUAC A CUAUGUGA | 525 | TCACATAG GGCTAGCTACAACGA CTAAAAAA | 1846 |
| 2395 | UUUACACU A UGUGAUUU | 526 | AAATCACA GGCTAGCTACAACGA ACTCTAAA | 1847 |
| 2397 | UACACUAU G UGAUUUAU | 527 | ATAAATCA GGCTAGCTACAACGA ATAGTGTA | 1848 |
| 2400 | ACUAUGUG A UUUAUAUU | 528 | AATATAAA GGCTAGCTACAACGA CACATAGT | 1849 |
| 2404 | UGUGAUUU A UAUUCCAU | 529 | ATCGAATA GGCTAGCTACAACGA AAATCACA | 1850 |
| 2406 | UGAUUUAU A UUCCAUUU | 530 | AAATGGAA GGCTAGCTACAACGA ATAAATCA | 1851 |
| 2411 | UAUAUUCC A UUUACAUA | 531 | TATGTAAA GGCTAGCTACAACGA GGAATATA | 1852 |
| 2415 | UUCCAUUU A CAUAAGGA | 532 | TCCTTATG GGCTAGCTACAACGA AAATGGAA | 1853 |
| 2417 | CCAUUUAC A UAACCAUA | 533 | TATCCTTA GGCTAGCTACAACGA GTAAATGG | 1854 |
| 2423 | ACAUAGCG A UACACUUA | 534 | TAAGTGTA GGCTAGCTACAACGA CCTTATGT | 1855 |
| 2425 | AUAAGGAU A CACUUAUU | 535 | AATAAGTG GGCTAGCTACAACGA ATCCTTAT | 1856 |
| 2427 | AAGGAUAC A CUUAUUUG | 536 | CAAATAAG GGCTAGCTACAACGA GTATCCTT | 1857 |
| 2431 | AUACACUU A UUUGUCAA | 537 | TTGACAAA GGCTAGCTACAACGA AAGTGTAT | 1858 |
| 2435 | ACUUAUUU G UCAAGCUC | 538 | GAGCTTGA GGCTAGCTACAACGA AAATAAGT | 1859 |
| 2440 | UUUGUCAA G CUCAGCAC | 539 | GTGCTGAG GGCTAGCTACAACGA TTGACAAA | 1860 |
| 2445 | CAAGCUCA G CACAAUCU | 540 | AGATTGTG GGCTAGCTACAACGA TGAGCTTG | 1861 |
| 2447 | AGCUCAGC A CAAUCUGU | 541 | ACAGATTG GGCTAGCTACAACGA GCTGAGCT | 1862 |
| 2450 | UCACCACA A UCUCUAAA | 542 | TTTACAGA GGCTAGCTACAACGA TGTGCTGA | 1863 |
| 2454 | CACAAUCU G UAAAUUUU | 543 | AAAATTTA GGCTAGCTACAACGA AGATTGTG | 1864 |
| 2458 | AUCUGUAA A UUUUUAAC | 544 | GTTAAAAA GGCTAGCTACAACGA TTACAGAT | 1865 |
| 2465 | AAUUUUUA A CCUAUGUU | 545 | AACATAGG GGCTAGCTACAACGA TAAAAATT | 1866 |
| 2469 | UUUAACCU A UGGUACAC | 546 | GTGTAACA GGCTAGCTACAACGA AGGTTAAA | 1867 |
| 2471 | UAACCUAU G UUACACCA | 547 | TGGTGTAA GGCTAGCTACAACGA ATAGGTTA | 1868 |
| 2474 | CCUAUGUU A CACCAUCU | 548 | AGATGGTG GGCTAGCTACAACGA AACATAGG | 1869 |
| 2476 | UAUGUUAC A CCAUCUUC | 549 | GAAGATGG GGCTAGCTACAACGA GTAACATA | 1870 |
| 2479 | GUUACACC A UCUUCACU | 550 | ACTGAAGA GGCTAGCTACAACGA GGTGTAAC | 1871 |
| 2486 | CAUCUUCA G UGCCAGUC | 551 | GACTGGCA GGCTAGCTACAACGA TGAAGATG | 1872 |
| 2488 | UCUUCAGU G CCAGUCUU | 552 | AAGACTGG GGCTAGCTACAACGA ACTGAAGA | 1873 |
| 2492 | CAGUGCCA G UCUUGGGC | 553 | GCCCAAGA GGCTAGCTACAACGA TGGCACTG | 1874 |
| 2499 | AGUCUUGG G CAAAAUUC | 554 | CAATTTTG GGCTAGCTACAACGA CCAAGACT | 1875 |
| 2504 | UGGGCAAA A UUGUGCAA | 555 | TTGCACAA GGCTAGCTACAACGA TTTGCCCA | 1876 |
| 2507 | GCAAAAUU G UGCAAGAG | 556 | CTCTTGCA GGCTAGCTACAACGA AATTTTGC | 1877 |
| 2509 | AAAAUUGU G CAAGAGGU | 557 | ACCTCTTG GGCTAGCTACAACGA ACAATTTT | 1878 |
| 2516 | UGCAAGAG G UGAAGUUU | 558 | AAACTTCA GGCTAGCTACAACGA CTCTTGCA | 1879 |
| 2521 | GAGGUGAA G UUUAUAUU | 559 | AATATAAA GGCTAGCTACAACGA TTCACCTC | 1880 |
| 2525 | UGAAGUUU A UAUUUGAA | 560 | TTCAAATA GGCTAGCTACAACGA AAACTTCA | 1881 |
| 2527 | AAGUUUAU A UUUGAAUA | 561 | TATTCAAA GGCTAGCTACAACGA ATAAACTT | 1882 |
| 2533 | AUAUUUGA A UAUCCAUU | 562 | AATGGATA GGCTAGCTACAACGA TCAAATAT | 1883 |
| 2535 | AUUUGAAU A UCCAUUCU | 563 | AGAATGGA GGCTAGCTACAACGA ATTCAAAT | 1884 |
| 2539 | GAAUAUCC A UUCUCGUU | 564 | AACGAGAA GGCTAGCTACAACGA GGATATTC | 1885 |
| 2545 | CCAUUCUC G UUUUAGGA | 565 | TCCTAAAA GGCTAGCTACAACGA GAGAATGG | 1886 |
| 2553 | GUUUUAGG A CUCUUCUU | 566 | AAGAAGAG GGCTAGCTACAACGA CCTAAAAC | 1887 |
| 2564 | CUUCUUCC A UAUUAGUG | 567 | CACTAATA GGCTAGCTACAACGA GGAAGAAG | 1888 |
| 2566 | UCUUCCAU A UUAGUGUC | 568 | GACACTAA GGCTAGCTACAACGA ATGGAAGA | 1889 |
| 2570 | CCAUAUUA G UGUCAUCU | 569 | AGATGACA GGCTAGCTACAACGA TAATATGG | 1890 |
| 2572 | AUAUUAGU G UCAUCUUG | 570 | CAAGATGA GGCTAGCTACAACGA ACTAATAT | 1891 |
| 2575 | UUAGUGUC A UCUUGCCU | 571 | AGGCAAGA GGCTAGCTACAACGA GACACTAA | 1892 |
| 2580 | GUCAUCUU G CCUCCCUA | 572 | TAGGGAGG GGCTAGCTACAACGA AAGATGAC | 1893 |
| 2588 | GCCUCCCU A CCUUCCAC | 573 | GTGGAAGG GGCTAGCTACAACGA AGGGAGGC | 1894 |
| 2595 | UACCUUCC A CAUGCCCC | 574 | GGGGCATG GGCTAGCTACAACGA GGAAGGTA | 1895 |
| 2597 | CCUUCCAC A UGCCCCAU | 575 | ATGGGGCA GGCTAGCTACAACGA GTGGAAGG | 1896 |
| 2599 | UUCCACAU G CCCCAUGA | 576 | TCATGGGG GGCTAGCTACAACGA ATGTGGAA | 1897 |
| 2604 | CAUGCCCC A UGACUUCA | 577 | TCAAGTCA GGCTAGCTACAACGA GGGGCATG | 1898 |
| 2607 | GCCCCAUG A CUUGAUGC | 578 | GCATCAAG GGCTAGCTACAACGA CATGGGGC | 1899 |
| 2612 | AUGACUUG A UGCAGUUU | 579 | AAACTGCA GGCTAGCTACAACGA CAAGTCAT | 1900 |
| 2614 | GACUUGAU G CAGUUUUA | 580 | TAAAACTG GGCTAGCTACAACGA ATCAAGTC | 1901 |
| 2617 | UUGAUGCA G UUUUAAUA | 581 | TATTAAAA GGCTAGCTACAACGA TGCATCAA | 1902 |
| 2623 | CAGUUUUA A UACUUGUA | 582 | TACAAGTA GGCTAGCTACAACGA TAAAACTG | 1903 |
| 2625 | GUUUUAAU A CUUGUAAU | 583 | ATTACAAG GGCTAGCTACAACGA ATTAAAAC | 1904 |
| 2629 | UAAUACUU G UAAUUCCC | 584 | GGGAATTA GGCTAGCTACAACGA AAGTATTA | 1905 |
| 2632 | UACUUCUA A UUCCCCUA | 585 | TAGGGGAA GGCTAGCTACAACGA TACAAGTA | 1906 |
| 2641 | UUCCCCUA A CCAUAAGA | 586 | TCTTATGG GGCTAGCTACAACGA TAGGGGAA | 1907 |
| 2644 | CCCUAACC A UAACAUUU | 587 | AAATCTTA GGCTAGCTACAACGA GGTTAGGG | 1908 |
| 2649 | ACCAUAAG A UUUACUGC | 588 | GCAGTAAA GGCTAGCTACAACGA CTTATGGT | 1909 |
| 2653 | UAAGAUUU A CUGCUGCU | 589 | AGCAGCAC GGCTAGCTACAACGA AAATCTTA | 1910 |
| 2656 | GAUUUACU G CUGCUGUG | 590 | CACAGCAG GGCTAGCTACAACGA AGTAAATC | 1911 |
| 2659 | UUACUGCU G CUGUGGAU | 591 | ATCCACAG GGCTAGCTACAACGA AGCAGTAA | 1912 |
| 2662 | CUGCUGCU G UGGAUAUC | 592 | GATATCCA GGCTAGCTACAACGA AGGAGCAG | 1913 |
| 2666 | UGCUGUGG A UAUCUCCA | 593 | TGGAGATA GGCTAGCTACAACGA CCACAGCA | 1914 |
| 2668 | CUGUGGAU A UCUCCAUG | 594 | CATGGAGA GGCTAGCTACAACGA ATCCACAG | 1915 |
| 2674 | AUAUCUCC A UGAAGUUU | 595 | AAACTTCA GGCTAGCTACAACGA GGAGATAT | 1916 |
| 2679 | UCCAUGAA G UUUUCCCA | 596 | TGGGAAAA GGCTAGCTACAACGA TTCATGGA | 1917 |
| 2687 | GUUUUCCC A CUGAGUCA | 597 | TGACTCAG GGCTAGCTACAACGA GGGAAAAC | 1918 |
| 2692 | CCCACUGA G UCACAUCA | 598 | TGATGTGA GGCTAGCTACAACGA TCAGTGGG | 1919 |
| 2695 | ACUGACUC A CAUCAGAA | 599 | TTCTGATG GGCTAGCTACAACGA GACTCAGT | 1920 |
| 2697 | UGAGUCAC A UCAGAAAU | 600 | ATTTCTGA GGCTAGCTACAACGA GTCACTCA | 1921 |
| 2704 | CAUCAGAA A UGCCCUAC | 601 | GTAGGGCA GGCTAGCTACAACGA TTCTGATG | 1922 |
| 2706 | UCAGAAAU G CCCUACAU | 602 | ATGTAGGG GGCTAGCTACAACGA ATTTCTGA | 1923 |
| 2711 | AAUGCCCU A CAUCUUAU | 603 | ATAAGATG GGCTAGCTACAACGA AGGGCATT | 1924 |
| 2713 | UGCCCUAC A UCUUAUUU | 604 | AAATAAGA GGCTAGCTACAACGA GTAGGGCA | 1925 |
| 2718 | UACAUCUU A UUUUCCUC | 605 | GAGGAAAA GGCTAGCTACAACGA AAGATGTA | 1926 |
| 2730 | UCCUCAGG G CUCAAGAG | 606 | CTCTTGAG GGCTAGCTACAACGA CCTGAGGA | 1927 |
| 2740 | UCAAGAGA A UCUGACAG | 607 | CTGTCAGA GGCTAGCTACAACGA TCTCTTGA | 1928 |
| 2745 | AGAAUCUG A CAGAUACC | 608 | GGTATCTG GGCTAGCTACAACGA CAGATTCT | 1929 |
| 2749 | UCUGACAG A UACCAUAA | 609 | TTATGGTA GGCTAGCTACAACGA CTGTCAGA | 1930 |
| 2751 | UGACAGAU A CCAUAAAG | 610 | CTTTATGG GGCTAGCTACAACGA ATCTGTCA | 1931 |
| 2754 | CAGAUACC A UAAAGGGA | 611 | TCCCTTTA GGCTAGCTACAACGA GGTATCTG | 1932 |
| 2762 | AUAAAGGG A UUUGACCU | 612 | AGGTCAAA GGCTAGCTACAACGA CCCTTTAT | 1933 |
| 2767 | GGGAUUUG A CCUAAUCA | 613 | TGATTAGG GGCTAGCTACAACGA CAAATCCC | 1934 |
| 2772 | UUGACCUA A UCACUAAU | 614 | ATTAGTGA GGCTAGCTACAACGA TAGGTCAA | 1935 |
| 2775 | ACCUAAUC A CUAAUUUU | 615 | AAAATTAG GGCTAGCTACAACGA GATTAGGT | 1936 |
| 2779 | AAUCACUA A UUUUCAGG | 616 | CCTGAAAA GGCTAGCTACAACGA TAGTGATT | 1937 |
| 2787 | AUUUUCAG G UGGUGGCU | 617 | AGCCACCA GGCTAGCTACAACGA CTGAAAAT | 1938 |
| 2790 | UUCAGGUG G UGGCUGAU | 618 | ATCAGCCA GGCTAGCTACAACGA CACCTGAA | 1939 |
| 2793 | AGGUGGUG G CUGAUGCU | 619 | AGCATCAG GGCTAGCTACAACGA CACCACCT | 1940 |
| 2797 | GGUGGCUG A UGCUUUGA | 620 | TCAAAGCA GGCTAGCTACAACGA CAGCCACC | 1941 |
| 2799 | UGGCUGAU G CUUUGAAC | 621 | GTTCAAAG GGCTAGCTACAACGA ATCAGCCA | 1942 |
| 2806 | UGCUUUGA A CAUCUCUU | 622 | AAGAGATG GGCTAGCTACAACGA TCAAAGCA | 1943 |
| 2808 | CUUUGAAC A UCUCUUUG | 623 | CAAAGAGA GGCTAGCTACAACGA GTTCAAAG | 1944 |
| 2816 | AUCUCUUU G CUGCCCAA | 624 | TTGGGCAG GGCTAGCTACAACGA AAAGAGAT | 1945 |
| 2819 | UCUUUGCU G CCCAAUCC | 625 | GGATTGGG GGCTAGCTACAACGA AGCAAAGA | 1946 |
| 2824 | GCUGCCCA A UCCAUUAG | 626 | CTAATGGA GGCTAGCTACAACGA TGGGCAGC | 1947 |
| 2828 | CCCAAUCC A UUAGCGAC | 627 | GTCGCTAA GGCTAGCTACAACGA GGATTGGG | 1948 |
| 2832 | AUCCAUUA G CGACAGUA | 628 | TACTGTCG GGCTAGCTACAACGA TAATGGAT | 1949 |
| 2835 | CAUUAGCG A CAGUAGGA | 629 | TCCTACTG GGCTAGCTACAACGA CGCTAATG | 1950 |
| 2838 | UAGCGACA U UAGGAUUU | 630 | AAATCCTA GGCTAGCTACAACGA TGTCGCTA | 1951 |
| 2843 | ACAGUAGG A UUUUUCAA | 631 | TTGAAAAA GGCTAGCTACAACGA CCTACTGT | 1952 |
| 2851 | AUUUUUCA A CCCUGGUA | 632 | TACCAGGG GGCTAGCTACAACGA TGAAAAAT | 1953 |
| 2857 | CAACCCUG G UAUGAAUA | 633 | TATTCATA GGCTAGCTACAACGA CAGGGTTG | 1954 |
| 2859 | ACCCUGGU A UGAAUAGA | 634 | TCTATTCA GGCTAGCTACAACGA ACCAGGGT | 1955 |
| 2863 | UGGUAUGA A UAGACAGA | 635 | TCTGTCTA GGCTAGCTACAACGA TCATACCA | 1956 |
| 2867 | AUGAAUAG A CAGAACCC | 636 | GGGTTCTG GGCTAGCTACAACGA CTATTCAT | 1957 |
| 2872 | UAGACAGA A CCCUAUCC | 637 | GGATAGGG GGCTAGCTACAACGA TCTGTCTA | 1958 |
| 2877 | AGAACCCU A UCCAGUGG | 638 | CCACTGGA GGCTAGCTACAACGA AGGGTTCT | 1959 |
| 2882 | CCUAUCCA G UGGAAGGA | 639 | TCCTTCCA GGCTAGCTACAACGA TGGATAGG | 1960 |
| 2893 | GAAGGAGA A UUUAAUAA | 640 | TTATTAAA GGCTAGCTACAACGA TCTCCTTC | 1961 |
| 2898 | AGAAUUUA A UAAAGAUA | 641 | TATCTTTA GGCTAGCTACAACGA TAAATTCT | 1962 |
| 2904 | UAAUAAAG A UAGUGCAG | 642 | CTGCACTA GGCTAGCTACAACGA CTTTATTA | 1963 |
| 2907 | UAAAGAUA G UGCAGAAA | 643 | TTTCTGCA GGCTAGCTACAACGA TATCTTTA | 1964 |
| 2909 | AAGAUAGU G CAGAAAGA | 644 | TCTTTCTG GGCTAGCTACAACGA ACTATCTT | 1965 |
| 2918 | CAGAAAGA A UUCCUUAG | 645 | CTAAGGAA GGCTAGCTACAACGA TCTTTCTG | 1966 |
| 2927 | UUCCUUAG G UAAUCUAU | 646 | ATAGATTA GGCTAGCTACAACGA CTAAGGAA | 1967 |
| 2930 | CUUAGGUA A UCUAUAAC | 647 | GTTATAGA GGCTAGCTACAACGA TACCTAAG | 1968 |
| 2934 | GGUAAUCU A UAACUAGG | 648 | CCTAGTTA GGCTAGCTACAACGA AGATTACC | 1969 |
| 2937 | AAUCUAUA A CUAGGACU | 649 | AGTCCTAG GGCTAGCTACAACGA TATAGATT | 1970 |
| 2943 | UAACUAGG A CUACUCCU | 650 | AGGACTAG GGCTAGCTACAACGA CCTAGTTA | 1971 |
| 2946 | CUAGGACU A CUCCUGGU | 651 | ACCAGGAG GGCTAGCTACAACGA AGTCCTAG | 1972 |
| 2953 | UACUCCUG G UAACAGUA | 652 | TACTGTTA GGCTAGCTACAACGA CAGGACTA | 1973 |
| 2956 | UCCUGGUA A CAGUAAUA | 653 | TATTACTG GGCTAGCTACAACGA TACCAGGA | 1974 |
| 2959 | UGGUAACA G UAAUACAU | 654 | ATGTATTA GGCTAGCTACAACGA TGTTACCA | 1975 |
| 2962 | UAACAGUA A UACAUUCC | 655 | GGAATGTA GGCTAGCTACAACGA TACTGTTA | 1976 |
| 2964 | ACAGUAAU A CAUUCCAU | 656 | ATGGAATG GGCTAGCTACAACGA ATTACTGT | 1977 |
| 2966 | AGUAAUAC A UUCCAUUG | 657 | CAATGGAA GGCTAGCTACAACGA GTATTACT | 1978 |
| 2971 | UACAUUCC A UUGUUUUA | 658 | TAAAACAA GGCTAGCTACAACGA GGAATGTA | 1979 |
| 2974 | AUUCCAUU G UUUUAGUA | 659 | TACTAAAA GGCTAGCTACAACGA AATGGAAT | 1980 |
| 2980 | UUGUUUUA G UAACCAGA | 660 | TCTGGTTA GGCTAGCTACAACGA TAAAACAA | 1981 |
| 2983 | UUUUAGUA A CCACAAAU | 661 | ATTTCTGG GGCTAGCTACAACGA TACTAAAA | 1982 |
| 2990 | AACCAGAA A UCUUCAUG | 662 | CATGAAGA GGCTAGCTACAACGA TTCTGCTT | 1983 |
| 2996 | AAAUCUUC A UGCAAUCA | 663 | TCATTCCA GGCTAGCTACAACGA GAACATTT | 1984 |
| 2998 | AUCUUCAU G CAAUGAAA | 664 | TTTCATTG GGCTAGCTACAACGA ATCAACAT | 1985 |
| 3001 | UUCAUGCA A UGAAAAAU | 665 | ATTTTTCA GGCTAGCTACAACGA TGCATGAA | 1986 |
| 3008 | AAUGAAAA A UACUUUAA | 666 | TTAAACTA GGCTAGCTACAACGA TTTTCATT | 1987 |
| 3010 | UGAAAAAU A CUUUAAUU | 667 | AATTAAAC GGCTAGCTACAACGA ATTTTTCA | 1988 |
| 3016 | AUACUUUA A UUCAUGAA | 668 | TTCATGAA GGCTAGCTACAACGA TAAAGTAT | 1989 |
| 3020 | UUUAAUUC A UGAACCUU | 669 | AAGCTTCA GGCTAGCTACAACGA CAATTAAA | 1990 |
| 3025 | UUCAUGAA G CUUACUUU | 670 | AAACTAAC GGCTAGCTACAACGA TTCATGAA | 1991 |
| 3029 | UCAACCUU A CUUUUUUU | 671 | AAAAAAAG GGCTAGCTACAACGA AAGCTTCA | 1992 |
| 3044 | UUUUUUUG G UGUCAGAG | 672 | CTCTGACA GGCTAGCTACAACGA CAAAAAAA | 1993 |
| 3046 | UUUUUGGU G UCACAGUC | 673 | GACTCTGA GGCTAGCTACAACGA ACCAAAAA | 1994 |
| 3052 | GUCUCAGA G UCUCGCUC | 674 | GAGCGAGA GGCTAGCTACAACGA TCTGACAC | 1995 |
| 3057 | AGAGUCUC G CUCUUGUC | 675 | GACAAGAG GGCTAGCTACAACGA GAGACTCT | 1996 |
| 3063 | UCCCUCUU G UCACCCAG | 676 | CTGGGTCA GGCTAGCTACAACGA AAGAGCGA | 1997 |
| 3066 | CUCUGGUC A CCCAGGCU | 677 | AGCCTGGG GGCTAGCTACAACGA GACAAGAG | 1998 |
| 3072 | UCACCCAG G CUGGAAUG | 678 | CATTCCAG GGCTAGCTACAACGA CTGGGTGA | 1999 |
| 3078 | AGGCUGGA A UGCAGUGG | 679 | CCACTGCA GGCTAGCTACAACGA TCCAGCCT | 2000 |
| 3080 | GCUGGAAU G CAGUGGCG | 680 | CGCCACTG GGCTAGCTACAACGA ATTCCAGC | 2001 |
| 3083 | GGAAUGCA G UGGCGCCA | 681 | TGGCGCCA GGCTAGCTACAACGA TGCATTCC | 2002 |
| 3086 | AUGCAGUG G CGCCAUCU | 682 | AGATGGCG GGCTAGCTACAACGA CACTGCAT | 2003 |
| 3088 | GCAGUGGC G CCAUCUCA | 683 | TGAGATGG GGCTAGCTACAACGA GCCACTGC | 2004 |
| 3091 | GUGGCGCC A UCUCAGCU | 684 | AGCTGAGA GGCTAGCTACAACGA GGCGCCAC | 2005 |
| 3097 | CCAUCUCA G CUCACUGC | 685 | GCAGTGAC GGCTAGCTACAACGA TGAGATGG | 2006 |
| 3101 | CUCAGCUC A CUGCAACC | 686 | GGTTGCAG GGCTAGCTACAACGA GAGCTGAG | 2007 |
| 3104 | AGCUCACU G CAACCUUC | 687 | GAAGGTTG GGCTAGCTACAACGA AGTGAGCT | 2008 |
| 3107 | UCACUGCA A CCUUCCAU | 688 | ATGGAAGG GGCTAGCTACAACGA TGCAGTGA | 2009 |
| 3114 | AACCUUCC A UCUUCCCA | 689 | TGGGAAGA GGCTAGCTACAACGA GGAAGGTT | 2010 |
| 3124 | CUUCCCAG G UUCAAGCG | 690 | CGCTTGAA GGCTAGCTACAACGA CTGGGAAG | 2011 |
| 3130 | AGGUUCAA G CGAUUCUC | 691 | GAGAATCG GGCTAGCTACAACGA TTGAACCT | 2012 |
| 3133 | UUCAAGCG A UUCUCGUG | 692 | CACGAGAA GGCTAGCTACAACGA CGCTTGAA | 2013 |
| 3139 | CGAUUCUC G UGCCUCGG | 693 | CCGAGCCA GGCTAGCTACAACGA GAGAATCG | 2014 |
| 3141 | AUUCUCGU G CCUCGGCC | 694 | GGCCGAGG GGCTAGCTACAACGA ACGAGAAT | 2015 |
| 3147 | GUGCCUCG G CCUCCUGA | 695 | TCAGGAGG GGCTAGCTACAACGA CGAGGCAC | 2016 |
| 3156 | CCUCCUGA G UAGCUGGG | 696 | CCCAGCTA GGCTAGCTACAACGA TCAGGAGG | 2017 |
| 3159 | CCUGAGUA G CUGGGAUU | 697 | AATCCCAG GGCTAGCTACAACGA TACTCAGG | 2018 |
| 3165 | UAGCUGGG A UUACAGGC | 698 | GCCTGTAA GGCTAGCTACAACGA CCCAGCTA | 2019 |
| 3168 | CUGGGAUU A CAGGCGUG | 699 | CACGCCTC GGCTAGCTACAACGA AATCCCAG | 2020 |
| 3172 | GAUUACAG G CGUGUGCA | 700 | TGCACACG GGCTAGCTACAACGA CTGTAATC | 2021 |
| 3174 | UUACAGGC G UGUGCACU | 701 | AGTGCACA GGCTAGCTACAACGA GCCTGTAA | 2022 |
| 3176 | ACAGGCGU G UGCACUAC | 702 | GTACTGCA GGCTAGCTACAACGA ACGCCTGT | 2023 |
| 3178 | AGGCGUGU G CACUACAC | 703 | GTCTAGTG GGCTAGCTACAACGA ACACGCCT | 2024 |
| 3180 | GCGUGUGC A CUACACUC | 704 | GAGTGTAG GGCTAGCTACAACGA GCACACGC | 2025 |
| 3183 | UGUGCACU A CACUCAAC | 705 | GTTGAGTG GGCTAGCTACAACGA AGTGCACA | 2026 |
| 3185 | UGCACUAC A CUCAACUA | 706 | TAGTTGAG GGCTAGCTACAACGA GTAGTGCA | 2027 |
| 3190 | UACACUCA A CUAAUUUU | 707 | AAAATTAG GGCTAGCTACAACGA TGAGTGTA | 2028 |
| 3194 | CUCAACUA A UUUUUCUA | 708 | TACAAAAA GGCTAGCTACAACGA TAGTTGAG | 2029 |
| 3200 | UAAUUUUU G UAUUUUUA | 709 | TAAAAATA GGCTAGCTACAACGA AAAAATTA | 2030 |
| 3202 | AUUUUUGU A UUUUUAGG | 710 | CCTAAAAA GGCTAGCTACAACGA ACAAAAAT | 2031 |
| 3215 | UAGGAGAG A CGGGGUUU | 711 | AAACCCCG GGCTAGCTACAACGA CTCTCCTA | 2032 |
| 3220 | GAGACGGG G UUUCACCU | 712 | AGGTGAAA GGCTAGCTACAACGA CCCGTCTC | 2033 |
| 3225 | GGGGUUUC A CCUGUUGG | 713 | CCAACAGG GGCTAGCTACAACGA GAAACCCC | 2034 |
| 3229 | UUUCACCU G UUGGCCAG | 714 | CTGGCCAA GGCTAGCTACAACGA AGGTGAAA | 2035 |
| 3233 | ACCUGUUG G CCAGGCUG | 715 | CAGCCTGG GGCTAGCTACAACGA CAACAGCT | 2036 |
| 3238 | UUGGCCAG G CUGGUCUC | 716 | GAGACCAG GGCTAGCTACAACGA CTGGCCAA | 2037 |
| 3242 | CCAGGCUG G UCUCGAAC | 717 | GTTCGAGA GGCTAGCTACAACGA CAGCCTGG | 2038 |
| 3249 | GGUCUCGA A CUCCUGAC | 718 | GTCAGGAG GGCTAGCTACAACGA TCGAGACC | 2039 |
| 3256 | AACUCCUG A CCUCAAGU | 719 | ACTTGAGG GGCTAGCTACAACGA CAGGAGTT | 2040 |
| 3263 | GACCUCAA G UGAUUCAC | 720 | GTGAATCA GGCTAGCTACAACGA TTGAGGTC | 2041 |
| 3266 | CUCAAGUG A UUCACCCA | 721 | TGGGTGAA GGCTAGCTACAACGA CACTTGAG | 2042 |
| 3270 | AGUGAUUC A CCCACCUU | 722 | AAGGTGGG GGCTAGCTACAACGA GAATCACT | 2043 |
| 3274 | AUUCACCC A CCUUGGCC | 723 | GGCCAAGG GGCTAGCTACAACGA GGGTGAAT | 2044 |
| 3280 | CCACCUUG G CCUCAUAA | 724 | TTATGAGG GGCTAGCTACAACGA CAAGGTGG | 2045 |
| 3285 | UUGGCCUC A UAAACCUG | 725 | CAGGTTTA GGCTAGCTACAACGA GAGGCCAA | 2046 |
| 3289 | CCUCAUAA A CCUGUUUU | 726 | AAAACAGG GGCTAGCTACAACGA TTATGAGG | 2047 |
| 3293 | AUAAACCU G UUUUGCAG | 727 | CTGCAAAA GGCTAGCTACAACGA AGGTTTAT | 2048 |
| 3298 | CCUGUUUU G CAGAACUC | 728 | GAGTTCTG GGCTAGCTACAACGA AAAACAGG | 2049 |
| 3303 | UUUGCAGA A CUCAUUUA | 729 | TAAATGAG GGCTAGCTACAACGA TCTGCAAA | 2050 |
| 3307 | CAGAACUC A UUUAUUCA | 730 | TGAATAAA GGCTAGCTACAACGA GAGTTCTG | 2051 |
| 3311 | ACUCAUUU A UUCAGCAA | 731 | TTGCTGAA GGCTAGCTACAACGA AAATGAGT | 2052 |
| 3316 | UUUAUUCA G CAAAUAUU | 732 | AATATTTG GGCTAGCTACAACGA TGAATAAA | 2053 |
| 3320 | UUCAGCAA A UAUUUAUU | 733 | AATAAATA GGCTAGCTACAACGA TTGCTGAA | 2054 |
| 3322 | CAGCAAAU A UUUAUUGA | 734 | TCAATAAA GGCTAGCTACAACGA ATTTGCTG | 2055 |
| 3326 | AAAUAUUU A UUGAGUGC | 735 | GCACTCAA GGCTAGCTACAACGA AAATATTT | 2056 |
| 3331 | UUUAUUGA G UGCCUACC | 736 | GGTAGGCA GGCTAGCTACAACGA TCAATAAA | 2057 |
| 3333 | UAUUGAGU G CCUACCAG | 737 | CTGGTAGG GGCTAGCTACAACGA ACTCAATA | 2058 |
| 3337 | GAGUGCCU A CCAGAUGC | 738 | GCATCTGG GGCTAGCTACAACGA AGGCACTC | 2059 |
| 3342 | CCUACCAG A UGCCAGUC | 739 | GACTGGCA GGCTAGCTACAACGA CTGGTAGG | 2060 |
| 3344 | UACCAGAU G CCAGUCAC | 740 | GTGACTGG GGCTAGCTACAACGA ATCTGGTA | 2061 |
| 3348 | AGAUGCCA G UCACCGCA | 741 | TGCGGTGA GGCTAGCTACAACGA TGGCATCT | 2062 |
| 3351 | UGCCAGUC A CCGCACAA | 742 | TTGTGCGG GGCTAGCTACAACGA GACTGGCA | 2063 |
| 3354 | CAGUCACC G CACAAGGC | 743 | GCCTTGTG GGCTAGCTACAACGA GGTGACTG | 2064 |
| 3356 | GUCACCGC A CAAGGCAC | 744 | GTGCCTTG GGCTAGCTACAACGA GCGGTGAC | 2065 |
| 3361 | CGCACAAG G CACUGGGU | 745 | ACCCAGTG GGCTAGCTACAACGA CTTGTGCG | 2066 |
| 3363 | CACAAGGC A CUGGGUAU | 746 | ATACCCAG GGCTAGCTACAACGA GCCTTGTG | 2067 |
| 3368 | GGCACUGG G UAUAUGGU | 747 | ACCATATA GGCTAGCTACAACGA CCAGTGCC | 2068 |
| 3370 | CACUGGGU A UAUGGUAU | 748 | ATACCATA GGCTAGCTACAACGA ACCCAGTG | 2069 |
| 3372 | CUGGGUAU A UGGUAUCC | 749 | GGATACCA GGCTAGCTACAACGA ATACCCAG | 2070 |
| 3375 | GGUAUAUG G UAUCCCCA | 750 | TGGGGATA GGCTAGCTACAACGA CATATACC | 2071 |
| 3377 | UAUAUGGU A UCCCCAAA | 751 | TTTGGGGA GGCTAGCTACAACGA ACCATATA | 2072 |
| 3385 | AUCCCCAA A CAAGAGAC | 752 | GTCTCTTG GGCTAGCTACAACGA TTGGGGAT | 2073 |
| 3392 | AACAAGAG A CAUAAUCC | 753 | GGATTATG GGCTAGCTACAACGA CTCTTGTT | 2074 |
| 3394 | CAAGAGAC A UAAUCCCG | 754 | CGGGATTA GCCTAGCTACAACGA GTCTCTTG | 2075 |
| 3397 | GAGACAUA A UCCCGGUC | 755 | GACCGGGA GGCTAGCTACAACGA TATGTCTC | 2076 |
| 3403 | UAAUCCCG G UCCUUAGG | 756 | CCTAAGGA GGCTAGCTACAACGA CGGGATTA | 2077 |
| 3411 | GUCCUUAG G UACUGCUA | 757 | TACCACTA GGCTAGCTACAACGA CTAAGGAC | 2078 |
| 3413 | CCUUAGGU A CUGCUAGU | 758 | ACTAGCAG GCCTAGCTACAACGA ACCTAAGG | 2079 |
| 3416 | UAGGUACU G CUAGUGUG | 759 | CACACTAG GGCTACCTACAACGA AGTACCTA | 2080 |
| 3420 | UACUGCUA G UGUGGUCU | 760 | AGACCACA GGCTAGCTACAACGA TAGCAGTA | 2081 |
| 3422 | CUGCUAGU G UGGUCUCU | 761 | ACAGACCA GGCTAGCTACAACGA ACTAGCAG | 2082 |
| 3425 | CUAGUGUG G UCUGUAAU | 762 | ATTACAGA GGCTAGCTACAACGA CACACTAG | 2083 |
| 3429 | UGUGGUCU G UAAUAUCU | 763 | ACATATTA GGCTAGCTACAACGA AGACCACA | 2084 |
| 3432 | GGUCUCUA A UAUCUUAC | 764 | GTAAGATA GGCTAGCTACAACGA TACAGACC | 2085 |
| 3434 | UCUGUAAU A UCUUACUA | 765 | TAGTAAGA GGCTAGCTACAACGA ATTACAGA | 2086 |
| 3439 | AAUAUCUU A CUAAGGCC | 766 | GGCCTTAG GGCTAGCTACAACGA AAGATATT | 2087 |
| 3445 | UUACUAAG G CCUUUGGU | 767 | ACCAAAGG GGCTAGCTACAACGA CTTAGTAA | 2088 |
| 3452 | GGCCUUUG G UAUACGAC | 768 | GTCGTATA GGCTAGCTACAACGA CAAAGGCC | 2089 |
| 3454 | CCUUUCGU A UACCACCC | 769 | GGGTCGTA GGCTAGCTACAACGA ACCAAAGG | 2090 |
| 3456 | UUUGGUAU A CGACCCAG | 770 | CTGGGTCG GGCTAGCTACAACGA ATACCAAA | 2091 |
| 3459 | CGUAUACG A CCCAGAGA | 771 | TCTCTGGG GGCTAGCTACAACGA CGTATACC | 2092 |
| 3467 | ACCCAGAG A UAACACCA | 772 | TCGTGTTA GGCTAGCTACAACGA CTCTGGGT | 2093 |
| 3470 | CAGAGAUA A CACGAUGC | 773 | GCATCGTG GGCTAGCTACAACGA TATCTCTG | 2094 |
| 3472 | GAGAUAAC A CGAUGCGU | 774 | ACGCATCG GGCTAGCTACAACGA GTTATCTC | 2095 |
| 3475 | AUAACACG A UGCGUAUU | 775 | AATACGCA GGCTAGCTACAACGA CGTGTTAT | 2096 |
| 3477 | AACACGAU G CGUAUUUU | 776 | AAAATACC GGCTACCTACAACGA ATCGTGTT | 2097 |
| 3479 | CACGAUCC G UAUUUUAG | 777 | CTAAAATA GGCTAGCTACAACGA GCATCGTG | 2098 |
| 3481 | CGAUGCGU A UUUUAGUU | 778 | AACTAAAA GGCTAGCTACAACGA ACGCATCG | 2099 |
| 3487 | GUAUUUUA G UUUUGCAA | 779 | TTGCAAAA GGCTAGCTACAACGA TAAAATAC | 2100 |
| 3492 | UUAGUUUU G CAAAGAAG | 780 | CTTCTTTG GGCTAGCTACAACGA AAAACTAA | 2101 |
| 3503 | AAGAACGG G UUUGGUCU | 781 | AGACCAAA GGCTAGCTACAACGA CCCTTCTT | 2102 |
| 3508 | GGGGUUUC G UCUCUGUG | 782 | CACAGAGA GGCTAGCTACAACGA CAAACCCC | 2103 |
| 3514 | UGGUCUCU G UGCCAGCU | 783 | AGCTGGCA GGCTAGCTACAACGA AGAGACCA | 2104 |
| 3516 | GUCUCUGU G CCAGCUCU | 784 | AGAGCTGG GGCTAGCTACAACGA ACAGAGAC | 2105 |
| 3520 | CUGUGCCA G CUCUAUAA | 785 | TTATAGAG GGCTAGCTACAACGA TGGCACAG | 2106 |
| 3525 | CCAGCUCU A UAAUUGUU | 786 | AACAATTA GGCTAGCTACAACGA AGAGCTGG | 2107 |
| 3528 | GCUCUAUA A UUGUUUUG | 787 | CAAAACAA GGCTAGCTACAACGA TATAGAGC | 2108 |
| 3531 | CUAUAAUU G UUUUGCUA | 788 | TAGCAAAA GGCTAGCTACAACGA AATTATAG | 2109 |
| 3536 | AUUGUUUU G CUACGAUU | 789 | AATCGTAG GGCTAGCTACAACGA AAAACAAT | 2110 |
| 3539 | GUUUUGCU A CGAUUCCA | 790 | TGGAATCG GGCTAGCTACAACGA AGCAAAAC | 2111 |
| 3542 | UUGCUACG A UUCCACUG | 791 | CAGTGGAA GGCTAGCTACAACGA CGTAGCAA | 2112 |
| 3547 | ACGAUUCC A CUCAAACU | 792 | AGTTTCAG GGCTAGCTACAACGA GGAATCGT | 2113 |
| 3553 | CCACUGAA A CUCUUCGA | 793 | TCGAAGAG GGCTAGCTACAACGA TTCAGTGG | 2114 |
| 3561 | ACUCUUCG A UCAAGCUA | 794 | TAGCTTGA GGCTAGCTACAACGA CGAAGAGT | 2115 |
| 3566 | UCGAUCAA G CUACUUUA | 795 | TAAAGTAC GGCTAGCTACAACGA TTGATCGA | 2116 |
| 3569 | AUCAAGCU A CUUUAUGU | 796 | ACATAAAG GGCTAGCTACAACGA AGCTTGAT | 2117 |
| 3574 | GCUACUUU A UGUAAAUC | 797 | GATTTACA GGCTAGCTACAACGA AAAGTAGC | 2118 |
| 3576 | UACUUUAU G UAAAUCAC | 798 | GTGATTTA GGCTAGCTACAACGA ATAAAGTA | 2119 |
| 3580 | UUAUGUAA A UCACUUCA | 799 | TGAAGTGA GGCTAGCTACAACGA TTACATAA | 2120 |
| 3583 | UGUAAAUC A CUUCAUUG | 800 | CAATGAAG GGCTAGCTACAACGA GATTTACA | 2121 |
| 3588 | AUCACUUC A UUGUUUUA | 801 | TAAAACAA GGCTAGCTACAACGA GAAGTGAT | 2122 |
| 3591 | ACUUCAUU G UUUUAAAG | 802 | CTTTAAAA GGCTAGCTACAACGA AATGAACT | 2123 |
| 3602 | UUAAAGGA A UAAACUUG | 803 | CAAGTTTA GGCTAGCTACAACGA TCCTTTAA | 2124 |
| 3606 | AGGAAUAA A CUUGAUUA | 804 | TAATCAAG GGCTAGCTACAACGA TTATTCCT | 2125 |
| 3611 | UAAACUUG A UUAUAUUG | 805 | CAATATAA GGCTAGCTACAACGA CAAGTTTA | 2126 |
| 3614 | ACUUGAUU A UAUUGUUU | 806 | AAACAATA GCCTAGCTACAACGA AATCAAGT | 2127 |
| 3616 | UUGAUUAU A UUCUUUUU | 807 | AAAAACAA GGCTAGCTACAACGA ATAATCAA | 2128 |
| 3619 | AUUAUAUU G UUUUUUUA | 808 | TAAAAAAA GGCTAGCTACAACGA AATATAAT | 2129 |
| 3627 | GUUUUUUU A UUUGGCAU | 809 | ATGCCAAA GGCTAGCTACAACGA AAAAAAAC | 2130 |
| 3632 | UUUAUUUG G CAUAACUG | 810 | CAGTTATG GGCTAGCTACAACGA CAAATAAA | 2131 |
| 3634 | UAUUUGGC A UAACUGUG | 811 | CACAGTTA GGCTAGCTACAACGA GCCAAATA | 2132 |
| 3637 | UUGGCAUA A CUGUGAUU | 812 | AATCACAG GGCTAGCTACAACGA TATGCCAA | 2133 |
| 3640 | GCAUAACU G UGAUUCUU | 813 | AAGAATCA GGCTAGCTACAACGA AGTTATGC | 2134 |
| 3643 | UAACUGUG A UUCUUUUA | 814 | TAAAAGAA GGCTAGCTACAACGA CACAGTTA | 2135 |
| 3654 | CUUUUAGG A CAAUUACU | 815 | AGTAATTG GGCTAGCTACAACGA CCTAAAAG | 2136 |
| 3657 | UUAGGACA A UUACUGUA | 816 | TACAGTAA GGCTAGCTACAACGA TGTCCTAA | 2137 |
| 3660 | GGACAAUU A CUGUACAC | 817 | GTGTACAG GGCTAGCTACAACGA AATTGTCC | 2138 |
| 3663 | CAAUUACU G UACACAUU | 818 | AATGTGTA GGCTAGCTACAACGA AGTAATTG | 2139 |
| 3665 | AUUACUGU A CACAUUAA | 819 | TTAATGTG GGCTAGCTACAACGA ACAGTAAT | 2140 |
| 3667 | UACUGUAC A CAUUAAGG | 820 | CCTTAATG GGCTAGCTACAACGA GTACAGTA | 2141 |
| 3669 | CUGUACAC A UUAAGGUG | 821 | CACCTTAA GGCTAGCTACAACGA GTGTACAG | 2142 |
| 3675 | ACAUUAAG G UGUAUGUC | 822 | GACATACA GGCTAGCTACAACGA CTTAATGT | 2143 |
| 3677 | AUUAAGGU U UAUGUCAG | 823 | CTGACATA GGCTAGCTACAACGA ACCTTAAT | 2144 |
| 3679 | UAAUGUGU A UGUCAGAU | 824 | ATCTGACA GGCTAGCTACAACGA ACACCTTA | 2145 |
| 3681 | AGGUGUAU G UCAGAUAU | 825 | ATATCTGA GGCTAGCTACAACGA ATACACCT | 2146 |
| 3686 | UAUGUCAG A UAUUCAUA | 826 | TATGAATA GGCTAGCTACAACGA CTGACATA | 2147 |
| 3688 | UGUCAGAU A UUCAUAUU | 827 | AATATGAA GGCTAGCTACAACGA ATCTGACA | 2148 |
| 3692 | AGAUAUUC A UAUUGACC | 828 | GGTCAATA GGCTAGCTACAACGA GAATATCT | 2149 |
| 3694 | AUAUUCAU A UUGACCCA | 829 | TGGGTCAA GGCTAGCTACAACGA ATGAATAT | 2150 |
| 3698 | UCAUAUUG A CCCAAAUG | 830 | CATTTGGG GGCTAGCTACAACGA CAATATGA | 2151 |
| 3704 | UGACCCAA A UGUGUAAU | 831 | ATTACACA GGCTAGCTACAACGA TTGGGTCA | 2152 |
| 3706 | ACCCAAAU G UGUAAUAU | 832 | ATATTACA GGCTAGCTACAACGA ATTTGGGT | 2153 |
| 3708 | CCAAAUGU G UAAUAUUC | 833 | GAATATTA GGCTAGCTACAACGA ACATTTGG | 2154 |
| 3711 | AAUGUGUA A UAUUCCAG | 834 | CTGGAATA GGCTAGCTACAACGA TACACATT | 2155 |
| 3713 | UGUGUAAU A UUCCAGUU | 835 | AACTGGAA GGCTAGCTACAACGA ATTACACA | 2156 |
| 3719 | AUAUUCCA G UUUUCUCU | 836 | AGAGAAAA GGCTAGCTACAACGA TGGAATAT | 2157 |
| 3728 | UUUUCUCU G CAUAAGUA | 837 | TACTTATG GGCTAGCTACAACGA AGAGAAAA | 2158 |
| 3730 | UUCUCUGC A UAACUAAU | 838 | ATTACTTA GGCTAGCTACAACGA GCAGAGAA | 2159 |
| 3734 | CUGCAUAA G UAAUUAAA | 839 | TTTAATTA GGCTAGCTACAACGA TTATGCAG | 2160 |
| 3737 | CAUAAGUA A UUAAAAUA | 840 | TATTTTAA GGCTAGCTACAACGA TACTTATG | 2161 |
| 3743 | UAAUUAAA A UAUACUUA | 841 | TAAGTATA GGCTAGCTACAACGA TTTAATTA | 2162 |
| 3745 | AUUAAAAU A UACUUAAA | 842 | TTTAAGTA GGCTAGCTACAACGA ATTTTAAT | 2163 |
| 3747 | UAAAAUAU A CUUAAAAA | 843 | TTTTTAAG GGCTAGCTACAACGA ATATTTTA | 2164 |
| 3755 | ACUUAAAA A UUAAUAGU | 844 | ACTATTAA GGCTAGCTACAACGA TTTTAAGT | 2165 |
| 3759 | AAAAAUUA A UAGUUUUA | 845 | TAAAACTA GGCTAGCTACAACGA TAATTTTT | 2166 |
| 3762 | AAUUAAUA G UUUUAUCU | 846 | AGATAAAA GGCTAGCTACAACGA TATTAATT | 2167 |
| 3767 | AUAGUUUU A UCUGGGUA | 847 | TACCCAGA GGCTAGCTACAACGA AAAACTAT | 2168 |
| 3773 | UUAUCUGG G UACAAAUA | 848 | TATTTGTA GGCTAGCTACAACGA CCAGATAA | 2169 |
| 3775 | AUCUGGGU A CAAAUAAA | 849 | TTTATTTG GGCTAGCTACAACGA ACCCAGAT | 2170 |
| 3779 | GGGUACAA A UAAACAGU | 850 | ACTGTTTA GGCTAGCTACAACGA TTGTACCC | 2171 |
| 3783 | ACAAAUAA A CAGUGCCU | 851 | AGGCACTG GGCTAGCTACAACGA TTATTTGT | 2172 |
| 3786 | AAUAAACA G UGCCUGAA | 852 | TTCAGGCA GGCTAGCTACAACGA TGTTTATT | 2173 |
| 3788 | UAAACAGU U CCUGAACU | 853 | AGTTCAGG GGCTAGCTACAACGA ACTGTTTA | 2174 |
| 3794 | GUGCCUGA A CUAGUUCA | 854 | TGAACTAG GGCTAGCTACAACGA TCAGGCAC | 2175 |
| 3798 | CUGAACUA G UUCACAGA | 855 | TCTGTGAA GGCTAGCTACAACGA TAGTTCAG | 2176 |
| 3802 | ACUAGUUC A CAGACAAG | 856 | CTTGTCTG GGCTAGCTACAACGA GAACTAGT | 2177 |
| 3806 | GUUCACAG A CAAGGGAA | 857 | TTCCCTTG GGCTAGCTACAACGA CTGTGAAC | 2178 |
| 3815 | CAAGGGAA A CUUCUAUG | 858 | CATAGAAG GGCTAGCTACAACGA TTCCCTTG | 2179 |
| 3821 | AAACUUCU A UGUAAAAA | 859 | TTTTTACA GGCTAGCTACAACGA AGAAGTTT | 2180 |
| 3823 | ACUUCUAU G UAAAAAUC | 860 | GATTTTTA GGCTAGCTACAACGA ATAGAAGT | 2181 |
| 3829 | AUGUAAAA A UCACUAUG | 861 | CATAGTGA GGCTAGCTACAACGA TTTTACAT | 2182 |
| 3832 | UAAAAAUC A CUAUGAUU | 862 | AATGATAG GGCTAGCTACAACGA GATTTTTA | 2183 |
| 3835 | AAAUCACU A UGAUUUCU | 863 | AGAAATCA GGCTAGCTACAACGA AGTGATTT | 2284 |
| 3838 | UCACUAUG A UUUCUGAA | 864 | TTCAGAAA GGCTAGCTACAACGA CATAGTGA | 2185 |
| 3846 | AUUUCUGA A UUGCUAUG | 865 | CATAGCAA GGCTAGCTACAACGA TCAGAAAT | 2186 |
| 3849 | UCUGAAUU G CUAUGUGA | 866 | TCACATAG GGCTAGCTACAACGA AATTCAGA | 2187 |
| 3852 | GAAUUGCU A UGUGAAAC | 867 | GTTTCACA GGCTAGCTACAACGA AGCAATTC | 2188 |
| 3854 | AUUGCUAU G UGAAACUA | 868 | TAGTTTCA GGCTAGCTACAACGA ATAGCAAT | 2189 |
| 3859 | UAUGUGAA A CUACAGAU | 869 | ATCTGTAG GGCTAGCTACAACGA TTCACATA | 2190 |
| 3862 | GUCAAACU A CAGAUCUU | 870 | AAGATCTG GGCTAGCTACAACGA AGTTTCAC | 2191 |
| 3866 | AACUACAG A UCUUUGGA | 871 | TCCAAAGA GGCTAGCTACAACGA CTGTAGTT | 2192 |
| 3875 | UCUUUGGA A CACUGUUU | 872 | AAACAGTG GGCTAGCTACAACGA TCCAAAGA | 2193 |
| 3877 | UUUGGAAC A CUGUUUAG | 873 | CTAAACAG GGCTAGCTACAACGA GTTCCAAA | 2194 |
| 3880 | GGAACACU G UUUAGGUA | 874 | TACCTAAA GGCTAGCTACAACGA AGTGTTCC | 2195 |
| 3886 | CUGUUUAG G UACGGUCU | 875 | ACACCCTA GGCTAGCTACAACGA CTAAACAG | 2196 |
| 3891 | UAGGUAGU G UGUUAAGA | 876 | TCTTAACA GGCTAGCTACAACGA CCTACCTA | 2197 |
| 3893 | GGUAGGGU G UUAAGACU | 877 | AGTCTTAA GGCTAGCTACAACGA ACCCTACC | 2198 |
| 3899 | GUGUUAAG A CUUGACAC | 878 | GTGTCAAG GGCTAGCTACAACGA CTTAACAC | 2199 |
| 3904 | AAGACUUG A CACAGUAC | 879 | GTACTGTG GGCTAGCTACAACGA CAAGTCTT | 2200 |
| 3906 | GACUUGAC A CAGUACCU | 880 | AGGTACTG GGCTAGCTACAACGA GTCAAGTC | 2201 |
| 3909 | UUGACACA G UACCUCGU | 881 | ACGAGGTA GGCTAGCTACAACGA TGTGTCAA | 2202 |
| 3911 | GACACAGU A CCUCGUUU | 882 | AAACGAGG GGCTAGCTACAACGA ACTGTGTC | 2203 |
| 3916 | AGUACCUC G UUUCUACA | 883 | TGTAGAAA GGCTAGCTACAACGA GAGGTACT | 2204 |
| 3922 | UCGUUUCU A CACAGAGA | 884 | TCTCTGTG GGCTAGCTACAACGA AGAAACGA | 2205 |
| 3924 | GUUUCUAC A CAGAGAAA | 885 | TTTCTCTG GGCTAGCTACAACGA GTAGAAAC | 2206 |
| 3936 | AGAAAGAA A UGGCCAUA | 886 | TATGGCCA GGCTAGCTACAACGA TTCTTTCT | 2207 |
| 3939 | AAGAAAUG G CCAUACUU | 887 | AACTATGG GGCTAGCTACAACGA CATTTCTT | 2208 |
| 3942 | AAAUGGCC A UACUUCAG | 888 | CTGAAGTA GGCTAGCTACAACGA GGCCATTT | 2209 |
| 3944 | AUGGCCAU A CUUCAGGA | 889 | TCCTGAAG GGCTAGCTACAACGA ATGGCCAT | 2210 |
| 3953 | CUUCAGGA A CUGCAGUG | 890 | CACTGCAG GGCTAGCTACAACGA TCCTGAAG | 2211 |
| 3956 | CAGGAACU G CAGUGCUU | 891 | AAGCACTG GGCTAGCTACAACGA AGTTCCTG | 2212 |
| 3959 | GAACUGCA G UGCUUAUG | 892 | CATAAGCA GGCTAGCTACAACGA TGCAGTTC | 2213 |
| 3961 | ACUCCAGU G CUUAUGAG | 893 | CTCATAAG GGCTAGCTACAACGA ACTGCAGT | 2214 |
| 3965 | CAGUGCUU A UGAGGGGA | 894 | TCCCCTCA GGCTAGCTACAACGA AAGCACTG | 2215 |
| 3973 | AUGAGGGG A UAUUUAGG | 895 | CCTAAATA GGCTAGCTACAACGA CCCCTCAT | 2216 |
| 3975 | GAGCGGAU A UUUACGCC | 896 | GGCCTAAA GGCTAGCTACAACGA ATCCCCTC | 2217 |
| 3981 | AUAUUUAG G CCUCUUGA | 897 | TCAAGAGG GGCTAGCTACAACGA CTAAATAT | 2218 |
| 3990 | CCUCUUGA A UUUUUGAU | 898 | ATCAAAAA GGCTAGCTACAACGA TCAAGAGG | 2219 |
| 3997 | AAUUUUUC A UGUAGAUG | 899 | CATCTACA GGCTAGCTACAACGA CAAAAATT | 2220 |
| 3999 | UUUUUGAU G UAGAUGGG | 900 | CCCATCTA GGCTAGCTACAACGA ATCAAAAA | 2221 |
| 4003 | UGAUGUAG A UGGGCAUU | 901 | AATGCCCA GGCTAGCTACAACGA CTACATCA | 2222 |
| 4007 | GUAGAUGG G CAUUUUUU | 902 | AAAAAATG GGCTAGCTACAACGA CCATCTAC | 2223 |
| 4009 | AGAUGGGC A UUUUUUUA | 903 | TAAAAAAA GGCTAGCTACAACGA GCCCATCT | 2224 |
| 4020 | UUUUUAAG G UAGUGGUU | 904 | AACCACTA GGCTAGCTACAACGA CTTAAAAA | 2225 |
| 4023 | UUAAGGUA G UGGUUAAU | 905 | ATTAACCA GGCTAGCTACAACGA TACCTTAA | 2226 |
| 4026 | AGGUAGUG G UUAAUUAC | 906 | GTAATTAA GGCTAGCTACAACGA CACTACCT | 2227 |
| 4030 | AGUGGUUA A UUACCUUU | 907 | AAAGGTAA GGCTAGCTACAACGA TAACCACT | 2228 |
| 4033 | GGUUAAUU A CCUUUAUG | 908 | CATAAAGG GGCTAGCTACAACGA AATTAACC | 2229 |
| 4039 | UUACCUUU A UGUGAACU | 909 | AGTTCACA GGCTAGCTACAACGA AAAGGTAA | 2230 |
| 4041 | ACCUUUAU G UCAACUUU | 910 | AAAGTTCA GGCTAGCTACAACGA ATAAAGGT | 2231 |
| 4045 | UGAUGUGA A CUUUGAAU | 911 | ATTCAAAG GGCTAGCTACAACGA TCACATAA | 2232 |
| 4052 | AACUUUGA A UCCUUUAA | 912 | TTAAACCA GGCTAGCTACAACGA TCAAACTT | 2233 |
| 4055 | UUUGAAUG G UUUAACAA | 913 | TTGTTAAA GGCTAGCTACAACGA CATTCAAA | 2234 |
| 4060 | AUGGUUUA A CAAAAGAU | 914 | ATCTTTTG GGCTAGCTACAACGA TAAACCAT | 2235 |
| 4067 | AACAAAAG A UUUGUUUU | 915 | AAAACAAA GGCTAGCTACAACGA CTTTTGTT | 2236 |
| 4071 | AAAGAUUU G UUUUUGUA | 916 | TACAAAAA GGCTAGCTACAACGA AAATCTTT | 2237 |
| 4077 | UUGUUUUU G UAGAGAUU | 917 | AATCTCTA GGCTAGCTACAACGA AAAAACAA | 2238 |
| 4083 | UUGUAGAG A UUUUAAAG | 918 | CTTTAAAA GGCTAGCTACAACGA CTCTACAA | 2239 |
| 4099 | GGGGGAGA A UUCUAGAA | 919 | TTCTAGAA GGCTAGCTACAACGA TCTCCCCC | 2240 |
| 4108 | UUCUAGAA A UAAAUGUU | 920 | AACATTTA GGCTAGCTACAACGA TTCTAGAA | 2241 |
| 4112 | AGAAAUAA A UGUUACCU | 921 | AGGTAAGA GGCTAGCTACAACGA TTATTTCT | 2242 |
| 4114 | AAAUAAAU G UUACCUAA | 922 | TTAGGTAA GGCTAGCTACAACGA ATTTATTT | 2243 |
| 4117 | UAAAUGUU A CCUAAUUA | 923 | TAATTAGG GGCTAGCTACAACGA AACATTTA | 2244 |
| 4122 | GUUACCUA A UUAUUACA | 924 | TGTAATAA GGCTAGCTACAACGA TAGGTAAC | 2245 |
| 4125 | ACCUAAUU A UUACAGCC | 925 | GGCTGTAA GGCTAGCTACAACGA AATTAGGT | 2246 |
| 4128 | UAAUUAUU A CACCCUGA | 926 | TAAGGCTG GGCTAGCTACAACGA AATAATTA | 2247 |
| 4131 | UUAUUACA G CCUUAAAG | 927 | CTTTAAGG GGCTAGCTACAACGA TGTAATAA | 2248 |
| 4140 | CCUUAAAG A CAAAAAUC | 928 | GATTTTTG GGCTAGCTACAACGA CTTTAAGG | 2249 |
| 4146 | AGACAAAA A UCCUUGUU | 929 | AACAAGGA GGCTAGCTACAACGA TTTTGTCT | 2250 |
| 4152 | AAAUCCUU G UUGAAGUU | 930 | AACTTCAA GGCTAGCTACAACGA AAGGATTT | 2251 |
| 4158 | UUGUUGAA G UUUUUUUA | 931 | TAAAAAAA GGCTAGCTACAACGA TTCAACAA | 2252 |
| 4174 | AAAAAAAG A CUAAAUUA | 932 | TAATTTAG GGCTAGCTACAACGA CTTTTTTT | 2253 |
| 4179 | AAGACUAA A UUACAUAG | 933 | CTATGTAA GGCTAGCTACAACGA TTAGTCTT | 2254 |
| 4182 | ACUAAAUU A CAUAGACU | 934 | AGTCTATG GGCTAGCTACAACGA AATTTAGT | 2255 |
| 4184 | UAAAUUAC A UAGACUUA | 935 | TAAGTCTA GGCTAGCTACAACGA GTAATTTA | 2256 |
| 4188 | UUACAUAG A CUUAGGCA | 936 | TGCCTAAG GGCTAGCTACAACGA CTATGTAA | 2257 |
| 4194 | ACACUUAG G CAUUAACA | 937 | TGTTAATG GGCTAGCTACAACGA CTAAGTCT | 2258 |
| 4196 | ACUUAGGC A UUAACAUC | 938 | CATGTTAA GGCTAGCTACAACGA GCCTAAGT | 2259 |
| 4200 | AGGCAUUA A CAUGUUUG | 939 | CAAACATG GGCTAGCTACAACGA TAATGCCT | 2260 |
| 4202 | GCAUUAAC A UGUUUGUG | 940 | CACAAACA GGCTAGCTACAACGA GTTAATGC | 2261 |
| 4204 | AUUAACAU G UUUGUGGA | 941 | TCCACAAA GGCTAGCTACAACGA ATGTTAAT | 2262 |
| 4208 | ACAUGUUU G UGGAACAA | 942 | TTCTTCCA GGCTAGCTACAACGA AAACATGT | 2263 |
| 4216 | GUGGAAGA A UAUAGCAG | 943 | CTGCTATA GCCTAGCTACAACGA TCTTCCAC | 2264 |
| 4218 | GGAAGAAU A UAGCAGAC | 944 | GTCTGCTA GGCTAGCTACAACGA ATTCTTCC | 2265 |
| 4221 | AGAAUAUA G CAGACGUA | 945 | TACGTCTG GGCTAGCTACAACGA TATATTCT | 2266 |
| 4225 | UAUAGCAG A CGUAUAUU | 946 | AATATACG GGCTAGCTACAACGA CTGCTATA | 2267 |
| 4227 | UAGCAGAC G UAUAUUGU | 947 | ACAATATA GGCTAGCTACAACGA GTCTGCTA | 2268 |
| 4229 | GCAGACGU A UAUUGUAU | 948 | ATACAATA GGCTAGCTACAACGA ACGTCTGC | 2269 |
| 4231 | AGACGUAC A UUGUAUCA | 949 | TGATACAA GGCTAGCTACAACGA ATACGTCT | 2270 |
| 4234 | CGUAUAUU G UAUCAUUU | 950 | AAATGATA GGCTAGCTACAACGA AATATACG | 2271 |
| 4236 | UAUAUUGU A UCAUUUGA | 951 | TCAAATGA GGCTAGCTACAACGA ACAATATA | 2272 |
| 4239 | AUUGUAUC A UUUGAGUG | 952 | CACTCAAA GGCTAGCTACAACGA GATACAAT | 2273 |
| 4245 | UCAUUUGA G UGAAUGUU | 953 | AACATTCA GGCTAGCTACAACGA TCAAATGA | 2274 |
| 4249 | UUGAGUGA A UGUUCCCA | 954 | TGGGAACA GGCTAGCTACAACGA TCACTCAA | 2275 |
| 4251 | GAGUGAAU G UUCCCAAG | 955 | CTTGGGAA GGCTAGCTACAACGA ATTCACTC | 2276 |
| 4259 | GUUCCCAA G UAGGCAUU | 956 | AATCCCTA GGCTAGCTACAACGA TTGGGAAC | 2277 |
| 4263 | CCAAGUAG G CAUUCUAG | 957 | CTAGAATG GGCTAGCTACAACGA CTACTTGG | 2278 |
| 4265 | AACUAGGC A UUCUAGGC | 958 | GCCTAGAA GGCTAGCTACAACGA GCCTACTT | 2279 |
| 4272 | CAUUCUAG G CUCUAUUU | 959 | AAATAGAG GGCTAGCTACAACGA CTAGAATG | 2280 |
| 4277 | UAGGCUCU A UUUAACUG | 960 | CAGTTAAA GGCTAGCTACAACGA AGAGCCTA | 2281 |
| 4282 | UCUAUUUA A CUGAGUCA | 961 | TGACTCAG GGCTAGCTACAACGA TAAATAGA | 2282 |
| 4287 | UUAACUGA G UCACACUG | 962 | CAGTGTGA GGCTAGCTACAACGA TCAGTTAA | 2283 |
| 4290 | ACUGAGUC A CACUGCAU | 963 | ATGCAGTG GGCTAGCTACAACGA GACTCAGT | 2284 |
| 4292 | UGAGUCAC A CUGCAUAG | 964 | CTATGCAG GGCTAGCTACAACGA GTGACTCA | 2285 |
| 4295 | GUCACACU G CAUAGGAA | 965 | TTCCTATG GGCTAGCTACAACGA AGTGTGAC | 2286 |
| 4297 | CACACUGC A UAGGAAUU | 966 | AATTCCTA GGCTAGCTACAACGA GCAGTGTG | 2287 |
| 4303 | GCAUAGGA A UUUAGAAC | 967 | GTTCTAAA GGCTAGCTACAACGA TCCTATGC | 2288 |
| 4310 | AAUUUAGA A CCUAACUU | 968 | AAGTTAGG GGCTAGCTACAACGA TCTAAATT | 2289 |
| 4315 | AGAACCUA A CUUUUAUA | 969 | TATAAAAG GGCTAGCTACAACGA TAGGTTCT | 2290 |
| 4321 | UAACUUUU A UAGGUUAU | 970 | ATAACCTA GGCTAGCTACAACGA AAAAGTTA | 2291 |
| 4325 | UUUUAUAG G UUAUCAAA | 971 | TTTGATAA GGCTAGCTACAACGA CTATAAAA | 2292 |
| 4328 | UAUAGGUU A UCAAAACU | 972 | AGTTTTGA GGCTAGCTACAACGA AACCTATA | 2293 |
| 4334 | UUAUCAAA A CUGUUGUC | 973 | GACAACAG GGCTAGCTACAACGA TTTGATAA | 2294 |
| 4337 | UCAAAACU G UUGUCACC | 974 | GGTGACAA GGCTAGCTACAACGA AGTTTTGA | 2295 |
| 4340 | AAACUGUU G UCACCAUU | 975 | AATGGTGA GGCTAGCTACAACGA AACAGTTT | 2296 |
| 4343 | CUGUUGUC A CCAUUGCA | 976 | TGCAATGG GGCTAGCTACAACGA GAGAACAG | 2297 |
| 4346 | UUGUCACC A UUGCACAA | 977 | TTGTGCAA GGCTAGCTACAACGA GGTGACAA | 2298 |
| 4349 | UCACCAUU G CACAAUUU | 978 | AAATTGTG GGCTAGCTACAACGA AATGGTGA | 2299 |
| 4351 | ACCAUUGC A CAAUUUUG | 979 | CAAAATTG GGCTAGCTACAACGA GCAATGGT | 2300 |
| 4354 | AUUGCACA A UUUUGUCC | 980 | GGACAAAA GGCTAGCTACAACGA TGTGCAAT | 2301 |
| 4359 | ACAAUUUU G UCCUAAUA | 981 | TATTAGGA GGCTAGCTACAACGA AAAATTGT | 2302 |
| 4365 | UUGUCCUA A UAUAUACA | 982 | TGTATATA GGCTAGCTACAACGA TAGGACAA | 2303 |
| 4367 | GUCCUAAU A UAUACAUA | 983 | TATGTATA GGCTAGCTACAACGA ATTAGGAC | 2304 |
| 4369 | CCUAAUAU A UACAUAGA | 984 | TCTATGTA GGCTAGCTACAACGA ATATTAGG | 2305 |
| 4371 | UAAUAUAU A CAUAGAAA | 985 | TTTCTATG GGCTAGCTACAACGA ATATATTA | 2306 |
| 4373 | AUAUAUAC A UAGAAACU | 986 | AGTTTCTA GGCTAGCTACAACGA GTATATAT | 2307 |
| 4379 | ACAUAGAA A CUUUGUGG | 987 | CCACAAAG GGCTAGCTACAACGA TTCTATGT | 2308 |
| 4384 | GAAACUUU G UGGGGCAU | 988 | ATGCCCCA GGCTAGCTACAACGA AAAGTTTC | 2309 |
| 4389 | UUUGUGGG G CAUGUUAA | 989 | TTAACATG GGCTAGCTACAACGA CCCACAAA | 2310 |
| 4391 | UGUGGGGC A UGUUAAGU | 990 | ACTTAACA GGCTAGCTACAACGA GCCCCACA | 2311 |
| 4393 | UGGGGCAU G UUAAGUUA | 991 | TAACTTAA GGCTAGCTACAACGA ATGCCCCA | 2312 |
| 4398 | CAUGUUAA G UUACAGUU | 992 | AACTGTAA GGCTAGCTACAACGA TTAACATG | 2313 |
| 4401 | GUUAAGUU A CAGUUUGC | 993 | GCAAACTG GGCTAGCTACAACGA AACTTAAC | 2314 |
| 4404 | AAGUUACA G UUUGCACA | 994 | TGTCCAAA GGCTAGCTACAACGA TGTAACTT | 2315 |
| 4408 | UACAGUUU G CACAAGUU | 995 | AACTTGTG GGCTAGCTACAACGA AAACTGTA | 2316 |
| 4410 | CAGUUUGC A CAAGUUCA | 996 | TGAACTTG GGCTAGCTACAACGA GCAAACTG | 2317 |
| 4414 | UUGCACAA G UUCAUCUC | 997 | GAGATGAA GGCTAGCTACAACGA TTGTGCAA | 2318 |
| 4418 | ACAAGUUC A UCUCAUUU | 998 | AAATGAGA GGCTAGCTACAACGA GAACTTGT | 2319 |
| 4423 | UUCAUCUC A UUUGUAUU | 999 | AATACAAA GGCTAGCTACAACGA GAGATGAA | 2320 |
| 4427 | UCUCAUUU G UAUUCCAU | 1000 | ATGGAATA GGCTACCTACAACGA AAATGAGA | 2321 |
| 4429 | UCAUUUGU A UUCCAUUG | 1001 | CAATGGAA GGCTAGCTACAACGA ACAAATGA | 2322 |
| 4434 | UGUAUUCC A UUGAUUUU | 1002 | AAAATCAA GGCTAGCTACAACGA GGAATACA | 2323 |
| 4438 | UUCCAUUG A UUUUUUUU | 1003 | AAAAAAAA GGCTAGCTACAACGA CAATGGAA | 2324 |
| 4457 | UCUUCUAA A CAUUUUUU | 1004 | AAAAAATG GGCTAGCTACAACGA TTAGAAGA | 2325 |
| 4459 | UUCUAAAC A UUUUUUCU | 1005 | AGAAAAAA GGCTAGCTACAACGA GTTTAGAA | 2326 |
| 4473 | UCUUCAAA A CAGUAUAU | 1006 | ATATACTG GGCTAGCTACAACGA TTTGAAGA | 2327 |
| 4476 | UCAAAACA G UAUAUAUA | 1007 | TATATATA GGCTAGCTACAACGA TGTTTTGA | 2328 |
| 4478 | AAAACAGU A UAUAUAAC | 1008 | GTTATATA GGCTAGCTACAACGA ACTGTTTT | 2329 |
| 4480 | AACAGUAU A UAUAACUU | 1009 | AAGTTATA GGCTAGCTACAACGA ATACTGTT | 2330 |
| 4482 | CAGUAUAU A UAACUUUU | 1010 | AAAAGTTA GGCTAGCTACAACGA ATATACTG | 2331 |
| 4485 | UAUAUAUA A CUUUUUUU | 1011 | AAAAAAAG GGCTAGCTACAACGA TATATATA | 2332 |
| 4499 | UUUAGGGG A UUUUUUUU | 1012 | AAAAAAAA GGCTAGCTACAACGA CCCCTAAA | 2333 |
| 4510 | UUUUUUAG A CAGCAAAA | 1013 | TTTTGCTG GGCTAGCTACAACGA CTAAAAAA | 2334 |
| 4513 | UUUAGACA G CAAAAAAC | 1014 | GTTTTTTG GGCTAGCTACAACGA TGTCTAAA | 2335 |
| 4520 | AGCAAAAA A CUAUCUGA | 1015 | TCAGATAG GGCTAGCTACAACGA TTTTTGCT | 2336 |
| 4523 | AAAAAACU A UCUGAAGA | 1016 | TCTTCAGA GGCTAGCTACAACGA AGTTTTTT | 2337 |
| 4531 | AUCUGAAG A UUUCCAUU | 1017 | AATGGAAA GGCTAGCTACAACGA CTTCAGAT | 2338 |
| 4537 | AGAUUUCC A UUUGUCAA | 1018 | TTGACAAA GGCTAGCTACAACGA GGAAATCT | 2339 |
| 4541 | UUCCAUUU G UCAAAAAG | 1019 | CTTTTTGA GGCTAGCTACAACGA AAATGGAA | 2340 |
| 4549 | GUCAAAAA G UAAUGAUU | 1020 | AATCATTA GGCTAGCTACAACGA TTTTTGAC | 2341 |
| 4552 | AAAAAGUA A UGAUUUCU | 1021 | AGAAATCA GGCTAGCTACAACGA TACTTTTT | 2342 |
| 4555 | AAGUAAUG A UUUCUUGA | 1022 | TCAAGAAA GGCTAGCTACAACGA CATTACTT | 2343 |
| 4563 | AUUUCUUG A UAAUUGUG | 1023 | CACAATTA GGCTAGCTACAACGA CAAGAAAT | 2344 |
| 4566 | UCUUGAUA A UUGUGUAG | 1024 | CTACACAA GGCTAGCTACAACGA TATCAAGA | 2345 |
| 4569 | UGAUAAUU G UGUAGUGA | 1025 | TCACTACA GGCTAGCTACAACGA AATTATCA | 2346 |
| 4571 | AUAAUUGU G UAGUGAAU | 1026 | ATTCACTA GGCTAGCTACAACGA ACAATTAT | 2347 |
| 4574 | AUUGUGUA G UGAAUGUU | 1027 | AACATTCA GGCTAGCTACAACGA TACACAAT | 2348 |
| 4578 | UGUAGUGA A UGUUUUUU | 1028 | AAAAAACA GGCTAGCTACAACGA TCACTACA | 2349 |
| 4580 | UAGUGAAU G UUUUUUAG | 1029 | CTAAAAAA GGCTAGCTACAACGA ATTCACTA | 2350 |
| 4590 | UUUUUAGA A CCCAGCAG | 1030 | CTGCTGGG GGCTAGCTACAACGA TCTAAAAA | 2351 |
| 4595 | AGAACCCA G CAGUUACC | 1031 | GGTAACTG GGCTAGCTACAACGA TGGGTTCT | 2352 |
| 4598 | ACCCAGCA G UUACCUUG | 1032 | CAAGGTAA GGCTAGCTACAACGA TGCTGGCT | 2353 |
| 4601 | CAGCAGUU A CCUUGAAA | 1033 | TTTCAAGG GGCTAGCTACAACGA AACTGCTG | 2354 |
| 4610 | CCUUGAAA G CUGAAUUU | 1034 | AAATTCAG GGCTAGCTACAACGA TTTCAAGG | 2355 |
| 4615 | AAAGCUGA A UUUAUAUU | 1035 | AATATAAA GGCTAGCTACAACGA TCAGCTTT | 2356 |
| 4619 | CUGAAUUU A UAUUUAGU | 1036 | ACTAAATA GGCTAGCTACAACGA AAATTCAG | 2357 |
| 4621 | GAAUUUAU A UUUAGUAA | 1037 | TTACTAAA GGCTAGCTACAACGA ATAAATTC | 2358 |
| 4626 | UAUAUUUA G UAACUUCU | 1038 | AGAAGTTA GGCTAGCTACAACGA TAAATATA | 2359 |
| 4629 | AUUUAGUA A CUUCUGUG | 1039 | CACAGAAG GGCTAGCTACAACGA TACTAAAT | 2360 |
| 4635 | UAACUUCU G UGUUAAUA | 1040 | TATTAACA GGCTAGCTACAACGA AGAAGTTA | 2361 |
| 4637 | ACUUCUGU G UUAAUACU | 1041 | AGTATTAA GGCTAGCTACAACGA ACAGAAGT | 2362 |
| 4641 | CUGUGUUA A UACUGGAU | 1042 | ATCCAGTA GGCTAGCTACAACGA TAACACAG | 2363 |
| 4643 | GUGUUAAU A CUGGAUAG | 1043 | CTATCCAG GGCTAGCTACAACGA ATTAACAC | 2364 |
| 4648 | AAUACUGG A UAGCAUGA | 1044 | TCATGCTA GGCTAGCTACAACGA CCAGTATT | 2365 |
| 4651 | ACUGGAUA G CAUGAAUU | 1045 | AATTCATG GGCTAGCTACAACGA TATCCAGT | 2366 |
| 4653 | UGGAUAGC A UGAAUUCU | 1046 | AGAATTCA GGCTAGCTACAACGA GCTATCCA | 2367 |
| 4657 | UAGCAUGA A UUCUGCAU | 1047 | ATGCAGAA GGCTAGCTACAACGA TCATGCTA | 2368 |
| 4662 | UGAAUUCU G CAUUGAGA | 1048 | TCTCAATG GGCTAGCTACAACGA AGAATTCA | 2369 |
| 4664 | AAUUCUGC A UUGAGAAA | 1049 | TTTCTCAA GGCTAGCTACAACGA GCAGAATT | 2370 |
| 4672 | AUUGAGAA A CUGAAUAG | 1050 | CTATTCAG GGCTAGCTACAACGA TTCTCAAT | 2371 |
| 4677 | GAAACUGA A UAGCUGUC | 1051 | GACAGCTA GGCTAGCTACAACGA TCAGTTTC | 2372 |
| 4680 | ACUGAAUA G CUGUCAUA | 1052 | TATGACAG GGCTAGCTACAACGA TATTCAGT | 2373 |
| 4683 | GAAUAGCU G UCAUAAAA | 1053 | TTTTATGA GGCTAGCTACAACGA AGCTATTC | 2374 |
| 4686 | UAGCUGUC A UAAAAUCC | 1054 | GCATTTTA GGCTAGCTACAACGA GACAGCTA | 2375 |
| 4691 | GUCAUAAA A UCCUUUCU | 1055 | AGAAAGCA GGCTAGCTACAACGA TTTATGAC | 2376 |
| 4693 | CAUAAAAU G CUUUCUUU | 1056 | AAAGAAAG GGCTAGCTACAACGA ATTTTATG | 2377 |
| 4713 | AAAGAAAG A UACUCACA | 1057 | TGTGAGTA GGCTAGCTACAACGA CTTTCTTT | 2378 |
| 4715 | AGAAAGAU A CUCACAUG | 1058 | CATGTGAG GGCTAGCTACAACGA ATCTTTCT | 2379 |
| 4719 | AGAUACUC A CAUGACUU | 1059 | AACTCATG GGCTAGCTACAACGA GAGTATCT | 2380 |
| 4721 | AUACUCAC A UGAGUUCU | 1060 | AGAACTCA GGCTAGCTACAACGA GTGAGTAT | 2381 |
| 4725 | UCACAUGA G UUCUUGAA | 1061 | TTCAAGAA GGCTAGCTACAACGA TCATGTGA | 2382 |
| 4736 | CUUGAAGA A UAGUCAUA | 1062 | TATGACTA GGCTAGCTACAACGA TCTTCAAG | 2383 |
| 4739 | GAAGAAUA G UCAUAACU | 1063 | AGTTATGA GGCTAGCTACAACGA TATTCTTC | 2384 |
| 4742 | GAAUAGUC A UAACUAGA | 1064 | TCTAGTTA GGCTAGCTACAACGA GACTATTC | 2385 |
| 4745 | UAGUCAUA A CUAGAUUA | 1065 | TAATCTAG GGCTAGCTACAACGA TATGACTA | 2386 |
| 4750 | AUAACUAG A UUAAGAUC | 1066 | GATCTTAA GGCTAGCTACAACGA CTAGTTAT | 2387 |
| 4756 | AGAUUAAG A UCUGUGUU | 1067 | AACACAGA GGCTAGCTACAACGA CTTAATCT | 2388 |
| 4760 | UAAGAUCU G UGUUUUAC | 1068 | CTAAAACA GGCTAGCTACAACGA AGATCTTA | 2389 |
| 4762 | AGAUCUGU G UUUUAGUU | 1069 | AACTAAAA GGCTAGCTACAACGA ACAGATCT | 2390 |
| 4768 | GUGUUUUA G UUUAAUAG | 1070 | CTATTAAA GGCTAGCTACAACGA TAAAACAC | 2391 |
| 4773 | UUAGUUUA A UAGUUUGA | 1071 | TCAAACTA GGCTAGCTACAACGA TAAACTAA | 2392 |
| 4776 | GUUUAAUA G UUUGAAGU | 1072 | ACTTCAAA GGCTAGCTACAACGA TATTAAAC | 2393 |
| 4783 | AGUUUGAA G UGCCUGUU | 1073 | AACAGGCA GGCTAGCTACAACGA TTCAAACT | 2394 |
| 4785 | UUUGAAGU G CCUGUUUG | 1074 | CAAACAGG GGCTAGCTACAACGA ACTTCAAA | 2395 |
| 4789 | AAGUGCCU G UUUGGGAU | 1075 | ATCCCAAA GGCTAGCTACAACGA AGGCACTT | 2396 |
| 4796 | UGUUUGGG A UAAUGAUA | 1076 | TATCATTA GGCTAGCTACAACGA CCCAAACA | 2397 |
| 4799 | UUGGGAUA A UGAUAGGU | 1077 | ACCTATCA GGCTAGCTACAACGA TATCCCAA | 2398 |
| 4802 | GGAUAAUG A UAGGUAAU | 1078 | ATTACCTA GGCTAGCTACAACGA CATTATCC | 2399 |
| 4806 | AAUGAUAG G UAAUUUAG | 1079 | CTAAATTA GGCTAGCTACAACGA CTATCATT | 2400 |
| 4809 | GAUAGGUA A UUUAGAUG | 1080 | CATCTAAA GGCTAGCTACAACGA TACCTATC | 2401 |
| 4815 | UAAUUUAG A UGAAUUUA | 1081 | TAAATTCA GGCTAGCTACAACGA CTAAATTA | 2402 |
| 4819 | UUAGAUGA A UUUAGGGG | 1082 | CCCCTAAA GGCTAGCTACAACGA TCATCTAA | 2403 |
| 4836 | AAAAAAAA G UUAUCUGC | 1083 | GCAGATAA GGCTAGCTACAACGA TTTTTTTT | 2404 |
| 4839 | AAAAAGUU A UCUGCAGU | 1084 | ACTGCAGA GGCTAGCTACAACGA AACTTTTT | 2405 |
| 4843 | AGUUAUCU G CAGUUAUG | 1085 | CATAACTG GGCTAGCTACAACGA AGATAACT | 2406 |
| 4846 | UAUCUGCA G UUAUGUUG | 1086 | CAACATAA GGCTAGCTACAACGA TGCAGATA | 2407 |
| 4849 | CUGCAGUU A UGUUGAGG | 1087 | CCTCAACA GGCTAGCTACAACGA AACTGCAG | 2408 |
| 4851 | GCAGUUAU G UUGAGGGC | 1088 | GCCCTCAA GGCTAGCTACAACGA ATAACTGC | 2409 |
| 4858 | UGUUGACG G CCCAUCUC | 1089 | GAGATGGG GGCTAGCTACAACGA CCTCAACA | 2410 |
| 4862 | GAGGGCCC A UCUCUCCC | 1090 | GGGAGAGA GGCTAGCTACAACGA GGGCCCTC | 2411 |
| 4874 | CUCCCCCC A CACCCCCA | 1091 | TGGGGGTG GGCTAGCTACAACGA GGGGGGAG | 2412 |
| 4876 | CCCCCCAC A CCCCCACA | 1092 | TGTGGGGG GGCTAGCTACAACGA GTGGGGGG | 2413 |
| 4882 | ACACCCCC A CAGAGCUA | 1093 | TAGCTCTG GGCTAGCTACAACGA GGGGGTGT | 2414 |
| 4887 | CCCACAGA G CUAACUGG | 1094 | CCAGTTAC GGCTAGCTACAACGA TCTGTGGG | 2415 |
| 4891 | CAGAGCUA A CUGGGUUA | 1095 | TAACCCAG GGCTAGCTACAACGA TAGCTCTG | 2416 |
| 4896 | CUAACUGG G UUACAGUG | 1096 | CACTGTAA GGCTAGCTACAACGA CCAGTTAG | 2417 |
| 4899 | ACUGGGUU A CAGUGUUU | 1097 | AAACACTG GGCTAGCTACAACGA AACCCAGT | 2418 |
| 4902 | GGGUUACA G UGUUUUAU | 1098 | ATAAAACA GGCTAGCTACAACGA TGTAACCC | 2419 |
| 4904 | GUUACAGU G UUUUAUCC | 1099 | GGATAAAA GGCTAGCTACAACGA ACTGTAAC | 2420 |
| 4909 | AGUCUUUU A UCCGAAAG | 1100 | CTTTCGGA GGCTAGCTACAACGA AAAACACT | 2421 |
| 4917 | AUCCGAAA G UUUCCAAU | 1101 | ATTGGAAA GGCTAGCTACAACGA TTTCGGAT | 2422 |
| 4924 | AGUUUCCA A UUCCACUG | 1102 | CAGTGGAA GGCTAGCTACAACGA TGGAAACT | 2423 |
| 4929 | CCAAUUCC A CUGUCUUG | 1103 | CAAGAGAG GGCTAGCTACAACGA GGAATTGG | 2424 |
| 4932 | AUUCCACU G UCUUGUGU | 1104 | ACACAAGA GGCTAGCTACAACGA AGTGGAAT | 2425 |
| 4937 | ACUGUCUU G UGUUUUCA | 1105 | TGAAAACA GGCTAGCTACAACGA AAGACAGT | 2426 |
| 4939 | UGUCUUGU G UUUUCAUG | 1106 | CATGAAAA GGCTAGCTACAACGA ACAAGACA | 2427 |
| 4945 | GUGUUUUC A UGUUGAAA | 1107 | TTTCAACA GGCTAGCTACAACGA GAAAACAC | 2428 |
| 4947 | GUUUUCAU G UUGAAAAU | 1108 | ATTTTCAA GGCTAGCTACAACGA ATGAAAAC | 2429 |
| 4954 | UGUUGAAA A UACUUUUG | 1109 | CAAAAGTA GGCTAGCTACAACGA TTTCAACA | 2430 |
| 4956 | UUGAAAAU A CUUUUGCA | 1110 | TGCAAAAG GGCTAGCTACAACGA ATTTTCAA | 2431 |
| 4962 | AUACUUUU G CAUUUUUC | 1111 | GAAAAATG GGCTAGCTACAACGA AAAAGTAT | 2432 |
| 4964 | ACUUUUGC A UUUUUCCU | 1112 | AGGAAAAA GGCTAGCTACAACGA GCAAAAGT | 2433 |
| 4977 | UCCUUUGA G UGCCAAUU | 1113 | AATTGGCA GGCTAGCTACAACGA TCAAAGGA | 2434 |
| 4979 | CUUUCACU G CCAAUUUC | 1114 | GAAATTGG GGCTAGCTACAACGA ACTCAAAG | 2435 |
| 4983 | GAGUGCCA A UUUCUUAC | 1115 | GTAAGAAA GGCTAGCTACAACGA TGGCACTC | 2436 |
| 4990 | AAUUUCUU A CUAGUACU | 1116 | AGTACTAG GGCTAGCTACAACGA AAGAAATT | 2437 |
| 4994 | UCUUACUA G UACUAUUU | 1117 | AAATAGTA GGCTAGCTACAACGA TAGTAAGA | 2438 |
| 4996 | UUACUAGU A CUAUUUCU | 1118 | AGAAATAG GGCTAGCTACAACGA ACTAGTAA | 2439 |
| 4999 | CUAGUACU A UUUCUUAA | 1119 | TTAAGAAA GGCTAGCTACAACGA AGTACTAG | 2440 |
| 5007 | AUUUCUUA A UGUAACAU | 1120 | ATGTTACA GGCTAGCTACAACGA TAAGAAAT | 2441 |
| 5009 | UUCUUAAU G UAACAUGU | 1121 | ACATGTTA GGCTAGCTACAACGA ATTAAGAA | 2442 |
| 5012 | UUAAUGUA A CAUGUUUA | 1122 | TAAACATG GGCTAGCTACAACGA TACATTAA | 2443 |
| 5014 | AAUGUAAC A UGUUUACC | 1123 | GGTAAACA GGCTAGCTACAACGA GTTACATT | 2444 |
| 5016 | UGUAACAU G UUUACCUG | 1124 | CAGGTAAA GGCTAGCTACAACGA ATGTTACA | 2445 |
| 5020 | ACAUGUUU A CCUGGCCU | 1125 | AGGCCAGG GGCTAGCTACAACGA AAACATGT | 2446 |
| 5025 | UUUACCUG G CCUGUCUU | 1126 | AAGACAGG GGCTAGCTACAACGA CACGTAAA | 2447 |
| 5029 | CCUGGCCU G UCUUUUAA | 1127 | TTAAAAGA GGCTAGCTACAACGA AGGCCAGG | 2448 |
| 5037 | GUCUUUUA A CUAUUUUU | 1128 | AAAAATAG GGCTACCTACAACCA TAAAAGAC | 2449 |
| 5040 | UUUUAACU A UUUUUCUA | 1129 | TACAAAAA GGCTAGCTACAACGA AGTTAAAA | 2450 |
| 5046 | CUAUUUUU G UAUAGUGU | 1130 | ACACTATA GGCTAGCTACAACGA AAAAATAG | 2451 |
| 5048 | AUUUUUCU A UAGUGUAA | 1131 | TTACACTA GGCTAGCTACAACGA ACAAAAAT | 2452 |
| 5051 | UUUGUAUA G UCUAAACU | 1132 | AGTTTACA GGCTAGCTACAACGA TATACAAA | 2453 |
| 5053 | UGUAUACU G UAAACUGA | 1133 | TCAGTTTA GGCTACCTACAACGA ACTATACA | 2454 |
| 5057 | UAGUGUAA A CUGAAACA | 1134 | TGTTTCAG GGCTAGCTACAACGA TTACACTA | 2455 |
| 5063 | AAACUGAA A CAUGCACA | 1135 | TGTGCATG GGCTAGCTACAACGA TTCAGTTT | 2456 |
| 5065 | ACUGAAAC A UGCACAUU | 1136 | AATGTGCA GGCTAGCTACAACGA GTTTCAGT | 2457 |
| 5067 | UGAAACAU G CACAUUUU | 1137 | AAAATGTG GGCTAGCTACAACGA ATGTTTCA | 2458 |
| 5069 | AAACAUGC A CAUUUUGU | 1138 | ACAAAATG GGCTAGCTACAACGA GCATGTTT | 2459 |
| 5071 | ACAUGCAC A UUUUGUAC | 1139 | GTACAAAA GGCTAGCTACAACGA GTGCATGT | 2460 |
| 5076 | CACAUUUU G UACAUUGU | 1140 | ACAATGTA GGCTAGCTACAACGA AAAATGTG | 2461 |
| 5078 | CAUUUUGU A CAUUGUGC | 1141 | GCACAATG GGCTAGCTACAACGA ACAAAATG | 2462 |
| 5080 | UUUUGUAC A UUGUGCUU | 1142 | AAGCACAA GGCTAGCTACAACGA GTACAAAA | 2463 |
| 5083 | UGUACAUU G UGCUUUCU | 1143 | AGAAAGCA GGCTAGCTACAACGA AATGTACA | 2464 |
| 5085 | UACAUUCU G CUUUCUUU | 1144 | AAAGAAAC GGCTAGCTACAACGA ACAATGTA | 2465 |
| 5095 | UUUCUUUU G UGGGUCAU | 1145 | ATGACCCA GGCTAGCTACAACGA AAAAGAAA | 2466 |
| 5099 | UUUUGUGG G UCAUAUGC | 1146 | GCATATGA GGCTAGCTACAACGA CCACAAAA | 2467 |
| 5102 | UCUGGGUC A UAUGCAGU | 1147 | ACTGCATA GGCTAGCTACAACGA GACCCACA | 2468 |
| 5104 | UGGGUCAU A UGCAGUGU | 1148 | ACACTGCA GGCTAGCTACAACGA ATGACCCA | 2469 |
| 5106 | GGUCAUAU G CAGUGUGA | 1149 | TCACACTG GGCTAGCTACAACGA ATATGACC | 2470 |
| 5109 | CAUAUGCA G UGUGAUCC | 1150 | GGATCACA GGCTAGCTACAACGA TGCATATG | 2471 |
| 5111 | UAUGCACU G UGAUCCAG | 1151 | CTGGATCA GGCTAGCTACAACGA ACTGCATA | 2472 |
| 5114 | GCAGUGUG A UCCAGUUG | 1152 | CAACTGGA GGCTAGCTACAACGA CACACTGC | 2473 |
| 5119 | GUGAUCCA G UUCUUUUC | 1153 | GAAAACAA GGCTAGCTACAACGA TGGATCAC | 2474 |
| 5122 | AUCCAGUU G UUUUCCAU | 1154 | ATGGAAAA GGCTAGCTACAACGA AACTGGAT | 2475 |
| 5129 | UGUUUUCC A UCAUUUGG | 1155 | CCAAATGA GGCTAGCTACAACGA GGAAAACA | 2476 |
| 5132 | UUUCCAUC A UUUGGUUG | 1156 | CAACCAAA GGCTAGCTACAACGA GATGGAAA | 2477 |
| 5137 | AUCAUUUG G UUGCGCUG | 1157 | CAGCGCAA GGCTAGCTACAACGA CAAATGAT | 2478 |
| 5140 | AUUGGUUU G CGCUGACC | 1158 | GGTCAGCG GGCTAGCTACAACGA AACCAAAT | 2479 |
| 5142 | UUGGUUGC G CUGACCUA | 1159 | TAGGTCAG GGCTAGCTACAACGA GCAACCAA | 2480 |
| 5146 | UUGCGCUG A CCUAGGAA | 1160 | TTCCTAGG GGCTAGCTACAACGA CAGCGCAA | 2481 |
| 5154 | ACCUAGGA A UGUUGGUC | 1161 | GACCAACA GGCTAGCTACAACGA TCCTAGGT | 2482 |
| 5156 | CUAGGAAU G UUGGUCAU | 1162 | ATGACCAA GGCTAGCTACAACGA ATTCCTAG | 2483 |
| 5160 | GAAUGUUG G UCAUAUCA | 1163 | TGATATGA GGCTAGCTACAACGA CAACATTC | 2484 |
| 5163 | UGUUGGUC A UAUCAAAC | 1164 | GTTTGATA GGCTAGCTACAACGA GACCAACA | 2485 |
| 5165 | UUGGUCAU A UCAAACAU | 1165 | ATGTTTGA GGCTAGCTACAACGA ATGACCAA | 2486 |
| 5170 | CAUAUCAA A CAUUAAAA | 1166 | TTTTAATG GGCTAGCTACAACGA TTGATATG | 2487 |
| 5172 | UAUCAAAC A UUAAAAAU | 1167 | ATTTTTAA GGCTAGCTACAACGA GTTTGATA | 2488 |
| 5179 | CAUUAAAA A UGACCACU | 1168 | AGTGGTCA GGCTAGCTACAACGA TTTTAATG | 2489 |
| 5182 | UAAAAAUG A CCACUCUU | 1169 | AAGAGTGG GGCTAGCTACAACGA CATTTTTA | 2490 |
| 5185 | AAAUGACC A CUCUUUUA | 1170 | TAAAAGAG GGCTAGCTACAACGA GGTCATTT | 2491 |
| 5194 | CUCUUUUA A UGAAAUUA | 1171 | TAATTTCA GGCTAGCTACAACGA TAAAAGAG | 2492 |
| 5199 | UUAAUGAA A UUAACUUU | 1172 | AAAGTTAA GGCTAGCTACAACGA TTCATTAA | 2493 |
| 5203 | UGAAAUUA A CUUUUAAA | 1173 | TTTAAAAG GGCTAGCTACAACGA TAATTTCA | 2494 |
| 5211 | ACUUUUAA A UGUUUAUA | 1174 | TATAAACA GGCTAGCTACAACGA TTAAAAGT | 2495 |
| 5213 | UUUUAAAU G UUUAUAGG | 1175 | CCTATAAA GGCTAGCTACAACGA ATTTAAAA | 2496 |
| 5217 | AAAUCUUU A UAGGAGUA | 1176 | TACTCCTA GGCTAGCTACAACGA AAACATTT | 2497 |
| 5223 | UUAUAGGA G UAUGUGCU | 1177 | AGCACATA GGCTAGCTACAACGA TCCTATAA | 2498 |
| 5225 | AUAGGAGU A UGUGCUGU | 1178 | ACAGCACA GGCTAGCTACAACGA ACTCCTAT | 2499 |
| 5227 | AGGAGUAU G UGCUGUGA | 1179 | TCACAGCA GGCTAGCTACAACGA ATACTCCT | 2500 |
| 5229 | GAGUAUGU G CUGUGAAG | 1180 | CTTCACAG GGCTAGCTACAACGA ACATACTC | 2501 |
| 5232 | UAUGUGCU G UGAAGUGA | 1181 | TCACTTCA GGCTAGCTACAACGA AGCACATA | 2502 |
| 5237 | GCUGUGAA G UGAUCUAA | 1182 | TTAGATCA GGCTAGCTACAACGA TTCACAGC | 2503 |
| 5240 | GUGAAGUG A UCUAAAAU | 1183 | ATTTTAGA GGCTAGCTACAACGA CACTTCAC | 2504 |
| 5247 | GAUCUAAA A UUUGUAAU | 1184 | ATTACAAA GGCTAGCTACAACGA TTTAGATC | 2505 |
| 5251 | UAAAAUUU G UAAUAUUU | 1185 | AAATATTA GGCTAGCTACAACGA AAATTTTA | 2506 |
| 5254 | AAUUUGUA A UAUUUUUG | 1186 | CAAAAATA GGCTAGCTACAACGA TACAAATT | 2507 |
| 5256 | UUUGUAAU A UUUUUGUC | 1187 | GACAAAAA GGCTAGCTACAACGA ATTACAAA | 2508 |
| 5262 | AUAUUUUU G UCAUGAAC | 1188 | GTTCATGA GGCTAGCTACAACGA AAAAATAT | 2509 |
| 5265 | UUUUUGUC A UGAACUGU | 1189 | ACAGTTCA GGCTAGCTACAACGA GACAAAAA | 2510 |
| 5269 | UGUCAUGA A CUGUACUA | 1190 | TAGTACAG GGCTAGCTACAACGA TCATGACA | 2511 |
| 5272 | CAUGAACU G UACUACUC | 1191 | GAGTAGTA GGCTAGCTACAACGA AGTTCATG | 2512 |
| 5274 | UGAACUGU A CUACUCCU | 1192 | AGGAGTAG GGCTAGCTACAACGA ACAGTTCA | 2513 |
| 5277 | ACUGUACU A CUCCUAAU | 1193 | ATTAGGAG GGCTAGCTACAACGA AGTACAGT | 2514 |
| 5284 | UACUCCUA A UUAUUGUA | 1194 | TACAATAA GGCTAGCTACAACGA TAGGAGTA | 2515 |
| 5287 | UCCUAAUU A UUGUAAUG | 1195 | CATTACAA GGCTAGCTACAACGA AATTAGGA | 2516 |
| 5290 | UAAUUAUU G UAAUGUAA | 1196 | TTACATTA GGCTAGCTACAACGA AATAATTA | 2517 |
| 5293 | UUAUUGUA A UGUAAUAA | 1197 | TTATTACA GGCTAGCTACAACGA TACAATAA | 2518 |
| 5295 | AUUGUAAU G UAAUAAAA | 1198 | TTTTATTA GGCTAGCTACAACGA ATTACAAT | 2519 |
| 5298 | GUAAUGUA A UAAAAAUA | 1199 | TATTTTTA GGCTAGCTACAACGA TACATTAC | 2520 |
| 5304 | UAAUAAAA A UAGUUACA | 1200 | TGTAACTA GGCTAGCTACAACGA TTTTATTA | 2521 |
| 5307 | UAAAAAUA G UUACAGUG | 1201 | CACTGTAA GGCTAGCTACAACGA TATTTTTA | 2522 |
| 5310 | AAAUAGUU A CAGUGACU | 1202 | AGTCACTG GGCTAGCTACAACGA AACTATTT | 2523 |
| 5313 | UAGUUACA G UGACUAUG | 1203 | CATAGTCA GGCTAGCTACAACGA TGTAACTA | 2524 |
| 5316 | UUACAGUG A CUAUGAGU | 1204 | ACTCATAG GGCTAGCTACAACGA CACTGTAA | 2525 |
| 5319 | CAGUGACU A UGAGUGUG | 1205 | CACACTCA GGCTAGCTACAACGA AGTCACTG | 2526 |
| 5323 | GACUAUGA G UGUGGAUG | 1206 | AATACACA GGCTAGCTACAACGA TCATAGTC | 2527 |
| 5325 | CUAUGAGU G UGUAUUUA | 1207 | TAAATACA GGCTAGCTACAACGA ACTCATAG | 2528 |
| 5327 | AUGAGUGU G UAUUUAUU | 1208 | AATAAATA GGCTAGCTACAACGA ACACTCAT | 2529 |
| 5329 | GAGUGUGU A UUUAUUCA | 1209 | TGAATAAA GGCTAGCTACAACGA ACACACTC | 2530 |
| 5333 | GUGUAGUG A UUCAUGCA | 1210 | TGCATGAA GGCTAGCTACAACGA AAATACAC | 2531 |
| 5337 | AUUUAUUC A UGCAAAUU | 1211 | AATTTGCA GGCTAGCTACAACGA GAATAAAT | 2532 |
| 5339 | UUAUUCAU G CAAAUUUG | 1212 | CAAATTTG GGCTAGCTACAACGA ATGAATAA | 2533 |
| 5343 | UCAUGCAA A UUUGAACU | 1213 | AGTTCAAA GGCTAGCTACAACGA TTGCATGA | 2534 |
| 5349 | AAAUUUGA A CUGUUUGC | 1214 | GCAAACAG GGCTAGCTACAACGA TCAAATTT | 2535 |
| 5352 | UUUGAACU G UUUGCCCC | 1215 | GGGGCAAA GGCTAGCTACAACGA AGTTCAAA | 2536 |
| 5356 | AACUGUUU G CCCCGAAA | 1216 | TTTCGGGG GGCTAGCTACAACGA AAACAGTT | 2537 |
| 5364 | GCCCCGAA A UGGAUAUG | 1217 | CATATCCA GGCTAGCTACAACGA TTCGGGGC | 2538 |
| 5368 | CGAAAUGG A UAUGGAUA | 1218 | TATCCATA GGCTAGCTACAACGA CCATTTCG | 2539 |
| 5370 | AAAUGGAU A UGGAUACU | 1219 | AGTATCCA GGCTAGCTACAACGA ATCCATTT | 2540 |
| 5374 | GGAUAUGG A UACUUUAU | 1220 | ATAAAGTA GGCTAGCTACAACGA CCATATCC | 2541 |
| 5376 | AUAUGGAU A CUUUAUAA | 1221 | TTATAAAG GGCTAGCTACAACGA ATCCATAT | 2542 |
| 5381 | GAUACUUU A UAAGCCAU | 1222 | ATGGCTTA GGCTAGCTACAACGA AAAGTATC | 2543 |
| 5385 | CUUUAUAA G CCAUAGAC | 1223 | GTCTATGG GGCTAGCTACAACGA TTATAAAG | 2544 |
| 5388 | UAUAAGCC A UAGACACU | 1224 | AGTGTCTA GGCTAGCTACAACGA GGCTTATA | 2545 |
| 5392 | AGCCAUAG A CACUAUAG | 1225 | CTATAGTG GGCTAGCTACAACGA CTATGGCT | 2546 |
| 5394 | CCAUAGAC A CUAUAGUA | 1226 | TACTATAG GGCTAGCTACAACGA GTCTATGG | 2547 |
| 5397 | UAGACACU A UAGUAUAC | 1227 | GTATACTA GGCTAGCTACAACGA AGTGTCTA | 2548 |
| 5400 | ACACUAUA G UAUACCAG | 1228 | CTGGTATA GGCTAGCTACAACGA TATAGTGT | 2549 |
| 5402 | ACUAUAGU A UACCAGUG | 1229 | CACTGGTA GGCTAGCTACAACGA ACTATAGT | 2550 |
| 5404 | UAUAGUAU A CCAGUGAA | 1230 | TTCACTGG GGCTAGCTACAACGA ATACTATA | 2551 |
| 5408 | GUAUACCA G UGAAUCUU | 1231 | AAGATTCA GGCTAGCTACAACGA TGGTATAC | 2552 |
| 5412 | ACCAGUGA A UCUUUUAU | 1232 | ATAAAAGA GGCTAGCTACAACGA TCACTGGT | 2553 |
| 5419 | AAUCUUUU A UGCAGCUU | 1233 | AAGCTGCA GGCTAGCTACAACGA AAAAGATT | 2554 |
| 5421 | UCUUUUAU G CAGCUUGU | 1234 | ACAAGCTG GGCTAGCTACAACGA ATAAAAGA | 2555 |
| 5424 | UUUAUGCA G CUUGUGAG | 1235 | CTAACAAG GGCTAGCTACAACGA TGCATAAA | 2556 |
| 5428 | UGCAGCUU G UUAGAAGU | 1236 | ACTTCTAA GGCTAGCTACAACGA AAGCTGCA | 2557 |
| 5435 | UGUUAGAA G UAUCCUUU | 1237 | AAAGGATA GGCTAGCTACAACGA TTCTAACA | 2558 |
| 5437 | UUAGAAGU A UCCUUUUA | 1238 | TAAAAGGA GGCTAGCTACAACGA ACTTCTAA | 2559 |
| 5445 | AUCCUUUU A UUUUCUAA | 1239 | TTAGAAAA GGCTAGCTACAACGA AAAAGGAT | 2560 |
| 5457 | UCUAAAAG G UGCUGUGG | 1240 | CCACAGCA GGCTAGCTACAACGA CTTTTAGA | 2561 |
| 5459 | UAAAAGGU G CUGUGGAU | 1241 | ATCCACAG GGCTAGCTACAACGA ACCTTTTA | 2562 |
| 5462 | AAGGUGCU G UGGAUAUU | 1242 | AATATCCA GGCTAGCTACAACGA AGCACCTT | 2563 |
| 5466 | UGCUGUGG A UAUUAUGU | 1243 | ACATAATA GGCTAGCTACAACGA CCACAGCA | 2564 |
| 5468 | CUGUGGAU A UUAUGUAA | 1244 | TTACATAA GGCTAGCTACAACGA ATCCACAG | 2565 |
| 5471 | UGGAUAUU A UGUAAAGG | 1245 | CCTTTACA GGCTAGCTACAACGA AATATCCA | 2566 |
| 5473 | GAUAUUAU G UAAAGGCG | 1246 | CGCCTTTA GGCTAGCTACAACGA ATAATATC | 2567 |
| 5479 | AUGUAAAG G CGUGUUUG | 1247 | CAAACACG GGCTAGCTACAACGA CTTTACAT | 2568 |
| 5481 | GUAAAGGC G UGUUUGCU | 1248 | AGCAAACA GGCTAGCTACAACGA GCCTTTAC | 2569 |
| 5483 | AAAGGCGU G UUUGCUUA | 1249 | TAAGCAAA GGCTAGCTACAACGA ACGCCTTT | 2570 |
| 5487 | GCGUGUUU G CUUAAACA | 1250 | TGTTTAAG GGCTAGCTACAACGA AAACACGC | 2571 |
| 5493 | UUGCUUAA A CAAUUUUC | 1251 | GAAAATTG GGCTAGCTACAACGA TTAAGCAA | 2572 |
| 5496 | CUUAAACA A UUUUCCAU | 1252 | ATGGAAAA GGCTAGCTACAACGA TGTTTAAG | 2573 |
| 5503 | AAUUUUCC A UAUUUAGA | 1253 | TCTAAATA GGCTAGCTACAACGA GGAAAATT | 2574 |
| 5505 | UUUUCCAU A UUUAGAAG | 1254 | CTTCTAAA GGCTAGCTACAACGA ATGGAAAA | 2575 |
| 5513 | AUUUAGAA G UAGAUGCA | 1255 | TGCATCTA GGCTAGCTACAACGA TTCTAAAT | 2576 |
| 5517 | AGAAGUAG A UGCAAAAC | 1256 | GTTTTGCA GGCTAGCTACAACGA CTACTTCT | 2577 |
| 5519 | AAGUAGAU G CAAAACAA | 1257 | TTGTTTTG GGCTAGCTACAACGA ATCTACTT | 2578 |
| 5524 | GAUGCAAA A CAAAUCUG | 1258 | CAGATTTG GGCTAGCTACAACGA TTTGCATC | 2579 |
| 5528 | CAAAACAA A UCUGCCUU | 1259 | AAGGCAGA GGCTAGCTACAACGA TTGTTTTG | 2580 |
| 5532 | ACAAAUCU G CCUUUAUG | 1260 | CATAAAGG GGCTAGCTACAACGA AGATTTGT | 2581 |
| 5538 | CUGCCUUU A UGACAAAA | 1261 | TTTTGTCA GGCTAGCTACAACGA AAAGGCAG | 2582 |
| 5541 | CCUUUAUG A CAAAAAAA | 1262 | TTTTTTTG GGCTAGCTACAACGA CATAAAGG | 2583 |
| 5549 | ACAAAAAA A UAGGAUAA | 1263 | TTATCCTA GGCTAGCTACAACGA TTTTTTGT | 2584 |
| 5554 | AAAAUAGG A UAACAUUA | 1264 | TAATGTTA GGCTAGCTACAACGA CCTATTTT | 2585 |
| 5557 | AUAGGAUA A CAUUAUUU | 1265 | AAATAATG GGCTAGCTACAACGA TATCCTAT | 2586 |
| 5559 | AGGAUAAC A UUAUUUAU | 1266 | ATAAATAA GGCTAGCTACAACGA GTTATCCT | 2587 |
| 5562 | AUAACAUU A UUUAUUUA | 1267 | TAAATAAA GGCTAGCTACAACGA AATGTTAT | 2588 |
| 5566 | CAUUAUUU A UUUAUUUC | 1268 | GAAATAAA GGCTAGCTACAACGA AAATAATG | 2589 |
| 5570 | AUUUAUUU A UUUCCUUU | 1269 | AAAGGAAA GGCTAGCTACAACGA AAATAAAT | 2590 |
| 5580 | UUCCUUUU A UCAAUAAG | 1270 | CTTATTGA GGCTAGCTACAACGA AAAAGGAA | 2591 |
| 5584 | UUUUAUCA A UAAGGUAA | 1271 | TTACCTTA GGCTAGCTACAACGA TGATAAAA | 2592 |
| 5589 | UCAAUAAG G UAAUUCAU | 1272 | ATCAATTA GGCTAGCTACAACGA CTTATTGA | 2593 |
| 5592 | AUAAGGUA A UUGAUACA | 1273 | TGTATCAA GGCTAGCTACAACGA TACCTTAT | 2594 |
| 5596 | GGUAAUUG A UACACAAC | 1274 | GTTGTGTA GGCTAGCTACAACGA CAATTACC | 2595 |
| 5598 | UAAUUGAU A CACAACAG | 1275 | CTGTTGTG GGCTAGCTACAACGA ATCAATTA | 2596 |
| 5600 | AUUGAUAC A CAACAGGU | 1276 | ACCTGTTG GGCTAGCTACAACGA GTATCAAT | 2597 |
| 5603 | GAUACACA A CAGGUGAC | 1277 | GTCACCTG GGCTAGCTACAACGA TGTGTATC | 2598 |
| 5607 | CACAACAG G UGACUUGG | 1278 | CCAAGTCA GGCTAGCTACAACGA CTGTTGTG | 2599 |
| 5610 | AACAGGUG A CUUGGUUU | 1279 | AAACCAAG GGCTAGCTACAACGA CACCTGTT | 2600 |
| 5615 | GUGACUUG G UUUUAGGC | 1280 | GCCTAAAA GGCTAGCTACAACGA CAAGTCAC | 2601 |
| 5622 | GGUUUUAG G CCCAAAGG | 1281 | CCTTTGGG GGCTAGCTACAACGA CTAAAACC | 2602 |
| 5630 | GCCCAAAG G UAGCAGCA | 1282 | TGCTGCTA GGCTAGCTACAACGA CTTTGGGC | 2603 |
| 5633 | CAAAGGUA G CAGCAGCA | 1283 | TCCTGCTG GGCTAGCTACAACGA TACCTTTG | 2604 |
| 5636 | AGGUAGCA G CAGCAACA | 1284 | TGTTGCTG GGCTAGCTACAACGA TGCTACCT | 2605 |
| 5639 | UAGCAGCA G CAACAUUA | 1285 | TAATGTTG GGCTAGCTACAACGA TGCTGCTA | 2606 |
| 5642 | CAGCAGCA A CAUUAAUA | 1286 | TATTAATG GGCTAGCTACAACGA TGCTGCTG | 2607 |
| 5644 | GCAGCAAC A UUAAUAAU | 1287 | ATTATTAA GGCTAGCTACAACGA GTTGCTGC | 2608 |
| 5648 | CAACAUUA A UAAUGGAA | 1288 | TTCCATTA GGCTAGCTACAACGA TAATGTTG | 2609 |
| 5651 | CAUUAAUA A UGGAAAUA | 1289 | TATTTCCA GGCTAGCTACAACGA TATTAATG | 2610 |
| 5657 | UAAUGGAA A UAAUUGAA | 1290 | TTCAATTA GGCTAGCTACAACGA TTCCATTA | 2611 |
| 5660 | UGGAAAUA A UUGAAUAG | 1291 | CTATTCAA GGCTAGCTACAACGA TATTTCCA | 2612 |
| 5665 | AUAAUUGA A UAGUUAGU | 1292 | ACTAACTA GGCTAGCTACAACGA TCAATTAT | 2613 |
| 5668 | AUUGAAUA G UUAGUUAU | 1293 | ATAACTAA GGCTAGCTACAACGA TATTCAAT | 2614 |
| 5672 | AAUAGUUA G UUAUGUAU | 1294 | ATACATAA GGCTAGCTACAACGA TAACTATT | 2615 |
| 5675 | AGUUAGUU A UGUAUGUU | 1295 | AACATACA GGCTAGCTACAACGA AACTAACT | 2616 |
| 5677 | UUAGUUAU G UAUGUUAA | 1296 | TTAACATA GGCTAGCTACAACGA ATAACTAA | 2617 |
| 5679 | AGUUAUCU A UGUUAAUG | 1297 | CATTAACA GCCTAGCTACAACGA ACATAACT | 2618 |
| 5681 | UUAUGUAU G UUAAUGCC | 1298 | GGCATTAA GGCTAGCTACAACGA ATACATAA | 2619 |
| 5685 | GUAUGUUA A UGCCAGUC | 1299 | GACTGGCA GGCTAGCTACAACGA TAACATAC | 2620 |
| 5687 | AUGUUAAU G CCAGUCAC | 1300 | GTGACTGG GGCTAGCTACAACGA ATTAACAT | 2621 |
| 5691 | UAAUGCCA G UCACCAGC | 1301 | GCTGGTCA GGCTAGCTACAACGA TGGCATTA | 2622 |
| 5694 | UGCCAGUC A CCAGCAGG | 1302 | CCTGCTGG GGCTAGCTACAACGA GACTGGCA | 2623 |
| 5698 | AGUCACCA G CAGGCUAU | 1303 | ATAGCCTG GGCTAGCTACAACGA TGGTGACT | 2624 |
| 5702 | ACCAGCAG G CUAUUUCA | 1304 | TGAAATAG GGCTAGCTACAACGA CTGCTGGT | 2625 |
| 5705 | AGCAGGCU A UUUCAAGG | 1305 | CCTTGAAA GGCTAGCTACAACGA AGCCTGCT | 2626 |
| 5713 | AUUUCAAG G UCAGAAGU | 1306 | ACTTCTGA GGCTAGCTACAACGA CTTGAAAT | 2627 |
| 5720 | GGUCAGAA G UAAUGACU | 1307 | AGTCATTA GGCTAGCTACAACGA TTCTGACC | 2628 |
| 5723 | CAGAAGUA A UGACUCCA | 1308 | TGGAGTCA GGCTAGCTACAACGA TACTTCTG | 2629 |
| 5726 | AAGUAAUG A CUCCAUAC | 1309 | GTATGGAG GGCTAGCTACAACGA CATTACTT | 2630 |
| 5731 | AUGACUCC A UACAUAUU | 1310 | AATATGTA GGCTAGCTACAACGA GGAGTCAT | 2631 |
| 5733 | GACUCCAU A CAUAUUAU | 1311 | ATAATATG GGCTAGCTACAACGA ATGGAGTC | 2632 |
| 5735 | CUCCAUAC A UAUUAUUU | 1312 | AAATAATA GGCTAGCTACAACGA GTATGGAG | 2633 |
| 5737 | CCAUACAU A UUAUUUAU | 1313 | ATAAATAA GGCTAGCTACAACGA ATGTATGG | 2634 |
| 5740 | UACAUAUU A UUUAUUUC | 1314 | GAAATAAA GGCTAGCTACAACGA AATATGTA | 2635 |
| 5744 | UAUUAUUU A UUUCUAUA | 1315 | TATAGAAA GGCTAGCTACAACGA AAATAATA | 2636 |
| 5750 | UUAUUUCU A UAACUACA | 1316 | TGTAGTTA GGCTAGCTACAACGA AGAAATAA | 2637 |
| 5753 | UUUCUAUA A CUACAUUU | 1317 | AAATGTAG GGCTAGCTACAACGA TATAGAAA | 2638 |
| 5756 | CUAUAACU A CAUUUAAA | 1318 | TTTAAATG GGCTAGCTACAACGA AGTTATAG | 2639 |
| 5758 | AUAACUAC A UUUAAAUC | 1319 | GATTTAAA GGCTAGCTACAACGA GTAGTTAT | 2640 |
| 5764 | ACAUUUAA A UCAUUACC | 1320 | GGTAATGA GGCTAGCTACAACGA TTAAATGT | 2641 |
| 5767 | UUUAAAUC A UUACCAGG | 1321 | CCTGGTAA GGCTAGCTACAACGA GATTTAAA | 2642 |

Input Sequence = NM_004985. Cut Site = R/Y
Arm Length = 8. Core Sequence = GGCTAGCTACAACGA
NM_004985 (Homo sapiens v-Ki-ras2 Kirsten rat sarcoma 2 viral oncogene
homolog (KRas2) mRNA; 5775 nt)

TABLE III
Human H-Ras DNAzyme and Target molecules

| | | Seq | | Seq |
|---|---|---|---|---|
| Pos | Substrate | ID | DNAzyme | ID |
| 9 | GGAUCCCA G CCUUUCCC | 2643 | GGGAAAGG GGCTAGCTACAACGA TGGGATCC | 3650 |
| 20 | UUUCCCCA G CCCGUAGC | 2644 | GCTACGGG GGCTAGCTACAACGA TGGGGAAA | 3651 |
| 24 | CCCAGCCC G UAGCCCCG | 2645 | CGGGGCTA GGCTAGCTACAACGA GGGCTGGG | 3652 |
| 27 | AGCCCGUA G CCCCGGGA | 2646 | TCCCGGGG GGCTAGCTACAACGA TACGGGCT | 3653 |
| 35 | GCCCCGGG A CCUCCGCG | 2647 | CGCGGAGG GGCTAGCTACAACGA CCCGGGGC | 3654 |
| 41 | GGACCUCC G CGGUGGGC | 2648 | GCCCACCG GGCTAGCTACAACGA GGAGGTCC | 3655 |
| 44 | CCUCCGCG G UGGGCGGC | 2649 | GCCGCCCA GGCTAGCTACAACGA CGCGGAGG | 3656 |
| 48 | CGCGGUGG C CGGCGCCG | 2650 | CGGCGCCG GGCTAGCTACAACGA CCACCGCG | 3657 |
| 51 | GGUGGGCG G CGCCGCGC | 2651 | GCGCGGCG GGCTAGCTACAACGA CGCCCACC | 3658 |
| 53 | UGGGCGGC G CCGCGCUG | 2652 | CAGCGCGG GGCTAGCTACAACGA GCCGCCCA | 3659 |
| 56 | GCGGCGCC G CGCUGCCG | 2653 | CGGCAGCG GGCTAGCTACAACGA GGCGCCGC | 3660 |
| 58 | GGCGCCGC G CUGCCGGC | 2654 | GCCGGCAG GGCTAGCTACAACGA GCGGCGCC | 3661 |
| 61 | GCCGCGCU G CCGGCGCA | 2655 | TGCGCCGG GGCTAGCTACAACGA AGCGCGGC | 3662 |
| 65 | CGCUGCCG G CGCAGGGA | 2656 | TCCCTGCG GGCTAGCTACAACGA CGGCAGCG | 3663 |
| 67 | CUGCCGGC G CAGGGAGG | 2657 | CCTCCCTG GGCTAGCTACAACGA GCCGGCAG | 3664 |
| 76 | CAGGGAGG G CCUCUGGU | 2658 | ACCAGAGG GGCTAGCTACAACGA CCTCCCTG | 3665 |
| 83 | GGCCUCUG G UGCACCGG | 2659 | CCGGTGCA GGCTAGCTACAACGA CAGAGGCC | 3666 |
| 85 | CCUCUGGU G CACCGGCA | 2660 | TGCCGGTG GGCTAGCTACAACGA ACCAGAGG | 3667 |
| 87 | UCUGGUGC A CCGGCACC | 2661 | GGTGCCGG GGCTAGCTACAACGA GCACCAGA | 3668 |
| 91 | GUGCACCG G CACCGCUG | 2662 | CAGCGGTG GGCTAGCTACAACGA CGGTGCAC | 3669 |
| 93 | GCACCGGC A CCGCUGAG | 2663 | CTCAGCGG GGCTAGCTACAACGA GCCGGTGC | 3670 |
| 96 | CCGGCACC G CUGAGUCG | 2664 | CGACTCAG GGCTAGCTACAACGA GGTGCCGG | 3671 |
| 101 | ACCGCUGA G UCGGGUUC | 2665 | GAACCCGA GGCTAGCTACAACGA TCAGCGGT | 3672 |
| 106 | UGAGUCGG G UUCUCUCG | 2666 | CGAGAGAA GGCTAGCTACAACGA CCGACTCA | 3673 |
| 114 | GUUCUCUC G CCGGCCUG | 2667 | CAGGCCGG GGCTAGCTACAACGA GAGAGAAC | 3674 |
| 118 | UCUCGCCG G CCUGUUCC | 2668 | GGAACAGG GGCTAGCTACAACGA CGGCGAGA | 3675 |
| 122 | GCCGGCCU G UUCCCGGG | 2669 | CCCGGGAA GGCTAGCTACAACGA AGGCCGGC | 3676 |
| 134 | CCGGGAGA G CCCGGGGC | 2670 | GCCCCGGG GGCTAGCTACAACGA TCTCCCGG | 3677 |
| 141 | AGCCCGGG G CCCUGCUC | 2671 | GAGCAGGG GGCTAGCTACAACGA CCCGGGCT | 3678 |
| 146 | GGGGCCCU G CUCGGAGA | 2672 | TCTCCGAG GGCTAGCTACAACGA AGGGCCCC | 3679 |
| 154 | GCUCGGAG A UGCCGCCC | 2673 | GGGCGGCA GGCTAGCTACAACGA CTCCGAGC | 3680 |
| 156 | UCGGAGAU G CCGCCCCG | 2674 | CGGGGCGG GGCTAGCTACAACGA ATCTCCGA | 3681 |
| 159 | GAGAUGCC G CCCCGGGC | 2675 | GCCCGGGG GGCTAGCTACAACGA GGCATCTC | 3682 |
| 166 | CGCCCCGG G CCCCCAGA | 2676 | TCTGGGGG GGCTAGCTACAACGA CCGGGGCG | 3683 |
| 174 | GCCCCCAG A CACCGGCU | 2677 | AGCCGGTG GGCTAGCTACAACGA CTGGGGGC | 3684 |
| 176 | CCCCAGAC A CCGGCUCC | 2678 | GGAGCCGG GGCTAGCTACAACGA GTCTGGGG | 3685 |
| 180 | AGACACCG G CUCCCUGG | 2679 | CCAGGGAG GGCTAGCTACAACGA CGGTGTCT | 3686 |
| 188 | GCUCCCUG G CCUUCCUC | 2680 | GAGGAAGG GGCTAGCTACAACGA CAGGGAGC | 3687 |
| 199 | UUCCUCGA G CAACCCCG | 2681 | CGGGGTTG GGCTAGCTACAACGA TCGAGGAA | 3688 |
| 202 | CUCGAGCA A CCCCGAGC | 2682 | GCTCGGGG GGCTAGCTACAACGA TGCTCGAG | 3689 |
| 209 | AACCCCGA G CUCGGCUC | 2683 | GAGCCGAG GGCTAGCTACAACGA TCGGGGTT | 3690 |
| 214 | CGAGCUCG G CUCCGGUC | 2684 | GACCGGAG GGCTAGCTACAACGA CGAGCTCG | 3691 |
| 220 | CGGCUCCG G UCUCCAGC | 2685 | GCTGGAGA GGCTAGCTACAACGA CGGAGCCG | 3692 |
| 227 | GGUCUCCA G CCAAGCCC | 2686 | GGGCTTGG GGCTAGCTACAACGA TGGAGACC | 3693 |
| 232 | CCAGCCAA G CCCAACCC | 2687 | GGGTTGGG GGCTAGCTACAACGA TTGGCTGG | 3694 |
| 237 | CAAGCCCA A CCCCGAGA | 2688 | TCTCGGGG GGCTAGCTACAACGA TGGGCTTG | 3695 |
| 247 | CCCGAGAG G CCGCGGCC | 2689 | GCCCGCGG GGCTAGCTACAACGA CTCTCGGG | 3696 |
| 250 | GAGAGGCC G CGGCCCUA | 2690 | TAGGGCCG GGCTAGCTACAACGA GGCCTCTC | 3697 |
| 253 | AGGCCGCG G CCCUACUG | 2691 | CAGTAGGG GGCTAGCTACAACGA CGCGGCCT | 3698 |
| 258 | GCGGCCCU A CUGGCUCC | 2692 | GGAGCCAG GGCTAGCTACAACGA AGGGCCGC | 3699 |
| 262 | CCCUACUG G CUCCGCCU | 2693 | AGGCGGAG GGCTAGCTACAACGA CAGTAGGG | 3700 |
| 267 | CUGGCUCC G CCUCCCGC | 2694 | GCGGGAGG GGCTAGCTACAACGA GGAGCCAG | 3701 |
| 274 | CGCCUCCC G CGUUGCUC | 2695 | GAGCAACG GGCTAGCTACAACGA GGGAGGCG | 3702 |
| 276 | CCUCCCGC G UUGCUCCC | 2696 | GGGAGCAA GGCTAGCTACAACGA GCGGGAGG | 3703 |
| 279 | CCCGCGUU G CUCCCGGA | 2697 | TCCGGGAG GGCTAGCTACAACGA AACGCGGG | 3704 |
| 289 | UCCCGGAA G CCCCGCCC | 2698 | GGGCGGGG GGCTAGCTACAACGA TTCCGGGA | 3705 |
| 294 | GAAGCCCC G CCCGACCG | 2699 | CGGTCGGG GGCTAGCTACAACGA GGGGCTTC | 3706 |
| 299 | CCCGCCCG A CCGCGGCU | 2700 | AGCCGCGG GGCTAGCTACAACGA CGGGCGGG | 3707 |
| 302 | GCCCGACC G CGGCUCCU | 2701 | AGGAGCCG GGCTAGCTACAACGA GGTCGGGC | 3708 |
| 305 | CGACCGCG G CUCCUGAC | 2702 | GTCAGGAG GGCTAGCTACAACGA CGCGGTCG | 3709 |
| 312 | GGCUCCUG A CAGACGGG | 2703 | CCCGTCTG GGCTAGCTACAACGA CAGGAGCC | 3710 |
| 316 | CCUGACAG A CGGGCCGC | 2704 | GCGGCCCG GGCTAGCTACAACGA CTGTCAGG | 3711 |
| 320 | ACAGACGG G CCGCUCAG | 2705 | CTGAGCGG GGCTAGCTACAACGA CCGTCTGT | 3712 |
| 323 | GACGGGCC G CUCAGCCA | 2706 | TGGCTGAG GGCTAGCTACAACGA GGCCCGTC | 3713 |
| 328 | GCCGCUCA G CCAACCGG | 2707 | CCGGTTGG GGCTAGCTACAACGA TGAGCGGC | 3714 |
| 332 | CUCAGCCA A CCGGGGUG | 2708 | CACCCCGG GGCTAGCTACAACGA TGGCTGAG | 3715 |
| 338 | CAACCGGG G UGGGGCGG | 2709 | CCGCCCCA GGCTAGCTACAACGA CCCGGTTG | 3716 |
| 343 | GGGGUGGG G CGGGGCCC | 2710 | GGGCCCCG GGCTAGCTACAACGA CCCACCCC | 3717 |
| 348 | GGGGCGGG G CCCGAUGG | 2711 | CCATCGGG GGCTAGCTACAACGA CCCGCCCC | 3718 |
| 353 | GGGGCCCG A UGGCGCGC | 2712 | GCGCGCCA GGCTAGCTACAACGA CGGGCCCC | 3719 |
| 356 | GCCCGAUG G CGCGCAGC | 2713 | GCTGCGCG GGCTAGCTACAACGA CATCGGGC | 3720 |
| 358 | CCGAUGGC G CGCAGCCA | 2714 | TGGCTGCG GGCTAGCTACAACGA GCCATCGG | 3721 |
| 360 | GAUGGCGC G CAGCCAAU | 2715 | ATTGGCTG GGCTAGCTACAACGA GCGCCATC | 3722 |
| 363 | GGCGCGCA G CCAAUGGU | 2716 | ACCATTGG GGCTAGCTACAACGA TGCGCGCC | 3723 |
| 367 | CGCAGCCA A UGGUAGGC | 2717 | GCCTACCA GGCTAGCTACAACGA TGGCTGCG | 3724 |
| 370 | AGCCAAUG G UAGGCCGC | 2718 | GCGGCCTA GGCTAGCTACAACGA CATTGGCT | 3725 |
| 374 | AAUGGUAG G CCGCGCCU | 2719 | AGGCGCGG GGCTAGCTACAACGA CTACCATT | 3726 |
| 377 | GGUAGGCC G CGCCUGGC | 2720 | GCCAGGCG GGCTAGCTACAACGA GGCCTACC | 3727 |
| 379 | UAGGCCGC G CCUGGCAG | 2721 | CTGCCAGG GGCTAGCTACAACGA GCGGCCTA | 3728 |
| 384 | CGCGCCUG G CAGACGGA | 2722 | TCCGTCTG GGCTAGCTACAACGA CAGGCGCG | 3729 |
| 388 | CCUGGCAG A CGGACGGG | 2723 | CCCGTCCG GGCTAGCTACAACGA CTGCCAGG | 3730 |
| 392 | GCAGACGG A CGGGCGCG | 2724 | CGCGCCCG GGCTAGCTACAACGA CCGTCTGC | 3731 |
| 396 | ACGGACGG G CGCGGGGC | 2725 | GCCCCGCG GGCTAGCTACAACGA CCGTCCGT | 3732 |
| 398 | GGACGGGC G CGGGGCGG | 2726 | CCGCCCCG GGCTAGCTACAACGA GCCCGTCC | 3733 |
| 403 | GGCGCGGG G CGGGGCGU | 2727 | ACGCCCCG GGCTAGCTACAACGA CCCGCGCC | 3734 |
| 408 | GGGGCGGG G CGUGCGCA | 2728 | TGCGCACG GGCTAGCTACAACGA CCCGCCCC | 3735 |
| 410 | GGCGGGGC G UGCGCAGG | 2729 | CCTGCGCA GGCTAGCTACAACGA GCCCCGCC | 3736 |
| 412 | CGGGGCGU G CGCAGGCC | 2730 | GGCCTGCG GGCTAGCTACAACGA ACGCCCCG | 3737 |
| 414 | GGGCGUGC G CAGGCCCG | 2731 | CGGGCCTG GGCTAGCTACAACGA GCACGCCC | 3738 |
| 418 | GUGCGCAG G CCCGCCCG | 2732 | CGGGCGGG GGCTAGCTACAACGA CTGCGCAC | 3739 |
| 422 | GCAGGCCC G CCCGAGUC | 2733 | GACTCGGG GGCTAGCTACAACGA GGGCCTGC | 3740 |
| 428 | CCGCCCGA G UCUCCGCC | 2734 | GGCGGAGA GGCTAGCTACAACGA TCGGGCGG | 3741 |
| 434 | GAGUCUCC G CCGCCCGU | 2735 | ACGGGCGG GGCTAGCTACAACGA GGAGACTC | 3742 |
| 437 | UCUCCGCC G CCCGUGCC | 2736 | GGCACGGG GGCTAGCTACAACGA GGCGGAGA | 3743 |
| 441 | CGCCGCCC G UGCCCUGC | 2737 | GCAGGGCA GGCTAGCTACAACGA GGGCGGCG | 3744 |
| 443 | CCGCCCGU G CCCUGCGC | 2738 | GCGCAGGG GGCTAGCTACAACGA ACGGGCGG | 3745 |
| 448 | CGUGCCCU G CGCCCGCA | 2739 | TGCGGGCG GGCTAGCTACAACGA AGGGCACG | 3746 |
| 450 | UGCCCUGC G CCCGCAAC | 2740 | GTTGCGGG GGCTAGCTACAACGA GCAGGGCA | 3747 |
| 454 | CUGCGCCC G CAACCCGA | 2741 | TCGGGTTG GGCTAGCTACAACGA GGGCGCAG | 3748 |
| 457 | CGCCCGCA A CCCGAGCC | 2742 | GGCTCGGG GGCTAGCTACAACGA TGCGGGCG | 3749 |
| 463 | CAACCCGA G CCGCACCC | 2743 | GGGTGCGG GGCTAGCTACAACGA TCGGGTTG | 3750 |
| 466 | CCCGAGCC G CACCCGCC | 2744 | GGCGGGTG GGCTAGCTACAACGA GGCTCGGG | 3751 |
| 468 | CGAGCCGC A CCCGCCGC | 2745 | GCGGCGGG GGCTAGCTACAACGA GCGGCTCG | 3752 |
| 472 | CCGCACCC G CCGCGGAC | 2746 | GTCCGCGG GGCTAGCTACAACGA GGGTGCGG | 3753 |
| 475 | CACCCGCC G CGGACGGA | 2747 | TCCGTCCG GGCTAGCTACAACGA GGCGGGTG | 3754 |
| 479 | CGCCGCGG A CGGAGCCC | 2748 | GGGCTCCG GGCTAGCTACAACGA CCGCGGCG | 3755 |
| 484 | CGGACGGA G CCCAUGCG | 2749 | CGCATGGG GGCTAGCTACAACGA TCCGTCCG | 3756 |
| 488 | CGGAGCCC A UGCGCGGG | 2750 | CCCGCGCA GGCTAGCTACAACGA GGGCTCCG | 3757 |
| 490 | GAGCCCAU G CGCGGGGC | 2751 | GCCCCGCG GGCTAGCTACAACGA ATGGGCTC | 3758 |
| 492 | GCCCAUGC G CGGGGCGA | 2752 | TCGCCCCG GGCTAGCTACAACGA GCATGGGC | 3759 |
| 497 | UGCGCGGG G CGAACCGC | 2753 | GCGGTTCG GGCTAGCTACAACGA CCCGCGCA | 3760 |
| 501 | CGGGGCGA A CCGCGCGC | 2754 | GCGCGCGG GGCTAGCTACAACGA TCGCCCCG | 3761 |
| 504 | GGCGAACC G CGCGCCCC | 2755 | GGGGCGCG GGCTAGCTACAACGA GGTTCGCC | 3762 |
| 506 | CGAACCGC G CGCCCCCG | 2756 | CGGGGGCG GGCTAGCTACAACGA GCGGTTCG | 3763 |
| 508 | AACCGCGC G CCCCCGCC | 2757 | GGCGGGGG GGCTAGCTACAACGA GCGCGGTT | 3764 |
| 514 | GCGCCCCC G CCCCCGCC | 2758 | GGCGGGGG GGCTAGCTACAACGA GGGGGCGC | 3765 |
| 520 | CCGCCCCC G CCCCGCCC | 2759 | GGGCGGGG GGCTAGCTACAACGA GGGGGCGG | 3766 |
| 525 | CCCGCCCC G CCCCGGCC | 2760 | GGCCGGGG GGCTAGCTACAACGA GGGGCGGG | 3767 |
| 531 | CCGCCCCG G CCUCGGCC | 2761 | GGCCGAGG GGCTAGCTACAACGA CGGGGCGG | 3768 |
| 537 | CGGCCUCG G CCCCGGCC | 2762 | GGCCGGGG GGCTAGCTACAACGA CGAGGCCG | 3769 |
| 543 | CGGCCCCG G CCCUGGCC | 2763 | GGCCAGGG GGCTAGCTACAACGA CGGGGCCG | 3770 |
| 549 | CGGCCCUG G CCCCGGGG | 2764 | CCCCGGGG GGCTAGCTACAACGA CAGGGCCG | 3771 |
| 558 | CCCCGGGG G CAGUCGCG | 2765 | CGCGACTG GGCTAGCTACAACGA CCCCGGGG | 3772 |
| 561 | CGGGGGCA G UCGCGCCU | 2766 | AGGCGCGA GGCTAGCTACAACGA TGCCCCCG | 3773 |
| 564 | GGGCAGUC G CGCCUGUG | 2767 | CACAGGCG GGCTAGCTACAACGA GACTGCCC | 3774 |
| 566 | GCAGUCGC G CCUGUGAA | 2768 | TTCACAGG GGCTAGCTACAACGA GCGACTGC | 3775 |
| 570 | UCGCGCCU G UGAACGGU | 2769 | ACCGTTCA GGCTAGCTACAACGA AGGCGCGA | 3776 |
| 574 | GCCUGUGA A CGGUGAGU | 2770 | ACTCACCG GGCTAGCTACAACGA TCACAGGC | 3777 |
| 577 | UGUGAACG G UGAGUGCG | 2771 | CGCACTCA GGCTAGCTACAACGA CGTTCACA | 3778 |
| 581 | AACGGUGA G UGCGGGCA | 2772 | TGCCCGCA GGCTAGCTACAACGA TCACCGTT | 3779 |
| 583 | CGGUGAGU G CGGGCAGG | 2773 | CCTGCCCG GGCTAGCTACAACGA ACTCACCG | 3780 |
| 587 | GAGUGCGG G CAGGGAUC | 2774 | GATCCCTG GGCTAGCTACAACGA CCGCACTC | 3781 |
| 593 | GGGCAGGG A UCGGCCGG | 2775 | CCGGCCGA GGCTAGCTACAACGA CCCTGCCC | 3782 |
| 597 | AGGGAUCG G CCGGGCCG | 2776 | CGGCCCGG GGCTAGCTACAACGA CGATCCCT | 3783 |
| 602 | UCGGCCGG G CCGCGCGC | 2777 | GCGCGCGG GGCTAGCTACAACGA CCGGCCGA | 3784 |
| 605 | GCCGGGCC G CGCGCCCU | 2778 | AGGGCGCG GGCTAGCTACAACGA GGCCCGGC | 3785 |
| 607 | CGGGCCGC G CGCCCUCC | 2779 | GGAGGGCG GGCTAGCTACAACGA GCGGCCCG | 3786 |
| 609 | GGCCGCGC G CCCUCCUC | 2780 | GAGGAGGG GGCTAGCTACAACGA GCGCGGCC | 3787 |
| 618 | CCCUCCUC G CCCCCAGG | 2781 | CCTGGGGG GGCTAGCTACAACGA GAGGAGGG | 3788 |
| 626 | GCCCCCAG G CGGCAGCA | 2782 | TGCTGCCG GGCTAGCTACAACGA CTGGGGGC | 3789 |
| 629 | CCCAGGCG G CAGCAAUA | 2783 | TATTGCTG GGCTAGCTACAACGA CGCCTGGG | 3790 |
| 632 | AGGCGGCA G CAAUACGC | 2784 | GCGTATTG GGCTAGCTACAACGA TGCCGCCT | 3791 |
| 635 | CGGCAGCA A UACGCGCG | 2785 | CGCGCGTA GGCTAGCTACAACGA TGCTGCCG | 3792 |
| 637 | GCAGCAAU A CGCGCGGC | 2786 | GCCGCGCG GGCTAGCTACAACGA ATTGCTGC | 3793 |
| 639 | AGCAAUAC G CGCGGCGC | 2787 | GCGCCGCG GGCTAGCTACAACGA GTATTGCT | 3794 |
| 641 | CAAUACGC G CGGCGCGG | 2788 | CCGCGCCG GGCTAGCTACAACGA GCGTATTG | 3795 |
| 644 | UACGCGCG G CGCGGGCC | 2789 | GGCCCGCG GGCTAGCTACAACGA CGCGCGTA | 3796 |
| 646 | CGCGCGGC G CGGGCCGG | 2790 | CCGGCCCG GGCTAGCTACAACGA GCCGCGCG | 3797 |
| 650 | CGGCGCGG G CCGGGGGC | 2791 | GCCCCCGG GGCTAGCTACAACGA CCGCGCCG | 3798 |
| 657 | GGCCGGGG G CGCGGGGC | 2792 | GCCCCGCG GGCTAGCTACAACGA CCCCGGCC | 3799 |
| 659 | CCGGGGGC G CGGGGCCG | 2793 | CGGCCCCG GGCTAGCTACAACGA GCCCCCGG | 3800 |
| 664 | GGCGCGGG G CCGGCGGG | 2794 | CCCGCCGG GGCTAGCTACAACGA CCCGCGCC | 3801 |
| 668 | CGGGGCCG G CGGGCGUA | 2795 | TACGCCCG GGCTAGCTACAACGA CGGCCCCG | 3802 |
| 672 | GCCGGCGG G CGUAAGCG | 2796 | CGCTTACG GGCTAGCTACAACGA CCGCCGGC | 3803 |
| 674 | CGGCGGGC G UAAGCGGC | 2797 | GCCGCTTA GGCTAGCTACAACGA GCCCGCCG | 3804 |
| 678 | GGGCGUAA G CGGCGGCG | 2798 | CGCCGCCG GGCTAGCTACAACGA TTACGCCC | 3805 |
| 681 | CGUAAGCG G CGGCGGCG | 2799 | CGCCGCCG GGCTAGCTACAACGA CGCTTACG | 3806 |
| 684 | AAGCGGCG G CGGCGGCG | 2800 | CGCCGCCG GGCTAGCTACAACGA CGCCGCTT | 3807 |
| 687 | CGGCGGCG G CGGCGGCG | 2801 | CGCCGCCG GGCTAGCTACAACGA CGCCGCCG | 3808 |
| 690 | CGGCGGCG G CGGCGGGU | 2802 | ACCCGCCG GGCTAGCTACAACGA CGCCGCCG | 3809 |
| 693 | CGGCGGCG G CGGGUGGG | 2803 | CCCACCCG GGCTAGCTACAACGA CGCCGCCG | 3810 |
| 697 | GGCGGCGG G UGGGUGGG | 2804 | CCCACCCA GGCTAGCTACAACGA CCGCCGCC | 3811 |
| 701 | GCGGGUGG G UGGGGCCG | 2805 | CGGCCCCA GGCTAGCTACAACGA CCACCCGC | 3812 |
| 706 | UGGGUGGG G CCGGGCGG | 2806 | CCGCCCGG GGCTAGCTACAACGA CCCACCCA | 3813 |
| 711 | GGGGCCGG G CGGGGCCC | 2807 | GGGCCCCG GGCTAGCTACAACGA CCGGCCCC | 3814 |
| 716 | CGGGCGGG G CCCGCGGG | 2808 | CCCGCGGG GGCTAGCTACAACGA CCCGCCCG | 3815 |
| 720 | CGGGGCCC G CGGGCACA | 2809 | TGTGCCCG GGCTAGCTACAACGA GGGCCCCG | 3816 |
| 724 | GCCCGCGG G CACAGGUG | 2810 | CACCTGTG GGCTAGCTACAACGA CCGCGGGC | 3817 |
| 726 | CCGCGGGC A CAGGUGAG | 2811 | CTCACCTG GGCTAGCTACAACGA GCCCGCGG | 3818 |
| 730 | GGGCACAG G UGAGCGGG | 2812 | CCCGCTCA GGCTAGCTACAACGA CTGTGCCC | 3819 |
| 734 | ACAGGUGA G CGGGCGUC | 2813 | GACGCCCG GGCTAGCTACAACGA TCACCTGT | 3820 |
| 738 | GUGAGCGG G CGUCGGGG | 2814 | CCCCGACG GGCTAGCTACAACGA CCGCTCAC | 3821 |
| 740 | GAGCGGGC G UCGGGGGC | 2815 | GCCCCCGA GGCTAGCTACAACGA GCCCGCTC | 3822 |
| 747 | CGUCGGGG G CUGCGGCG | 2816 | CGCCGCAG GGCTAGCTACAACGA CCCCGACG | 3823 |
| 750 | CGGGGGCU G CGGCGGGC | 2817 | GCCCGCCG GGCTAGCTACAACGA AGCCCCCG | 3824 |
| 753 | GGGCUGCG G CGGGCGGG | 2818 | CCCGCCCG GGCTAGCTACAACGA CGCAGCCC | 3825 |
| 757 | UGCGGCGG G CGGGGGCC | 2819 | GGCCCCCG GGCTAGCTACAACGA CCGCCGCA | 3826 |
| 763 | GGGCGGGG G CCCCUUCC | 2820 | GGAAGGGG GGCTAGCTACAACGA CCCCGCCC | 3827 |
| 780 | UCCCUGGG G CCUGCGGG | 2821 | CCCGCAGG GGCTAGCTACAACGA CCCAGGGA | 3828 |
| 784 | UGGGGCCU G CGGGAAUC | 2822 | GATTCCCG GGCTAGCTACAACGA AGGCCCCA | 3829 |
| 790 | CUGCGGGA A UCCGGGCC | 2823 | GGCCCCGG GGCTAGCTACAACGA TCCCGCAG | 3830 |
| 796 | GAAUCCGG G CCCCACCC | 2824 | GGGTGGGG GGCTAGCTACAACGA CCGGATTC | 3831 |
| 801 | CGGGCCCC A CCCGUGGC | 2825 | GCCACGGG GGCTAGCTACAACGA GGGGCCCG | 3832 |
| 805 | CCCCACCC G UGGCCUCG | 2826 | CGAGGCCA GGCTAGCTACAACGA GGGTGGGG | 3833 |
| 808 | CACCCGUG G CCUCGCGC | 2827 | GCGCGAGG GGCTAGCTACAACGA CACGGGTG | 3834 |
| 813 | GUGGCCUC G CGCUGGGC | 2828 | GCCCAGCG GGCTAGCTACAACGA GAGGCCAC | 3835 |
| 815 | GGCCUCGC G CUGGGCAC | 2829 | GTGCCCAG GGCTAGCTACAACGA GCGAGGCC | 3836 |
| 820 | CGCGCUGG G CACGGUCC | 2830 | GGACCGTG GGCTAGCTACAACGA CCAGCGCG | 3837 |
| 822 | CGCUGGGC A CGGUCCCC | 2831 | GGGGACCG GGCTAGCTACAACGA GCCCAGCG | 3838 |
| 825 | UGGGCACG G UCCCCACG | 2832 | CGTGGGGA GGCTAGCTACAACGA CGTGCCCA | 3839 |
| 831 | CGGUCCCC A CGCCGGCG | 2833 | CGCCGGCG GGCTAGCTACAACGA GGGGACCG | 3840 |
| 833 | GUCCCCAC G CCGGCGUA | 2834 | TACGCCGG GGCTAGCTACAACGA GTGGGGAC | 3841 |
| 837 | CCACGCCG G CGUACCCG | 2835 | CGGGTACG GGCTAGCTACAACGA CGGCGTGG | 3842 |
| 839 | ACGCCGGC G UACCCGGG | 2836 | CCCGGGTA GGCTAGCTACAACGA GCCGGCGT | 3843 |
| 841 | GCCGGCGU A CCCGGGAG | 2837 | CTCCCGGG GGCTAGCTACAACGA ACGCCGGC | 3844 |
| 849 | ACCCGGGA G CCICGGGC | 2838 | GCCCGAGG GGCTAGCTACAACGA TCCCGGGT | 3845 |
| 856 | AGCCUCGG G CCCGGCGC | 2839 | GCGCCGGG GGCTAGCTACAACGA CCGAGGCT | 3846 |
| 861 | CGGGCCCG G CGCCCUCA | 2840 | TGAGGGCG GGCTAGCTACAACGA CGGGCCCG | 3847 |
| 863 | GGCCCGGC G CCCUCACA | 2841 | TGTGAGGG GGCTAGCTACAACGA GCCGGGCC | 3848 |
| 869 | GCGCCCUC A CACCCGGG | 2842 | CCCGGGTG GGCTAGCTACAACGA GAGGGCGC | 3849 |
| 871 | GCCCUCAC A CCCGGGGG | 2843 | CCCCCGGG GGCTAGCTACAACGA GTGAGGGC | 3850 |
| 879 | ACCCGGGG G CGUCUGGG | 2844 | CCCAGACG GGCTAGCTACAACGA CCCCGGGT | 3851 |
| 881 | CCGGGGGC G UCUGGGAG | 2845 | CTCCCAGA GGCTAGCTACAACGA GCCCCCGG | 3852 |
| 893 | GGGAGGAG G CGGCCGCG | 2846 | CGCGGCCG GGCTAGCTACAACGA CTCCTCCC | 3853 |
| 896 | AGGAGGCG G CCGCGGCC | 2847 | GGCCGCGG GGCTAGCTACAACGA CGCCTCCT | 3854 |
| 899 | AGGCGGCC G CGGCCACG | 2848 | CGTGGCCG GGCTAGCTACAACGA GGCCGCCT | 3855 |
| 902 | CGGCCGCG G CCACGGCA | 2849 | TGCCGTGG GGCTAGCTACAACGA CGCGGCCG | 3856 |
| 905 | CCGCGGCC A CGGCACGC | 2850 | GCGTGCCG GGCTAGCTACAACGA GGCCGCGG | 3857 |
| 908 | CGGCCACG G CACGCCCG | 2851 | CGGGCGTG GGCTAGCTACAACGA CGTGGCCG | 3858 |
| 910 | GCCACGGC A CGCCCGGG | 2852 | CCCGGGCG GGCTAGCTACAACGA GCCGTGGC | 3859 |
| 912 | CACGGCAC G CCCGGGCA | 2853 | TGCCCGGG GGCTAGCTACAACGA GTGCCGTG | 3860 |
| 918 | ACGCCCGG G CACCCCCG | 2854 | CGGGGGTG GGCTAGCTACAACGA CCGGGCGT | 3861 |
| 920 | GCCCGGGC A CCCCCGAU | 2855 | ATCGGGGG GGCTAGCTACAACGA GCCCGGGC | 3862 |
| 927 | CACCCCCG A UUCAGCAU | 2856 | ATGCTGAA GGCTAGCTACAACGA CGGGGGTG | 3863 |
| 932 | CCGAUUCA G CAUCACAG | 2857 | CTGTGATG GGCTAGCTACAACGA TGAATCGG | 3864 |
| 934 | GAUUCAGC A UCACAGGU | 2858 | ACCTGTGA GGCTAGCTACAACGA GCTGAATC | 3865 |
| 937 | UCAGCAUC A CAGGUCGC | 2859 | GCGACCTG GGCTAGCTACAACGA GATGCTGA | 3866 |
| 941 | CAUCACAG G UCGCGGAC | 2860 | GTCCGCGA GGCTAGCTACAACGA CTGTGATG | 3867 |
| 944 | CACAGGUC G CGGACCAG | 2861 | CTGGTCCG GGCTAGCTACAACGA GACCTGTG | 3868 |
| 948 | GGUCGCGG A CCAGGCCG | 2862 | CGGCCTGG GGCTAGCTACAACGA CCGCGACC | 3869 |
| 953 | CGGACCAG G CCGGGGGC | 2863 | GCCCCCGG GGCTAGCTACAACGA CTGGTCCG | 3870 |
| 960 | GGCCGGGG G CCUCAGCC | 2864 | GGCTGAGG GGCTAGCTACAACGA CCCCGGCC | 3871 |
| 966 | GGGCCUCA G CCCCAGUG | 2865 | CACTGGGG GGCTAGCTACAACGA TGAGGCCC | 3872 |
| 972 | CAGCCCCA G UGCCUUUU | 2866 | AAAAGGCA GGCTAGCTACAACGA TGGGGCTG | 3873 |
| 974 | GCCCCAGU G CCUUUUCC | 2867 | GGAAAAGG GGCTAGCTACAACGA ACTGGGGC | 3874 |
| 991 | CUCUCCGG G UCUCCCGC | 2868 | GCGGGAGA GGCTAGCTACAACGA CCGGAGAG | 3875 |
| 998 | GGUCUCCC G CGCCGCUU | 2869 | AAGCGGCG GGCTAGCTACAACGA GGGAGACC | 3876 |
| 1000 | UCUCCCGC G CCGCUUCU | 2870 | AGAAGCGG GGCTAGCTACAACGA GCGGGAGA | 3877 |
| 1003 | CCCGCGCC G CUUCUCGG | 2871 | CCGAGAAG GGCTAGCTACAACGA GGCGCGGG | 3878 |
| 1011 | GCUUCUCG G CCCCUUCC | 2872 | GGAAGGGG GGCTAGCTACAACGA CGAGAAGC | 3879 |
| 1021 | CCCUUCCU G UCGCUCAG | 2873 | CTGAGCGA GGCTAGCTACAACGA AGGAAGGG | 3880 |
| 1024 | UUCCUGUC G CUCAGUCC | 2874 | GGACTGAG GGCTAGCTACAACGA GACAGGAA | 3881 |
| 1029 | GUCGCUCA G UCCCUGCU | 2875 | AGCAGGGA GGCTAGCTACAACGA TGAGCGAC | 3882 |
| 1035 | CAGUCCCU G CUUCCCAG | 2876 | CTGGGAAG GGCTAGCTACAACGA AGGGACTG | 3883 |
| 1046 | UCCCAGGA G CUCCUCUG | 2877 | CAGAGGAG GGCTAGCTACAACGA TCCTGGGA | 3884 |
| 1054 | GCUCCUCU G UCUUCUCC | 2878 | GGAGAAGA GGCTAGCTACAACGA AGAGGAGC | 3885 |
| 1064 | CUUCUCCA G CUUUCUGU | 2879 | ACAGAAAG GGCTAGCTACAACGA TGGAGAAG | 3886 |
| 1071 | AGCUUUCU G UGGCUGAA | 2880 | TTCAGCCA GGCTAGCTACAACGA AGAAAGCT | 3887 |
| 1074 | UUUCUGUG G CUGAAAGA | 2881 | TCTTTCAG GGCTAGCTACAACGA CACAGAAA | 3888 |
| 1082 | GCUGAAAG A UGCCCCCG | 2882 | CGGGGGCA GGCTAGCTACAACGA CTTTCAGC | 3889 |
| 1084 | UGAAAGAU G CCCCCGGU | 2883 | ACCGGGGG GGCTAGCTACAACGA ATCTTTCA | 3890 |
| 1091 | UGCCCCCG G UUCCCCGC | 2884 | GCGGGGAA GGCTAGCTACAACGA CGGGGGCA | 3891 |
| 1098 | GGUUCCCC G CCGGGGGU | 2885 | ACCCCCGG GGCTAGCTACAACGA GGGGAACC | 3892 |
| 1105 | CGCCGGGG G UGCGGGGC | 2886 | GCCCCGCA GGCTAGCTACAACGA CCCCGGCG | 3893 |
| 1107 | CCGGGGGU G CGGGGCGC | 2887 | GCGCCCCG GGCTAGCTACAACGA ACCCCCGG | 3894 |
| 1112 | GGUGCGGG G CGCUGCCC | 2888 | GGGCAGCG GGCTAGCTACAACGA CCCGCACC | 3895 |
| 1114 | UGCGGGGC G CUGCCCGG | 2889 | CCGGGCAG GGCTAGCTACAACGA GCCCCGCA | 3896 |
| 1117 | GGGGCGCU G CCCGGGUC | 2890 | GACCCGGG GGCTAGCTACAACGA AGCGCCCC | 3897 |
| 1123 | CUGCCCGG G UCUGCCCU | 2891 | AGGGCAGA GGCTAGCTACAACGA CCGGGCAG | 3898 |
| 1127 | CCGGGUCU G CCCUCCCC | 2892 | GGGGAGGG GGCTAGCTACAACGA AGACCCGG | 3899 |
| 1139 | UCCCCUCG G CGGCGCCU | 2893 | AGGCGCCG GGCTAGCTACAACGA CGAGGGGA | 3900 |
| 1142 | CCUCGGCG G CGCCUAGU | 2894 | ACTAGGCG GGCTAGCTACAACGA CGCCGAGG | 3901 |
| 1144 | UCGGCGGC G CCUAGUAC | 2895 | GTACTAGG GGCTAGCTACAACGA GCCGCCGA | 3902 |
| 1149 | GGCGCCUA G UACGCAGU | 2896 | ACTGCGTA GGCTAGCTACAACGA TAGGCGCC | 3903 |
| 1151 | CGCCUAGU A CGCAGUAG | 2897 | CTACTGCG GGCTAGCTACAACGA ACTAGGCG | 3904 |
| 1153 | CCUAGUAC G CAGUAGGC | 2898 | GCCTACTG GGCTAGCTACAACGA GTACTAGG | 3905 |
| 1156 | AGUACGCA G UAGGCGCU | 2899 | AGCGCCTA GGCTAGCTACAACGA TGCGTACT | 3906 |
| 1160 | CGCAGUAG G CGCUCAGC | 2900 | GCTGAGCG GGCTAGCTACAACGA CTACTGCG | 3907 |
| 1162 | CAGUAGGC G CUCAGCAA | 2901 | TTGCTGAG GGCTAGCTACAACGA GCCTACTG | 3908 |
| 1167 | GGCGCUCA G CAAAUACU | 2902 | AGTATTTG GGCTAGCTACAACGA TGAGCGCC | 3909 |
| 1171 | CUCAGCAA A UACUUGUC | 2903 | GACAAGTA GGCTAGCTACAACGA TTGCTGAG | 3910 |
| 1173 | CAGCAAAU A CUUGUCGG | 2904 | CCGACAAG GGCTAGCTACAACGA ATTTGCTG | 3911 |
| 1177 | AAAUACUU G UCGGAGGC | 2905 | GCCTCCGA GGCTAGCTACAACGA AAGTATTT | 3912 |
| 1184 | UGUCGGAG G CACCAGCG | 2906 | CGCTGGTG GGCTAGCTACAACGA CTCCGACA | 3913 |
| 1186 | UCGGAGGC A CCAGCGCC | 2907 | GGCGCTGG GGCTAGCTACAACGA GCCTCCGA | 3914 |
| 1190 | AGGCACCA G CGCCGCGG | 2908 | CCGCGGCG GGCTAGCTACAACGA TGGTGCCT | 3915 |
| 1192 | GCACCAGC G CCGCGGGG | 2909 | CCCCGCGG GGCTAGCTACAACGA GCTGGTGC | 3916 |
| 1195 | CCAGCGCC G CGGGGCCU | 2910 | AGGCCCCG GGCTAGCTACAACGA GGCGCTGG | 3917 |
| 1200 | GCCGCGGG G CCUGCAGG | 2911 | CCTGCAGG GGCTAGCTACAACGA CCCGCGGC | 3918 |
| 1204 | CGGGGCCU G CAGGCUGG | 2912 | CCAGCCTG GGCTAGCTACAACGA AGGCCCCG | 3919 |
| 1208 | GCCUGCAG G CUGGCACU | 2913 | AGTGCCAG GGCTAGCTACAACGA CTGCAGGC | 3920 |
| 1212 | GCAGGCUG G CACUAGCC | 2914 | GGCTAGTG GGCTAGCTACAACGA CAGCCTGC | 3921 |
| 1214 | AGGCUGGC A CUAGCCUG | 2915 | CAGGCTAG GGCTAGCTACAACGA GCCAGCCT | 3922 |
| 1218 | UGGCACUA G CCUGCCCG | 2916 | CGGGCAGG GGCTAGCTACAACGA TAGTGCCA | 3923 |
| 1222 | ACUAGCCU G CCCGGGCA | 2917 | TGCCCGGG GGCTAGCTACAACGA AGGCTAGT | 3924 |
| 1228 | CUGCCCGG G CACGCCGU | 2918 | ACGGCGTG GGCTAGCTACAACGA CCGGGCAG | 3925 |
| 1230 | GCCCGGGC A CGCCGUGG | 2919 | CCACGGCG GGCTAGCTACAACGA GCCCGGGC | 3926 |
| 1232 | CCGGGCAC G CCGUGGCG | 2920 | CGCCACGG GGCTAGCTACAACGA GTGCCCGG | 3927 |
| 1235 | GGCACGCC G UGGCGCGC | 2921 | GCGCGCCA GGCTAGCTACAACGA GGCGTGCC | 3928 |
| 1238 | ACGCCGUG G CGCGCUCC | 2922 | GGAGCGCG GGCTAGCTACAACGA CACGGCGT | 3929 |
| 1240 | GCCGUGGC G CGCUCCGC | 2923 | GCGGAGCG GGCTAGCTACAACGA GCCACGGC | 3930 |
| 1242 | CGUGGCGC G CUCCGCCG | 2924 | CGGCGGAG GGCTAGCTACAACGA GCGCCACG | 3931 |
| 1247 | CGCGCUCC G CCGUGGCC | 2925 | GGCCACGG GGCTAGCTACAACGA GGAGCGCG | 3932 |
| 1250 | GCUCCGCC G UGGCCAGA | 2926 | TCTGGCCA GGCTAGCTACAACGA GGCGGAGC | 3933 |
| 1253 | CCGCCGUG G CCAGACCU | 2927 | AGGTCTGG GGCTAGCTACAACGA CACGGCGG | 3934 |
| 1258 | GUGGCCAG A CCUGUUCU | 2928 | AGAACAGG GGCTAGCTACAACGA CTGGCCAC | 3935 |
| 1262 | CCAGACCU G UUCUGGAG | 2929 | CTCCAGAA GGCTAGCTACAACGA AGGTCTGG | 3936 |
| 1272 | UCUGGAGG A CGGUAACC | 2930 | GGTTACCG GGCTAGCTACAACGA CCTCCAGA | 3937 |
| 1275 | GGAGGACG G UAACCUCA | 2931 | TGAGGTTA GGCTAGCTACAACGA CGTCCTCC | 3938 |
| 1278 | GGACGGUA A CCUCAGCC | 2932 | GGCTGAGG GGCTAGCTACAACGA TACCGTCC | 3939 |
| 1284 | UAACCUCA G CCCUCGGG | 2933 | CCCGAGGG GGCTAGCTACAACGA TGAGGTTA | 3940 |
| 1292 | GCCCUCGG G CGCCUCCC | 2934 | GGGAGGCG GGCTAGCTACAACGA CCGAGGGC | 3941 |
| 1294 | CCUCGGGC G CCUCCCUU | 2935 | AAGGGAGG GGCTAGCTACAACGA GCCCGAGG | 3942 |
| 1305 | UCCCUUUA G CCUUUCUG | 2936 | CAGAAAGG GGCTAGCTACAACGA TAAAGGGA | 3943 |
| 1313 | GCCUUUCU G CCGACCCA | 2937 | TGGGTCGG GGCTAGCTACAACGA AGAAAGGC | 3944 |
| 1317 | UUCUGCCG A CCCAGCAG | 2938 | CTGCTGGG GGCTAGCTACAACGA CGGCAGAA | 3945 |
| 1322 | CCGACCCA G CAGCUUCU | 2939 | AGAAGCTG GGCTAGCTACAACGA TGGGTCGG | 3946 |
| 1325 | ACCCAGCA G CUUCUAAU | 2940 | ATTAGAAG GGCTAGCTACAACGA TGCTGGGT | 3947 |
| 1332 | AGCUUCUA A UUUGGGUG | 2941 | CACCCAAA GGCTAGCTACAACGA TAGAAGCT | 3948 |
| 1338 | UAAUUUGG G UGCGUGGU | 2942 | ACCACGCA GGCTAGCTACAACGA CCAAATTA | 3949 |
| 1340 | AUUUGGGU G CGUGGUUG | 2943 | CAACCACG GGCTAGCTACAACGA ACCCAAAT | 3950 |
| 1342 | UUGGGUGC G UGGUUGAG | 2944 | CTCAACCA GGCTAGCTACAACGA GCACCCAA | 3951 |
| 1345 | GGUGCGUG G UUGAGAGC | 2945 | GCTCTCAA GGCTAGCTACAACGA CACGCACC | 3952 |
| 1352 | GGUUGAGA G CGCUCAGC | 2946 | GCTGAGCG GGCTAGCTACAACGA TCTCAACC | 3953 |
| 1354 | UUGAGAGC G CUCAGCUG | 2947 | CAGCTGAG GGCTAGCTACAACGA GCTCTCAA | 3954 |
| 1359 | AGCGCUCA G CUGUCAGC | 2948 | GCTGACAg GGCTAGCTACAACGA TGAGCGCT | 3955 |
| 1362 | GCUCAGCU G UCAGCCCU | 2949 | AGGGCTGA GGCTAGCTACAACGA AGCTGAGC | 3956 |
| 1366 | AGCUGUCA G CCCUGCCU | 2950 | AGGCAGGG GGCTAGCTACAACGA TGACAGCT | 3957 |
| 1371 | UCAGCCCU G CCUUUGAG | 2951 | CTCAAAGG GGCTAGCTACAACGA AGGGCTGA | 3958 |
| 1381 | CUUUGAGG G CUGGGUCC | 2952 | GGACCCAG GGCTAGCTACAACGA CCTCAAAG | 3959 |
| 1386 | AGGGCUGG G UCCCUUUU | 2953 | AAAAGGGA GGCTAGCTACAACGA CCAGCCCT | 3960 |
| 1398 | CUUUUCCC A UCACUGGG | 2954 | CCCAGTGA GGCTAGCTACAACGA GGGAAAAG | 3961 |
| 1401 | UUCCCAUC A CUGGGUCA | 2955 | TGACCCAG GGCTAGCTACAACGA GATGGGAA | 3962 |
| 1406 | AUCACUGG G UCAUUAAG | 2956 | CTTAATGA GGCTAGCTACAACGA CCAGTGAT | 3963 |
| 1409 | ACUGGGUC A UUAAGAGC | 2957 | GCTCTTAA GGCTAGCTACAACGA GACCCAGT | 3964 |
| 1416 | CAUUAAGA G CAAGUGGG | 2958 | CCCACTTG GGCTAGCTACAACGA TCTTAATG | 3965 |
| 1420 | AAGAGCAA G UGGGGGCG | 2959 | CGCCCCCA GGCTAGCTACAACGA TTGCTCTT | 3966 |
| 1426 | AAGUGGGG G CGAGGCGA | 2960 | TCGCCTCG GGCTAGCTACAACGA CCCCACTT | 3967 |
| 1431 | GGGGCGAG G CGACAGCC | 2961 | GGCTGTCG GGCTAGCTACAACGA CTCGCCCC | 3968 |
| 1434 | GCGAGGCG A CAGCCCUC | 2962 | GAGGGCTG GGCTAGCTACAACGA CGCCTCGC | 3969 |
| 1437 | AGGCGACA G CCCUCCCG | 2963 | CGGGAGGG GGCTAGCTACAACGA TGTCGCCT | 3970 |
| 1445 | GCCCUCCC G CACGCUGG | 2964 | CCAGCGTG GGCTAGCTACAACGA GGGAGGGC | 3971 |
| 1447 | CCUCCCGC A CGCUGGGU | 2965 | ACCCAGCG GGCTAGCTACAACGA GCGGGAGG | 3972 |
| 1449 | UCCCGCAC G CUGGGUUG | 2966 | CAACCCAG GGCTAGCTACAACGA GTGCGGGA | 3973 |
| 1454 | CACGCUGG G UUGCAGCU | 2967 | AGCTGCAA GGCTAGCTACAACGA CCAGCGTG | 3974 |
| 1457 | GCUGGGUU G CAGCUGCA | 2968 | TGCAGCTG GGCTAGCTACAACGA AACCCAGC | 3975 |
| 1460 | GGGUUGCA G CUGCACAG | 2969 | CTGTGCAG GGCTAGCTACAACGA TGCAACCC | 3976 |
| 1463 | UUGCAGCU G CACAGGUA | 2970 | TACCTGTG GGCTAGCTACAACGA AGCTGCAA | 3977 |
| 1465 | GCAGCUGC A CAGGUAGG | 2971 | CCTACCTG GGCTAGCTACAACGA GCAGCTGC | 3978 |
| 1469 | CUGCACAG G UAGGCACG | 2972 | CGTGCCTA GGCTAGCTACAACGA CTGTGCAG | 3979 |
| 1473 | ACAGGUAG G CACGCUGC | 2973 | GCAGCGTG GGCTAGCTACAACGA CTACCTGT | 3980 |
| 1475 | AGGUAGGC A CGCUGCAG | 2974 | CTGCAGCG GGCTAGCTACAACGA GCCTACCT | 3981 |
| 1477 | GUAGGCAC G CUGCAGUC | 2975 | GACTGCAG GGCTAGCTACAACGA GTGCCTAC | 3982 |
| 1480 | GGCACGCU G CAGUCCUU | 2976 | AAGGACTG GGCTAGCTACAACGA AGCGTGCC | 3983 |
| 1483 | ACGCUGCA G UCCUUGCU | 2977 | AGCAAGGA GGCTAGCTACAACGA TGCAGCGT | 3984 |
| 1489 | CAGUCCUU G CUGCCUGG | 2978 | CCAGGCAG GGCTAGCTACAACGA AAGGACTG | 3985 |
| 1492 | UCCUUGCU G CCUGGCGU | 2979 | ACGCCAGG GGCTAGCTACAACGA AGCAAGGA | 3986 |
| 1497 | GCUGCCUG G CGUUGGGG | 2980 | CCCCAACG GGCTAGCTACAACGA CAGGCAGC | 3987 |
| 1499 | UGCCUGGC G UUGGGGCC | 2981 | GGCCCCAA GGCTAGCTACAACGA GCCAGGCA | 3988 |
| 1505 | GCGUUGGG G CCCAGGGA | 2982 | TCCCTGGG GGCTAGCTACAACGA CCCAACGC | 3989 |
| 1513 | GCCCAGGG A CCGCUGUG | 2983 | CACAGCGG GGCTAGCTACAACGA CCCTGGGC | 3990 |
| 1516 | CAGGGACC G CUGUGGGU | 2984 | ACCCACAG GGCTAGCTACAACGA GGTCCCTG | 3991 |
| 1519 | GGACCGCU G UGGGUUUG | 2985 | CAAACCCA GGCTAGCTACAACGA AGCGGTCC | 3992 |
| 1523 | CGCUGUGG G UUUGCCCU | 2986 | AGGGCAAA GGCTAGCTACAACGA CCACAGCG | 3993 |
| 1527 | GUGGGUUU G CCCUUCAG | 2987 | CTGAAGGG GGCTAGCTACAACGA AAACCCAC | 3994 |
| 1536 | CCCUUCAG A UGGCCCUG | 2988 | CAGGGCCA GGCTAGCTACAACGA CTGAAGGG | 3995 |
| 1539 | UUCAGAUG G CCCUGCCA | 2989 | TGGCAGGG GGCTAGCTACAACGA CATCTGAA | 3996 |
| 1544 | AUGGCCCU G CCAGCAGC | 2990 | GCTGCTGG GGCTAGCTACAACGA AGGGCCAT | 3997 |
| 1548 | CCCUGCCA G CAGCUGCC | 2991 | GGCAGCTG GGCTAGCTACAACGA TGGCAGGG | 3998 |
| 1551 | UGCCAGCA G CUGCCCUG | 2992 | CAGGGCAG GGCTAGCTACAACGA TGCTGGCA | 3999 |
| 1554 | CAGCAGCU G CCCUGUGG | 2993 | CCACAGGG GGCTAGCTACAACGA AGCTGCTG | 4000 |
| 1559 | GCUGCCCU G UGGGGCCU | 2994 | AGGCCCCA GGCTAGCTACAACGA AGGGCAGC | 4001 |
| 1564 | CCUGUGGG G CCUGGGGC | 2995 | GCCCCAGG GGCTAGCTACAACGA CCCACAGG | 4002 |
| 1571 | GGCCUGGG G CUGGGCCU | 2996 | AGGCCCAG GGCTAGCTACAACGA CCCAGGCC | 4003 |
| 1576 | GGGGCUGG G CCUGGGCC | 2997 | GGCCCAGG GGCTAGCTACAACGA CCAGCCCC | 4004 |
| 1582 | GGGCCUGG G CCUGGCUG | 2998 | CAGCCAGG GGCTAGCTACAACGA CCAGGCCC | 4005 |
| 1587 | UGGGCCUG G CUGAGCAG | 2999 | CTGCTCAG GGCTAGCTACAACGA CAGGCCCA | 4006 |
| 1592 | CUGGCUGA G CAGGGCCC | 3000 | CGGCCCTG GGCTAGCTACAACGA TCAGCCAG | 4007 |
| 1597 | UGAGCAGG G CCCUCCUU | 3001 | AAGGAGGG GGCTAGCTACAACGA CCTGCTCA | 4008 |
| 1607 | CCUCCUUG G CAGGUGGG | 3002 | CCCACCTG GGCTAGCTACAACGA CAAGGAGG | 4009 |
| 1611 | CUUGGCAG G UGGGGCAG | 3003 | CTGCCCCA GGCTAGCTACAACGA CTGCCAAG | 4010 |
| 1616 | CAGGUGGG G CAGGAGAC | 3004 | GTCTCCTG GGCTAGCTACAACGA CCCACCTG | 4011 |
| 1623 | GGCAGGAG A CCCUGUAG | 3005 | CTACAGGG GGCTAGCTACAACGA CTCCTGCC | 4012 |
| 1628 | GAGACCCU G UAGGAGGA | 3006 | TCCTCCTA GGCTAGCTACAACGA AGGGTCTC | 4013 |
| 1636 | GUAGGAGG A CCCCGGGC | 3007 | GCCCGGGG GGCTAGCTACAACGA CCTCCTAC | 4014 |
| 1643 | GACCCCGG G CCGCAGGC | 3008 | GCCTGCGG GGCTAGCTACAACGA CCGGGGTC | 4015 |
| 1646 | CCCGGGCC G CAGGCCCC | 3009 | GGGGCCTG GGCTAGCTACAACGA GGCCCGGG | 4016 |
| 1650 | GGCCGCAG G CCCCUGAG | 3010 | CTCAGGGG GGCTAGCTACAACGA CTGCGGCC | 4017 |
| 1661 | CCUGAGGA G CGAUGACG | 3011 | CGTCATCG GGCTAGCTACAACGA TCCTCAGG | 4018 |
| 1664 | GAGGAGCG A UGACGGAA | 3012 | TTCCGTCA GGCTAGCTACAACGA CGCTCCTC | 4019 |
| 1667 | GAGCGAUG A CGGAAUAU | 3013 | ATATTCCG GGCTAGCTACAACGA CATCGCTC | 4020 |
| 1672 | AUGACGGA A UAUAAGCU | 3014 | AGCTTATA GGCTAGCTACAACGA TCCGTCAT | 4021 |
| 1674 | GACGGAAU A UAAGCUGG | 3015 | CCAGCTTA GGCTAGCTACAACGA ATTCCGTC | 4022 |
| 1678 | GAAUAUAA G CUGGUGGU | 3016 | ACCACCAG GGCTAGCTACAACGA TTATATTC | 4023 |
| 1682 | AUAAGCUG G UGGUGGUG | 3017 | CACCACCA GGCTAGCTACAACGA CAGCTTAT | 4024 |
| 1685 | AGCUGGUG G UGGUGGGC | 3018 | GCCCACCA GGCTAGCTACAACGA CACCAGCT | 4025 |
| 1688 | UGGUGGUG G UGGGCGCC | 3019 | GGCGCCCA GGCTAGCTACAACGA CACCACCA | 4026 |
| 1692 | GGUGGUGG G CGCCGGCG | 3020 | CGCCGGCG GGCTAGCTACAACGA CCACCACC | 4027 |
| 1694 | UGGUGGGC G CCGGCGGU | 3021 | ACCGCCGG GGCTAGCTACAACGA GCCCACCA | 4028 |
| 1698 | GGGCGCCG G CGGUGUGG | 3022 | CCACACCG GGCTAGCTACAACGA CGGCGCCC | 4029 |
| 1701 | CGCCGGCG G UGUGGGCA | 3023 | TGCCCACA GGCTAGCTACAACGA CGCCGGCG | 4030 |
| 1703 | CCGGCGGU G UGGGCAAG | 3024 | CTTGCCCA GGCTAGCTACAACGA ACCGCCGG | 4031 |
| 1707 | CGGUGUGG G CAAGAGUG | 3025 | CACTCTTG GGCTAGCTACAACGA CCACACCG | 4032 |
| 1713 | GGGCAAGA G UGCGCUGA | 3026 | TCAGCGCA GGCTAGCTACAACGA TCTTGCCC | 4033 |
| 1715 | GCAAGAGU G CGCUGACC | 3027 | GGTCAGCG GGCTAGCTACAACGA ACTCTTGC | 4034 |
| 1717 | AAGAGUGC G CUGACCAU | 3028 | ATGGTCAG GGCTAGCTACAACGA GCACTCTT | 4035 |
| 1721 | GUGCGCUG A CCAUCCAG | 3029 | CTGGATGG GGCTAGCTACAACGA CAGCGCAC | 4036 |
| 1724 | CGCUGACC A UCCAGCUG | 3030 | CAGCTGGA GGCTAGCTACAACGA GGTCAGCG | 4037 |
| 1729 | ACCAUCCA G CUGAUCCA | 3031 | TGGATCAG GGCTAGCTACAACGA TGGATGGT | 4038 |
| 1733 | UCCAGCUG A UCCAGAAC | 3032 | GTTCTGGA GGCTAGCTACAACGA CAGCTGGA | 4039 |
| 1740 | GAUCCAGA A CCAUUUUG | 3033 | CAAAATGG GGCTAGCTACAACGA TCTGGATC | 4040 |
| 1743 | CCAGAACC A UUUUGUGG | 3034 | CCACAAAA GGCTAGCTACAACGA GGTTCTGG | 4041 |
| 1748 | ACCAUUUU G UGGACGAA | 3035 | TTCGTCCA GGCTAGCTACAACGA AAAATGGT | 4042 |
| 1752 | UUUUGUGG A CGAAUACG | 3036 | CGTATTCG GGCTAGCTACAACGA CCACAAAA | 4043 |
| 1756 | GUGGACGA A UACGACCC | 3037 | GGGTCGTA GGCTAGCTACAACGA TCGTCCAC | 4044 |
| 1758 | GGACGAAU A CGACCCCA | 3038 | TGGGGTCG GGCTAGCTACAACGA ATTCGTCC | 4045 |
| 1761 | CGAAUACG A CCCCACUA | 3039 | TAGTGGGG GGCTAGCTACAACGA CGTATTCG | 4046 |
| 1766 | ACGACCCC A CUAUAGAG | 3040 | CTCTATAG GGCTAGCTACAACGA GGGGTCGT | 4047 |
| 1769 | ACCCCACU A UAGAGGAU | 3041 | ATCCTCTA GGCTAGCTACAACGA AGTGGGGT | 4048 |
| 1776 | UAUAGAGG A UUCCUACC | 3042 | GGTAGGAA GGCTAGCTACAACGA CCTCTATA | 4049 |
| 1782 | GGAUUCCU A CCGGAAGC | 3043 | GCTTCCGG GGCTAGCTACAACGA AGGAATCC | 4050 |
| 1789 | UACCGGAA G CAGGUGGU | 3044 | ACCACCTC GGCTAGCTACAACGA TTCCGGTA | 4051 |
| 1793 | GGAAGCAG G UGGUCAUU | 3045 | AATGACCA GGCTAGCTACAACGA CTGCTTCC | 4052 |
| 1796 | AGCAGGUG G UCAUUGAU | 3046 | ATCAATGA GGCTAGCTACAACGA CACCTGCT | 4053 |
| 1799 | AGGUGGUC A UUGAUGGG | 3047 | CCCATCAA GGCTAGCTACAACGA GACCACCT | 4054 |
| 1803 | GGUCAUUG A UGGGGAGA | 3048 | TCTCCCCA GGCTAGCTACAACGA CAATGACC | 4055 |
| 1811 | AUGGGGAG A CGUGCCUG | 3049 | CAGGCACG GGCTAGCTACAACGA CTCCCCAT | 4056 |
| 1813 | GGGGAGAC G UGCCUGUU | 3050 | AACAGGCA GGCTAGCTACAACGA GTCTCCCC | 4057 |
| 1815 | GGAGACGU C CCUGUUGG | 3051 | CCAACAGG GGCTAGCTACAACGA ACGTCTCC | 4058 |
| 1819 | ACGUGCCU G UUGGACAU | 3052 | ATGTCCAA GGCTAGCTACAACGA AGGCACGT | 4059 |
| 1824 | CCUGUUGG A CAUCCUGG | 3053 | CCAGGATG GGCTAGCTACAACGA CCAACAGG | 4060 |
| 1826 | UGUUGGAC A UCCUGGAU | 3054 | ATCCAGGA GGCTAGCTACAACGA GTCCAACA | 4061 |
| 1833 | CAUCCUGG A UACCGCCG | 3055 | CGGCGGTA GGCTAGCTACAACGA CCAGGATG | 4062 |
| 1835 | UCCUGGAU A CCGCCGGC | 3056 | GCCGGCGG GGCTAGCTACAACGA ATCCAGGA | 4063 |
| 1838 | UGGAUACC G CCGGCCAG | 3057 | CTGGCCGG GGCTAGCTACAACGA GGTATCCA | 4064 |
| 1842 | UACCGCCG G CCAGGAGG | 3058 | CCTCCTGG GGCTAGCTACAACGA CGGCGGTA | 4065 |
| 1852 | CAGGAGGA G UACAGCGC | 3059 | GCGCTGTA GGCTAGCTACAACGA TCCTCCTG | 4066 |
| 1854 | GGAGGAGU A CAGCGCCA | 3060 | TGGCGCTG GGCTAGCTACAACGA ACTCCTCC | 4067 |
| 1857 | GGAGUACA G CGCCAUGC | 3061 | GCATGGCG GGCTAGCTACAACGA TGTACTCC | 4068 |
| 1859 | AGUACAGC G CCAUGCGG | 3062 | CCGCATGG GGCTAGCTACAACGA GCTGTACT | 4069 |
| 1862 | ACAGCGCC A UGCGGGAC | 3063 | GTCCCGCA GGCTAGCTACAACGA GGCGCTGT | 4070 |
| 1864 | AGCGCCAU G CGGGACCA | 3064 | TGGTCCCG GGCTAGCTACAACGA ATGGCGCT | 4071 |
| 1869 | CAUGCGGG A CCAGUACA | 3065 | TGTACTGG GGCTAGCTACAACGA CCCGCATG | 4072 |
| 1873 | CGGGACCA G UACAUGCG | 3066 | CGCATGTA GGCTAGCTACAACGA TGGTCCCG | 4073 |
| 1875 | GGACCAGU A CAUGCGCA | 3067 | TGCGCATG GGCTAGCTACAACGA ACTGGTCC | 4074 |
| 1877 | ACCAGUAC A UGCGCACC | 3068 | GGTGCGCA GGCTAGCTACAACGA GTACTGGT | 4075 |
| 1879 | CAGUACAU G CGCACCGG | 3069 | CCGGTGCG GGCTAGCTACAACGA ATGTACTG | 4076 |
| 1881 | GUACAUGC G CACCGGGG | 3070 | CCCCGGTG GGCTAGCTACAACGA GCATGTAC | 4077 |
| 1883 | ACAUGCGC A CCGGGGAG | 3071 | CTCCCCGG GGCTAGCTACAACGA GCGCATGT | 4078 |
| 1893 | CGGGGAGG G CUUCCUGU | 3072 | ACAGGAAG GGCTAGCTACAACGA CCTCCCCG | 4079 |
| 1900 | GGCUUCCU G UGUGUGUU | 3073 | AACACACA GGCTAGCTACAACGA AGGAAGCC | 4080 |
| 1902 | CUUCCUGU G UGUGUUUG | 3074 | CAAACACA GGCTAGCTACAACGA ACAGGAAG | 4081 |
| 1904 | UCCUGUGU G UGUUUGCC | 3075 | GGCAAACA GGCTAGCTACAACGA ACACAGGA | 4082 |
| 1906 | CUGUGUGU G UUUGCCAU | 3076 | ATGGCAAA GGCTAGCTACAACGA ACACACAG | 4083 |
| 1910 | GUGUGUUU G CCAUCAAC | 3077 | GTTGATGG GGCTAGCTACAACGA AAACACAC | 4084 |
| 1913 | UGUUUGCC A UCAACAAC | 3078 | GTTGTTGA GGCTAGCTACAACGA GGCAAACA | 4085 |
| 1917 | UGCCAUCA A CAACACCA | 3079 | TGGTGTTG GGCTAGCTACAACGA TGATGGCA | 4086 |
| 1920 | CAUCAACA A CACCAAGU | 3080 | ACTTGGTG GGCTAGCTACAACGA TGTTGATG | 4087 |
| 1922 | UCAACAAC A CCAAGUCU | 3081 | AGACTTGG GGCTAGCTACAACGA GTTGTTGA | 4088 |
| 1927 | AACACCAA G UCUUUUGA | 3082 | TCAAAAGA GGCTAGCTACAACGA TTGGTGTT | 4089 |
| 1938 | UUUUGAGG A CAUCCACC | 3083 | GGTGGATG GGCTAGCTACAACGA CCTCAAAA | 4090 |
| 1940 | UUGAGGAC A UCCACCAG | 3084 | CTGGTGGA GGCTAGCTACAACGA GTCCTCAA | 4091 |
| 1944 | GGACAUCC A CCAGUACA | 3085 | TGTACTGG GGCTAGCTACAACGA GGATGTCC | 4092 |
| 1948 | AUCCACCA G UACAGGGA | 3086 | TCCCTGTA GGCTAGCTACAACGA TGGTGGAT | 4093 |
| 1950 | CCACCAGU A CAGGGAGC | 3087 | GCTCCCTG GGCTAGCTACAACGA ACTGGTGG | 4094 |
| 1957 | UACAGGGA G CAGAUCAA | 3088 | TTGATCTG GGCTAGCTACAACGA TCCCTGTA | 4095 |
| 1961 | GGGAGCAG A UCAAACGG | 3089 | CCGTTTGA GGCTAGCTACAACGA CTGCTCCC | 4096 |
| 1966 | CAGAUCAA A CGGGUGAA | 3090 | TTCACCCG GGCTAGCTACAACGA TTGATCTG | 4097 |
| 1970 | UCAAACGG G UGAAGGAC | 3091 | GTCCTTCA GGCTAGCTACAACGA CCGTTTGA | 4098 |
| 1977 | GGUGAAGG A CUCGGAUG | 3092 | CATCCGAG GGCTAGCTACAACGA CCTTCACC | 4099 |
| 1983 | GGACUCGG A UGACGUGC | 3093 | GCACGTCA GGCTAGCTACAACGA CCGAGTCC | 4100 |
| 1986 | CUCGGAUG A CGUGCCCA | 3094 | TGGGCACG GGCTAGCTACAACGA CATCCGAG | 4101 |
| 1988 | CCGAUGAC G UGCCCAUG | 3095 | CATGGGCA GGCTAGCTACAACGA GTCATCCG | 4102 |
| 1990 | GAUGACGU G CCCAUGGU | 3096 | ACCATGGG GGCTAGCTACAACGA ACGTCATC | 4103 |
| 1994 | ACGUGCCC A UGGUGCUG | 3097 | CAGCACCA GGCTAGCTACAACGA GGGCACGT | 4104 |
| 1997 | UGCCCAUG G UGCUGGUG | 3098 | CACCAGCA GGCTAGCTACAACGA CATGGGCA | 4105 |
| 1999 | CCCAUGGU G CUGGUGGG | 3099 | CCCACCAG GGCTAGCTACAACGA ACCATGGG | 4106 |
| 2003 | UGGUGCUG G UGGGGAAC | 3100 | GTTCCCCA GGCTAGCTACAACGA CAGCACCA | 4107 |
| 2010 | GGUGGGGA A CAAGUGUG | 3101 | CACACTTG GGCTAGCTACAACGA TCCCCACC | 4108 |
| 2014 | GGGAACAA G UGUGACCU | 3102 | AGGTCACA GGCTAGCTACAACGA TTGTTCCC | 4109 |
| 2016 | GAACAAGU G UGACCUGG | 3103 | CCAGGTCA GGCTAGCTACAACGA ACTTGTTC | 4110 |
| 2019 | CAAGUGUG A CCUGGCUG | 3104 | CAGCCAGG GGCTAGCTACAACGA CACACTTG | 4111 |
| 2024 | GUGACCUG G CUGCACGC | 3105 | GCGTGCAG GGCTAGCTACAACGA CAGGTCAC | 4112 |
| 2027 | ACCUGGCU G CACGCACU | 3106 | AGTGCGTG GGCTAGCTACAACGA AGCCAGGT | 4113 |
| 2029 | CUGGCUGC A CGCACUGU | 3107 | ACAGTGCG GGCTAGCTACAACGA GCAGCCAG | 4114 |
| 2031 | GGCUGCAC G CACUGUGG | 3108 | CCACAGTG GGCTAGCTACAACGA GTGCAGCC | 4115 |
| 2033 | CUGCACGC A CUGUGGAA | 3109 | TTCCACAG GGCTAGCTACAACGA GCGTGCAG | 4116 |
| 2036 | CACGCACU G UGGAAUCU | 3110 | AGATTCCA GGCTAGCTACAACGA AGTGCGTG | 4117 |
| 2041 | ACUGUGGA A UCUCGGCA | 3111 | TGCCGAGA GGCTAGCTACAACGA TCCACAGT | 4118 |
| 2047 | GAAUCUCG G CAGGCUCA | 3112 | TGAGCCTG GGCTAGCTACAACGA CGAGATTC | 4119 |
| 2051 | CUCGGCAG G CUCAGGAC | 3113 | GTCCTGAG GGCTAGCTACAACGA CTGCCGAG | 4120 |
| 2058 | GGCUCAGG A CCUCGCCC | 3114 | GGGCGAGG GGCTAGCTACAACGA CCTGAGCC | 4121 |
| 2063 | AGGACCUC G CCCGAAGC | 3115 | GCTTCGGG GGCTAGCTACAACGA GAGGTCCT | 4122 |
| 2070 | CGCCCGAA G CUACGGCA | 3116 | TGCCGTAG GGCTAGCTACAACGA TTCGGGCG | 4123 |
| 2073 | CCGAAGCU A CGGCAUCC | 3117 | GGATGCCG GGCTAGCTACAACGA AGCTTCGG | 4124 |
| 2076 | AAGCUACG G CAUCCCCU | 3118 | AGGGGATG GGCTAGCTACAACGA CGTAGCTT | 4125 |
| 2078 | GCUACGGC A UCCCCUAC | 3119 | GTAGGGGA GGCTAGCTACAACGA GCCGTAGC | 4126 |
| 2085 | CAUCCCCU A CAUCGAGA | 3120 | TCTCGATG GGCTAGCTACAACGA AGGGGATG | 4127 |
| 2087 | UCCCCUAC A UCGAGACC | 3121 | GGTCTCGA GGCTAGCTACAACGA GTAGGGGA | 4128 |
| 2093 | ACAUCGAG A CCUCGGCC | 3122 | GGCCGAGG GGCTAGCTACAACGA CTCGATGT | 4129 |
| 2099 | AGACCUCG G CCAAGACC | 3123 | GGTCTTGG GGCTAGCTACAACGA CGAGGTCT | 4130 |
| 2105 | CGGCCAAG A CCCGGCAG | 3124 | CTGCCGGG GGCTAGCTACAACGA CTTGGCCG | 4131 |
| 2110 | AAGACCCG G CAGGGAGU | 3125 | ACTCCCTG GGCTAGCTACAACGA CGGGTCTT | 4132 |
| 2117 | GGCAGGGA G UGGAGGAU | 3126 | ATCCTCCA GGCTAGCTACAACGA TCCCTGCC | 4133 |
| 2124 | AGUGGAGG A UGCCUUCU | 3127 | AGAAGGCA GGCTAGCTACAACGA CCTCCACT | 4134 |
| 2126 | UGGAGGAU G CCUUCUAC | 3128 | GTAGAAGG GGCTAGCTACAACGA ATCCTCCA | 4135 |
| 2133 | UGCCUUCU A CACGUUGG | 3129 | CCAACGTG GGCTAGCTACAACGA AGAAGGCA | 4136 |
| 2135 | CCUUCUAC A CGUUGGUG | 3130 | CACCAACG GGCTAGCTACAACGA GTAGAAGG | 4137 |
| 2137 | UUCUACAC G UUGGUGCG | 3131 | CGCACCAA GGCTAGCTACAACGA GTGTAGAA | 4138 |
| 2141 | ACACGUUG G UGCGUGAG | 3132 | CTCACGCA GGCTAGCTACAACGA CAACGTGT | 4139 |
| 2143 | ACGUUGGU G CGUGAGAU | 3133 | ATCTCACG GGCTAGCTACAACGA ACCAACGT | 4140 |
| 2145 | GUUGGUGC G UGAGAUCC | 3134 | GGATCTCA GGCTAGCTACAACGA GCACCAAC | 4141 |
| 2150 | UGCGUGAG A UCCGGCAG | 3135 | CTGCCGGA GGCTAGCTACAACGA CTCACGCA | 4142 |
| 2155 | GAGAUCCG G CAGCACAA | 3136 | TTGTGCTG GGCTAGCTACAACGA CGGATCTC | 4143 |
| 2158 | AUCCGGCA G CACAAGCU | 3137 | AGCTTGTG GGCTAGCTACAACGA TGCCGGAT | 4144 |
| 2160 | CCGGCAGC A CAAGCUGC | 3138 | GCAGCTTG GGCTAGCTACAACGA GCTGCCGG | 4145 |
| 2164 | CAGCACAA G CUGCGGAA | 3139 | TTCCGCAG GGCTAGCTACAACGA TTGTGCTG | 4146 |
| 2167 | CACAAGCU G CGGAAGCU | 3140 | AGCTTCCG GGCTAGCTACAACGA AGCTTGTG | 4147 |
| 2173 | CUGCGGAA G CUGAACCC | 3141 | GGGTTCAG GGCTAGCTACAACGA TTCCGCAG | 4148 |
| 2178 | GAAGCUGA A CCCUCCUG | 3142 | CAGGAGGG GGCTAGCTACAACGA TCAGCTTC | 4149 |
| 2187 | CCCUCCUG A UGAGAGUG | 3143 | CACTCTCA GGCTAGCTACAACGA CAGGAGGG | 4150 |
| 2193 | UGAUGAGA G UGGCCCCG | 3144 | CGGGGCCA GGCTAGCTACAACGA TCTCATCA | 4151 |
| 2196 | UGAGAGUG G CCCCGGCU | 3145 | AGCCGGGG GGCTAGCTACAACGA CACTCTCA | 4152 |
| 2202 | UGGCCCCG G CUGCAUGA | 3146 | TCATGCAG GGCTAGCTACAACGA CGGGGCCA | 4153 |
| 2205 | CCCCGGCU G CAUGAGCU | 3147 | AGCTCATG GGCTAGCTACAACGA AGCCGGGG | 4154 |
| 2207 | CCGGCUGC A UGAGCUGC | 3148 | GCAGCTCA GGCTAGCTACAACGA GCAGCCGG | 4155 |
| 2211 | CUGCAUGA G CUGCAAGU | 3149 | ACTTGCAG GGCTAGCTACAACGA TCATGCAG | 4156 |
| 2214 | CAUGAGCU G CAAGUGUG | 3150 | CACACTTG GGCTAGCTACAACGA AGCTCATG | 4157 |
| 2218 | AGCUGCAA G UGUGUGCU | 3151 | AGCACACA GGCTAGCTACAACGA TTGCAGCT | 4158 |
| 2220 | CUGCAAGU G UGUGCUCU | 3152 | AGAGCACA GGCTAGCTACAACGA ACTTGCAG | 4159 |
| 2222 | GCAAGUGU G UGCUCUCC | 3153 | GGAGAGCA GGCTAGCTACAACGA ACACTTGC | 4160 |
| 2224 | AAGUGUGU G CUCUCCUG | 3154 | CAGGAGAG GGCTAGCTACAACGA ACACACTT | 4161 |
| 2233 | CUCUCCUG A CGCAGGUG | 3155 | CACCTGCG GGCTAGCTACAACGA CAGGAGAG | 4162 |
| 2235 | CUCCUGAC G CAGGUGAG | 3156 | CTCACCTG GGCTAGCTACAACGA GTCAGGAG | 4163 |
| 2239 | UGACGCAG G UGAGGGGG | 3157 | CCCCCTCA GGCTAGCTACAACGA CTGCGTCA | 4164 |
| 2248 | UGAGGGGG A CUCCCAGG | 3158 | CCTGGGAG GGCTAGCTACAACGA CCCCCTCA | 4165 |
| 2257 | CUCCCAGG G CGGCCGCC | 3159 | GGCGGCCG GGCTAGCTACAACGA CCTGGGAG | 4166 |
| 2260 | CCAGGGCG G CCGCCACG | 3160 | CGTGGCGG GGCTAGCTACAACGA CGCCCTGG | 4167 |
| 2263 | GGGCGGCC G CCACGCCC | 3161 | GGGCGTGG GGCTAGCTACAACGA GGCCGCCC | 4168 |
| 2266 | CGGCCGCC A CGCCCACC | 3162 | GGTGGGCG GGCTAGCTACAACGA GGCGGCCG | 4169 |
| 2268 | GCCGCCAC G CCCACCGG | 3163 | CCGGTGGG GGCTAGCTACAACGA GTGGCGGC | 4170 |
| 2272 | CCACGCCC A CCGGAUGA | 3164 | TCATCCGG GGCTAGCTACAACGA GGGCGTGG | 4171 |
| 2277 | CCCACCGG A UGACCCCG | 3165 | CGGGGTCA GGCTAGCTACAACGA CCGGTGGG | 4172 |
| 2280 | ACCGGAUG A CCCCGGCU | 3166 | AGCCGGGG GGCTAGCTACAACGA CATCCGGT | 4173 |
| 2286 | UGACCCCG G CUCCCCGC | 3167 | GCGGGGAG GGCTAGCTACAACGA CGGGGTCA | 4174 |
| 2293 | GGCUCCCC G CCCCUGCC | 3168 | GGCAGGGG GGCTAGCTACAACGA GGGGAGCC | 4175 |
| 2299 | CCGCCCCU G CCGGUCUC | 3169 | GAGACCGG GGCTAGCTACAACGA AGGGGCGG | 4176 |
| 2303 | CCCUGCCG G UCUCCUGG | 3170 | CCAGGAGA GGCTAGCTACAACGA CGGCAGGG | 4177 |
| 2311 | GUCUCCUG G CCUGCGGU | 3171 | ACCGCAGG GGCTAGCTACAACGA CAGGAGAC | 4178 |
| 2315 | CCUGGCCU G CGGUCAGC | 3172 | GCTGACCG GGCTAGCTACAACGA AGGCCAGG | 4179 |
| 2318 | GGCCUGCG G UCAGCAGC | 3173 | GCTGCTGA GGCTAGCTACAACGA CGCAGGCC | 4180 |
| 2322 | UGCGGUCA G CAGCCUCC | 3174 | GGAGGCTG GGCTAGCTACAACGA TGACCGCA | 4181 |
| 2325 | GGUCAGCA G CCUCCCUU | 3175 | AAGGGAGG GGCTAGCTACAACGA TGCTGACC | 4182 |
| 2334 | CCUCCCUU G UGCCCCGC | 3176 | GCGGGGCA GGCTAGCTACAACGA AAGGGAGG | 4183 |
| 2336 | UCCCUUGU G CCCCGCCC | 3177 | GGGCGGGG GGCTAGCTACAACGA ACAAGGGA | 4184 |
| 2341 | UGUGCCCC G CCCAGCAC | 3178 | GTGCTGGG GGCTAGCTACAACGA GGGGCACA | 4185 |
| 2346 | CCCGCCCA G CACAAGCU | 3179 | AGCTTGTG GGCTAGCTACAACGA TGGGCGGG | 4186 |
| 2348 | CGCCCAGC A CAAGCUCA | 3180 | TGAGCTTG GGCTAGCTACAACGA GCTGGGCG | 4187 |
| 2352 | CAGCACAA G CUCAGGAC | 3181 | GTCCTGAG GGCTAGCTACAACGA TTGTGCTG | 4188 |
| 2359 | AGCUCAGG A CAUGGAGG | 3182 | CCTCCATG GGCTAGCTACAACGA CCTGAGCT | 4189 |
| 2361 | CUCAGGAC A UGGAGGUG | 3183 | CACCTCCA GGCTAGCTACAACGA GTCCTGAG | 4190 |
| 2367 | ACAUGGAG G UGCCGGAU | 3184 | ATCCGGCA GGCTAGCTACAACGA CTCCATGT | 4191 |
| 2369 | AUGGAGGU G CCGGAUGC | 3185 | GCATCCGG GGCTAGCTACAACGA ACCTCCAT | 4192 |
| 2374 | GGUGCCGG A UGCAGGAA | 3186 | TTCCTGCA GGCTAGCTACAACGA CCGGCACC | 4193 |
| 2376 | UGCCGGAU G CAGGAAGG | 3187 | CCTTCCTG GGCTAGCTACAACGA ATCCGGCA | 4194 |
| 2387 | GGAAGGAG G UGCAGACG | 3188 | CGTCTGCA GGCTAGCTACAACGA CTCCTTCC | 4195 |
| 2389 | AAGGAGGU G CAGACGGA | 3189 | TCCGTCTG GGCTAGCTACAACGA ACCTCCTT | 4196 |
| 2393 | AGGUGCAG A CGGAAGGA | 3190 | TCCTTCCG GGCTAGCTACAACGA CTGCACCT | 4197 |
| 2415 | AAGGAAGG A CGGAAGCA | 3191 | TGCTTCCG GGCTAGCTACAACGA CCTTCCTT | 4198 |
| 2421 | GGACGGAA G CAAGGAAG | 3192 | CTTCCTTG GGCTAGCTACAACGA TTCCGTCC | 4199 |
| 2439 | AAGGAAGG G CUGCUGGA | 3193 | TCCAGCAG GGCTAGCTACAACGA CCTTCCTT | 4200 |
| 2442 | GAAGGGCU G CUGGAGCC | 3194 | GGCTCCAG GGCTAGCTACAACGA AGCCCTTC | 4201 |
| 2448 | CUGCUGGA G CCCAGUCA | 3195 | TGACTGGG GGCTAGCTACAACGA TCCAGCAG | 4202 |
| 2453 | GGAGCCCA G UCACCCCG | 3196 | CGGGGTGA GGCTAGCTACAACGA TGGGCTCC | 4203 |
| 2456 | GCCCAGUC A CCCCGGGA | 3197 | TCCCGGGG GGCTAGCTACAACGA GACTGGGC | 4204 |
| 2464 | ACCCCGGG A CCGUGGGC | 3198 | GCCCACGG GGCTAGCTACAACGA CCCGGGGT | 4205 |
| 2467 | CCGGGACC G UGGGCCGA | 3199 | TCGGCCCA GGCTAGCTACAACGA GGTCCCGG | 4206 |
| 2471 | GACCGUGG G CCGAGGUG | 3200 | CACCTCGG GGCTAGCTACAACGA CCACGGTC | 4207 |
| 2477 | GGGCCGAG G UGACUGCA | 3201 | TGCAGTCA GGCTAGCTACAACGA CTCGGCCC | 4208 |
| 2480 | CCGAGGUG A CUGCAGAC | 3202 | GTCTGCAG GGCTAGCTACAACGA CACCTCGG | 4209 |
| 2483 | AGGUGACU G CAGACCCU | 3203 | AGGGTCTG GGCTAGCTACAACGA AGTCACCT | 4210 |
| 2487 | GACUGCAG A CCCUCCCA | 3204 | TGGGAGGG GGCTAGCTACAACGA CTGCAGTC | 4211 |
| 2501 | CCAGGGAG G CUGUGCAC | 3205 | GTGCACAG GGCTAGCTACAACGA CTCCCTGG | 4212 |
| 2504 | GGGAGGCU G UGCACAGA | 3206 | TCTGTGCA GGCTAGCTACAACGA AGCCTCCC | 4213 |
| 2506 | GAGGCUGU G CACAGACU | 3207 | AGTCTGTG GGCTAGCTACAACGA ACAGCCTC | 4214 |
| 2508 | GGCUGUGC A CAGACUGU | 3208 | ACAGTCTG GGCTAGCTACAACGA GCACAGCC | 4215 |
| 2512 | GUGCACAG A CUGUCUUG | 3209 | CAAGACAG GGCTAGCTACAACGA CTGTGCAC | 4216 |
| 2515 | CACAGACU G UCUUGAAC | 3210 | GTTCAAGA GGCTAGCTACAACGA AGTCTGTG | 4217 |
| 2522 | UGUCUUGA A CAUCCCAA | 3211 | TTGGGATG GGCTAGCTACAACGA TCAAGACA | 4218 |
| 2524 | UCUUGAAC A UCCCAAAU | 3212 | ATTTGGGA GGCTAGCTACAACGA GTTCAAGA | 4219 |
| 2531 | CAUCCCAA A UGCCACCG | 3213 | CGGTGGCA GGCTAGCTACAACGA TTGGGATG | 4220 |
| 2533 | UCCCAAAU G CCACCGGA | 3214 | TCCGGTGG GGCTAGCTACAACGA ATTTGGGA | 4221 |
| 2536 | CAAAUGCC A CCGGAACC | 3215 | GGTTCCGG GGCTAGCTACAACGA GGCATTTG | 4222 |
| 2542 | CCACCGGA A CCCCAGCC | 3216 | GGCTGGGG GGCTAGCTACAACGA TCCGGTGG | 4223 |
| 2548 | GAACCCCA G CCCUUAGC | 3217 | GCTAAGGG GGCTAGCTACAACGA TGGGGTTC | 4224 |
| 2555 | AGCCCUUA G CUCCCCUC | 3218 | GAGGGGAG GGCTAGCTACAACGA TAAGGGCT | 4225 |
| 2568 | CCUCCCAG G CCUCUGUG | 3219 | CACAGAGG GGCTAGCTACAACGA CTGGGAGG | 4226 |
| 2574 | AGGCCUCU G UGGGCCCU | 3220 | AGGGCCCA GGCTAGCTACAACGA AGAGGCCT | 4227 |
| 2578 | CUCUGUGG G CCCUUGUC | 3221 | GACAAGGG GGCTAGCTACAACGA CCACAGAG | 4228 |
| 2584 | GGGCCCUU G UCGGGCAC | 3222 | GTGCCCGA GGCTAGCTACAACGA AAGGGCCC | 4229 |
| 2589 | CUUGUCGG G CACAGAUG | 3223 | CATCTGTG GGCTAGCTACAACGA CCGACAAG | 4230 |
| 2591 | UGUCGGGC A CAGAUGGG | 3224 | CCCATCTG GGCTAGCTACAACGA GCCCGACA | 4231 |
| 2595 | GGGCACAG A UGGGAUCA | 3225 | TGATCCCA GGCTAGCTACAACGA CTGTGCCC | 4232 |
| 2600 | CAGAUGGG A UCACAGUA | 3226 | TACTGTGA GGCTAGCTACAACGA CCCATCTG | 4233 |
| 2603 | AUGGGAUC A CAGUAAAU | 3227 | ATTTACTG GGCTAGCTACAACGA GATCCCAT | 4234 |
| 2606 | GGAUCACA G UAAAUUAU | 3228 | ATAATTTA GGCTAGCTACAACGA TGTGATCC | 4235 |
| 2610 | CACAGUAA A UUAUUGGA | 3229 | TCCAATAA GGCTAGCTACAACGA TTACTGTG | 4236 |
| 2613 | AGUAAAUU A UUGGAUGG | 3230 | CCATCCAA GGCTAGCTACAACGA AATTTACT | 4237 |
| 2618 | AUUAUUGG A UGGUCUUG | 3231 | CAAGACCA GGCTAGCTACAACGA CCAATAAT | 4238 |
| 2621 | AUUGGAUG G UCUUGAUC | 3232 | GATCAAGA GGCTAGCTACAACGA CATCCAAT | 4239 |
| 2627 | UGGUCUUG A UCUUGGUU | 3233 | AACCAAGA GGCTAGCTACAACGA CAAGACCA | 4240 |
| 2633 | UGAUCUUG G UUUUCGGC | 3234 | GCCGAAAA GGCTAGCTACAACGA CAAGATCA | 4241 |
| 2640 | GGUUUUCG G CUGAGGGU | 3235 | ACCCTCAG GGCTAGCTACAACGA CGAAAACC | 4242 |
| 2647 | GGCUGAGG G UGGGACAC | 3236 | GTGTCCCA GGCTAGCTACAACGA CCTCAGCC | 4243 |
| 2652 | AGGGUGGG A CACGGUGC | 3237 | GCACCGTG GGCTAGCTACAACGA CCCACCCT | 4244 |
| 2654 | GGUGGGAC A CGGUGCGC | 3238 | GCGCACCG GGCTAGCTACAACGA GTCCCACC | 4245 |
| 2657 | GGGACACG G UGCGCGUG | 3239 | CACGCGCA GGCTAGCTACAACGA CGTGTCCC | 4246 |
| 2659 | GACACGGU G CGCGUGUG | 3240 | CACACGCG GGCTAGCTACAACGA ACCGTGTC | 4247 |
| 2661 | CACGGUGC G CGUGUGGC | 3241 | GCCACACG GGCTAGCTACAACGA GCACCGTG | 4248 |
| 2663 | CGGUGCGC G UGUGGCCU | 3242 | AGGCCACA GGCTAGCTACAACGA GCGCACCG | 4249 |
| 2665 | GUGCGCGU G UGGCCUGG | 3243 | CCAGGCCA GGCTAGCTACAACGA ACGCGCAC | 4250 |
| 2668 | CGCGUGUG G CCUGGCAU | 3244 | ATGCCAGG GGCTAGCTACAACGA CACACGCG | 4251 |
| 2673 | GUGGCCUG G CAUGAGGU | 3245 | ACCTCATG GGCTAGCTACAACGA CAGGCCAC | 4252 |
| 2675 | GGCCUGGC A UGAGGUAU | 3246 | ATACCTCA GGCTAGCTACAACGA GCCAGGCC | 4253 |
| 2680 | GGCAUGAG G UAUGUCGG | 3247 | CCGACATA GGCTAGCTACAACGA CTCATGCC | 4254 |
| 2682 | CAUGAGGU A UGUCGGAA | 3248 | TTCCGACA GGCTAGCTACAACGA ACCTCATG | 4255 |
| 2684 | UGAGGUAU G UCGGAACC | 3249 | GGTTCCGA GGCTAGCTACAACGA ATACCTCA | 4256 |
| 2690 | AUGUCGGA A CCUCAGGC | 3250 | GCCTGAGG GGCTAGCTACAACGA TCCGACAT | 4257 |
| 2697 | AACCUCAG G CCUGUCCA | 3251 | TGGACAGG GGCTAGCTACAACGA CTGAGGTT | 4258 |
| 2701 | UCAGGCCU G UCCAGCCC | 3252 | GGGCTGGA GGCTAGCTACAACGA AGGCCTGA | 4259 |
| 2706 | CCUGUCCA G CCCUGGGC | 3253 | GCCCAGGG GGCTAGCTACAACGA TGGACAGG | 4260 |
| 2713 | AGCCCUGG G CUCUCCAU | 3254 | ATGGAGAG GGCTAGCTACAACGA CCAGGGCT | 4261 |
| 2720 | GGCUCUCC A UAGCCUUU | 3255 | AAAGGCTA GGCTAGCTACAACGA GGAGAGCC | 4262 |
| 2723 | UCUCCAUA G CCUUUGGG | 3256 | CCCAAAGG GGCTAGCTACAACGA TATGGAGA | 4263 |
| 2740 | AGGGGGAG G UUGGGAGA | 3257 | TCTCCCAA GGCTAGCTACAACGA CTCCCCCT | 4264 |
| 2750 | UGGGAGAG G CCGGUCAG | 3258 | CTGACCGG GGCTAGCTACAACGA CTCTCCCA | 4265 |
| 2754 | AGAGGCCG G UCAGGGGU | 3259 | ACCCCTGA GGCTAGCTACAACGA CGGCCTCT | 4266 |
| 2761 | GGUCAGGG G UCUGGGCU | 3260 | AGCCCAGA GGCTAGCTACAACGA CCCTGACC | 4267 |
| 2767 | GGGUCUGG G CUGUGGUG | 3261 | CACCACAG GGCTAGCTACAACGA CCAGACCC | 4268 |
| 2770 | UCUGGGCU G UGGUGCUC | 3262 | GAGCACCA GGCTAGCTACAACGA AGCCCAGA | 4269 |
| 2773 | GGGCUGUG G UGCUCUCU | 3263 | AGAGAGCA GGCTAGCTACAACGA CACAGCCC | 4270 |
| 2775 | GCUGUGGU G CUCUCUCC | 3264 | GGAGAGAG GGCTAGCTACAACGA ACCACAGC | 4271 |
| 2788 | CUCCUCCC G CCUGCCCC | 3265 | GGGGCAGG GGCTAGCTACAACGA GGGAGGAG | 4272 |
| 2792 | UCCCGCCU G CCCCAGUG | 3266 | CACTGGGG GGCTAGCTACAACGA AGGCGGGA | 4273 |
| 2798 | CUGCCCCA G UGUCCACG | 3267 | CGTGGACA GGCTAGCTACAACGA TGGGGCAG | 4274 |
| 2800 | GCCCCAGU G UCCACGGC | 3268 | GCCGTGGA GGCTAGCTACAACGA ACTGGGGC | 4275 |
| 2804 | CAGUGUCC A CGGCUUCU | 3269 | AGAAGCCG GGCTAGCTACAACGA GGACACTG | 4276 |
| 2807 | UGUCCACG G CUUCUGGC | 3270 | GCCAGAAG GGCTAGCTACAACGA CGTGGACA | 4277 |
| 2814 | GGCUUCUG G CAGAGAGC | 3271 | GCTCTCTG GGCTAGCTACAACGA CAGAAGCC | 4278 |
| 2821 | GGCAGAGA G CUCUGGAC | 3272 | GTCCAGAG GGCTAGCTACAACGA TCTCTGCC | 4279 |
| 2828 | AGCUCUGG A CAAGCAGG | 3273 | CCTGCTTG GGCTAGCTACAACGA CCAGAGCT | 4280 |
| 2832 | CUGGACAA G CAGGCAGA | 3274 | TCTGCCTG GGCTAGCTACAACGA TTGTCCAG | 4281 |
| 2836 | ACAAGCAG G CAGAUCAU | 3275 | ATGATCTG GGCTAGCTACAACGA CTGCTTGT | 4282 |
| 2840 | GCAGGCAG A UCAUAAGG | 3276 | CCTTATGA GGCTAGCTACAACGA CTGCCTGC | 4283 |
| 2843 | GGCAGAUC A UAAGGACA | 3277 | TGTCCTTA GGCTAGCTACAACGA GATCTGCC | 4284 |
| 2849 | UCAUAAGG A CAGAGAGC | 3278 | GCTCTCTG GGCTAGCTACAACGA CCTTATGA | 4285 |
| 2856 | GACAGAGA G CUUACUGU | 3279 | ACAGTAAG GGCTAGCTACAACGA TCTCTGTC | 4286 |
| 2860 | GAGAGCUU A CUGUGCUU | 3280 | AAGCACAG GGCTAGCTACAACGA AAGCTCTC | 4287 |
| 2863 | AGCUUACU G UGCUUCUA | 3281 | TAGAAGCA GGCTAGCTACAACGA AGTAAGCT | 4288 |
| 2865 | CUUACUGU G CUUCUACC | 3282 | GGTAGAAG GGCTAGCTACAACGA ACAGTAAG | 4289 |
| 2871 | GUGCUUCU A CCAACUAG | 3283 | CTAGTTGG GGCTAGCTACAACGA AGAAGCAC | 4290 |
| 2875 | UUCUACCA A CUAGGAGG | 3284 | CCTCCTAG GGCTAGCTACAACGA TGGTAGAA | 4291 |
| 2884 | CUAGGAGG G CGUCCUGG | 3285 | CCAGGACG GGCTAGCTACAACGA CCTCCTAG | 4292 |
| 2886 | AGGAGGGC G UCCUGGUC | 3286 | GACCAGGA GGCTAGCTACAACGA GCCCTCCT | 4293 |
| 2892 | GCGUCCUG G UCCUCCAG | 3287 | CTGGAGGA GGCTAGCTACAACGA CAGGACGC | 4294 |
| 2907 | AGAGGGAG G UGGUUUCA | 3288 | TGAAACCA GGCTAGCTACAACGA CTCCCTCT | 4295 |
| 2910 | GGGAGGUG G UUUCAGGG | 3289 | CCCTGAAA GGCTAGCTACAACGA CACCTCCC | 4296 |
| 2919 | UUUCAGGG G UUGGGGAU | 3290 | ATCCCCAA GGCTAGCTACAACGA CCCTGAAA | 4297 |
| 2926 | GGUUGGGG A UCUGUGCC | 3291 | GGCACAGA GGCTAGCTACAACGA CCCCAACC | 4298 |
| 2930 | GGGGAUCU G UGCCGGUG | 3292 | CACCGGCA GGCTAGCTACAACGA AGATCCCC | 4299 |
| 2932 | GGAUCUGU G CCGGUGGC | 3293 | GCCACCGG GGCTAGCTACAACGA ACAGATCC | 4300 |
| 2936 | CUGUGCCG G UGGCUCUG | 3294 | CAGAGCCA GGCTAGCTACAACGA CGGCACAG | 4301 |
| 2939 | UGCCGGUG G CUCUGGUC | 3295 | GACCAGAG GGCTAGCTACAACGA CACCGGCA | 4302 |
| 2945 | UGGCUCUG G UCUCUGCU | 3296 | AGCAGAGA GGCTAGCTACAACGA CAGAGCCA | 4303 |
| 2951 | UGGUCUCU G CUGGGAGC | 3297 | GCTCCCAG GGCTAGCTACAACGA AGAGACCA | 4304 |
| 2958 | UGCUGGGA G CCUUCUUG | 3298 | CAAGAAGG GGCTAGCTACAACGA TCCCAGCA | 4305 |
| 2967 | CCUUCUUG G CGGUGAGA | 3299 | TCTCACCG GGCTAGCTACAACGA CAAGAAGG | 4306 |
| 2970 | UCUUGGCG G UGAGAGGC | 3300 | GCCTCTCA GGCTAGCTACAACGA CGCCAAGA | 4307 |
| 2977 | GGUGAGAG G CAUCACCU | 3301 | AGGTGATG GGCTAGCTACAACGA CTCTCACC | 4308 |
| 2979 | UGAGAGGC A UCACCUUU | 3302 | AAAGGTGA GGCTAGCTACAACGA GCCTCTCA | 4309 |
| 2982 | GAGGCAUC A CCUUUCCU | 3303 | AGGAAAGG GGCTAGCTACAACGA GATGCCTC | 4310 |
| 2992 | CUUUCCUG A CUUGCUCC | 3304 | GGAGCAAG GGCTAGCTACAACGA CAGGAAAG | 4311 |
| 2996 | CCUGACUU G CUCCCAGC | 3305 | GCTGGGAG GGCTAGCTACAACGA AAGTCAGG | 4312 |
| 3003 | UGCUCCCA G CGUGAAAU | 3306 | ATTTCACG GGCTAGCTACAACGA TGGGAGCA | 4313 |
| 3005 | CUCCCAGC G UGAAAUGC | 3307 | GCATTTCA GGCTAGCTACAACGA GCTGGGAG | 4314 |
| 3010 | AGCGUGAA A UGCACCUG | 3308 | CAGGTGCA GGCTAGCTACAACGA TTCACGCT | 4315 |
| 3012 | CGUGAAAU G CACCUGCC | 3309 | GGCAGGTG GGCTAGCTACAACGA ATTTCACG | 4316 |
| 3014 | UGAAAUGC A CCUGCCAA | 3310 | TTGGCAGG GGCTAGCTACAACGA GCATTTCA | 4317 |
| 3018 | AUGCACCU G CCAAGAAU | 3311 | ATTCTTGG GGCTAGCTACAACGA AGGTGCAT | 4318 |
| 3025 | UGCCAAGA A UGGCAGAC | 3312 | GTCTGCCA GGCTAGCTACAACGA TCTTGGCA | 4319 |
| 3028 | CAAGAAUG G CAGACAUA | 3313 | TATGTCTG GGCTAGCTACAACGA CATTCTTG | 4320 |
| 3032 | AAUGGCAG A CAUAGGGA | 3314 | TCCCTATG GGCTAGCTACAACGA CTGCCATT | 4321 |
| 3034 | UGGCAGAC A UAGGGACC | 3315 | GGTCCCTA GGCTAGCTACAACGA GTCTGCCA | 4322 |
| 3040 | ACAUAGGG A CCCCGCCU | 3316 | AGGCGGGG GGCTAGCTACAACGA CCCTATGT | 4323 |
| 3045 | GGGACCCC G CCUCCUGG | 3317 | CCAGGAGG GGCTAGCTACAACGA GGGGTCCC | 4324 |
| 3054 | CCUCCUGG G CCUUCACA | 3318 | TGTGAAGG GGCTAGCTACAACGA CCAGGAGG | 4325 |
| 3060 | GGGCCUUC A CAUGCCCA | 3319 | TGGGCATG GGCTAGCTACAACGA GAAGGCCC | 4326 |
| 3062 | GCCUUCAC A UGCCCAGU | 3320 | ACTGGGCA GGCTAGCTACAACGA GTGAAGGC | 4327 |
| 3064 | CUUCACAU G CCCAGUUU | 3321 | AAACTGGG GGCTAGCTACAACGA ATGTGAAG | 4328 |
| 3069 | CAUGCCCA G UUUUCUUC | 3322 | GAAGAAAA GGCTAGCTACAACGA TGGGCATG | 4329 |
| 3079 | UUUCUUCG G CUCUGUGG | 3323 | CCACAGAG GGCTAGCTACAACGA CGAAGAAA | 4330 |
| 3084 | UCGGCUCU G UGGCCUGA | 3324 | TCAGGCCA GGCTAGCTACAACGA AGAGCCGA | 4331 |
| 3087 | GCUCUGUG G CCUGAAGC | 3325 | GCTTCAGG GGCTAGCTACAACGA CACAGAGC | 4332 |
| 3094 | GGCCUGAA G CGGUCUGU | 3326 | ACAGACCG GGCTAGCTACAACGA TTCAGGCC | 4333 |
| 3097 | CUGAAGCG G UCUGUGGA | 3327 | TCCACAGA GGCTAGCTACAACGA CGCTTCAG | 4334 |
| 3101 | AGCGGUCU G UGGACCUU | 3328 | AAGGTCCA GGCTAGCTACAACGA AGACCGCT | 4335 |
| 3105 | GUCUGUGG A CCUUGGAA | 3329 | TTCCAAGG GGCTAGCTACAACGA CCACAGAC | 4336 |
| 3114 | CCUUGGAA G UAGGGCUC | 3330 | GAGCCCTA GGCTAGCTACAACGA TTCCAAGG | 4337 |
| 3119 | GAAGUAGG G CUCCAGCA | 3331 | TGCTGGAG GGCTAGCTACAACGA CCTACTTC | 4338 |
| 3125 | GGGCUCCA G CACCGACU | 3332 | AGTCGGTG GGCTAGCTACAACGA TGGAGCCC | 4339 |
| 3127 | GCUCCAGC A CCGACUGG | 3333 | CCAGTCGG GGCTAGCTACAACGA GCTGGAGC | 4340 |
| 3131 | CAGCACCG A CUGGCCUC | 3334 | GAGGCCAG GGCTAGCTACAACGA CGGTGCTG | 4341 |
| 3135 | ACCGACUG G CCUCAGGC | 3335 | GCCTGAGG GGCTAGCTACAACGA CAGTCGGT | 4342 |
| 3142 | GGCCUCAG G CCUCUGCC | 3336 | GGCAGAGG GGCTAGCTACAACGA CTGAGGCC | 4343 |
| 3148 | AGGCCUCU G CCUCAUUG | 3337 | CAATGAGG GGCTAGCTACAACGA AGAGGCCT | 4344 |
| 3153 | UCUGCCUC A UUGGUGGU | 3338 | ACCACCAA GGCTAGCTACAACGA GAGGCAGA | 4345 |
| 3157 | CCUCAUUG G UGGUCGGG | 3339 | CCCGACCA GGCTAGCTACAACGA CAATGAGG | 4346 |
| 3160 | CAUUGGUG G UCGGGUAG | 3340 | CTACCCGA GGCTAGCTACAACGA CACCAATG | 4347 |
| 3165 | GUGGUCGG G UAGCGGCC | 3341 | GGCCGCTA GGCTAGCTACAACGA CCGACCAC | 4348 |
| 3168 | GUCGGGUA G CGGCCAGU | 3342 | ACTGGCCG GGCTAGCTACAACGA TACCCGAC | 4349 |
| 3171 | GGGUAGCG G CCAGUAGG | 3343 | CCTACTGG GGCTAGCTACAACGA CGCTACCC | 4350 |
| 3175 | AGCGGCCA G UAGGGCGU | 3344 | ACGCCCTA GGCTAGCTACAACGA TGGCCGCT | 4351 |
| 3180 | CCAGUAGG G CGUGGGAG | 3345 | CTCCCACG GGCTAGCTACAACGA CCTACTGG | 4352 |
| 3182 | AGUAGGGC G UGGGAGCC | 3346 | GGCTCCCA GGCTAGCTACAACGA GCCCTACT | 4353 |
| 3188 | GCGUGGGA G CCUGGCCA | 3347 | TGGCCAGG GGCTAGCTACAACGA TCCCACGC | 4354 |
| 3193 | GGAGCCUG G CCAUCCCU | 3348 | AGGGATGG GGCTAGCTACAACGA CAGGCTCC | 4355 |
| 3196 | GCCUGGCC A UCCCUGCC | 3349 | GGCAGGGA GGCTAGCTACAACGA GGCCAGGC | 4356 |
| 3202 | CCAUCCCU G CCUCCUGG | 3350 | CCAGGAGG GGCTAGCTACAACGA AGGGATGG | 4357 |
| 3212 | CUCCUGGA G UGGACGAG | 3351 | CTCGTCCA GGCTAGCTACAACGA TCCAGGAG | 4358 |
| 3216 | UGGAGUGG A CGAGGUUG | 3352 | CAACCTCG GGCTAGCTACAACGA CCACTCCA | 4359 |
| 3221 | UGGACGAG G UUGGCAGC | 3353 | GCTGCCAA GGCTAGCTACAACGA CTCGTCCA | 4360 |
| 3225 | CGAGGUUG G CAGCUGGU | 3354 | ACCAGCTG GGCTAGCTACAACGA CAACCTCG | 4361 |
| 3228 | GGUUGGCA G CUGGUCCG | 3355 | CGGACCAG GGCTAGCTACAACGA TGCCAACC | 4362 |
| 3232 | GGCAGCUG G UCCGUCUG | 3356 | CAGACGGA GGCTAGCTACAACGA CAGCTGCC | 4363 |
| 3236 | GCUGGUCC G UCUGCUCC | 3357 | GGAGCAGA GGCTAGCTACAACGA GGACCAGC | 4364 |
| 3240 | GUCCGUCU G CUCCUGCC | 3358 | GGCAGGAG GGCTAGCTACAACGA AGACGGAC | 4365 |
| 3246 | CUGCUCCU G CCCCACUC | 3359 | GAGTGGGG GGCTAGCTACAACGA AGGAGCAG | 4366 |
| 3251 | CCUGCCCC A CUCUCCCC | 3360 | GGGGAGAG GGCTAGCTACAACGA GGGGCAGG | 4367 |
| 3261 | UCUCCCCC G CCCCUGCC | 3361 | GGCAGGGG GGCTAGCTACAACGA GGGGGAGA | 4368 |
| 3267 | CCGCCCCU G CCCUCACC | 3362 | GGTGAGGG GGCTAGCTACAACGA AGGGGCGG | 4369 |
| 3273 | CUGCCCUC A CCCUACCC | 3363 | GGGTAGGG GGCTAGCTACAACGA GAGGGCAG | 4370 |
| 3278 | CUCACCCU A CCCUUGCC | 3364 | GGCAAGGG GGCTAGCTACAACGA AGGGTGAG | 4371 |
| 3284 | CUACCCUU G CCCCACGC | 3365 | GCGTGGGG GGCTAGCTACAACGA AAGGGTAG | 4372 |
| 3289 | CUUGCCCC A CGCCUGCC | 3366 | GGCAGGCG GGCTAGCTACAACGA GGGGCAAG | 4373 |
| 3291 | UGCCCCAC G CCUGCCUC | 3367 | GAGGCAGG GGCTAGCTACAACGA GTGGGGCA | 4374 |
| 3295 | CCACGCCU G CCUCAUGG | 3368 | CCATGAGG GGCTAGCTACAACGA AGGCGTGG | 4375 |
| 3300 | CCUGCCUC A UGGCUGGU | 3369 | ACCAGCCA GGCTAGCTACAACGA GAGGCAGG | 4376 |
| 3303 | GCCUCAUG G CUGGUUGC | 3370 | GCAACCAG GGCTAGCTACAACGA CATGAGGC | 4377 |
| 3307 | CAUGGCUG G UUGCUCUU | 3371 | AAGAGCAA GGCTAGCTACAACGA CAGCCATG | 4378 |
| 3310 | GGCUGGUU G CUCUUGGA | 3372 | TCCAAGAG GGCTAGCTACAACGA AACCAGCC | 4379 |
| 3319 | CUCUUGGA G CCUGGUAG | 3373 | CTACCAGG GGCTAGCTACAACGA TCCAAGAG | 4380 |
| 3324 | GGAGCCUG G UAGUGUCA | 3374 | TGACACTA GGCTAGCTACAACGA CAGGCTCC | 4381 |
| 3327 | GCCUGGUA G UGUCACUG | 3375 | CAGTGACA GGCTAGCTACAACGA TACCAGGC | 4382 |
| 3329 | CUGGUAGU G UCACUGGC | 3376 | GCCAGTGA GGCTAGCTACAACGA ACTACCAG | 4383 |
| 3332 | GUAGUGUC A CUGGCUCA | 3377 | TGAGCCAG GGCTAGCTACAACGA GACACTAC | 4384 |
| 3336 | UGUCACUG G CUCAGCCU | 3378 | AGGCTGAG GGCTAGCTACAACGA CAGTGACA | 4385 |
| 3341 | CUGGCUCA G CCUUGCUG | 3379 | CAGCAAGG GGCTAGCTACAACGA TGAGCCAG | 4386 |
| 3346 | UCAGCCUU G CUGGGUAU | 3380 | ATACCCAG GGCTAGCTACAACGA AAGGCTGA | 4387 |
| 3351 | CUUGCUGG G UAUACACA | 3381 | TGTGTATA GGCTAGCTACAACGA CCAGCAAG | 4388 |
| 3353 | UGCUGGGU A UACACAGG | 3382 | CCTGTGTA GGCTAGCTACAACGA ACCCAGCA | 4389 |
| 3355 | CUGGGUAU A CACAGGCU | 3383 | AGCCTGTG GGCTAGCTACAACGA ATACCCAG | 4390 |
| 3357 | GGGUAUAC A CAGGCUCU | 3384 | AGAGCCTG GGCTAGCTACAACGA GTATACCC | 4391 |
| 3361 | AUACACAG G CUCUGCCA | 3385 | TGGCAGAG GGCTAGCTACAACGA CTGTGTAT | 4392 |
| 3366 | CAGGCUCU G CCACCCAC | 3386 | GTGGGTGG GGCTAGCTACAACGA AGAGCCTG | 4393 |
| 3369 | GCUCUGCC A CCCACUCU | 3387 | AGAGTGGG GGCTAGCTACAACGA GGCAGAGC | 4394 |
| 3373 | UGCCACCC A CUCUGCUC | 3388 | GAGCAGAG GGCTAGCTACAACGA GGGTGGCA | 4395 |
| 3378 | CCCACUCU G CUCCAAGG | 3389 | CCTTGGAG GGCTAGCTACAACGA AGAGTGGG | 4396 |
| 3388 | UCCAAGGG G CUUGCCCU | 3390 | AGGGCAAG GGCTAGCTACAACGA CCCTTGGA | 4397 |
| 3392 | AGGGGCUU G CCCUGCCU | 3391 | AGGCAGGG GGCTAGCTACAACGA AAGCCCCT | 4398 |
| 3397 | CUUGCCCU G CCUUGGGC | 3392 | GCCCAAGG GGCTAGCTACAACGA AGGGCAAG | 4399 |
| 3404 | UGCCUUGG G CCAAGUUC | 3393 | GAACTTGG GGCTAGCTACAACGA CCAAGGCA | 4400 |
| 3409 | UGGGCCAA G UUCUAGGU | 3394 | ACCTAGAA GGCTAGCTACAACGA TTGGCCCA | 4401 |
| 3416 | AGUUCUAG G UCUGGCCA | 3395 | TGGCCAGA GGCTAGCTACAACGA CTAGAACT | 4402 |
| 3421 | UAGGUCUG G CCACAGCC | 3396 | GGCTGTGG GGCTAGCTACAACGA CAGACCTA | 4403 |
| 3424 | GUCCGGCC A CAGCCACA | 3397 | TGTGGCTG GGCTAGCTACAACGA GGCCAGAC | 4404 |
| 3427 | UGGCCACA G CCACAGAC | 3398 | GTCTGTGG GGCTAGCTACAACGA TGTGGCCA | 4405 |
| 3430 | CCACAGCC A CAGACAGC | 3399 | GCTGTCTG GGCTAGCTACAACGA GGCTGTGG | 4406 |
| 3434 | AGCCACAG A CAGCUCAG | 3400 | CTGAGCTG GGCTAGCTACAACGA CTGTGGCT | 4407 |
| 3437 | CACAGACA G CUCAGUCC | 3401 | GGACTGAG GGCTAGCTACAACGA TGTCTGTG | 4408 |
| 3442 | ACAGCUCA G UCCCCUGU | 3402 | ACAGGGGA GGCTAGCTACAACGA TGAGCTGT | 4409 |
| 3449 | AGUCCCCU G UGUGGUCA | 3403 | TGACCACA GGCTAGCTACAACGA AGGGGACT | 4410 |
| 3451 | UCCCCUGU G UGGUCAUC | 3404 | GATGACCA GGCTAGCTACAACGA ACAGGGGA | 4411 |
| 3454 | CCUGUGUG G UCAUCCUG | 3405 | CAGGATGA GGCTAGCTACAACGA CACACAGG | 4412 |
| 3457 | GUGUGGUC A UCCUGGCU | 3406 | AGCCAGGA GGCTAGCTACAACGA GACCACAC | 4413 |
| 3463 | UCAUCCUG G CUUCUGCU | 3407 | AGCAGAAG GGCTAGCTACAACGA CAGGATGA | 4414 |
| 3469 | UGGCUUCU G CUGGGGGC | 3408 | GCCCCCAG GGCTAGCTACAACGA AGAAGCCA | 4415 |
| 3476 | UGCUGGGG G CCCACAGC | 3409 | GCTGTGGG GGCTAGCTACAACGA CCCCAGCA | 4416 |
| 3480 | GGGGGCCC A CAGCGCCC | 3410 | GGGCGCTG GGCTAGCTACAACGA GGGCCCCC | 4417 |
| 3483 | GGCCCACA G CGCCCCUG | 3411 | CAGGGGCG GGCTAGCTACAACGA TGTGGGCC | 4418 |
| 3485 | CCCACAGC G CCCCUGGU | 3412 | ACCAGGGG GGCTAGCTACAACGA GCTGTGGG | 4419 |
| 3492 | CGCCCCUG G UGCCCCUC | 3413 | GAGGGGCA GGCTAGCTACAACGA CAGGGGCG | 4420 |
| 3494 | CCCCUGGU G CCCCUCCC | 3414 | GGGAGGGG GGCTAGCTACAACGA ACCAGGGG | 4421 |
| 3511 | CUCCCAGG G CCCGGGUU | 3415 | AACCCGGG GGCTAGCTACAACGA CCTGGGAG | 4422 |
| 3517 | GGGCCCGG G UUGAGGCU | 3416 | AGCCTCAA GGCTAGCTACAACGA CCGGGCCC | 4423 |
| 3523 | GGGUUGAG G CUGGGCCA | 3417 | TGGCCCAG GGCTAGCTACAACGA CTCAACCC | 4424 |
| 3528 | GAGGCUGG G CCAGGCCC | 3418 | GGGCCTGG GGCTAGCTACAACGA CCAGCCTC | 4425 |
| 3533 | UGGGCCAG G CCCUCUGG | 3419 | CCAGAGGG GGCTAGCTACAACGA CTGGCCCA | 4426 |
| 3543 | CCUCUGGG A CGGGGACU | 3420 | AGTCCCCG GGCTAGCTACAACGA CCCAGAGG | 4427 |
| 3549 | GGACGGGG A CUUGUGCC | 3421 | GGCACAAG GGCTAGCTACAACGA CCCCGTCC | 4428 |
| 3553 | GGGGACUU G UGCCCUGU | 3422 | ACAGGGCA GGCTAGCTACAACGA AAGTCCCC | 4429 |
| 3555 | GGACUUGU G CCCUGUCA | 3423 | TGACAGGG GGCTAGCTACAACGA ACAAGTCC | 4430 |
| 3560 | UGUGCCCU G UCAGGGUU | 3424 | AACCCTGA GGCTAGCTACAACGA AGGGCACA | 4431 |
| 3566 | CUGUCAGG G UUCCCUAU | 3425 | ATAGGGAA GGCTAGCTACAACGA CCTGACAG | 4432 |
| 3573 | GGUUCCCU A UCCCUGAG | 3426 | CTCAGGGA GGCTAGCTACAACGA AGGGAACC | 4433 |
| 3582 | UCCCUGAG G UUGGGGGA | 3427 | TCCCCCAA GGCTAGCTACAACGA CTCAGGGA | 4434 |
| 3593 | GGGGGAGA G CUAGCAGG | 3428 | CCTGCTAG GGCTAGCTACAACGA TCTCCCCC | 4435 |
| 3597 | GAGAGCUA G CAGGGCAU | 3429 | ATGCCCTG GGCTAGCTACAACGA TAGCTCTC | 4436 |
| 3602 | CUAGCAGG G CAUGCCGC | 3430 | GCGGCATG GGCTAGCTACAACGA CCTGCTAG | 4437 |
| 3604 | AGCAGGGC A UGCCGCUG | 3431 | CAGCGGCA GGCTAGCTACAACGA GCCCTGCT | 4438 |
| 3606 | CAGGGCAU G CCGCUGGC | 3432 | GCCAGCGG GGCTAGCTACAACGA ATGCCCTG | 4439 |
| 3609 | GGCAUGCC G CUGGCUGG | 3433 | CCAGCCAG GGCTAGCTACAACGA GGCATGCC | 4440 |
| 3613 | UGCCGCUG G CUGGCCAG | 3434 | CTGGCCAG GGCTAGCTACAACGA CAGCGGCA | 4441 |
| 3617 | GCUGGCUG G CCAGGGCU | 3435 | AGCCCTGG GGCTAGCTACAACGA CAGCCAGC | 4442 |
| 3623 | UGGCCAGG G CUGCAGGG | 3436 | CCCTGCAG GGCTAGCTACAACGA CCTGGCCA | 4443 |
| 3626 | CCAGGGCU G CAGGGACA | 3437 | TGTCCCTG GGCTAGCTACAACGA AGCCCTGG | 4444 |
| 3632 | CUGCAGGG A CACUCCCC | 3438 | GGGGAGTG GGCTAGCTACAACGA CCCTGCAG | 4445 |
| 3634 | GCAGGGAC A CUCCCCCU | 3439 | AGGGGGAG GGCTAGCTACAACGA GTCCCTGC | 4446 |
| 3646 | CCCCUUUU G UCCAGGGA | 3440 | TCCCTGGA GGCTAGCTACAACGA AAAAGGGG | 4447 |
| 3655 | UCCAGGGA A UACCACAC | 3441 | GTGTGGTA GGCTAGCTACAACGA TCCCTGGA | 4448 |
| 3657 | CAGGGAAU A CCACACUC | 3442 | GAGTGTGG GGCTAGCTACAACGA ATTCCCTG | 4449 |
| 3660 | GGAAUACC A CACUCGCC | 3443 | GGCGAGTG GGCTAGCTACAACGA GGTATTCC | 4450 |
| 3662 | AAUACCAC A CUCGCCCU | 3444 | AGGGCGAG GGCTAGCTACAACGA GTGGTATT | 4451 |
| 3666 | CCACACUC G CCCUUCUC | 3445 | GAGAAGGG GGCTAGCTACAACGA GAGTGTGG | 4452 |
| 3679 | UCUCUCCA G CGAACACC | 3446 | GGTGTTCG GGCTAGCTACAACGA TGGAGAGA | 4453 |
| 3683 | UCCAGCGA A CACCACAC | 3447 | GTGTGGTG GGCTAGCTACAACGA TCGCTGGA | 4454 |
| 3685 | CAGCGAAC A CCACACUC | 3448 | GAGTGTGG GGCTAGCTACAACGA GTTCGCTG | 4455 |
| 3688 | CGAACACC A CACUCGCC | 3449 | GGCGAGTG GGCTAGCTACAACGA GGTGTTCG | 4456 |
| 3690 | AACACCAC A CUCGCCCU | 3450 | AGGGCGAG GGCTAGCTACAACGA GTGGTGTT | 4457 |
| 3694 | CCACACUC G CCCUUCUC | 3445 | GAGAAGGG GGCTAGCTACAACGA GAGTGTGG | 4452 |
| 3711 | UCCAGGGG A CGCCACAC | 3451 | GTGTGGCG GGCTAGCTACAACGA CCCCTGGA | 4458 |
| 3713 | CAGGGGAC G CCACACUC | 3452 | GAGTGTGG GGCTAGCTACAACGA GTCCCCTG | 4459 |
| 3716 | GGGACGCC A CACUCCCC | 3453 | GGGGAGTG GGCTAGCTACAACGA GGCGTCCC | 4460 |
| 3718 | GACGCCAC A CUCCCCCU | 3454 | AGGGGGAG GGCTAGCTACAACGA GTGGCGTC | 4461 |
| 3730 | CCCCUUCU G UCCAGGGG | 3455 | CCCCTGGA GGCTAGCTACAACGA AGAAGGGG | 4462 |
| 3739 | UCCAGGGG A CGCCACAC | 3451 | GTGTGGCG GGCTAGCTACAACGA CCCCTGGA | 4458 |
| 3741 | CAGGGGAC G CCACACUC | 3452 | GAGTGTGG GGCTAGCTACAACGA GTCCCCTG | 4459 |
| 3744 | GGGACGCC A CACUCCCC | 3453 | GGGGAGTG GGCTAGCTACAACGA GGCGTCCC | 4460 |
| 3746 | GACGCCAC A CUCCCCCU | 3454 | AGGGGGAG GGCTAGCTACAACGA GTGGCGTC | 4461 |
| 3767 | UCCAGGGG A CGCCACAC | 3451 | GTGTGGCG GGCTAGCTACAACGA CCCCTGGA | 4458 |
| 3769 | CAGGGGAC G CCACACUC | 3452 | GAGTGTGG GGCTAGCTACAACGA GTCCCCTG | 4459 |
| 3772 | GGGACGCC A CACUCGCC | 3456 | GGCGAGTG GGCTAGCTACAACGA GGCGTCCC | 4463 |
| 3774 | GACGCCAC A CUCGCCCU | 3457 | AGGGCGAG GGCTAGCTACAACGA GTGGCGTC | 4464 |
| 3778 | CCACACUC G CCCUUCUC | 3445 | GAGAAGGG GGCTAGCTACAACGA GAGTGTGG | 4452 |
| 3795 | UCCAGGGG A CGCCACAC | 3451 | GTGTGGCG GGCTAGCTACAACGA CCCCTGGA | 4458 |
| 3797 | CAGGGGAC G CCACACUC | 3452 | GAGTGTGG GGCTAGCTACAACGA GTCCCCTG | 4459 |
| 3800 | GGGACGCC A CACUCGCC | 3456 | GGCGAGTG GGCTAGCTACAACGA GGCGTCCC | 4463 |
| 3802 | GACGCCAC A CUCGCCCU | 3457 | AGGGCGAG GGCTAGCTACAACGA GTGGCGTC | 4464 |
| 3806 | CCACACUC G CCCUUCUC | 3445 | GAGAAGGG GGCTAGCTACAACGA GAGTGTGG | 4452 |
| 3823 | UCCAGGGG A CGCCACAC | 3451 | GTGTGGCG GGCTAGCTACAACGA CCCCTGGA | 4458 |
| 3825 | CAGGGGAC G CCACACUC | 3452 | GAGTGTGG GGCTAGCTACAACGA GTCCCCTG | 4459 |
| 3828 | GGGACGCC A CACUCGCC | 3456 | GGCGAGTG GGCTAGCTACAACGA GGCGTCCC | 4463 |
| 3830 | GACGCCAC A CUCGCCCU | 3457 | AGGGCGAG GGCTAGCTACAACGA GTGGCGTC | 4464 |
| 3834 | CCACACUC G CCCUUCUG | 3458 | CAGAAGGG GGCTAGCTACAACGA GAGTGTGG | 4465 |
| 3842 | GCCCUUCU G UCCAGGGG | 3459 | CCCCTGGA GGCTAGCTACAACGA AGAAGGGC | 4466 |
| 3851 | UCCAGGGG A CGCCACAC | 3451 | GTGTGGCG GGCTAGCTACAACGA CCCCTGGA | 4458 |
| 3853 | CAGGGGAC G CCACACUC | 3452 | GAGTGTGG GGCTAGCTACAACGA GTCCCCTG | 4459 |
| 3856 | GGGACGCC A CACUCGCC | 3456 | GGCGAGTG GGCTAGCTACAACGA GGCGTCCC | 4463 |
| 3858 | GACGCCAC A CUCGCCCU | 3457 | AGGGCGAG GGCTAGCTACAACGA GTGGCGTC | 4464 |
| 3862 | CCACACUC G CCCUUCUC | 3445 | GAGAAGGG GGCTAGCTACAACGA GAGTGTGG | 4452 |
| 3879 | UCCAGGGG A CGCCACAC | 3451 | GTGTGGCG GGCTAGCTACAACGA CCCCTGGA | 4458 |
| 3881 | CAGGGGAC G CCACACUC | 3452 | GAGTGTGG GGCTAGCTACAACGA GTCCCCTG | 4459 |
| 3884 | GGGACGCC A CACUCGCC | 3456 | GGCGAGTG GGCTAGCTACAACGA GGCGTCCC | 4463 |
| 3886 | GACGCCAC A CUCGCCCU | 3457 | AGGGCGAG GGCTAGCTACAACGA GTGGCGTC | 4464 |
| 3890 | CCACACUC G CCCUUCUC | 3445 | GAGAAGGG GGCTAGCTACAACGA GAGTGTGG | 4452 |
| 3907 | UCCAGGGG A CGCCACAC | 3451 | GTGTGGCG GGCTAGCTACAACGA CCCCTGGA | 4458 |
| 3909 | CAGGGGAC G CCACACUC | 3452 | GAGTGTGG GGCTAGCTACAACGA GTCCCCTG | 4459 |
| 3912 | GGGACGCC A CACUCCCC | 3453 | GGGGAGTG GGCTAGCTACAACGA GGCGTCCC | 4460 |
| 3914 | GACGCCAC A CUCCCCCU | 3454 | AGGGGGAG GGCTAGCTACAACGA GTGGCGTC | 4461 |
| 3926 | CCCCUUCU G UCCAGGGG | 3455 | CCCCTGGA GGCTAGCTACAACGA AGAAGGGG | 4462 |
| 3935 | UCCAGGGG A CGCCACAC | 3451 | GTGTGGCG GGCTAGCTACAACGA CCCCTGGA | 4458 |
| 3937 | CAGGGGAC G CCACACUC | 3452 | GAGTGTGG GGCTAGCTACAACGA GTCCCCTG | 4459 |
| 3940 | GGGACGCC A CACUCCCC | 3453 | GGGGAGTG GGCTAGCTACAACGA GGCGTCCC | 4460 |
| 3942 | GACGCCAC A CUCCCCCU | 3454 | AGGGGGAG GGCTAGCTACAACGA GTGGCGTC | 4461 |
| 3963 | UCCAGGGG A CGCCACAC | 3451 | GTGTGGCG GGCTAGCTACAACGA CCCCTGGA | 4458 |
| 3965 | CAGGGGAC G CCACACUC | 3452 | GAGTGTGG GGCTAGCTACAACGA GTCCCCTG | 4459 |
| 3968 | GGGACGCC A CACUCCCC | 3453 | GGGGAGTG GGCTAGCTACAACGA GGCGTCCC | 4460 |
| 3970 | GACGCCAC A CUCCCCCU | 3454 | AGGGGGAG GGCTAGCTACAACGA GTGGCGTC | 4461 |
| 3991 | UCCAGGGG A CGCCACAC | 3451 | GTGTGGCG GGCTAGCTACAACGA CCCCTGGA | 4458 |
| 3993 | CAGGGGAC G CCACACUC | 3452 | GAGTGTGG GGCTAGCTACAACGA GTCCCCTG | 4459 |
| 3996 | GGGACGCC A CACUCGCC | 3456 | GGCGAGTG GGCTAGCTACAACGA GGCGTCCC | 4463 |
| 3998 | GACGCCAC A CUCGCCCU | 3457 | AGGGCGAG GGCTAGCTACAACGA GTGGCGTC | 4464 |
| 4002 | CCACACUC G CCCUUCUC | 3445 | GAGAAGGG GGCTAGCTACAACGA GAGTGTGG | 4452 |
| 4019 | UCCAGGGG A CGCCACAC | 3451 | GTGTGGCG GGCTAGCTACAACGA CCCCTGGA | 4458 |
| 4021 | CAGGGGAC G CCACACUC | 3452 | GAGTGTGG GGCTAGCTACAACGA GTCCCCTG | 4459 |
| 4024 | GGGACGCC A CACUCCCC | 3453 | GGGGAGTG GGCTAGCTACAACGA GGCGTCCC | 4460 |
| 4026 | GACGCCAC A CUCCCCCU | 3454 | AGGGGGAG GGCTAGCTACAACGA GTGGCGTC | 4461 |
| 4038 | CCCCUUCU G UCCAGGGG | 3455 | CCCCTGGA GGCTAGCTACAACGA AGAAGGGG | 4462 |
| 4047 | UCCAGGGG A CGCCACAC | 3451 | GTGTGGCG GGCTAGCTACAACGA CCCCTGGA | 4458 |
| 4049 | CAGGGGAC G CCACACUC | 3452 | GAGTGTGG GGCTAGCTACAACGA GTCCCCTG | 4459 |
| 4052 | GGGACGCC A CACUCGCC | 3456 | GGCGAGTG GGCTAGCTACAACGA GGCGTCCC | 4463 |
| 4054 | GACGCCAC A CUCGCCCU | 3457 | AGGGCGAG GGCTAGCTACAACGA GTGGCGTC | 4464 |
| 4058 | CCACACUC G CCCUUCUC | 3445 | GAGAAGGG GGCTAGCTACAACGA GAGTGTGG | 4452 |
| 4075 | UCCAGGGG A CGCCACAC | 3451 | GTGTGGCG GGCTAGCTACAACGA CCCCTGGA | 4458 |
| 4077 | CAGGGGAC G CCACACUC | 3452 | GAGTGTGG GGCTAGCTACAACGA GTCCCCTG | 4459 |
| 4080 | GGGACGCC A CACUCGCC | 3456 | GGCGAGTG GGCTAGCTACAACGA GGCGTCCC | 4463 |
| 4082 | GACGCCAC A CUCGCCCU | 3457 | AGGGCGAG GGCTAGCTACAACGA GTGGCGTC | 4464 |
| 4086 | CCACACUC G CCCUUCUC | 3445 | GAGAAGGG GGCTAGCTACAACGA GAGTGTGG | 4452 |
| 4103 | UCCAGGGG A CGCCACAC | 3451 | GTGTGGCG GGCTAGCTACAACGA CCCCTGGA | 4458 |
| 4105 | CAGGGGAC G CCACACUC | 3452 | GAGTGTGG GGCTAGCTACAACGA GTCCCCTG | 4459 |
| 4108 | GGGACGCC A CACUCCCC | 3453 | GGGGAGTG GGCTAGCTACAACGA GGCGTCCC | 4460 |
| 4110 | GACGCCAC A CUCCCCCU | 3454 | AGGGGGAG GGCTAGCTACAACGA GTGGCGTC | 4461 |
| 4131 | UCCAGGGG A CGCCACAC | 3451 | GTGTGGCG GGCTAGCTACAACGA CCCCTGGA | 4458 |
| 4133 | CAGGGGAC G CCACACUC | 3452 | GAGTGTGG GGCTAGCTACAACGA GTCCCCTG | 4459 |
| 4136 | GGGACGCC A CACUCCCC | 3453 | GGGGAGTG GGCTAGCTACAACGA GGCGTCCC | 4460 |
| 4138 | GACGCCAC A CUCCCCCU | 3454 | AGGGGGAG GGCTAGCTACAACGA GTGGCGTC | 4461 |
| 4159 | UCCAGGGG A CGCCACAC | 3451 | GTGTGGCG GGCTAGCTACAACGA CCCCTGGA | 4458 |
| 4161 | CAGGGGAC G CCACACUC | 3452 | GAGTGTGG GGCTAGCTACAACGA GTCCCCTG | 4459 |
| 4164 | GGGACGCC A CACUCCCC | 3453 | GGGGAGTG GGCTAGCTACAACGA GGCGTCCC | 4460 |
| 4166 | GACGCCAC A CUCCCCCU | 3454 | AGGGGGAG GGCTAGCTACAACGA GTGGCGTC | 4461 |
| 4178 | CCCCUUCU G UCCAGGGG | 3455 | CCCCTGGA GGCTAGCTACAACGA AGAAGGGG | 4462 |
| 4187 | UCCAGGGG A CGCCACAC | 3451 | GTGTGGCG GGCTAGCTACAACGA CCCCTGGA | 4458 |
| 4189 | CAGGGGAC G CCACACUC | 3452 | GAGTGTGG GGCTAGCTACAACGA GTCCCCTG | 4459 |
| 4192 | GGGACGCC A CACUCGCC | 3456 | GGCGAGTG GGCTAGCTACAACGA GGCGTCCC | 4463 |
| 4194 | GACGCCAC A CUCGCCCU | 3457 | AGGGCGAG GGCTAGCTACAACGA GTGGCGTC | 4464 |
| 4198 | CCACACUC G CCCUUCUC | 3445 | GAGAAGGG GGCTAGCTACAACGA GAGTGTGG | 4452 |
| 4215 | UCCAGGGG A CGCCACAC | 3451 | GTGTGGCG GGCTAGCTACAACGA CCCCTGGA | 4458 |
| 4217 | CAGGGGAC G CCACACUC | 3452 | GAGTGTGG GGCTAGCTACAACGA GTCCCCTG | 4459 |
| 4220 | GGGACGCC A CACUCCCC | 3453 | GGGGAGTG GGCTAGCTACAACGA GGCGTCCC | 4460 |
| 4222 | GACGCCAC A CUCCCCCU | 3454 | AGGGGGAG GGCTAGCTACAACGA GTGGCGTC | 4461 |
| 4243 | UCCAGGGG A CGCCACAC | 3451 | GTGTGGCG GGCTAGCTACAACGA CCCCTGGA | 4458 |
| 4245 | CAGGGGAC G CCACACUC | 3452 | GAGTGTGG GGCTAGCTACAACGA GTCCCCTG | 4459 |
| 4248 | GGGACGCC A CACUCCCC | 3453 | GGGGAGTG GGCTAGCTACAACGA GGCGTCCC | 4460 |
| 4250 | GACGCCAC A CUCCCCCU | 3454 | AGGGGGAG GGCTAGCTACAACGA GTGGCGTC | 4461 |
| 4271 | UCCAGGGG A CGCCACAC | 3451 | GTGTGGCG GGCTAGCTACAACGA CCCCTGGA | 4458 |
| 4273 | CAGGGGAC G CCACACUC | 3452 | GAGTGTGG GGCTAGCTACAACGA GTCCCCTG | 4459 |
| 4276 | GGGACGCC A CACUCCCC | 3453 | GGGGAGTG GGCTAGCTACAACGA GGCGTCCC | 4460 |
| 4278 | GACGCCAC A CUCCCCCU | 3454 | AGGGGGAG GGCTAGCTACAACGA GTGGCGTC | 4461 |
| 4290 | CCCCUUCU G UCCAGGGG | 3455 | CCCCTGGA GGCTAGCTACAACGA AGAAGGGG | 4462 |
| 4299 | UCCAGGGG A CGCCACAC | 3451 | GTGTGGCG GGCTAGCTACAACGA CCCCTGGA | 4458 |
| 4301 | CAGGGGAC G CCACACUC | 3452 | GAGTGTGG GGCTAGCTACAACGA GTCCCCTG | 4459 |
| 4304 | GGGACGCC A CACUCGCC | 3456 | GGCGAGTG GGCTAGCTACAACGA GGCGTCCC | 4463 |
| 4306 | GACGCCAC A CUCGCCCU | 3457 | AGGGCGAG GGCTAGCTACAACGA GTGGCGTC | 4464 |
| 4310 | CCACACUC G CCCUUCUC | 3445 | GAGAAGGG GGCTAGCTACAACGA GAGTGTGG | 4452 |
| 4327 | UCCAGGGG A CGCCACAC | 3451 | GTGTGGCG GGCTAGCTACAACGA CCCCTGGA | 4458 |
| 4329 | CAGGGGAC G CCACACUC | 3452 | GAGTGTGG GGCTAGCTACAACGA GTCCCCTG | 4459 |
| 4332 | GGGACGCC A CACUCGCC | 3456 | GGCGAGTG GGCTAGCTACAACGA GGCGTCCC | 4463 |
| 4334 | GACGCCAC A CUCGCCCU | 3457 | AGGGCGAG GGCTAGCTACAACGA GTGGCGTC | 4464 |
| 4338 | CCACACUC G CCCUUCUC | 3445 | GAGAAGGG GGCTAGCTACAACGA GAGTGTGG | 4452 |
| 4355 | UCCAGGGG A CGCCACAC | 3451 | GTGTGGCG GGCTAGCTACAACGA CCCCTGGA | 4458 |
| 4357 | CAGGGGAC G CCACACUC | 3452 | GAGTGTGG GGCTAGCTACAACGA GTCCCCTG | 4459 |
| 4360 | GGGACGCC A CACUCGCC | 3456 | GGCGAGTG GGCTAGCTACAACGA GGCGTCCC | 4463 |
| 4362 | GACGCCAC A CUCGCCCU | 3457 | AGGGCGAG GGCTAGCTACAACGA GTGGCGTC | 4464 |
| 4366 | CCACACUC G CCCUUCUC | 3445 | GAGAAGGG GGCTAGCTACAACGA GAGTGTGG | 4452 |
| 4383 | UCCAGGGG A CGCCACAC | 3451 | GTGTGGCG GGCTAGCTACAACGA CCCCTGGA | 4458 |
| 4385 | CAGGGGAC G CCACACUU | 3460 | AAGTGTGG GGCTAGCTACAACGA GTCCCCTG | 4467 |
| 4388 | GGGACGCC A CACUUGCC | 3461 | GGCAAGTG GGCTAGCTACAACGA GGCGTCCC | 4468 |
| 4390 | GACGCCAC A CUUGCCCU | 3462 | AGGGCAAG GGCTAGCTACAACGA GTGGCGTC | 4469 |
| 4394 | CCACACUU G CCCUUCUG | 3463 | CAGAAGGG GGCTAGCTACAACGA AAGTGTGG | 4470 |
| 4402 | GCCCUUCU G UCCAGGGA | 3464 | TCCCTGGA GGCTAGCTACAACGA AGAAGGGC | 4471 |
| 4411 | UCCAGGGA A UGCCACAC | 3465 | GTGTGGCA GGCTAGCTACAACGA TCCCTGGA | 4472 |
| 4413 | CAGGGAAU G CCACACUC | 3466 | GAGTGTGG GGCTAGCTACAACGA ATTCCCTG | 4449 |
| 4416 | GGAAUGCC A CACUCCCC | 3467 | GGGGAGTG GGCTAGCTACAACGA GGCATTCC | 4473 |
| 4418 | AAUGCCAC A CUCCCCCU | 3468 | AGGGGGAG GGCTAGCTACAACGA GTGGCATT | 4474 |
| 4435 | UCUCCCCA G CAGCCUCC | 3469 | GGAGGCTG GGCTAGCTACAACGA TGGGGAGA | 4475 |
| 4438 | CCCCAGCA G CCUCCGAG | 3470 | CTCGGAGG GGCTAGCTACAACGA TGCTGGGG | 4476 |
| 4446 | GCCUCCGA G UGACCAGC | 3471 | GCTGGTCA GGCTAGCTACAACGA TCGGAGGC | 4477 |
| 4449 | UCCGAGUG A CCAGCUUC | 3472 | GAAGCTGG GGCTAGCTACAACGA CACTCGGA | 4478 |
| 4453 | AGUGACCA G CUUCCCCA | 3473 | TGGGGAAG GGCTAGCTACAACGA TGGTCACT | 4479 |
| 4461 | GCUUCCCC A UCGAUAGA | 3474 | TCTATCGA GGCTAGCTACAACGA GGGGAAGC | 4480 |
| 4465 | CCCCAUCG A UAGACUUC | 3475 | GAAGTCTA GGCTAGCTACAACGA CGATGGGG | 4481 |
| 4469 | AUCGAUAG A CUUCCCGA | 3476 | TCGGGAAG GGCTAGCTACAACGA CTATCGAT | 4482 |
| 4479 | UUCCCGAG G CCAGGAGC | 3477 | GCTCCTGG GGCTAGCTACAACGA CTCGGGAA | 4483 |
| 4486 | GGCCAGGA G CCCUCUAG | 3478 | CTAGAGGG GGCTAGCTACAACGA TCCTGGCC | 4484 |
| 4496 | CCUCUAGG G CUGCCGGG | 3479 | CCCGGCAG GGCTAGCTACAACGA CCTAGAGG | 4485 |
| 4499 | CUAGGGCU G CCGGGUGC | 3480 | GCACCCGG GGCTAGCTACAACGA AGCCCTAG | 4486 |
| 4504 | GCUGCCGG G UGCCACCC | 3481 | GGGTGGCA GGCTAGCTACAACGA CCGGCAGC | 4487 |
| 4506 | UGCCGGGU G CCACCCUG | 3482 | CAGGGTGG GGCTAGCTACAACGA ACCCGGCA | 4488 |
| 4509 | CGGGUGCC A CCCUGGCU | 3483 | AGCCAGGG GGCTAGCTACAACGA GGCACCCG | 4489 |
| 4515 | CCACCCUG G CUCCUUCC | 3484 | GGAAGGAG GGCTAGCTACAACGA CAGGGTGG | 4490 |
| 4524 | CUCCUUCC A CACCGUGC | 3485 | GCACGGTG GGCTAGCTACAACGA GGAAGGAG | 4491 |
| 4526 | CCUUCCAC A CCGUGCUG | 3486 | CAGCACGG GGCTAGCTACAACGA GTGGAAGG | 4492 |
| 4529 | UCCACACC G UGCUGGUC | 3487 | GACCAGCA GGCTAGCTACAACGA GGTGTGGA | 4493 |
| 4531 | CACACCGU G CUGGUCAC | 3488 | GTGACCAG GGCTAGCTACAACGA ACGGTGTG | 4494 |
| 4535 | CCGUGCUG G UCACUGCC | 3489 | GGCAGTGA GGCTAGCTACAACGA CAGCACGG | 4495 |
| 4538 | UGCUGGUC A CUGCCUGC | 3490 | GCAGGCAG GGCTAGCTACAACGA GACCAGCA | 4496 |
| 4541 | UGGUCACU G CCUGCUGG | 3491 | CCAGCAGG GGCTAGCTACAACGA AGTGACCA | 4497 |
| 4545 | CACUGCCU G CUGGGGGC | 3492 | GCCCCCAG GGCTAGCTACAACGA AGGCAGTG | 4498 |
| 4552 | UGCUGGGG G CGUCAGAU | 3493 | ATCTGACG GGCTAGCTACAACGA CCCCAGCA | 4499 |
| 4554 | CUGGGGGC G UCAGAUGC | 3494 | GCATCTGA GGCTAGCTACAACGA GCCCCCAG | 4500 |
| 4559 | GGCGUCAG A UGCAGGUG | 3495 | CACCTGCA GGCTAGCTACAACGA CTGACGCC | 4501 |
| 4561 | CGUCAGAU G CAGGUGAC | 3496 | GTCACCTG GGCTAGCTACAACGA ATCTGACG | 4502 |
| 4565 | AGAUGCAG G UGACCCUG | 3497 | CAGGGTCA GGCTAGCTACAACGA CTGCATCT | 4503 |
| 4568 | UGCAGGUG A CCCUGUGC | 3498 | GCACAGGG GGCTAGCTACAACGA CACCTGCA | 4504 |
| 4573 | GUGACCCU G UGCAGGAG | 3499 | CTCCTGCA GGCTAGCTACAACGA AGGGTCAC | 4505 |
| 4575 | GACCCUGU G CAGGAGGU | 3500 | ACCTCCTG GGCTAGCTACAACGA ACAGGGTC | 4506 |
| 4582 | UGCAGGAG G UAUCUCUG | 3501 | CAGAGATA GGCTAGCTACAACGA CTCCTGCA | 4507 |
| 4584 | CAGGAGGU A UCUCUGGA | 3502 | TCCAGAGA GGCTAGCTACAACGA ACCTCCTG | 4508 |
| 4592 | AUCUCUGG A CCUGCCUC | 3503 | GAGGCAGG GGCTAGCTACAACGA CCAGAGAT | 4509 |
| 4596 | CUGGACCU G CCUCUUGG | 3504 | CCAAGAGG GGCTAGCTACAACGA AGGTCCAG | 4510 |
| 4604 | GCCUCUUG G UCAUUACG | 3505 | CGTAATGA GGCTAGCTACAACGA CAAGAGGC | 4511 |
| 4607 | UCUUGGUC A UUACGGGG | 3506 | CCCCGTAA GGCTAGCTACAACGA GACCAAGA | 4512 |
| 4610 | UGGUCAUU A CGGGGCUG | 3507 | CAGCCCCG GGCTAGCTACAACGA AATGACCA | 4513 |
| 4615 | AUUACGGG G CUGGGCAG | 3508 | CTGCCCAG GGCTAGCTACAACGA CCCGTAAT | 4514 |
| 4620 | GGGGCUGG G CAGGGCCU | 3509 | AGGCCCTG GGCTAGCTACAACGA CCAGCCCC | 4515 |
| 4625 | UGGGCAGG G CCUGGUAU | 3510 | ATACCAGG GGCTAGCTACAACGA CCTGCCCA | 4516 |
| 4630 | AGGGCCUG G UAUCAGGG | 3511 | CCCTGATA GGCTAGCTACAACGA CAGGCCCT | 4517 |
| 4632 | GGCCUGGU A UCAGGGCC | 3512 | GGCCCTGA GGCTAGCTACAACGA ACCAGGCC | 4518 |
| 4638 | GUAUCAGG G CCCCGCUG | 3513 | CAGCGGGG GGCTAGCTACAACGA CCTGATAC | 4519 |
| 4643 | AGGGCCCC G CUGGGGUU | 3514 | AACCCCAG GGCTAGCTACAACGA GGGGCCCT | 4520 |
| 4649 | CCGCUGGG G UUGCAGGG | 3515 | CCCTGCAA GGCTAGCTACAACGA CCCAGCGG | 4521 |
| 4652 | CUGGGGUU G CAGGGCUG | 3516 | CAGCCCTG GGCTAGCTACAACGA AACCCCAG | 4522 |
| 4657 | GUUGCAGG G CUGGGCCU | 3517 | AGGCCCAG GGCTAGCTACAACGA CCTGCAAC | 4523 |
| 4662 | AGGGCUGG G CCUGUGCU | 3518 | AGCACAGG GGCTAGCTACAACGA CCAGCCCT | 4524 |
| 4666 | CUGGGCCU G UGCUGUGG | 3519 | CCACAGCA GGCTAGCTACAACGA AGGCCCAG | 4525 |
| 4668 | GGGCCUGU G CUGUGGUC | 3520 | GACCACAG GGCTAGCTACAACGA ACAGGCCC | 4526 |
| 4671 | CCUGUGCU G UGGUCCUG | 3521 | CAGGACCA GGCTAGCTACAACGA AGCACAGG | 4527 |
| 4674 | GUGCUGUG G UCCUGGGG | 3522 | CCCCAGGA GGCTAGCTACAACGA CACAGCAC | 4528 |
| 4682 | GUCCUGGG G UGUCCAGG | 3523 | CCTGGACA GGCTAGCTACAACGA CCCAGGAC | 4529 |
| 4684 | CCUGGGGU G UCCAGGAC | 3524 | GTCCTGGA GGCTAGCTACAACGA ACCCCAGG | 4530 |
| 4691 | UGUCCAGG A CAGACGUG | 3525 | CACGTCTG GGCTAGCTACAACGA CCTGGACA | 4531 |
| 4695 | CAGGACAG A CGUGGAGG | 3526 | CCTCCACG GGCTAGCTACAACGA CTGTCCTG | 4532 |
| 4697 | GGACAGAC G UGGAGGGG | 3527 | CCCCTCCA GGCTAGCTACAACGA GTCTGTCC | 4533 |
| 4705 | GUGGAGGG G UCAGGGCC | 3528 | GGCCCTGA GGCTAGCTACAACGA CCCTCCAC | 4534 |
| 4711 | GGGUCAGG G CCCAGCAC | 3529 | GTGCTGGG GGCTAGCTACAACGA CCTGACCC | 4535 |
| 4716 | AGGGCCCA G CACCCCUG | 3530 | CAGGGGTG GGCTAGCTACAACGA TGGGCCCT | 4536 |
| 4718 | GGCCCAGC A CCCCUGCU | 3531 | AGCAGGGG GGCTAGCTACAACGA GCTGGGCC | 4537 |
| 4724 | GCACCCCU G CUCCAUGC | 3532 | GCATGGAG GGCTAGCTACAACGA AGGGGTGC | 4538 |
| 4729 | CCUGCUCC A UGCUGAAC | 3533 | GTTCAGCA GGCTAGCTACAACGA GGAGCAGG | 4539 |
| 4731 | UGCUCCAC G CUGAACUG | 3534 | CAGTTCAG GGCTAGCTACAACGA ATGGAGCA | 4540 |
| 4736 | CAUGCUGA A CUGUGGGA | 3535 | TCCCACAG GGCTAGCTACAACGA TCAGCATG | 4541 |
| 4739 | GCUGAACU G UGGGAAGC | 3536 | GCTTCCCA GGCTAGCTACAACGA AGTTCAGC | 4542 |
| 4746 | UGUGGGAA G CAUCCAGG | 3537 | CCTGGATG GGCTAGCTACAACGA TTCCCACA | 4543 |
| 4748 | UGGGAAGC A UCCAGGUC | 3538 | GACCTGGA GGCTAGCTACAACGA GCTTCCCA | 4544 |
| 4754 | GCAUCCAG G UCCCUGGG | 3539 | CCCAGGGA GGCTAGCTACAACGA CTGGATGC | 4545 |
| 4762 | GUCCCUGG G UGGCUUCA | 3540 | TGAAGCCA GGCTAGCTACAACGA CCAGGGAC | 4546 |
| 4765 | CCUGGGUG G CUUCAACA | 3541 | TGTTGAAG GGCTAGCTACAACGA CACCCAGG | 4547 |
| 4771 | UGGCUUCA A CAGGAGUU | 3542 | AACTCCTG GGCTAGCTACAACGA TGAAGCCA | 4548 |
| 4777 | CAACAGGA G UUCCAGCA | 3543 | TGCTGGAA GGCTAGCTACAACGA TCCTGTTG | 4549 |
| 4783 | GAGUUCCA G CACGGGAA | 3544 | TTCCCGTG GGCTAGCTACAACGA TGGAACTC | 4550 |
| 4785 | GUUCCAGC A CGGGAACC | 3545 | GGTTCCCG GGCTAGCTACAACGA GCTGGAAC | 4551 |
| 4791 | GCACGGGA A CCACUGGA | 3546 | TCCAGTGG GGCTAGCTACAACGA TCCCGTGC | 4552 |
| 4794 | CGGGAACC A CUGGACAA | 3547 | TTGTCCAG GGCTAGCTACAACGA GGTTCCCG | 4553 |
| 4799 | ACCACUGG A CAACCUGG | 3548 | CCAGGTTG GGCTAGCTACAACGA CCAGTGGT | 4554 |
| 4802 | ACUGGACA A CCUGGGGU | 3549 | ACCCCAGG GGCTAGCTACAACGA TGTCCAGT | 4555 |
| 4809 | AACCUGGG G UGUGUCCU | 3550 | AGGACACA GGCTAGCTACAACGA CCCAGGTT | 4556 |
| 4811 | CCUGGGGU G UGUCCUGA | 3551 | TCAGGACA GGCTAGCTACAACGA ACCCCAGG | 4557 |
| 4813 | UGGGGUGU G UCCUGAUC | 3552 | GATCAGGA GGCTAGCTACAACGA ACACCCCA | 4558 |
| 4819 | GUGUCCUG A UCUGGGGA | 3553 | TCCCCAGA GGCTAGCTACAACGA CAGGACAC | 4559 |
| 4827 | AUCUGGGG A CAGGCCAG | 3554 | CTGGCCTG GGCTAGCTACAACGA CCCCAGAT | 4560 |
| 4831 | GGGGACAG G CCAGCCAC | 3555 | GTGGCTGG GGCTAGCTACAACGA CTGTCCCC | 4561 |
| 4835 | ACAGGCCA G CCACACCC | 3556 | GGGTGTGG GGCTAGCTACAACGA TGGCCTGT | 4562 |
| 4838 | GGCCAGCC A CACCCCGA | 3557 | TCGGGGTG GGCTAGCTACAACGA GGCTGGCC | 4563 |
| 4840 | CCAGCCAC A CCCCGAGU | 3558 | ACTCGGGG GGCTAGCTACAACGA GTGGCTGG | 4564 |
| 4847 | CACCCCGA G UCCUAGGG | 3559 | CCCTAGGA GGCTAGCTACAACGA TCGGGGTG | 4565 |
| 4856 | UCCUAGGG A CUCCAGAG | 3560 | CTCTGGAG GGCTAGCTACAACGA CCCTAGGA | 4566 |
| 4866 | UCCAGAGA G CAGCCCAC | 3561 | GTGGGCTG GGCTAGCTACAACGA TCTCTGGA | 4567 |
| 4869 | AGAGAGCA G CCCACUGC | 3562 | GCAGTGGG GGCTAGCTACAACGA TGCTCTCT | 4568 |
| 4873 | AGCAGCCC A CUGCCCUG | 3563 | CAGGGCAG GGCTAGCTACAACGA GGGCTGCT | 4569 |
| 4876 | AGCCCACU G CCCUGGGC | 3564 | GCCCAGGG GGCTAGCTACAACGA AGTGGGCT | 4570 |
| 4883 | UGCCCUGG G CUCCACGG | 3565 | CCGTGGAG GGCTAGCTACAACGA CCAGGGCA | 4571 |
| 4888 | UGGGCUCC A CGGAAGCC | 3566 | GGCTTCCG GGCTAGCTACAACGA GGAGCCCA | 4572 |
| 4894 | CCACGGAA G CCCCCUCA | 3567 | TGAGGGGG GGCTAGCTACAACGA TTCCGTGG | 4573 |
| 4902 | GCCCCCUC A UGCCGCUA | 3568 | TAGCGGCA GGCTAGCTACAACGA GAGGGGGC | 4574 |
| 4904 | CCCCUCAU G CCGCUAGG | 3569 | CCTAGCGG GGCTAGCTACAACGA ATGAGGGG | 4575 |
| 4907 | CUCAUGCC G CUAGGCCU | 3570 | AGGCCTAG GGCTAGCTACAACGA GGCATGAG | 4576 |
| 4912 | GCCGCUAG G CCUUGGCC | 3571 | GGCCAAGG GGCTAGCTACAACGA CTAGCGGC | 4577 |
| 4918 | AGGCCUUG G CCUCGGGG | 3572 | CCCCGAGG GGCTAGCTACAACGA CAAGGCCT | 4578 |
| 4927 | CCUCGGGG A CAGCCCAG | 3573 | CTGGGCTG GGCTAGCTACAACGA CCCCGAGG | 4579 |
| 4930 | CGGGGACA G CCCAGCUA | 3574 | TAGCTGGG GGCTAGCTACAACGA TGTCCCCG | 4580 |
| 4935 | ACAGCCCA G CUAGGCCA | 3575 | TGGCCTAG GGCTAGCTACAACGA TGGGCTGT | 4581 |
| 4940 | CCAGCUAG G CCAGUGUG | 3576 | CACACTGG GGCTAGCTACAACGA CTAGCTGG | 4582 |
| 4944 | CUAGGCCA G UGUGUGGC | 3577 | GCCACACA GGCTAGCTACAACGA TGGCCTAG | 4583 |
| 4946 | AGGCCAGU G UGUGGCAG | 3578 | CTGCCACA GGCTAGCTACAACGA ACTGGCCT | 4584 |
| 4948 | GCCAGUGU G UGGCAGGA | 3579 | TCCTGCCA GGCTAGCTACAACGA ACACTGGC | 4585 |
| 4951 | AGUGUGUG G CAGGACCA | 3580 | TGGTCCTG GGCTAGCTACAACGA CACACACT | 4586 |
| 4956 | GUGGCAGG A CCAGGCCC | 3581 | GGGCCTGG GGCTAGCTACAACGA CCTGCCAC | 4587 |
| 4961 | AGGACCAG G CCCCCAUG | 3582 | CATGGGGG GGCTAGCTACAACGA CTGGTCCT | 4588 |
| 4967 | AGGCCCCC A UGUGGGAG | 3583 | CTCCCACA GGCTAGCTACAACGA GGGGGCCT | 4589 |
| 4969 | GCCCCCAU G UGGGAGCU | 3584 | AGCTCCCA GGCTAGCTACAACGA ATGGGGGC | 4590 |
| 4975 | AUGUGGGA G CUGACCCC | 3585 | GGGGTCAG GGCTAGCTACAACGA TCCCACAT | 4591 |
| 4979 | GGGAGCUG A CCCCUUGG | 3586 | CCAAGGGG GGCTAGCTACAACGA CAGCTCCC | 4592 |
| 4989 | CCCUUGGG A UUCUGGAG | 3587 | CTCCAGAA GGCTAGCTACAACGA CCCAAGGG | 4593 |
| 4997 | AUUCUGGA G CUGUGCUG | 3588 | CAGCACAG GGCTAGCTACAACGA TCCAGAAT | 4594 |
| 5000 | CUGGAGCU G UGCUGAUG | 3589 | CATCAGCA GGCTAGCTACAACGA AGCTCCAG | 4595 |
| 5002 | GGAGCUGU G CUGAUGGG | 3590 | CCCATCAG GGCTAGCTACAACGA ACAGCTCC | 4596 |
| 5006 | CUGUGCUG A UGGGCAGG | 3591 | CCTGCCCA GGCTAGCTACAACGA CAGCACAG | 4597 |
| 5010 | GCUGAUGG G CAGGGGAG | 3592 | CTCCCCTG GGCTAGCTACAACGA CCATCAGC | 4598 |
| 5020 | AGGGGAGA G CCAGCUCC | 3593 | GGAGCTGG GGCTAGCTACAACGA TCTCCCCT | 4599 |
| 5024 | GAGAGCCA G CUCCUCCC | 3594 | GGGAGGAG GGCTAGCTACAACGA TGGCTCTC | 4600 |
| 5044 | GAGGGAGG G UCUUGAUG | 3595 | CATCAAGA GGCTAGCTACAACGA CCTCCCTC | 4601 |
| 5050 | GGGUCUUG A UGCCUGGG | 3596 | CCCAGGCA GGCTAGCTACAACGA CAAGACCC | 4602 |
| 5052 | GUCUUGAU G CCUGGGGU | 3597 | ACCCCAGG GGCTAGCTACAACGA ATCAAGAC | 4603 |
| 5059 | UGCCUGGG G UUACCCGC | 3598 | GCGGGTAA GGCTAGCTACAACGA CCCAGGCA | 4604 |
| 5062 | CUGGGGUU A CCCGCAGA | 3599 | TCTGCGGG GGCTAGCTACAACGA AACCCCAG | 4605 |
| 5066 | GGUUACCC G CAGAGGCC | 3600 | GGCCTCTG GGCTAGCTACAACGA GGGTAACC | 4606 |
| 5072 | CCGCAGAG G CCUGGGUG | 3601 | CACCCAGG GGCTAGCTACAACGA CTCTGCGG | 4607 |
| 5078 | AGGCCUGG G UGCCGGGA | 3602 | TCCCGGCA GGCTAGCTACAACGA CCAGGCCT | 4608 |
| 5080 | GCCUGGGU G CCGGGACG | 3603 | CGTCCCGG GGCTAGCTACAACGA ACCCAGGC | 4609 |
| 5086 | GUGCCGGG A CGCUCCCC | 3604 | GGGGAGCG GGCTAGCTACAACGA CCCGGCAC | 4610 |
| 5088 | GCCGGGAC G CUCCCCGG | 3605 | CCGGGGAG GGCTAGCTACAACGA GTCCCGGC | 4611 |
| 5096 | GCUCCCCG G UUUGGCUG | 3606 | CAGCCAAA GGCTAGCTACAACGA CGGGGAGC | 4612 |
| 5101 | CCGGUUUG G CUGAAAGG | 3607 | CCTTTCAG GGCTAGCTACAACGA CAAACCGG | 4613 |
| 5113 | AAAGGAAA G CAGAUGUG | 3608 | CACATCTG GGCTAGCTACAACGA TTTCCTTT | 4614 |
| 5117 | GAAAGCAG A UGUGGUCA | 3609 | TGACCACA GGCTAGCTACAACGA CTGCTTTC | 4615 |
| 5119 | AAGCAGAU G UGGUCAGC | 3610 | GCTGACCA GGCTAGCTACAACGA ATCTGCTT | 4616 |
| 5222 | CAGAUGUG G UCAGCUUG | 3611 | GAAGCTGA GGCTAGCTACAACGA CACATCTG | 4617 |
| 5126 | UGUGGUCA G CUUCUCCA | 3612 | TGGAGAAG GGCTAGCTACAACGA TGACCACA | 4618 |
| 5134 | GCUUCUCC A CUGAGCCC | 3613 | GGGCTCAG GGCTAGCTACAACGA GGAGAAGC | 4619 |
| 5139 | UCCACUGA G CCCAUCUG | 3614 | CAGATGGG GGCTAGCTACAACGA TCAGTGGA | 4620 |
| 5143 | CUGAGCCC A UCUGGUCU | 3615 | AGACCAGA GGCTAGCTACAACGA GGGCTCAG | 4621 |
| 5148 | CCCAUCUG G UCUUCCCG | 3616 | CGGGAAGA GGCTAGCTACAACGA CAGATGGG | 4622 |
| 5159 | UUCCCGGG G CUGGGCCC | 3617 | GGGCCCAG GGCTAGCTACAACGA CCCGGGAA | 4623 |
| 5164 | GGGGCUGG G CCCCAUAG | 3618 | CTATGGGG GGCTAGCTACAACGA CCAGCCCC | 4624 |
| 5169 | UGGGCCCC A UAGAUCUG | 3619 | CAGATCTA GGCTAGCTACAACGA GGGGCCCA | 4625 |
| 5173 | CCCCAUAG A UCUGGGUC | 3620 | GACCCAGA GGCTAGCTACAACGA CTATGGGG | 4626 |
| 5179 | AGAUCUGG G UCCCUGUG | 3621 | CACAGGGA GGCTAGCTACAACGA CCAGATCT | 4627 |
| 5185 | GGGUCCCU G UGUGGCCC | 3622 | GGGCCACA GGCTAGCTACAACGA AGGGACCC | 4628 |
| 5187 | GUCCCUGU G UGGCCCCC | 3623 | GGGGGCCA GGCTAGCTACAACGA ACAGGGAC | 4629 |
| 5190 | CCUGUGUG G CCCCCCUG | 3624 | CAGGGGGG GGCTAGCTACAACGA CACACAGG | 4630 |
| 5199 | CCCCCCUG G UCUGAUGC | 3625 | GCATCAGA GGCTAGCTACAACGA CAGGGGGG | 4631 |
| 5204 | CUGGUCUG A UGCCGAGG | 3626 | CCTCGGCA GGCTAGCTACAACGA CAGACCAG | 4632 |
| 5206 | GGUCUGAU G CCGAGGAU | 3627 | ATCCTCGG GGCTAGCTACAACGA ATCAGACC | 4633 |
| 5213 | UGCCGAGG A UACCCCUG | 3628 | CAGGGGTA GGCTAGCTACAACGA CCTCGGCA | 4634 |
| 5215 | CCGAGGAU A CCCCUGCA | 3629 | TGCAGGGG GGCTAGCTACAACGA ATCCTCGG | 4635 |
| 5221 | AUACCCCU G CAAACUGC | 3630 | GCAGTTTG GGCTAGCTACAACGA AGGGGTAT | 4636 |
| 5225 | CCCUGCAA A CUGCCAAU | 3631 | ATTGGCAG GGCTAGCTACAACGA TTGCAGGG | 4637 |
| 5228 | UGCAAACU G CCAAUCCC | 3632 | GGGATTGG GGCTAGCTACAACGA AGTTTGCA | 4638 |
| 5232 | AACUGCCA A UCCCAGAG | 3633 | CTCTGGGA GGCTAGCTACAACGA TGGCAGTT | 4639 |
| 5242 | CCCAGAGG A CAAGACUG | 3634 | CAGTCTTG GGCTAGCTACAACGA CCTCTGGG | 4640 |
| 5247 | AGGACAAG A CUGGGAAG | 3635 | CTTCCCAG GGCTAGCTACAACGA CTTGTCCT | 4641 |
| 5255 | ACUGGGAA G UCCCUGCA | 3636 | TGCAGGGA GGCTAGCTACAACGA TTCCCAGT | 4642 |
| 5261 | AAGUCCCU G CAGGGAGA | 3637 | TCTCCCTG GGCTAGCTACAACGA AGGGACTT | 4643 |
| 5270 | CAGGGAGA G CCCAUCCC | 3638 | GGGATGGG GGCTAGCTACAACGA TCTCCCTG | 4644 |
| 5274 | GAGAGCCC A UCCCCGCA | 3639 | TGCGGGGA GGCTAGCTACAACGA GGGCTCTC | 4645 |
| 5280 | CCAUCCCC G CACCCUGA | 3640 | TCAGGGTG GGCTAGCTACAACGA GGGGATGG | 4646 |
| 5282 | AUCCCCGC A CCCUGACC | 3641 | GGTCAGGG GGCTAGCTACAACGA GCGGGGAT | 4647 |
| 5288 | GCACCCUG A CCCACAAG | 3642 | CTTGTGGG GGCTAGCTACAACGA CAGGGTGC | 4648 |
| 5292 | CCUGACCC A CAAGAGGG | 3643 | CCCTCTTG GGCTAGCTACAACGA GGGTCAGG | 4649 |
| 5301 | CAAGAGGG A CUCCUGCU | 3644 | AGCAGGAG GGCTAGCTACAACGA CCCTCTTG | 4650 |
| 5307 | GGACUCCU G CUGCCCAC | 3645 | GTGGGCAG GGCTAGCTACAACGA AGGAGTCC | 4651 |
| 5310 | CUCCUGCU G CCCACCAG | 3646 | CTGGTGGG GGCTAGCTACAACGA AGCAGGAG | 4652 |
| 5314 | UGCUGCCC A CCAGGCAU | 3647 | ATGCCTGG GGCTAGCTACAACGA GGGCAGCA | 4653 |
| 5319 | CCCACCAG G CAUCCCUC | 3648 | GAGGGATG GGCTAGCTACAACGA CTGGTGGG | 4654 |
| 5321 | CACCAGGC A UCCCUCCA | 3649 | TGGAGGGA GGCTAGCTACAACGA GCCTGGTG | 4655 |
| Input Sequence = HUMRasH_mRNA. | | | | |
| Cut Site = R/Y | | | | |
| Arm Length = 8. | | | | |
| Core Sequence = GGCTAGCTACAACGA | | | | |
| HUMRasH_mRNA (Human c-Ha-ras1 proto-oncogene, spliced mRNA sequence; 5336 nt) | | | | |

What we claim is:

1. A siRNA nucleic acid molecule that modulates expression of a nucleic acid molecule encoding K-Ras.

2. A siRNA nucleic acid molecule that modulates expression of a nucleic acid molecule encoding H-Ras or N-Ras.

3. An enzymatic nucleic acid molecule that modulates expression of a sequence encoding K-Ras.

4. An enzymatic nucleic acid molecule that modulates expression of a sequence encoding H-Ras or N-Ras.

5. An enzymatic nucleic acid molecule comprising a sequence of SEQ ID NOs: 1322-2642 or 3650-4655.

6. An enzymatic nucleic acid molecule comprising at least one binding arm wherein one or more of said binding arms comprises a sequence complementary to a sequence of SEQ ID NOs: 1-1321 or 2643-3649.

7. A siRNA nucleic acid molecule comprising a sequence complementary to a sequence of SEQ ID NOs: 1-1321 or 2643-3649.

8. The nucleic acid molecule of any of claims 1-7, wherein said nucleic acid molecule is adapted to treat cancer.

9. The enzymatic nucleic acid molecule of any of claims 3, 5, or 6, wherein said enzymatic nucleic acid molecule has an endonuclease activity to cleave RNA having a K-Ras sequence.

10. The enzymatic nucleic acid molecule of any of claims 4-6, wherein said enzymatic nucleic acid molecule has an endonuclease activity to cleave RNA having an H-Ras sequence.

11. The enzymatic nucleic acid molecule of claim 3 or claim 4, wherein said enzymatic nucleic acid molecule is a DNAzyme in a 10-23 configuration.

12. The enzymatic nucleic acid molecule of claim 11, wherein said enzymatic nucleic acid molecule comprises a sequence complementary to a sequence of SEQ ID NOs: 1-1321 or 2643-3649.

13. The enzymatic nucleic acid molecule of claim 11, wherein said enzymatic nucleic acid molecule comprises a sequence of SEQ ID NOs: 1322-2642 or 3650-4655.

14. The nucleic acid molecule of any of claims 1-7, wherein said nucleic acid molecule comprises between 12 and 100 bases complementary to an RNA having K-Ras, H-Ras and/or N-Ras sequence.

15. The nucleic acid molecule of any of claims 1-7, wherein said nucleic acid molecule comprises between 14 and 24 bases complementary to an RNA having K-Ras, H-Ras and/or N-Ras sequence.

16. The nucleic acid molecule of any of claims 1-7, wherein said nucleic acid molecule is chemically synthesized.

17. The nucleic acid molecule of any of claims 1-7, wherein said nucleic acid molecule comprises at least one 2′-sugar modification.

18. The nucleic acid molecule of any of claims 1-7, wherein said nucleic acid molecule comprises at least one nucleic acid base modification.

19. The nucleic acid molecule of any of claims 1-7, wherein said nucleic acid molecule comprises at least one phosphate backbone modification.

20. A mammalian cell comprising the nucleic acid molecule of any of claims 1-7.

21. The mammalian cell of claim 20, wherein said mammalian cell is a human cell.

22. A method of reducing K-Ras activity in a cell, comprising contacting said cell with the nucleic acid molecule of any of claims 1, 3, 5, 6, or 7, under conditions suitable for said reduction of K-Ras activity.

23. A method of reducing H-Ras activity in a cell, comprising contacting said cell with the nucleic acid molecule of any of claims 2, 4, 5, 6, or 7, under conditions suitable for said reduction of H-Ras activity.

24. A method of treatment of a subject having a condition associated with the level of K-Ras, comprising contacting cells of said subject with the nucleic acid molecule of any of claims 1, 3, 5, 6, or 7, under conditions suitable for said treatment.

25. A method of treatment of a subject having a condition associated with the level of H-Ras, comprising contacting cells of said subject with the nucleic acid molecule of any of claims 2, 4, 5, 6, or 7, under conditions suitable for said treatment

26. The method of claim 24 further comprising the use of one or more drug therapies under conditions suitable for said treatment.

27. The method of claim 25 further comprising the use of one or more drug therapies under conditions suitable for said treatment

28. A method of cleaving RNA having a K-Ras sequence comprising contacting a nucleic acid molecule of any of claims 1, 3, 5, 6, or 7, with said RNA under conditions suitable for the cleavage.

29. A method of cleaving RNA having a H-Ras sequence comprising contacting a nucleic acid molecule of any of claims 2, 4, 5, 6, or 7, with said RNA under conditions suitable for the cleavage.

30. The method of claim 28, wherein said cleavage is carried out in the presence of a divalent cation.

31. The method of claim 29, wherein said cleavage is carried out in the presence of a divalent cation.

32. The method of claim 30, wherein said divalent cation is Mg²⁺.

33. The method of claim 31, wherein said divalent cation is Mg²⁺.

34. The nucleic acid molecule of any of claims 1-7, wherein said nucleic acid molecule comprises a cap structure, wherein the cap structure is at the 5′-end, 3′-end, or both the 5′-end and the 3′-end of said nucleic acid molecule.

35. The nucleic acid molecule of claim 34, wherein the cap structure comprises a 3′,3′-linked or 5′,5′-linked deoxyabasic ribose derivative.

36. An expression vector comprising a nucleic acid sequence encoding at least one nucleic acid molecule of any of claims 1-7 in a manner that allows expression of the nucleic acid molecule.

37. A mammalian cell comprising an expression vector of claim 36.

38. The mammalian cell of claim 37, wherein said mammalian cell is a human cell.

39. The expression vector of claim 36, wherein said nucleic acid molecule is in a DNAzyme configuration.

40. The expression vector of claim 36, wherein said expression vector further comprises a sequence for a nucleic acid molecule complementary a nucleic acid molecule having a K-Ras sequence.

41. The expression vector of claim 36, wherein said expression vector further comprises a sequence for a nucleic acid molecule complementary to a nucleic acid molecule having a H-Ras sequence.

42. The expression vector of claim 36, wherein said expression vector comprises a nucleic acid sequence encoding two or more of said nucleic acid molecules, which may be the same or different.

43. The expression vector of claim 36, wherein said expression vector further comprises a sequence encoding an antisense nucleic acid molecule or siRNA nucleic acid molecule complementary to a nucleic acid molecule having a K-Ras sequence.

44. The expression vector of claim 36, wherein said expression vector further comprises a sequence encoding an antisense nucleic acid molecule or siRNA nucleic acid molecule complementary to a nucleic acid molecule having a H-Ras sequence.

45. A method for the treatment of cancer comprising administering to a subject the nucleic acid molecule of any of claims 1-7 under conditions suitable for said treatment.

46. The method of claim 45, wherein said cancer is colorectal cancer.

47. The method of claim 45, wherein said cancer is lung cancer.

48. The method of claim 45, wherein said cancer is prostate cancer.

49. The method of claim 45, wherein said cancer is bladder cancer.

50. The method of claim 45, wherein said cancer is breast cancer.

51. The method of claim 45, wherein said cancer is pancreatic cancer.

52. The method of claim 45, wherein said method further comprises administering to said patient one or more other therapies under conditions suitable for said treatment.

53. The method of claim 26 wherein said other drug therapies are chosen from monoclonal antibody therapy, chemotherapy, radiation therapy, and analgesic therapy.

54. The method of claim 27 wherein said other drug therapies are chosen from monoclonal antibody therapy, chemotherapy, radiation therapy, and analgesic therapy.

55. The method of claim 52 wherein said other drug therapies are chosen from monoclonal antibody therapy, chemotherapy, radiation therapy, or analgesic therapy.

56. The method of claim 53, wherein said chemotherapy is selected from the group consisting of paclitaxel (Taxol), docetaxel, cisplatin, methotrexate, cyclophosphamide, doxorubin, fluorouracil carboplatin, edatrexate, gemcitabine, and vinorelbine.

57. The method of claim 54, wherein said chemotherapy is selected from the group consisting of paclitaxel (Taxol), docetaxel, cisplatin, methotrexate, cyclophosphamide, doxorubin, fluorouracil carboplatin, edatrexate, gemcitabine, and vinorelbine.

58. The method of claim 55, wherein said chemotherapy is selected from the group consisting of paclitaxel (Taxol), docetaxel, cisplatin, methotrexate, cyclophosphamide, doxorubin, fluorouracil carboplatin, edatrexate, gemcitabine, and vinorelbine.

59. A composition comprising a nucleic acid molecule of any of claims 1-7 and a pharmaceutically acceptable carrier.

60. A method of administering to a cell a nucleic acid molecule of any of claims 1-7 comprising contacting said cell with the nucleic acid molecule under conditions suitable for said administration.

61. The method of claim 60, wherein said cell is a mammalian cell.

62. The method of claim 61, wherein said cell is a human cell.

63. The method of claim 60, wherein said administration is in the presence of a delivery reagent.

64. The method of claim 63, wherein said delivery reagent is a lipid.

65. The method of claim 64, wherein said lipid is a cationic lipid.

66. The method of claim 64, wherein said lipid is a phospholipid.

67. The method of claim 63, wherein said delivery reagent is a liposome.
